SEQUENCE LISTING (166 sequences)

SEQ ID NO: 1 - RNA, 21 nt, synthetic construct
  misc_feature 1..21: note = Description of Artificial Sequence: Synthetic oligonucleotide
  source 1..21: mol_type = other RNA
ctgtacaagtgctcagttcca

SEQ ID NO: 2 - RNA, 23 nt, synthetic construct
  misc_feature 1..23: note = Description of Artificial Sequence: Synthetic oligonucleotide
  source 1..23: mol_type = other RNA
tggaactgagcacttgtacagga

SEQ ID NO: 3 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = OTHER; note = 2'-O-C3H6-NH(CO)-C2H4-SS-C12H25 uridine
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 4 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluorocytidine
  modified_base 7: mod_base = um
  modified_base 8: mod_base = gm
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyladenosine
  source 1..23: mol_type = other RNA
tggaactgagcacttgtacagga

SEQ ID NO: 5 - RNA, 21 nt, synthetic construct
  misc_feature 1..21: note = Description of Artificial Sequence: Synthetic oligonucleotide
  modified_base 1: mod_base = OTHER; note = 2'-O-C3H6-NH(CO)-C2H4-SS-C12H25 cytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 6 - RNA, 21 nt, synthetic construct
  misc_feature 1..21: note = Description of Artificial Sequence: Synthetic oligonucleotide
  modified_base 1: mod_base = OTHER; note = 2'-O-C2H4-SS-C12H25 cytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 7 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-fluoroadenosine
  modified_base 6: mod_base = cm
  modified_base 7: mod_base = um
  modified_base 8: mod_base = OTHER; note = 2'-fluoroguanosine
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyladenosine
  source 1..23: mol_type = other RNA
tggaactgagcacttgtacagga

SEQ ID NO: 8 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = OTHER; note = 2'-O-C2H4-SS-C12H25 cytidine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 9 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 14: mod_base = cm
  modified_base 13: mod_base = um
  modified_base 12: mod_base = OTHER; note = 2'-O-C2H4-SS-C12H25 cytidine
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 10 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = OTHER; note = 2'-O-C2H4-SS-C12H25 cytidine
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 11 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-C2H4-SS-C12H25 cytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 12 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-C2H4-SS-C12H25 cytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 13 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-C2H4-SS-C12H25 uridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 14 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = OTHER; note = 2'-O-C2H4-SS-C12H25 uridine
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 15 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-O-C2H4-SS-C12H25 uridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 16 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = OTHER; note = 2'-O-C2H4-SS-C12H25 uridine
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 17 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = OTHER; note = 2'-O-C2H4-SS-C12H25 uridine
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 18 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = OTHER; note = 2'-O-C2H4-SS-C12H25 uridine
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 19 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = OTHER; note = 2'-O-C3H6-NH(CO)-C2H4-SS-C12H25 uridine
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 20 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = OTHER; note = 2'-O-C2H4-SS-C12H25 uridine
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 21 - RNA, 21 nt, synthetic construct
  misc_feature 1..21: note = Description of Artificial Sequence: Synthetic oligonucleotide
  source 1..21: mol_type = other RNA
gtggaagtaaaatctgagaaa

SEQ ID NO: 22 - RNA, 23 nt, synthetic construct
  misc_feature 1..23: note = Description of Artificial Sequence: Synthetic oligonucleotide
  source 1..23: mol_type = other RNA
tttctcagattttacttccacct

SEQ ID NO: 23 - RNA, 21 nt, synthetic construct
  misc_feature 1..21: note = Description of Artificial Sequence: Synthetic oligonucleotide
  source 1..21: mol_type = other RNA
ccaagtgtggctcattaggca

SEQ ID NO: 24 - RNA, 23 nt, synthetic construct
  misc_feature 1..23: note = Description of Artificial Sequence: Synthetic oligonucleotide
  source 1..23: mol_type = other RNA
tgcctaatgagccacacttggag

SEQ ID NO: 25 - RNA, 21 nt, synthetic construct
  misc_feature 1..21: note = Description of Artificial Sequence: Synthetic oligonucleotide
  source 1..21: mol_type = other RNA
tgcaaatagtctacaaaccaa

SEQ ID NO: 26 - RNA, 23 nt, synthetic construct
  misc_feature 1..23: note = Description of Artificial Sequence: Synthetic oligonucleotide
  source 1..23: mol_type = other RNA
ttggtttgtagactatttgcacc

SEQ ID NO: 27 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = um
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = OTHER; note = 2'-O-C3H6-NH(CO)-C2H4-SS-C12H25 uridine
  modified_base 14: mod_base = cm
  modified_base 15: mod_base = um
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
gtggaagtaaaatctgagaaa

SEQ ID NO: 28 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = um
  modified_base 4: mod_base = cm
  modified_base 5: mod_base = OTHER; note = 2'-fluorouridine
  modified_base 6: mod_base = cm
  modified_base 7: mod_base = OTHER; note = 2'-O-methyladenosine
  modified_base 8: mod_base = OTHER; note = 2'-fluoroguanosine
  modified_base 9: mod_base = OTHER; note = 2'-O-methyladenosine
  modified_base 10: mod_base = um
  modified_base 11: mod_base = um
  modified_base 12: mod_base = um
  modified_base 13: mod_base = um
  modified_base 14: mod_base = OTHER; note = 2'-fluoroadenosine
  modified_base 15: mod_base = cm
  modified_base 16: mod_base = OTHER; note = 2'-fluorouridine
  modified_base 17: mod_base = um
  modified_base 18: mod_base = cm
  modified_base 19: mod_base = cm
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylcytidine with a 3'-phosphorothioate linkage
  modified_base 23: mod_base = um
  source 1..23: mol_type = other RNA
tttctcagattttacttccacct

SEQ ID NO: 29 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = um
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorocytidine
  modified_base 12: mod_base = OTHER; note = 2'-O-C3H6-NH(CO)-C2H4-SS-C12H25 uridine
  modified_base 13: mod_base = cm
  modified_base 14: mod_base = OTHER; note = 2'-O-methyladenosine
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ccaagtgtggctcattaggca

SEQ ID NO: 30 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluorouridine
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = cm
  modified_base 18: mod_base = um
  modified_base 19: mod_base = um
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = gm
  source 1..23: mol_type = other RNA
tgcctaatgagccacacttggag

SEQ ID NO: 31 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-O-C3H6-NH(CO)-C2H4-SS-C12H25 uridine
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 32 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = 2'-fluorouridine
  modified_base 6: mod_base = um
  modified_base 7: mod_base = um
  modified_base 8: mod_base = OTHER; note = 2'-fluoroguanosine
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = cm
  source 1..23: mol_type = other RNA
ttggtttgtagactatttgcacc

SEQ ID NO: 33 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-C3H6-NH(CO)-C2H4-SS-C12H25 with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = um
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = um
  modified_base 14: mod_base = cm
  modified_base 15: mod_base = um
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
gtggaagtaaaatctgagaaa

SEQ ID NO: 34 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = um
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = OTHER; note = 2'-O-C3H6-NH(CO)-C2H4-SS-C12H25 cytidine
  modified_base 14: mod_base = OTHER; note = 2'-O-methyladenosine
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ccaagtgtggctcattaggca

SEQ ID NO: 35 - RNA, 21 nt, synthetic construct
  misc_feature 1..21: note = Description of Artificial Sequence: Synthetic oligonucleotide
  modified_base 1: mod_base = OTHER; note = 2'-O-C3H6-NH(CO)-C2H4-SS-C12H25 cytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = um
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ccaagtgtggctcattaggca

SEQ ID NO: 36 - RNA, 21 nt, synthetic construct
  misc_feature 1..21: note = Description of Artificial Sequence: Synthetic oligonucleotide
  modified_base 1: mod_base = OTHER; note = 2'-O-C3H6-NH(CO)-C2H4-SS-C12H25 uridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 37 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = OTHER; note = 2'-O-C2H4-SS-C2H4-6-(oxy)-2-((4R,8R)-4,8,12-trimeth yltridecyl)-1,2,3,4-tetrahydroisoquinoline uridine
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 38 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = OTHER; note = 2'-O-C2H4-SS-adamantan-1-yl uridine
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 39 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = um
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = OTHER; note = 2'-O-C2H4-SS-C12H25 uridine
  modified_base 14: mod_base = cm
  modified_base 15: mod_base = um
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
gtggaagtaaaatctgagaaa

SEQ ID NO: 40 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = um
  modified_base 9: mod_base = OTHER; note = n is an abasic nucleotide
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = OTHER; note = 2'-O-C2H4-SS-C12H25 uridine
  modified_base 14: mod_base = cm
  modified_base 15: mod_base = um
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
gtggaagtnaaatctgagaaa

SEQ ID NO: 41 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = um
  modified_base 4: mod_base = cm
  modified_base 5: mod_base = um
  modified_base 6: mod_base = OTHER; note = 2'-fluorocytidine
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = OTHER; note = 2'-fluorouridine
  modified_base 11: mod_base = um
  modified_base 12: mod_base = um
  modified_base 13: mod_base = um
  modified_base 14: mod_base = OTHER; note = 2'-fluoroadenosine
  modified_base 15: mod_base = cm
  modified_base 16: mod_base = OTHER; note = 2'-fluorouridine
  modified_base 17: mod_base = um
  modified_base 18: mod_base = OTHER; note = 2'-fluorocytidine
  modified_base 19: mod_base = cm
  modified_base 20: mod_base = OTHER; note = 2'-fluoroadenosine
  modified_base 21: mod_base = OTHER; note = 2'-O-methylcytidine with a 3'-phosphorothioate linkage
  modified_base 22: mod_base = OTHER; note = 2'-O-methylcytidine with a 3'-phosphorothioate linkage
  modified_base 23: mod_base = um
  source 1..23: mol_type = other RNA
tttctcagattttacttccacct

SEQ ID NO: 42 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = um
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorocytidine
  modified_base 12: mod_base = OTHER; note = 2'-O-C2H4-SS-C12H25 uridine
  modified_base 13: mod_base = cm
  modified_base 14: mod_base = OTHER; note = 2'-O-methyladenosine
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ccaagtgtggctcattaggca

SEQ ID NO: 43 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-O-C2H4-SS-C12H25 uridine
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 44 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = gm
  modified_base 5: mod_base = gm
  modified_base 6: mod_base = um
  modified_base 7: mod_base = gm
  modified_base 8: mod_base = gm
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroguanosine
  modified_base 12: mod_base = OTHER; note = 2'-O-C2H4-SS-C12H25 uridine
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methyladenosine
  modified_base 16: mod_base = OTHER; note = 2'-O-methyladenosine
  modified_base 17: mod_base = um
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
  source 1..21: mol_type = other RNA
ccaggtggaagtaaaatctga

SEQ ID NO: 45 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = gm
  modified_base 5: mod_base = OTHER; note = 2'-fluoroadenosine
  modified_base 6: mod_base = um
  modified_base 7: mod_base = um
  modified_base 8: mod_base = OTHER; note = 2'-fluorouridine
  modified_base 9: mod_base = um
  modified_base 10: mod_base = OTHER; note = 2'-O-methyladenosine
  modified_base 11: mod_base = cm
  modified_base 12: mod_base = um
  modified_base 13: mod_base = um
  modified_base 14: mod_base = OTHER; note = 2'-fluorocytidine
  modified_base 15: mod_base = cm
  modified_base 16: mod_base = OTHER; note = 2'-fluoroadenosine
  modified_base 17: mod_base = cm
  modified_base 18: mod_base = cm
  modified_base 19: mod_base = um
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = cm
  source 1..23: mol_type = other RNA
tgcctaatgagccacacttggag

SEQ ID NO: 46 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = gm
  modified_base 5: mod_base = gm
  modified_base 6: mod_base = um
  modified_base 7: mod_base = gm
  modified_base 8: mod_base = gm
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroguanosine
  modified_base 12: mod_base = um
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methyladenosine
  modified_base 16: mod_base = OTHER; note = 2'-O-methyladenosine
  modified_base 17: mod_base = um
  modified_base 18: mod_base = cm
  modified_base 19: mod_base = OTHER; note = 2'-O-C2H4-SS-C12H25 uridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
  source 1..21: mol_type = other RNA
ccaggtggaagtaaaatctga

SEQ ID NO: 47 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = OTHER; note = 2'-O-C2H4-SS-C12H25 uridine
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = um
  modified_base 14: mod_base = cm
  modified_base 15: mod_base = um
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
gtggaagtaaaatctgagaaa

SEQ ID NO: 48 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-C2H4-SS-C12H25 uridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = um
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = um
  modified_base 14: mod_base = cm
  modified_base 15: mod_base = um
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
gtggaagtaaaatctgagaaa

SEQ ID NO: 49 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = um
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = um
  modified_base 14: mod_base = cm
  modified_base 15: mod_base = OTHER; note = 2'-O-C2H4-SS-C12H25 uridine
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
gtggaagtaaaatctgagaaa

SEQ ID NO: 50 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = OTHER; note = 2'-O-C2H4-SS-C12H25 uridine
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ccaagtgtggctcattaggca

SEQ ID NO: 51 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = um
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = OTHER; note = 2'-O-C2H4-SS-C12H25 uridine
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ccaagtgtggctcattaggca

SEQ ID NO: 52 - RNA, 21 nt, synthetic construct
  misc_feature 1..21: note = Description of Artificial Sequence: Synthetic oligonucleotide
  modified_base 1: mod_base = OTHER; note = 2'-O-C2H4-SS-C12H25 uridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 53 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = um
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = OTHER; note = 2'-O-C2H4-SS-C12H25 cytidine
  modified_base 14: mod_base = OTHER; note = 2'-O-methyladenosine
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ccaagtgtggctcattaggca

SEQ ID NO: 54 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = cm
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = gm
  modified_base 10: mod_base = OTHER; note = 2'-fluoroadenosine
  modified_base 11: mod_base = gm
  modified_base 12: mod_base = cm
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = cm
  modified_base 18: mod_base = OTHER; note = 2'-fluorouridine
  modified_base 19: mod_base = um
  modified_base 20: mod_base = OTHER; note = 2'-fluoroguanosine
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = gm
  source 1..23: mol_type = other RNA
tgcctaatgagccacacttggag

SEQ ID NO: 55 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluoroadenosine
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = cm
  modified_base 18: mod_base = um
  modified_base 19: mod_base = um
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = gm
  source 1..23: mol_type = other RNA
tgcctaatgagccacacttggag

SEQ ID NO: 56 - RNA, 21 nt, synthetic construct
  misc_feature 1..21: note = Description of Artificial Sequence: Synthetic oligonucleotide
  source 1..21: mol_type = other RNA
ccaggtggaagtaaaatctga

SEQ ID NO: 57 - RNA, 23 nt, synthetic construct
  misc_feature 1..23: note = Description of Artificial Sequence: Synthetic oligonucleotide
  source 1..23: mol_type = other RNA
tcagattttacttccacctggcc

SEQ ID NO: 58 - RNA, 3177 nt, Homo sapiens
  source 1..3177: mol_type = mRNA
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 59 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 60 - RNA, 6644 nt, Homo sapiens
  source 1..6644: mol_type = mRNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 61 - protein, 776 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSATKQVQRRPPPAGPRSERGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 62 - RNA, 5552 nt, Homo sapiens
  source 1..5552: mol_type = mRNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 63 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 64 - RNA, 5372 nt, Homo sapiens
  source 1..5372: mol_type = mRNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 65 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 66 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = um
  modified_base 8: mod_base = gm
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyladenosine
  source 1..23: mol_type = other RNA
tggaactgagcacttgtacagga

SEQ ID NO: 67 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = OTHER; note = 2'-O-C3H6-NH(CO)-C2H4-SS-adamantyl uridine
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 68 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = OTHER; note = 2'-O-methyladenosine
  modified_base 4: mod_base = cm
  modified_base 6: mod_base = OTHER; note = 2'-O-methyladenosine
  modified_base 7: mod_base = OTHER; note = 2'-fluoroguanosine
  modified_base 9: mod_base = OTHER; note = 2'-fluoroguanosine
  modified_base 10: mod_base = cm
  modified_base 11: mod_base = OTHER; note = 2'-O-C3H6-NH(CO)-C2H4-SS-C12H25 uridine
  modified_base 12: mod_base = cm
  modified_base 13: mod_base = OTHER; note = 2'-O-methyladenosine
  modified_base 14: mod_base = gm
  modified_base 15: mod_base = um
  modified_base 16: mod_base = um
  modified_base 17: mod_base = OTHER; note = 2'-O-methylcytidine with a 3'-phosphorothioate linkage
  modified_base 18: mod_base = OTHER; note = 2'-O-methylcytidine with a 3'-phosphorothioate linkage
  modified_base 19: mod_base = OTHER; note = 2'-O-methyladenosine
  source 1..21: mol_type = other RNA
ctgtacaagtgctcagttcca

SEQ ID NO: 69 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-C3H6-NH(CO)-C2H4-SS-C12H25 with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 70 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = OTHER; note = 2'-O-C3H6-NH(CO)-C2H4-SS-C12H25 uridine
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctttacaagtgctcagttcca

SEQ ID NO: 71 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = OTHER; note = 2'-O-C3H6-NH(CO)-C2H4-SS-C12H25 uridine
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 72 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = OTHER; note = 2'-O-C3H6-NH(CO)-C2H4-SS-C12H25 cytidine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 73 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-O-C3H6-NH(CO)-C2H4-SS-C12H25 uridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 74 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = OTHER; note = 2'-O-C3H6-NH(CO)-C2H4-SS-C12H25 cytidine
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 75 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = OTHER; note = 2'-O-C3H6-NH(CO)-C2H4-SS-C12H25 cytidine
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 76 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = OTHER; note = 2'-O-C3H6-NH(CO)-C2H4-SS-C12H25 uridine
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcatttcca

SEQ ID NO: 77 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = OTHER; note = 2'-O-C3H6-NH(CO)-C2H4-SS-C12H25 uridine
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 78 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = OTHER; note = 2'-O-C3H6-NH(CO)-C2H4-SS-C12H25 uridine
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 79 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-C3H6-NH(CO)-C2H4-SS-C12H25 uridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagtttca

SEQ ID NO: 80 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-C3H6-NH(CO)-C2H4-SS-C12H25 uridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcta

SEQ ID NO: 81 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = OTHER; note = 2'-O-C2H4-SS-C16H33 uridine
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 82 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-fluoroadenosine
  modified_base 6: mod_base = cm
  modified_base 7: mod_base = OTHER; note = 2'-fluorouridine
  modified_base 8: mod_base = gm
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyladenosine
  source 1..23: mol_type = other RNA
tggaactgagcacttgtacagga

SEQ ID NO: 83 - RNA, 21 nt, synthetic construct
  source 1..21: mol_type = other RNA
tgtacaagtgctcagttccaa

SEQ ID NO: 84 - RNA, 23 nt, synthetic construct
  source 1..23: mol_type = other RNA
ttggaactgagcacttgtacagg

SEQ ID NO: 85 - RNA, 20 nt, synthetic construct
  source 1..20: mol_type = other RNA
gtacaagtgctcagttccaa

SEQ ID NO: 86 - RNA, 22 nt, synthetic construct
  source 1..22: mol_type = other RNA
ttggaactgagcacttgtacag

SEQ ID NO: 87 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = OTHER; note = 2'-O-C2H4-SS-C12H25 cytidine
  modified_base 14: mod_base = OTHER; note = 2'-O-methyladenosine
  modified_base 15: mod_base = gm
  modified_base 16: mod_base = um
  modified_base 17: mod_base = um
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
tgtacaagtgctcagttccaa

SEQ ID NO: 88 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-fluoroadenosine
  modified_base 7: mod_base = cm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluorouridine
  modified_base 17: mod_base = gm
  modified_base 18: mod_base = um
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
ttggaactgagcacttgtacagg

SEQ ID NO: 89 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = OTHER; note = 2'-O-C2H4-SS-C12H25 cytidine
  modified_base 14: mod_base = OTHER; note = 2'-O-methyladenosine
  modified_base 15: mod_base = gm
  modified_base 16: mod_base = um
  modified_base 17: mod_base = um
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
tgtacaagtgctcagttccaa

SEQ ID NO: 90 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = 2'-fluoroadenosine
  modified_base 6: mod_base = OTHER; note = 2'-O-methyladenosine
  modified_base 7: mod_base = cm
  modified_base 8: mod_base = OTHER; note = 2'-fluorouridine
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluorouridine
  modified_base 17: mod_base = gm
  modified_base 18: mod_base = um
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
ttggaactgagcacttgtacagg

SEQ ID NO: 91 - RNA, 21 nt, synthetic construct
  source 1..21: mol_type = other RNA
  modified_base 2: mod_base = OTHER; note = 2'-O-methylguanosine with a 3'-phosphorothioate linkage
  modified_base 3: mod_base = OTHER; note = 2'-O-methyluridine with a 3'-phosphorothioate linkage
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = OTHER; note = 2'-O-C2H4-SS-C12H25 cytidine
  modified_base 14: mod_base = OTHER; note = 2'-O-methyladenosine
  modified_base 15: mod_base = gm
  modified_base 16: mod_base = um
  modified_base 17: mod_base = um
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  modified_base 1: mod_base = OTHER; note = n is an inverted abasic nucleotide
ngtacaagtgctcagttccaa

SEQ ID NO: 92 - RNA, 22 nt, synthetic construct
  source 1..22: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = 2'-fluoroadenosine
  modified_base 6: mod_base = OTHER; note = 2'-O-methyladenosine
  modified_base 7: mod_base = cm
  modified_base 8: mod_base = OTHER; note = 2'-fluorouridine
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluorouridine
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methylcytidine with a 3'-phosphorothioate linkage
  modified_base 21: mod_base = OTHER; note = 2'-O-methyladenosine with a 3'-phosphorothioate linkage
  modified_base 22: mod_base = gm
ttggaactgagcacttgtacag

SEQ ID NO: 93 - RNA, 22 nt, synthetic construct
  source 1..22: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = cm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluorouridine
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methylcytidine with a 3'-phosphorothioate linkage
  modified_base 21: mod_base = OTHER; note = 2'-O-methyladenosine with a 3'-phosphorothioate linkage
  modified_base 22: mod_base = gm
ttggaactgagcacttgtacag

SEQ ID NO: 94 - RNA, 21 nt, synthetic construct
  source 1..21: mol_type = other RNA
agtgactaccacttatttcta

SEQ ID NO: 95 - RNA, 23 nt, synthetic construct
  source 1..23: mol_type = other RNA
tagaaataagtggtagtcactta

SEQ ID NO: 96 - RNA, 21 nt, synthetic construct
  source 1..21: mol_type = other RNA
gtgactaccacttatttctaa

SEQ ID NO: 97 - RNA, 23 nt, synthetic construct
  source 1..23: mol_type = other RNA
ttagaaataagtggtagtcactt

SEQ ID NO: 98 - RNA, 21 nt, synthetic construct
  source 1..21: mol_type = other RNA
gagcaagtgacaaatgttgga

SEQ ID NO: 99 - RNA, 23 nt, synthetic construct
  source 1..23: mol_type = other RNA
tccaacatttgtcacttgctctt

SEQ ID NO: 100 - RNA, 21 nt, synthetic construct
  source 1..21: mol_type = other RNA
ttccaatgtgcccagtcatga

SEQ ID NO: 101 - RNA, 23 nt, synthetic construct
  source 1..23: mol_type = other RNA
tcatgactgggcacattggaact

SEQ ID NO: 102 - RNA, 21 nt, synthetic construct
  source 1..21: mol_type = other RNA
aagtgactaccacttatttca

SEQ ID NO: 103 - RNA, 23 nt, synthetic construct
  source 1..23: mol_type = other RNA
tgaaataagtggtagtcacttag

SEQ ID NO: 104 - RNA, 21 nt, synthetic construct
  source 1..21: mol_type = other RNA
gaccaaagagcaagtgacaaa

SEQ ID NO: 105 - RNA, 23 nt, synthetic construct
  source 1..23: mol_type = other RNA
tttgtcacttgctctttggtctt

SEQ ID NO: 106 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = cm
  modified_base 7: mod_base = um
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroadenosine
  modified_base 12: mod_base = cm
  modified_base 13: mod_base = OTHER; note = 2'-O-C3H6-NH(CO)-C2H4-SS-C12H25 uridine
  modified_base 14: mod_base = um
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
agtgactaccacttatttcta

SEQ ID NO: 107 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-fluoroadenosine
  modified_base 6: mod_base = OTHER; note = 2'-O-methyladenosine
  modified_base 7: mod_base = um
  modified_base 8: mod_base = OTHER; note = 2'-fluoroadenosine
  modified_base 9: mod_base = OTHER; note = 2'-O-methyladenosine
  modified_base 10: mod_base = gm
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = cm
  modified_base 19: mod_base = OTHER; note = 2'-O-methyladenosine
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methyluridine with a 3'-phosphorothioate linkage
  modified_base 23: mod_base = OTHER; note = 2'-O-methyladenosine
tagaaataagtggtagtcactta

SEQ ID NO: 108 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = OTHER; note = 2'-O-methyladenosine
  modified_base 5: mod_base = cm
  modified_base 6: mod_base = um
  modified_base 7: mod_base = OTHER; note = 2'-O-methyladenosine
  modified_base 8: mod_base = cm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorocytidine
  modified_base 12: mod_base = um
  modified_base 13: mod_base = OTHER; note = 2'-O-C3H6-NH(CO)-C2H4-SS-C12H25 uridine
  modified_base 14: mod_base = OTHER; note = 2'-O-methyladenosine
  modified_base 15: mod_base = um
  modified_base 16: mod_base = um
  modified_base 17: mod_base = um
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
gtgactaccacttatttctaa

SEQ ID NO: 109 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = gm
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluorouridine
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluoroadenosine
  modified_base 17: mod_base = gm
  modified_base 18: mod_base = um
  modified_base 19: mod_base = cm
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
ttagaaataagtggtagtcactt

SEQ ID NO: 110 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluorocytidine
  modified_base 12: mod_base = OTHER; note = 2'-O-methyladenosine
  modified_base 13: mod_base = OTHER; note = 2'-O-C3H6-NH(CO)-C2H4-SS-C12H25 adenosine
  modified_base 14: mod_base = OTHER; note = 2'-O-methyladenosine
  modified_base 15: mod_base = um
  modified_base 16: mod_base = gm
  modified_base 17: mod_base = um
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
gagcaagtgacaaatgttgga

SEQ ID NO: 111 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-fluoroadenosine
  modified_base 6: mod_base = cm
  modified_base 7: mod_base = OTHER; note = 2'-O-methyladenosine
  modified_base 8: mod_base = OTHER; note = 2'-fluorouridine
  modified_base 9: mod_base = um
  modified_base 10: mod_base = um
  modified_base 11: mod_base = gm
  modified_base 12: mod_base = um
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
tccaacatttgtcacttgctctt

SEQ ID NO: 112 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyluridine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = gm
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorocytidine
  modified_base 12: mod_base = cm
  modified_base 13: mod_base = OTHER; note = 2'-O-C3H6-NH(CO)-C2H4-SS-C12H25 cytidine
  modified_base 14: mod_base = OTHER; note = 2'-O-methyladenosine
  modified_base 15: mod_base = gm
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
ttccaatgtgcccagtcatga

SEQ ID NO: 113 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = cm
  modified_base 8: mod_base = OTHER; note = 2'-fluorouridine
  modified_base 9: mod_base = gm
  modified_base 10: mod_base = gm
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = gm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = um
tcatgactgggcacattggaact

SEQ ID NO: 114 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = um
  modified_base 5: mod_base = gm
  modified_base 6: mod_base = OTHER; note = 2'-O-methyladenosine
  modified_base 7: mod_base = cm
  modified_base 8: mod_base = um
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = OTHER; note = 2'-O-C3H6-NH(CO)-C2H4-SS-C12H25 cytidine
  modified_base 14: mod_base = um
  modified_base 15: mod_base = um
  modified_base 16: mod_base = OTHER; note = 2'-O-methyladenosine
  modified_base 17: mod_base = um
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
aagtgactaccacttatttca

SEQ ID NO: 115 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-fluoroadenosine
  modified_base 6: mod_base = um
  modified_base 7: mod_base = OTHER; note = 2'-O-methyladenosine
  modified_base 8: mod_base = OTHER; note = 2'-fluoroadenosine
  modified_base 9: mod_base = gm
  modified_base 10: mod_base = um
  modified_base 11: mod_base = gm
  modified_base 12: mod_base = gm
  modified_base 13: mod_base = um
  modified_base 14: mod_base = OTHER; note = 2'-fluoroadenosine
  modified_base 15: mod_base = gm
  modified_base 16: mod_base = OTHER; note = 2'-fluorouridine
  modified_base 17: mod_base = cm
  modified_base 18: mod_base = OTHER; note = 2'-O-methyladenosine
  modified_base 19: mod_base = cm
  modified_base 20: mod_base = um
  modified_base 21: mod_base = OTHER; note = 2'-O-methyluridine with a 3'-phosphorothioate linkage
  modified_base 22: mod_base = OTHER; note = 2'-O-methyladenosine with a 3'-phosphorothioate linkage
  modified_base 23: mod_base = gm
tgaaataagtggtagtcacttag

SEQ ID NO: 116 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluorocytidine
  modified_base 12: mod_base = OTHER; note = 2'-O-methyladenosine
  modified_base 13: mod_base = OTHER; note = 2'-O-C3H6-NH(CO)-C2H4-SS-C12H25 adenosine
  modified_base 14: mod_base = gm
  modified_base 15: mod_base = um
  modified_base 16: mod_base = gm
  modified_base 17: mod_base = OTHER; note = 2'-O-methyladenosine
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
gaccaaagagcaagtgacaaa

SEQ ID NO: 117 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = um
  modified_base 4: mod_base = gm
  modified_base 5: mod_base = OTHER; note = 2'-fluorouridine
  modified_base 6: mod_base = cm
  modified_base 7: mod_base = OTHER; note = 2'-O-methyladenosine
  modified_base 8: mod_base = OTHER; note = 2'-fluorocytidine
  modified_base 9: mod_base = um
  modified_base 10: mod_base = um
  modified_base 11: mod_base = gm
  modified_base 12: mod_base = cm
  modified_base 13: mod_base = um
  modified_base 14: mod_base = OTHER; note = 2'-fluorocytidine
  modified_base 15: mod_base = um
  modified_base 16: mod_base = OTHER; note = 2'-fluorouridine
  modified_base 17: mod_base = um
  modified_base 18: mod_base = gm
  modified_base 19: mod_base = gm
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
tttgtcacttgctctttggtctt

SEQ ID NO: 118 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = OTHER; note = 2'-O-methyladenosine
  modified_base 5: mod_base = cm
  modified_base 6: mod_base = um
  modified_base 7: mod_base = OTHER; note = 2'-fluoroadenosine
  modified_base 8: mod_base = cm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorocytidine
  modified_base 12: mod_base = um
  modified_base 13: mod_base = OTHER; note = 2'-O-C3H6-NH(CO)-C2H4-SS-C12H25 uridine
  modified_base 14: mod_base = OTHER; note = 2'-O-methyladenosine
  modified_base 15: mod_base = um
  modified_base 16: mod_base = um
  modified_base 17: mod_base = um
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
gtgactaccacttatttctaa

SEQ ID NO: 119 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluoroadenosine
  modified_base 17: mod_base = gm
  modified_base 18: mod_base = um
  modified_base 19: mod_base = cm
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
ttagaaataagtggtagtcactt

SEQ ID NO: 120 - RNA, 22 nt, synthetic construct
  source 1..22: mol_type = other RNA
  modified_base 2: mod_base = OTHER; note = 2'-O-methyladenosine with a 3'-phosphorothioate linkage
  modified_base 3: mod_base = OTHER; note = 2'-O-methylguanosine with a 3'-phosphorothioate linkage
  modified_base 4: mod_base = um
  modified_base 5: mod_base = gm
  modified_base 6: mod_base = OTHER; note = 2'-O-methyladenosine
  modified_base 7: mod_base = cm
  modified_base 8: mod_base = OTHER; note = 2'-fluorouridine
  modified_base 9: mod_base = OTHER; note = 2'-O-methyladenosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-fluoroadenosine
  modified_base 13: mod_base = cm
  modified_base 14: mod_base = OTHER; note = 2'-O-C3H6-NH(CO)-C2H4-SS-C12H25 uridine
  modified_base 15: mod_base = um
  modified_base 16: mod_base = OTHER; note = 2'-O-methyladenosine
  modified_base 17: mod_base = um
  modified_base 18: mod_base = um
  modified_base 19: mod_base = um
  modified_base 20: mod_base = OTHER; note = 2'-O-methylcytidine with a 3'-phosphorothioate linkage
  modified_base 21: mod_base = OTHER; note = 2'-O-methyluridine with a 3'-phosphorothioate linkage
  modified_base 22: mod_base = OTHER; note = 2'-O-methyladenosine
  modified_base 1: mod_base = OTHER; note = n is an inverted abasic nucleotide
nagtgactaccacttatttcta

SEQ ID NO: 121 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = um
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = cm
  modified_base 19: mod_base = OTHER; note = 2'-O-methyladenosine
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methyluridine with a 3'-phosphorothioate linkage
  modified_base 23: mod_base = OTHER; note = 2'-O-methyladenosine
tagaaataagtggtagtcactta

SEQ ID NO: 122 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyladenosine
  modified_base 8: mod_base = um
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluoroadenosine
  modified_base 17: mod_base = gm
  modified_base 18: mod_base = um
  modified_base 19: mod_base = cm
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
ttagaaataagtggtagtcactt

SEQ ID NO: 123 - RNA, 22 nt, synthetic construct
  source 1..22: mol_type = other RNA
  modified_base 2: mod_base = OTHER; note = 2'-O-methyladenosine with a 3'-phosphorothioate linkage
  modified_base 3: mod_base = OTHER; note = 2'-O-methylguanosine with a 3'-phosphorothioate linkage
  modified_base 4: mod_base = um
  modified_base 5: mod_base = gm
  modified_base 6: mod_base = OTHER; note = 2'-O-methyladenosine
  modified_base 7: mod_base = cm
  modified_base 8: mod_base = um
  modified_base 9: mod_base = OTHER; note = 2'-O-methyladenosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-fluoroadenosine
  modified_base 13: mod_base = cm
  modified_base 14: mod_base = OTHER; note = 2'-O-C3H6-NH(CO)-C2H4-SS-C12H25 uridine
  modified_base 15: mod_base = um
  modified_base 16: mod_base = OTHER; note = 2'-O-methyladenosine
  modified_base 17: mod_base = um
  modified_base 18: mod_base = um
  modified_base 19: mod_base = um
  modified_base 20: mod_base = OTHER; note = 2'-O-methylcytidine with a 3'-phosphorothioate linkage
  modified_base 21: mod_base = OTHER; note = 2'-O-methyluridine with a 3'-phosphorothioate linkage
  modified_base 22: mod_base = OTHER; note = 2'-O-methyladenosine
  modified_base 1: mod_base = OTHER; note = n is an inverted abasic nucleotide
nagtgactaccacttatttcta

SEQ ID NO: 124 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = cm
  modified_base 19: mod_base = OTHER; note = 2'-O-methyladenosine
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methyluridine with a 3'-phosphorothioate linkage
  modified_base 23: mod_base = OTHER; note = 2'-O-methyladenosine
tagaaataagtggtagtcactta

SEQ ID NO: 125 - RNA, 21 nt, synthetic construct
  source 1..21: mol_type = other RNA
attaggcaacatccatcataa

SEQ ID NO: 126 - RNA, 23 nt, synthetic construct
  source 1..23: mol_type = other RNA
ttatgatggatgttgcctaatga

SEQ ID NO: 127 - RNA, 21 nt, synthetic construct
  source 1..21: mol_type = other RNA
ggctttggctcgggacttcaa

SEQ ID NO: 128 - RNA, 23 nt, synthetic construct
  source 1..23: mol_type = other RNA
ttgaagtcccgagccaaagccga

SEQ ID NO: 129 - RNA, 21 nt, synthetic construct
  source 1..21: mol_type = other RNA
gcaaatagtctacaaaccaga

SEQ ID NO: 130 - RNA, 23 nt, synthetic construct
  source 1..23: mol_type = other RNA
tctggtttgtagactatttgcac

SEQ ID NO: 131 - RNA, 21 nt, synthetic construct
  source 1..21: mol_type = other RNA
aaataaaaagattgaaaccca

SEQ ID NO: 132 - RNA, 23 nt, synthetic construct
  source 1..23: mol_type = other RNA
tgggtttcaatctttttatttcc

SEQ ID NO: 133 - RNA, 21 nt, synthetic construct
  source 1..21: mol_type = other RNA
gcaaggtgacctccaagtgta

SEQ ID NO: 134 - RNA, 23 nt, synthetic construct
  source 1..23: mol_type = other RNA
tacacttggaggtcaccttgctc

SEQ ID NO: 135 - RNA, 21 nt, synthetic construct
  source 1..21: mol_type = other RNA
agattgaaacccacaagctga

SEQ ID NO: 136 - RNA, 23 nt, synthetic construct
  source 1..23: mol_type = other RNA
tcagcttgtgggtttcaatcttt

SEQ ID NO: 137 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = OTHER; note = 2'-O-methyladenosine
  modified_base 5: mod_base = gm
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluoroadenosine
  modified_base 12: mod_base = um
  modified_base 13: mod_base = OTHER; note = 2'-O-C3H6-NH(CO)-C2H4-SS-C12H25 cytidine
  modified_base 14: mod_base = cm
  modified_base 15: mod_base = OTHER; note = 2'-O-methyladenosine
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
attaggcaacatccatcataa

SEQ ID NO: 138 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = um
  modified_base 5: mod_base = gm
  modified_base 6: mod_base = OTHER; note = 2'-O-methyladenosine
  modified_base 7: mod_base = um
  modified_base 8: mod_base = gm
  modified_base 9: mod_base = gm
  modified_base 10: mod_base = OTHER; note = 2'-O-methyladenosine
  modified_base 11: mod_base = um
  modified_base 12: mod_base = gm
  modified_base 13: mod_base = um
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyladenosine
ttatgatggatgttgcctaatga

SEQ ID NO: 139 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = um
  modified_base 5: mod_base = um
  modified_base 6: mod_base = um
  modified_base 7: mod_base = gm
  modified_base 8: mod_base = gm
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = gm
  modified_base 13: mod_base = OTHER; note = 2'-O-C3H6-NH(CO)-C2H4-SS-C12H25 guanosine
  modified_base 14: mod_base = gm
  modified_base 15: mod_base = OTHER; note = 2'-O-methyladenosine
  modified_base 16: mod_base = cm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
ggctttggctcgggacttcaa

SEQ ID NO: 140 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = gm
  modified_base 7: mod_base = um
  modified_base 8: mod_base = cm
  modified_base 9: mod_base = cm
  modified_base 10: mod_base = cm
  modified_base 11: mod_base = gm
  modified_base 12: mod_base = OTHER; note = 2'-O-methyladenosine
  modified_base 13: mod_base = gm
  modified_base 14: mod_base = OTHER; note = 2'-fluorocytidine
  modified_base 15: mod_base = cm
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = gm
  modified_base 20: mod_base = cm
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyladenosine
ttgaagtcccgagccaaagccga

SEQ ID NO: 141 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = um
  modified_base 7: mod_base = OTHER; note = 2'-O-methyladenosine
  modified_base 8: mod_base = gm
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorouridine
  modified_base 12: mod_base = OTHER; note = 2'-O-methyladenosine
  modified_base 13: mod_base = OTHER; note = 2'-O-C3H6-NH(CO)-C2H4-SS-C12H25 cytidine
  modified_base 14: mod_base = OTHER; note = 2'-O-methyladenosine
  modified_base 15: mod_base = OTHER; note = 2'-O-methyladenosine
  modified_base 16: mod_base = OTHER; note = 2'-O-methyladenosine
  modified_base 17: mod_base = cm
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
gcaaatagtctacaaaccaga

SEQ ID NO: 142 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = um
  modified_base 4: mod_base = gm
  modified_base 5: mod_base = gm
  modified_base 6: mod_base = um
  modified_base 7: mod_base = um
  modified_base 8: mod_base = um
  modified_base 9: mod_base = gm
  modified_base 10: mod_base = um
  modified_base 11: mod_base = OTHER; note = 2'-O-methyladenosine
  modified_base 12: mod_base = gm
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluoroadenosine
  modified_base 17: mod_base = um
  modified_base 18: mod_base = um
  modified_base 19: mod_base = um
  modified_base 20: mod_base = gm
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = cm
tctggtttgtagactatttgcac

SEQ ID NO: 143 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methyladenosine with a 3'-phosphorothioate linkage
  modified_base 3: mod_base = OTHER; note = 2'-O-methyladenosine
  modified_base 4: mod_base = um
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluoroadenosine
  modified_base 12: mod_base = um
  modified_base 13: mod_base = OTHER; note = 2'-O-C3H6-NH(CO)-C2H4-SS-C12H25 uridine
  modified_base 14: mod_base = gm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
aaataaaaagattgaaaccca

SEQ ID NO: 144 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = gm
  modified_base 5: mod_base = um
  modified_base 6: mod_base = um
  modified_base 7: mod_base = um
  modified_base 9: mod_base = OTHER; note = 2'-O-methyladenosine
  modified_base 8: mod_base = cm
  modified_base 10: mod_base = OTHER; note = 2'-O-methyladenosine
  modified_base 11: mod_base = um
  modified_base 12: mod_base = cm
  modified_base 13: mod_base = um
  modified_base 14: mod_base = OTHER; note = 2'-fluorouridine
  modified_base 15: mod_base = um
  modified_base 16: mod_base = OTHER; note = 2'-fluorouridine
  modified_base 17: mod_base = um
  modified_base 18: mod_base = OTHER; note = 2'-O-methyladenosine
  modified_base 19: mod_base = um
  modified_base 20: mod_base = um
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = cm
tgggtttcaatctttttatttcc

SEQ ID NO: 145 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = gm
  modified_base 7: mod_base = um
  modified_base 8: mod_base = gm
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = OTHER; note = 2'-O-C3H6-NH(CO)-C2H4-SS-C12H25 cytidine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = gm
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gcaaggtgacctccaagtgta

SEQ ID NO: 146 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = cm
  modified_base 4: mod_base = OTHER; note = 2'-O-methyladenosine
  modified_base 5: mod_base = cm
  modified_base 6: mod_base = um
  modified_base 7: mod_base = um
  modified_base 8: mod_base = gm
  modified_base 9: mod_base = gm
  modified_base 10: mod_base = OTHER; note = 2'-O-methyladenosine
  modified_base 11: mod_base = gm
  modified_base 12: mod_base = gm
  modified_base 13: mod_base = um
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluorocytidine
  modified_base 17: mod_base = cm
  modified_base 18: mod_base = um
  modified_base 19: mod_base = um
  modified_base 20: mod_base = gm
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = cm
tacacttggaggtcaccttgctc

SEQ ID NO: 147 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = OTHER; note = 2'-O-methyladenosine
  modified_base 4: mod_base = um
  modified_base 5: mod_base = um
  modified_base 6: mod_base = gm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorocytidine
  modified_base 12: mod_base = cm
  modified_base 13: mod_base = OTHER; note = 2'-O-C3H6-NH(CO)-C2H4-SS-C12H25 adenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = gm
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
agattgaaacccacaagctga

SEQ ID NO: 148 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = gm
  modified_base 5: mod_base = cm
  modified_base 6: mod_base = um
  modified_base 7: mod_base = um
  modified_base 8: mod_base = gm
  modified_base 9: mod_base = um
  modified_base 10: mod_base = gm
  modified_base 11: mod_base = gm
  modified_base 12: mod_base = gm
  modified_base 13: mod_base = um
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methyluridine with a 3'-phosphorothioate linkage
  modified_base 23: mod_base = um
tcagcttgtgggtttcaatcttt

SEQ ID NO: 149 - RNA, 22 nt, synthetic construct
  misc_feature 1..22: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluorouridine
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = cm
  modified_base 18: mod_base = um
  modified_base 19: mod_base = um
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine
  source 1..22: mol_type = other RNA
tgcctaatgagccacacttgga

SEQ ID NO: 150 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluorouridine
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = cm
  modified_base 18: mod_base = um
  modified_base 19: mod_base = um
  modified_base 20: mod_base = gm
  modified_base 21: mod_base = gm
  source 1..21: mol_type = other RNA
tgcctaatgagccacacttgg

SEQ ID NO: 151 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = cm
  modified_base 5: mod_base = um
  modified_base 6: mod_base = OTHER; note = 2'-O-methyladenosine
  modified_base 7: mod_base = OTHER; note = 2'-O-methyladenosine
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = cm
  modified_base 18: mod_base = um
  modified_base 19: mod_base = um
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = gm
  source 1..23: mol_type = other RNA
tgcctaatgagccacacttggag

SEQ ID NO: 152 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = um
  modified_base 6: mod_base = OTHER; note = 2'-fluorouridine
  modified_base 7: mod_base = um
  modified_base 8: mod_base = gm
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = cm
  source 1..23: mol_type = other RNA
ttggtttgtagactatttgcacc

SEQ ID NO: 153 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = t is thymine with a 3'-phosphorothioate linkage
  modified_base 3: mod_base = gm
  modified_base 4: mod_base = gm
  modified_base 5: mod_base = OTHER; note = t is thymine
  modified_base 6: mod_base = um
  modified_base 7: mod_base = um
  modified_base 8: mod_base = OTHER; note = 2'-fluoroguanosine
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = cm
  source 1..23: mol_type = other RNA
ttggtttgtagactatttgcacc

SEQ ID NO: 154 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-O-C3H6-NH(CO)-C2H4-SS-C16H33 uridine
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 155 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-C3H6-NH(CO)-C2H4-SS-C12H25 adenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 156 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = um
  modified_base 6: mod_base = um
  modified_base 7: mod_base = um
  modified_base 8: mod_base = gm
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = cm
  source 1..23: mol_type = other RNA
ttggtttgtagactatttgcacc

SEQ ID NO: 157 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = t is thymine with a 3'-phosphorothioate linkage
  modified_base 3: mod_base = gm
  modified_base 4: mod_base = gm
  modified_base 5: mod_base = OTHER; note = t is thymine
  modified_base 6: mod_base = um
  modified_base 7: mod_base = um
  modified_base 8: mod_base = OTHER; note = 2'-deoxyguanosine
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = cm
  source 1..23: mol_type = other RNA
ttggtttgtagactatttgcacc

SEQ ID NO: 158 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = n is an abasic nucleotide
  modified_base 6: mod_base = um
  modified_base 7: mod_base = um
  modified_base 8: mod_base = OTHER; note = 2'-fluoroguanosine
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = cm
  source 1..23: mol_type = other RNA
ttggnttgtagactatttgcacc

SEQ ID NO: 159 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = 2'-fluorouridine
  modified_base 6: mod_base = um
  modified_base 7: mod_base = OTHER; note = n is an abasic nucleotide
  modified_base 8: mod_base = OTHER; note = 2'-fluoroguanosine
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = cm
  source 1..23: mol_type = other RNA
ttggttngtagactatttgcacc

SEQ ID NO: 160 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-O-C3H6-NH(CO)-C2H4-SS-C12H25 uridine
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 161 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = OTHER; note = 2'-O-C3H6-NH(CO)-C2H4-SS-C12H25 uridine
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 162 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-C3H6-NH(CO)-C2H4-SS-C12H25 cytidine
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 163 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = OTHER; note = 2'-O-C3H6-NH(CO)-C2H4-SS-C12H25 cytidine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 164 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = 2'-fluorouridine
  modified_base 6: mod_base = um
  modified_base 7: mod_base = OTHER; note = 2'-fluorouridine
  modified_base 8: mod_base = gm
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = cm
  source 1..23: mol_type = other RNA
ttggtttgtagactatttgcacc

SEQ ID NO: 165 - RNA, 22 nt, synthetic construct
  misc_feature 1..22: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = 2'-fluorouridine
  modified_base 6: mod_base = um
  modified_base 7: mod_base = um
  modified_base 8: mod_base = OTHER; note = 2'-fluoroguanosine
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = um
  modified_base 19: mod_base = gm
  modified_base 22: mod_base = cm
  modified_base 21: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  source 1..22: mol_type = other RNA
ttggtttgtagactatttgcac

SEQ ID NO: 166 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-O-C3H6-NH(CO)-C2H4-SS-adamantyl uridine
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

(19) United States
(12) Patent Application Publication
(10) Pub. No.: US 2026/0201378 A1
(43) Pub. Date: Jul. 16, 2026

(54) RNAi AGENT WITH MODIFIED NUCLEOTIDES

(71) Applicant: ELI LILLY AND COMPANY, Indianapolis, IN (US)

(72) Inventors: Lacie Marie CHAUVIGNE-HINES, Plainfield, IN (US); Sarah Katharina FRITSCHI, Boston, MA (US); Isabel Cristina GONZALEZ-VALCARCEL, Indianapolis, IN (US); Erica Theresa GREENE, Tewksbury, MA (US); Katarina Lynn KEEL, Indianapolis, IN (US); Douglas Raymond PERKINS, New Palestine, IN (US); Aaron David WROBLESKI, Indianapolis, IN (US); Jeremy S. YORK, Noblesville, IN (US); Daniel Keith MILLER, Fishers, IN (US); Shawn HERRON, Cambridge, MA (US); Carolyn M. HURDLE, Indianapolis, IN (US); Feng LIU, Foster City, CA (US)

(21) Appl. No.: 19/137,491

(22) PCT Filed: Dec. 12, 2023
(86) PCT No.: PCT/US2023/083651

§ 371 Date: Jun. 10, 2025

Related U.S. Application Data
(60) Provisional application No. 63/387,000, filed on Dec. 12, 2022.

(51) Int. Cl. C12N 15/113; A61K 31/712; A61K 31/7125; A61K 31/713; A61P 25/28

(52) U.S. Cl. CPC C12N 15/113; A61K 31/712; A61K 31/7125; A61K 31/713; A61P 25/28; C12N 2310/14; C12N 2310/312; C12N 2310/315; C12N 2310/321; C12N 2310/322

(57) ABSTRACT

Provided herein are novel compounds and RNAi agents comprising modified nucleotides, compositions comprising such compounds or RNAi agents, and methods of using such compounds or RNAi agents.

### SEQUENCE LISTING

The present application is being filed along with a Sequence Listing in ST.26 XML format. The Sequence Listing is provided as a file titled “30457 WO” created Oct. 30, 2023 and is 1,380 kilobytes in size. The Sequence Listing information in the ST.26 XML format is incorporated herein by reference in its entirety.

### BACKGROUND

RNA interference (RNAi) is a highly conserved regulatory mechanism in which sequence-specific gene silencing is achieved by double-stranded RNA molecules (dsRNA) (Fire et al., Nature 391:806-811, 1998). Physiologically, RNAi is initiated by Dicer enzyme, which cleaves long dsRNA molecules into short double-stranded fragments of approximately 21 to 23 nucleotide siRNAs. After the siRNA unwinds, the antisense strand is loaded into the RNA-induced silencing complex (RISC) and hybridizes to a complementary sequence in a target mRNA, while the sense strand is degraded (Nakanishi, Wiley Interdiscip. Rev. RNA, Vol. 7: 637-660, 2016). Silencing of the target mRNA is then mediated by Ago2, the catalytic component of the RISC (Bobbin and Rossi, Annu. Rev. Pharmacol. Toxicol., Vol. 56:103-122, 2016).

RNAi agents are susceptible to nuclease degradation. One of the challenges for RNAi based therapies is the ability to deliver intact RNAi agent into the target tissues and cells. Chemical modifications and/or ligand conjugations can be used to improve stability and delivery of RNAi agent into target tissues and cells. However, some chemical modifications and/or ligand conjugations are not well tolerated and raise safety concerns in human patients (Chi, et al., Drug Discov. Today. 2017 May; 22(5):823-833).

There remains a need for safe and effective RNAi agents suitable for therapeutic uses, e.g., for the treatment of human diseases.

### SUMMARY OF INVENTION

Provided herein are novel compounds and RNAi agents comprising modified nucleotides with good tolerability, efficacy, and tissue distribution profiles in animal models, compositions comprising such compounds or RNAi agents, and methods of using such compounds or RNAi agents.

In one aspect, provided herein are compounds comprising any one of the following Formulae:

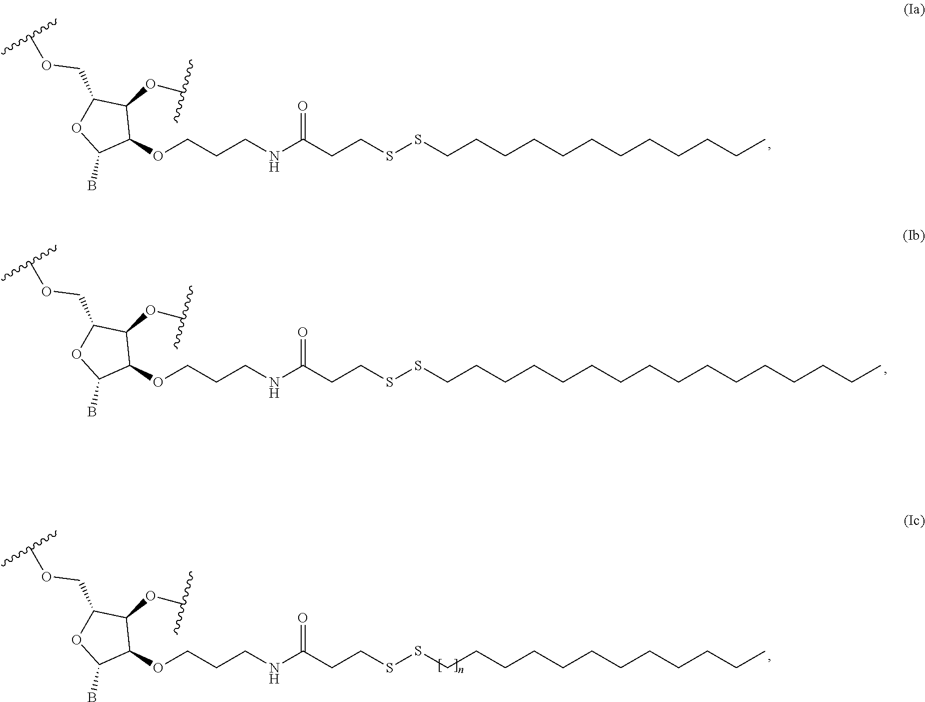

wherein n is an integer of 1-4,

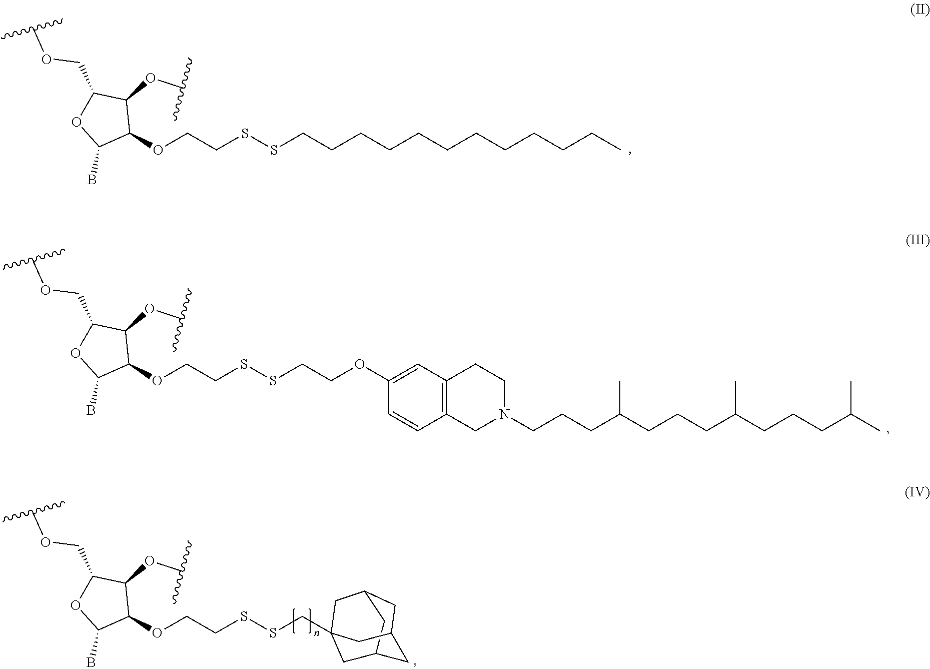

wherein n is an integer of 0-2,

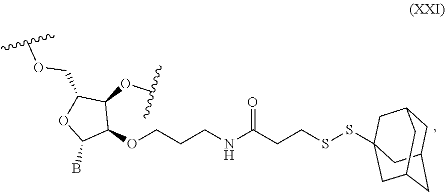

and
wherein B is a nucleobase selected from adenine (A), cytosine (C), guanine (G), thymine (T), uracil (U), or a derivative thereof. In some embodiments, n is 1 in Formula Ic. In some embodiments, n is 2 in Formula Ic. In some embodiments, n is 3 in Formula Ic. In some embodiments, n is 4 in Formula Ic. In some embodiments, n is 0 in Formula IV. In some embodiments, n is 1 in Formula IV. In some embodiments, n is 2 in Formula IV.

In some embodiments, the compound comprising any one of Formula Ia, Ib, Ic, II-IV or XXI is a nucleoside, nucleotide, or analog thereof.

In another aspect, provided herein are RNAi agent comprising a sense stand and an antisense strand, wherein the sense strand and the antisense strand form a duplex region, wherein the sense strand or the antisense strand comprises a modified nucleotide of any one of the following Formulae:

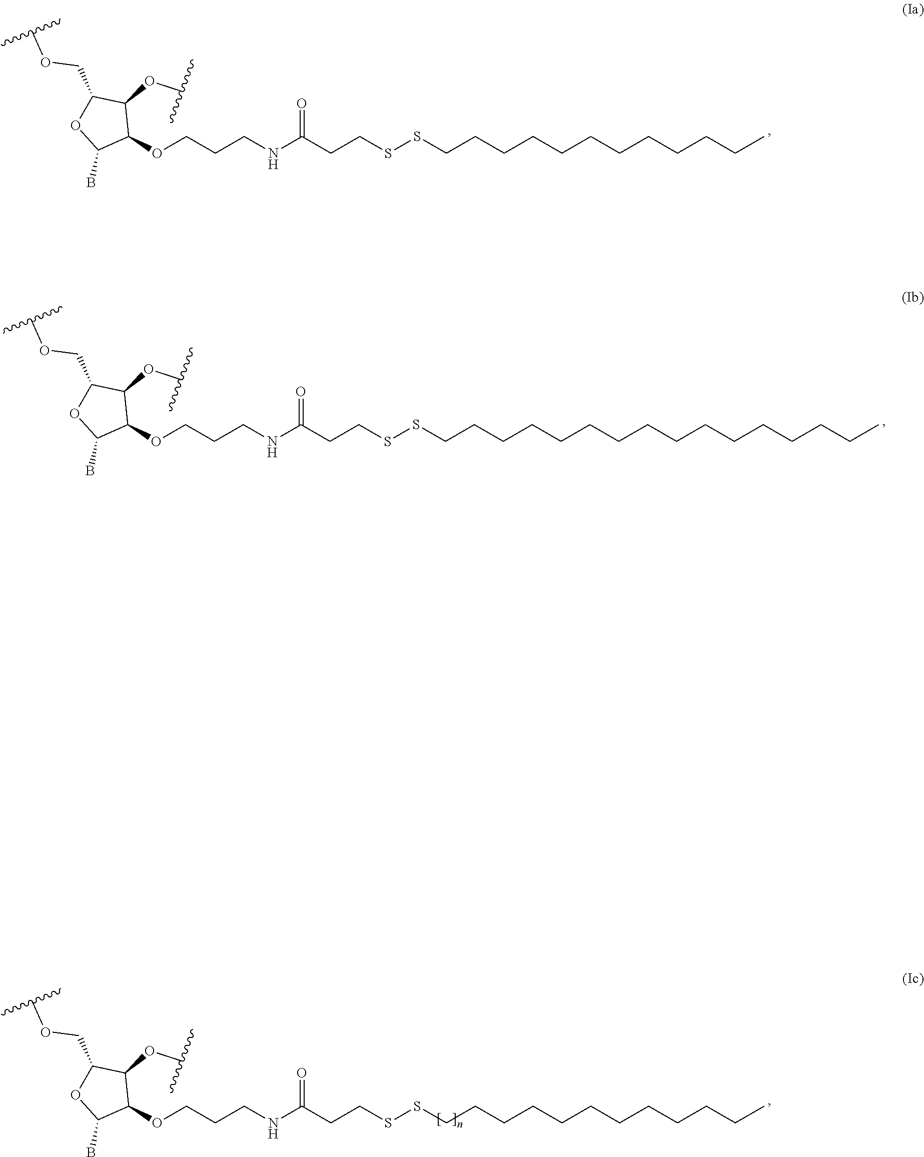

wherein n is an integer of 1-4.

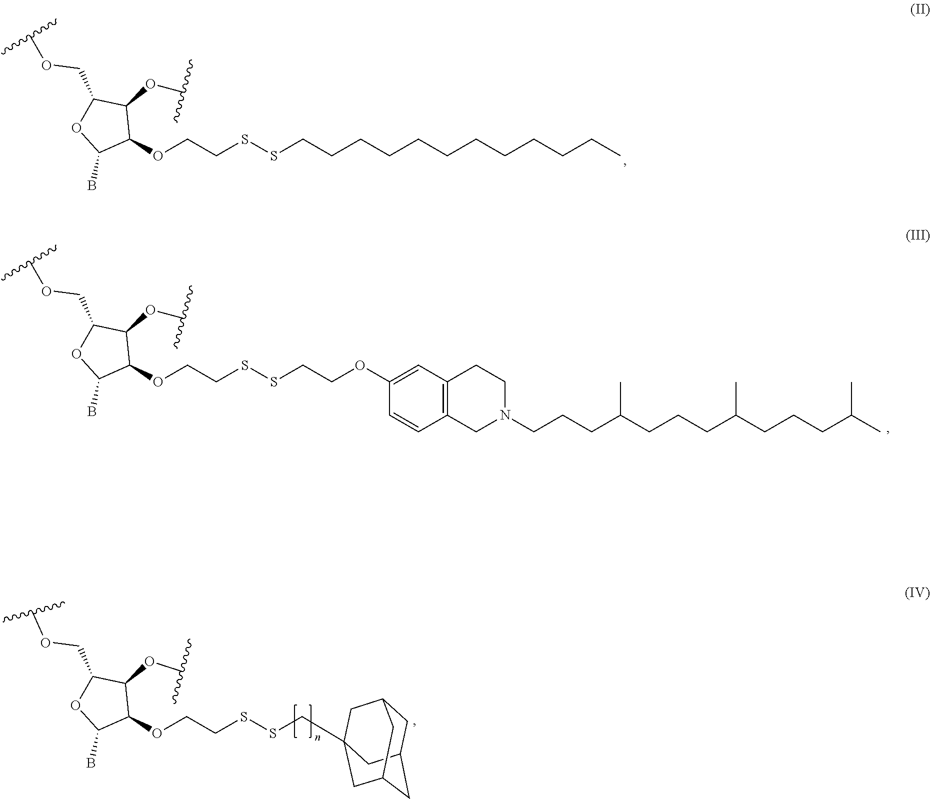

wherein n is an integer of 0-2,

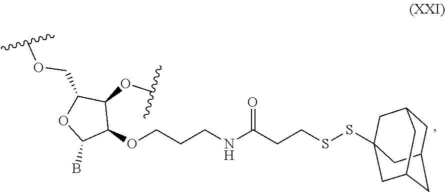

and
wherein B is a nucleobase selected from adenine, cytosine, guanine, thymine, uracil, or a derivative thereof. In some embodiments, n is 1 in Formula Ic. In some embodiments, n is 2 in Formula Ic. In some embodiments, n is 3 in Formula Ic. In some embodiments, n is 4 in Formula Ic. In some embodiments, n is 0 in Formula IV. In some embodiments, n is 1 in Formula IV. In some embodiments, n is 2 in Formula IV.

In some embodiments, the sense strand is 15 to 50 nucleotides in length. In some embodiments, the antisense strand is 15 to 30 nucleotides in length.

In some embodiments, the sense strand comprises the modified nucleotide of any one of Formula Ia, Ib, Ic, II-IV or XXI, e.g., at any one of positions 1-6 or 12-21 from the 5′ end. In some embodiments, the antisense strand comprises the modified nucleotide of any one of Formula Ia, Ib, Ic, II-IV or XXI, e.g., at any one of positions 6-10 or 15-18 from the 5′ end.

In some embodiments, the sense strand and antisense strand further comprise one or more 2′-fluoro modified nucleotides and 2′-O-methyl modified nucleotides. In some embodiments, the sense strand and the antisense strand comprise one or more modified internucleotide linkages, e.g., phosphorothioate linkages.

In some embodiments, the antisense strand comprises a phosphate analog (e.g., 5′-vinylphosphonate) at 5′ end. In some embodiments, the sense strand comprises an abasic moiety or inverted abasic moiety.

In some embodiments, the antisense strand is complementary to a target mRNA selected from SNCA, MAPT, APP, ATXN2, ATXN3, SARM1, APOE, BACE1, FMR1, LRRK2, HTT, SOD1, SCN10A, SCN9A or CACNA1B mRNA. In some embodiments, the antisense strand is complementary to SNCA mRNA. Exemplary RNAi agents targeting human SNCA mRNA are provided in Table 1. In some embodiments, the antisense strand is complementary to MAPT mRNA. Exemplary RNAi agents targeting human MAPT mRNA are provided in Table 2.

In another aspect, provided herein are pharmaceutical compositions comprising a compound or RNAi agent described herein and a pharmaceutically acceptable carrier.

In a further aspect, provided herein are methods of treating a neurodegenerative disease (e.g., synucleinopathy or tauopathy) in a patient in need thereof, such methods comprise administering to the patient an effective amount of a compound, RNAi agent or pharmaceutical composition described herein. In some embodiments, the compound, RNAi agent or pharmaceutical composition is administered to the patient intrathecally, intracerebroventricularly, or via intracisternal magna injection.

Also provided herein are methods of inhibiting or reducing a target mRNA in a cell, the method comprising contacting the cell comprising the target mRNA with the compound, RNAi agent, or pharmaceutical composition described herein.

In another aspect, provided herein are compounds, RNAi agents or pharmaceutical compositions for use in a therapy. Also provided herein are compounds, RNAi agents, or pharmaceutical compositions for use in the treatment of a neurodegenerative disease, e.g., synucleinopathy or tauopathy. Also provided herein are uses of compounds or RNAi agents in the manufacture of a medicament for the treatment of a neurodegenerative disease, e.g., synucleinopathy or tauopathy.

### DETAILED DESCRIPTION

Provided herein are novel compounds and RNAi agents comprising modified nucleotides with good tolerability, efficacy, and tissue distribution profiles in animal models, compositions comprising such compounds or RNAi agents, and methods of using such compounds or RNAi agents.

In one aspect, provided herein are compounds comprising any one of the following Formulae:

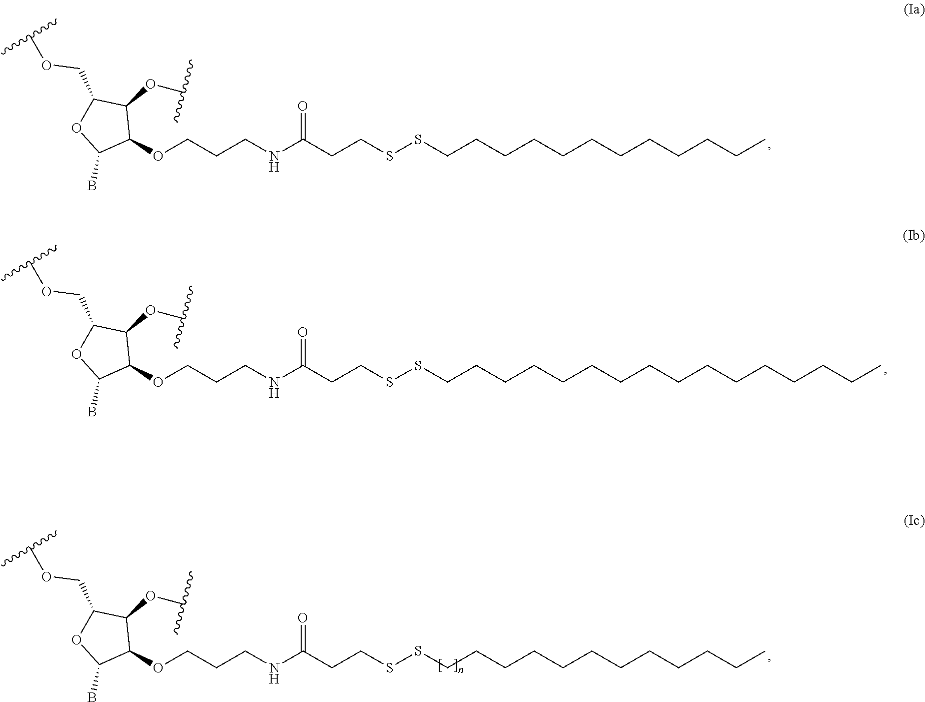

wherein n is an integer of 1-4.

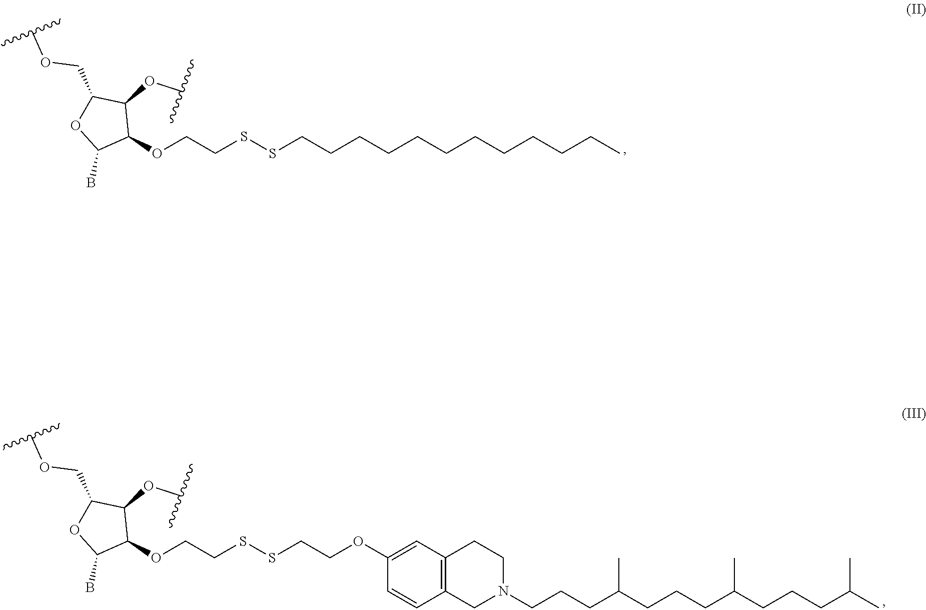

wherein n is an integer of 0-2,

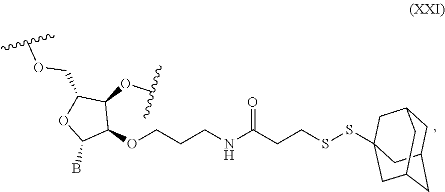

and
wherein B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G. In some embodiments, n is 1 in Formula Ic. In some embodiments, n is 2 in Formula Ic. In some embodiments, n is 3 in Formula Ic. In some embodiments, n is 4 in Formula Ic. In some embodiments, n is 0 in Formula IV. In some embodiments, n is 1 in Formula IV. In some embodiments, n is 2 in Formula IV.

In some embodiments, the compound comprising any one of Formula Ia, Ib, Ic, II-IV or XXI is a nucleoside, nucleotide, or analog thereof.

In some embodiments, provided herein are compounds comprising Formula Ia

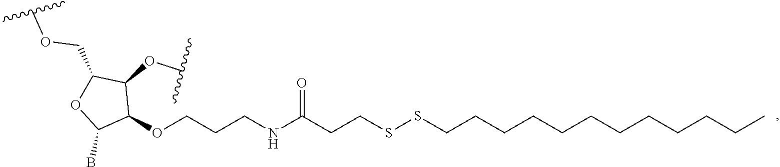

wherein B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G.

In some embodiments, provided herein are compounds comprising Formula Ib

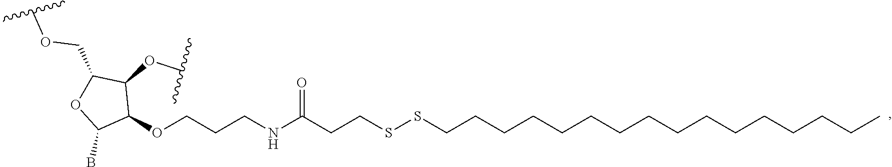

wherein B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G.

In some embodiments, provided herein are compounds comprising Formula Ic

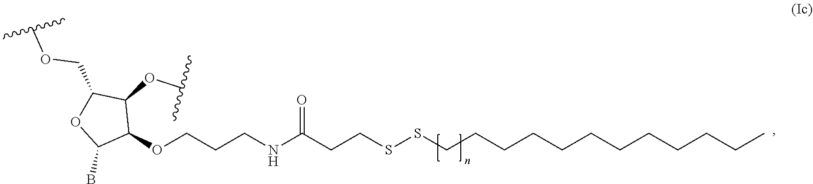

wherein n is an integer of 1-4, and
wherein B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G. In some embodiments, n is 1 in Formula Ic. In some embodiments, n is 2 in Formula Ic. In some embodiments, n is 3 in Formula Ic. In some embodiments, n is 4 in Formula Ic.

In some embodiments, provided herein are compounds comprising Formula II

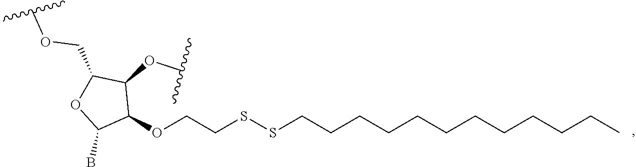

wherein B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G.

In some embodiments, provided herein are compounds comprising Formula III

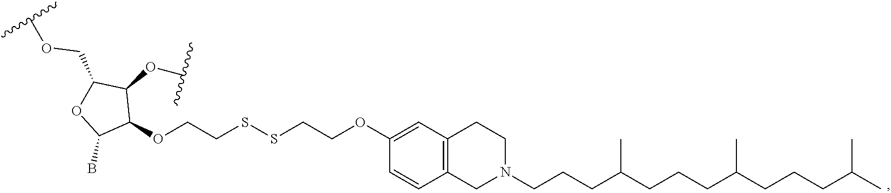

wherein B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G.

In some embodiments, provided herein are compounds comprising Formula IV

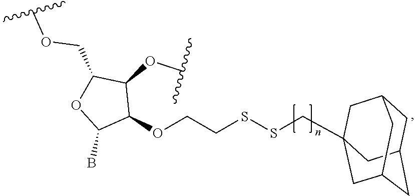

wherein n is an integer of 0-2, and wherein B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G. In some embodiments, n is 0 in Formula IV. In some embodiments, n is 1 in Formula IV. In some embodiments, n is 2 in Formula IV.

In some embodiments, provided herein are compounds comprising Formula XXI

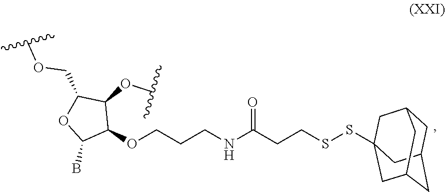

wherein B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G.

In some embodiments, provided herein are compounds comprising any one of Formula Va-VIIIa:

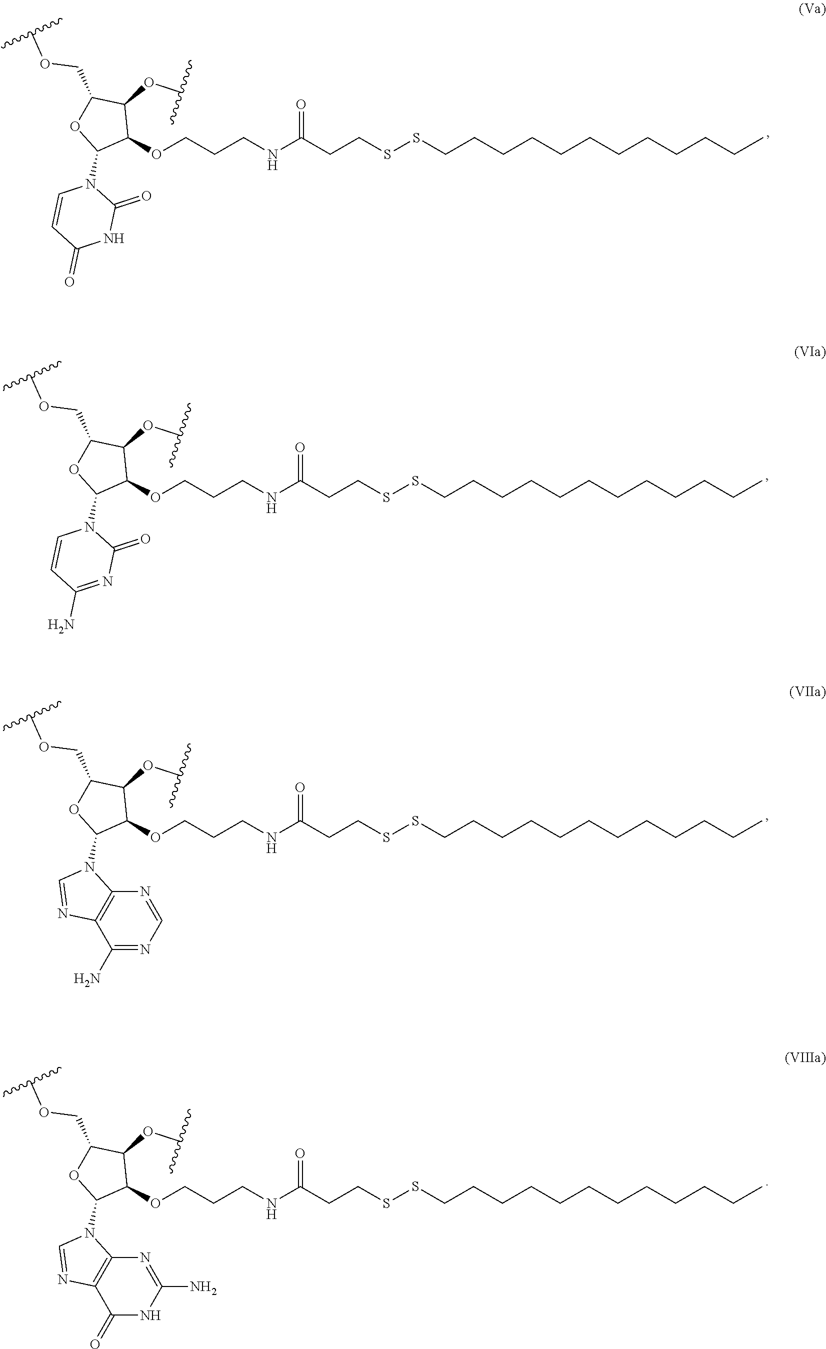 [SMILES: *OC[C@H]1O[C@@H](n2ccc(=O)[nH]c2=O)[C@H](OCCCNC(=O)CCSSCCCCCCCCCCCC)[C@@H]1O* ; *OC[C@H]1O[C@@H](n2ccc(N)nc2=O)[C@H](OCCCNC(=O)CCSSCCCCCCCCCCCC)[C@@H]1O* ; *OC[C@H]1O[C@@H](n2cnc3c(N)ncnc32)[C@H](OCCCNC(=O)CCSSCCCCCCCCCCCC)[C@@H]1O* ; *OC[C@H]1O[C@@H](n2cnc3c(=O)[nH]c(N)nc32)[C@H](OCCCNC(=O)CCSSCCCCCCCCCCCC)[C@@H]1O*]

In some embodiments, the compound comprising any one of Formula Va-VIIIa is a nucleoside, nucleotide, or analog thereof.

In some embodiments, provided herein are compounds comprising any one of Formula Vb-VIIIb:

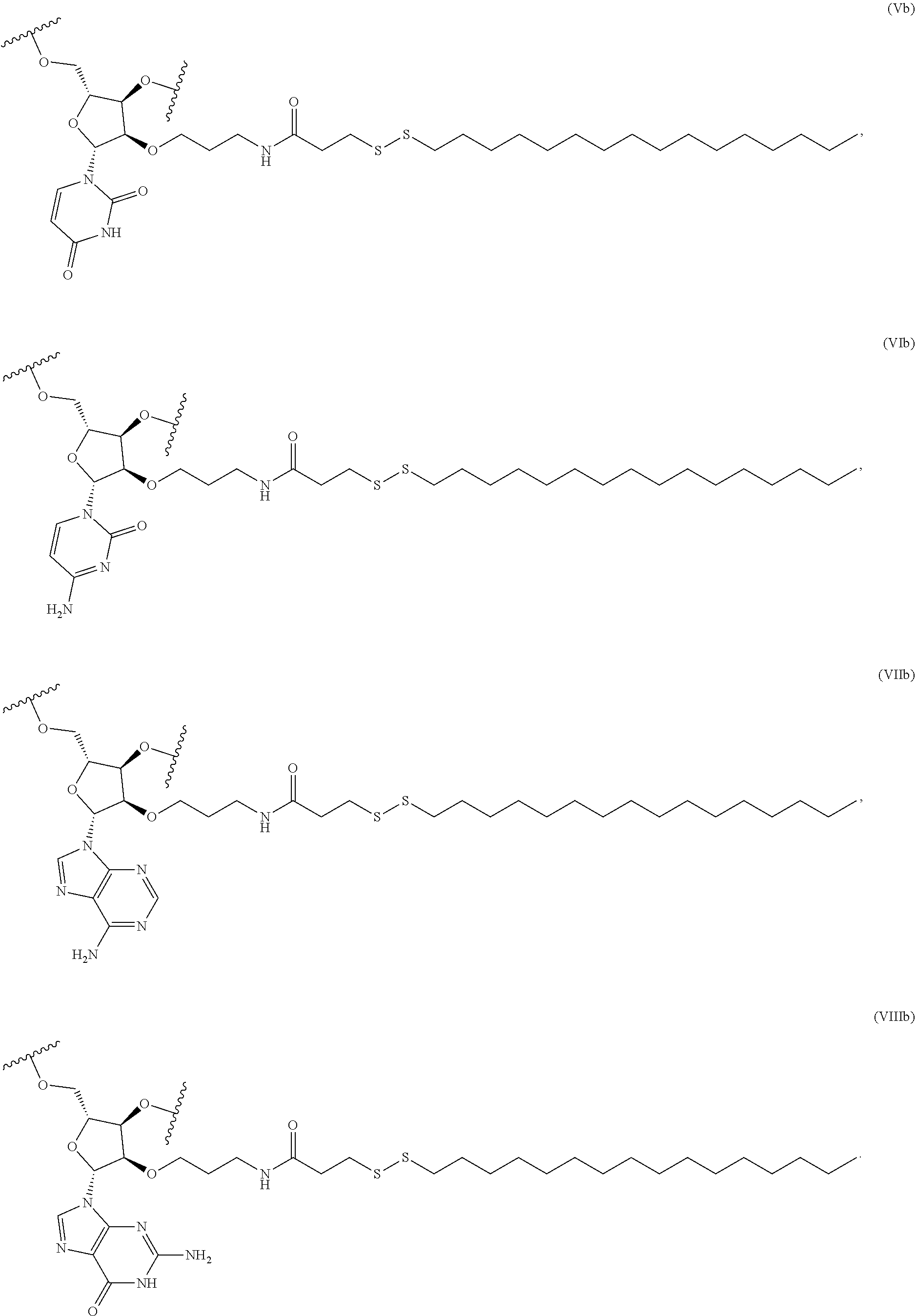 [SMILES: *OC[C@H]1O[C@@H](n2ccc(=O)[nH]c2=O)[C@H](OCCCNC(=O)CCSSCCCCCCCCCCCCCCCC)[C@@H]1O* ; *OC[C@H]1O[C@@H](n2ccc(N)nc2=O)[C@H](OCCCNC(=O)CCSSCCCCCCCCCCCCCCCC)[C@@H]1O* ; *OC[C@H]1O[C@@H](n2cnc3c(N)ncnc32)[C@H](OCCCNC(=O)CCSSCCCCCCCCCCCCCCCC)[C@@H]1O* ; *OC[C@H]1O[C@@H](n2cnc3c(=O)[nH]c(N)nc32)[C@H](OCCCNC(=O)CCSSCCCCCCCCCCCCCCCC)[C@@H]1O*]

In some embodiments, the compound comprising any one of Formula Vb-VIIIb is a nucleoside, nucleotide, or analog thereof.

In some embodiments, provided herein are compounds comprising any one of Formula Vc-VIIIc:

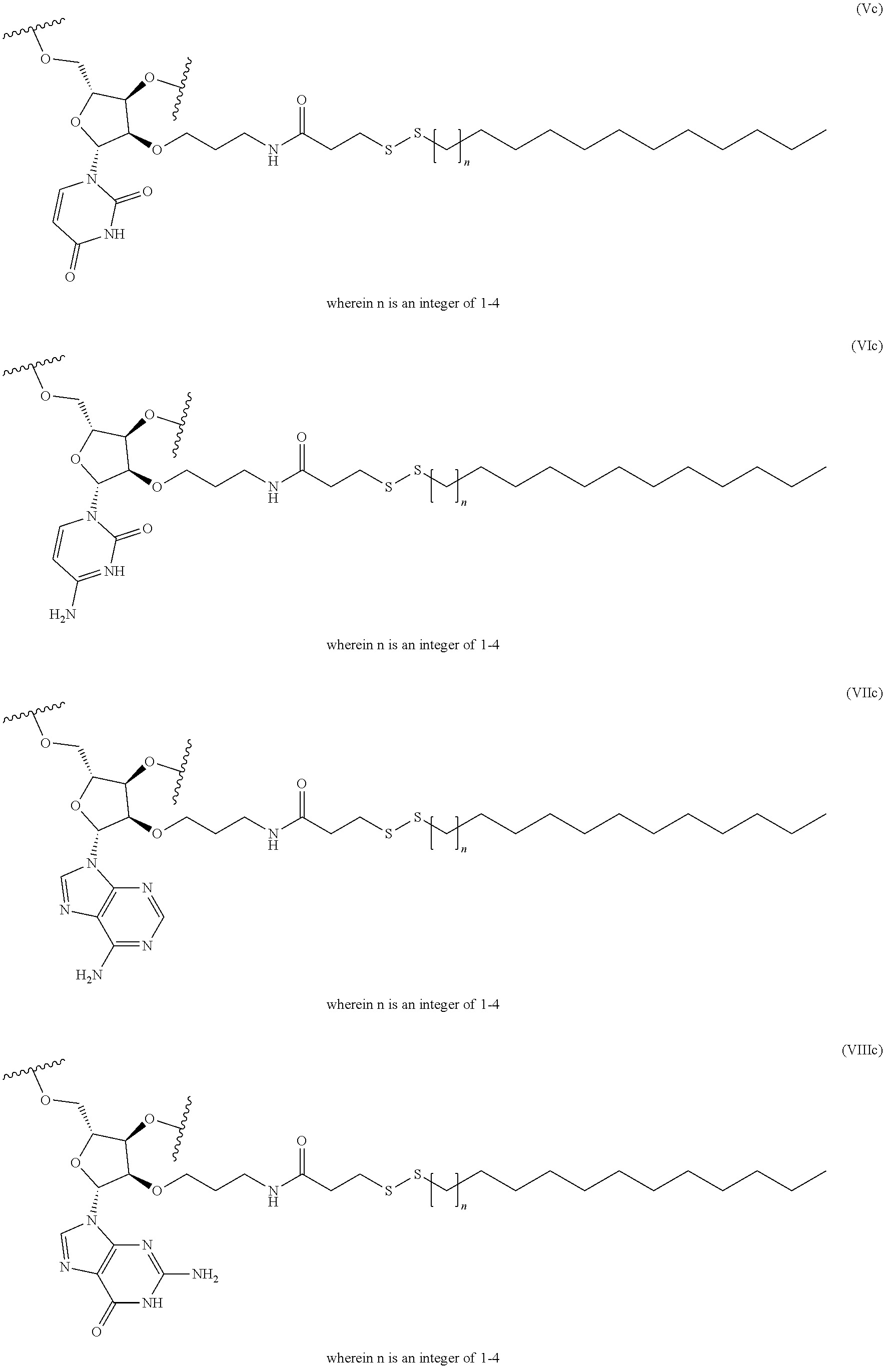

In some embodiments, n is 1 in Formula Vc-VIIIc. In some embodiments, n is 2 in Formula Vc-VIIIc. In some embodiments, n is 3 in Formula Vc-VIIIc. In some embodiments, n is 4 in Formula Vc-VIIIc.

In some embodiments, the compound comprising any one of Formula Vc-VIIIc is a nucleoside, nucleotide, or analog thereof.

In some embodiments, provided herein are compounds comprising any one of Formula IX-XII:

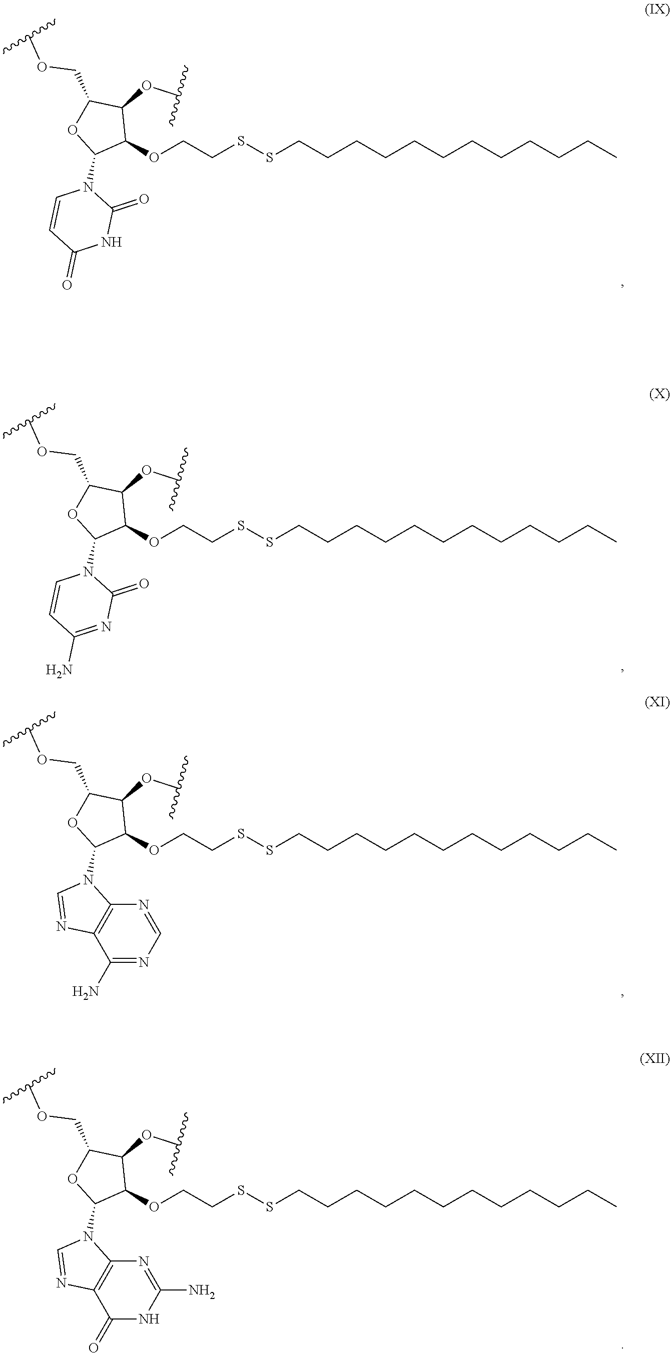 [SMILES: *OC[C@H]1O[C@@H](n2ccc(=O)[nH]c2=O)[C@H](OCCSSCCCCCCCCCCCC)[C@@H]1O* ; *OC[C@H]1O[C@@H](n2ccc(N)nc2=O)[C@H](OCCSSCCCCCCCCCCCC)[C@@H]1O* ; *OC[C@H]1O[C@@H](n2cnc3c(N)ncnc32)[C@H](OCCSSCCCCCCCCCCCC)[C@@H]1O* ; *OC[C@H]1O[C@@H](n2cnc3c(=O)[nH]c(N)nc32)[C@H](OCCSSCCCCCCCCCCCC)[C@@H]1O*]

In some embodiments, the compound comprising any one of Formula IX-XII is a nucleoside, nucleotide, or analog thereof.

In some embodiments, provided herein are compounds comprising any one of Formula XIII-XVI:

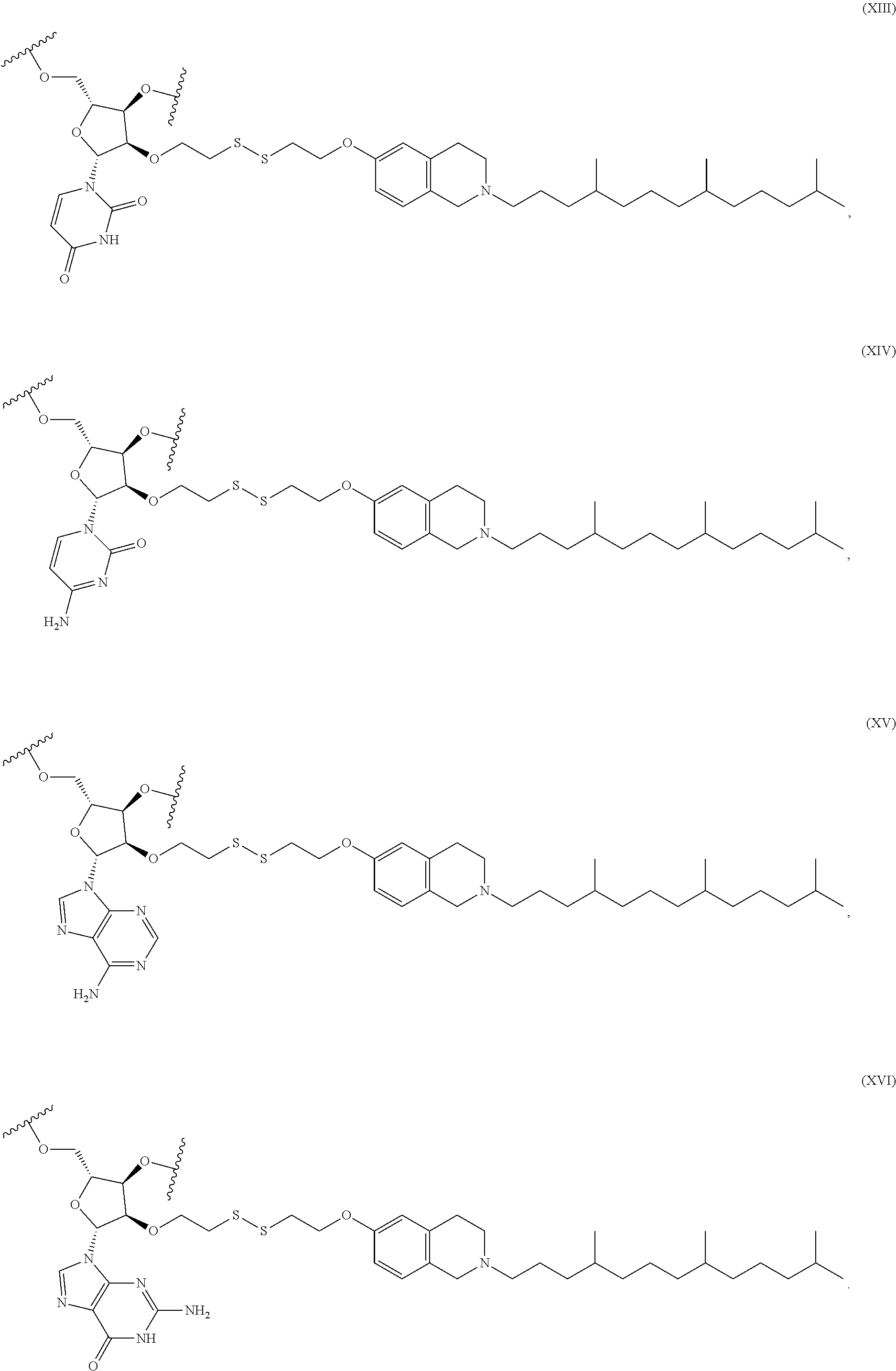 [SMILES: *OC[C@H]1O[C@@H](n2ccc(=O)[nH]c2=O)[C@H](OCCSSCCOc2ccc3c(c2)CCN(CCCC(C)CCCC(C)CCCC(C)C)C3)[C@@H]1O* ; *OC[C@H]1O[C@@H](n2ccc(N)nc2=O)[C@H](OCCSSCCOc2ccc3c(c2)CCN(CCCC(C)CCCC(C)CCCC(C)C)C3)[C@@H]1O* ; *OC[C@H]1O[C@@H](n2cnc3c(N)ncnc32)[C@H](OCCSSCCOc2ccc3c(c2)CCN(CCCC(C)CCCC(C)CCCC(C)C)C3)[C@@H]1O* ; *OC[C@H]1O[C@@H](n2cnc3c(=O)[nH]c(N)nc32)[C@H](OCCSSCCOc2ccc3c(c2)CCN(CCCC(C)CCCC(C)CCCC(C)C)C3)[C@@H]1O*]

In some embodiments, the compound comprising any one of Formula XIII-XVI is a nucleoside, nucleotide, or analog thereof.

In some embodiments, provided herein are compounds comprising any one of Formula XVII-XX:

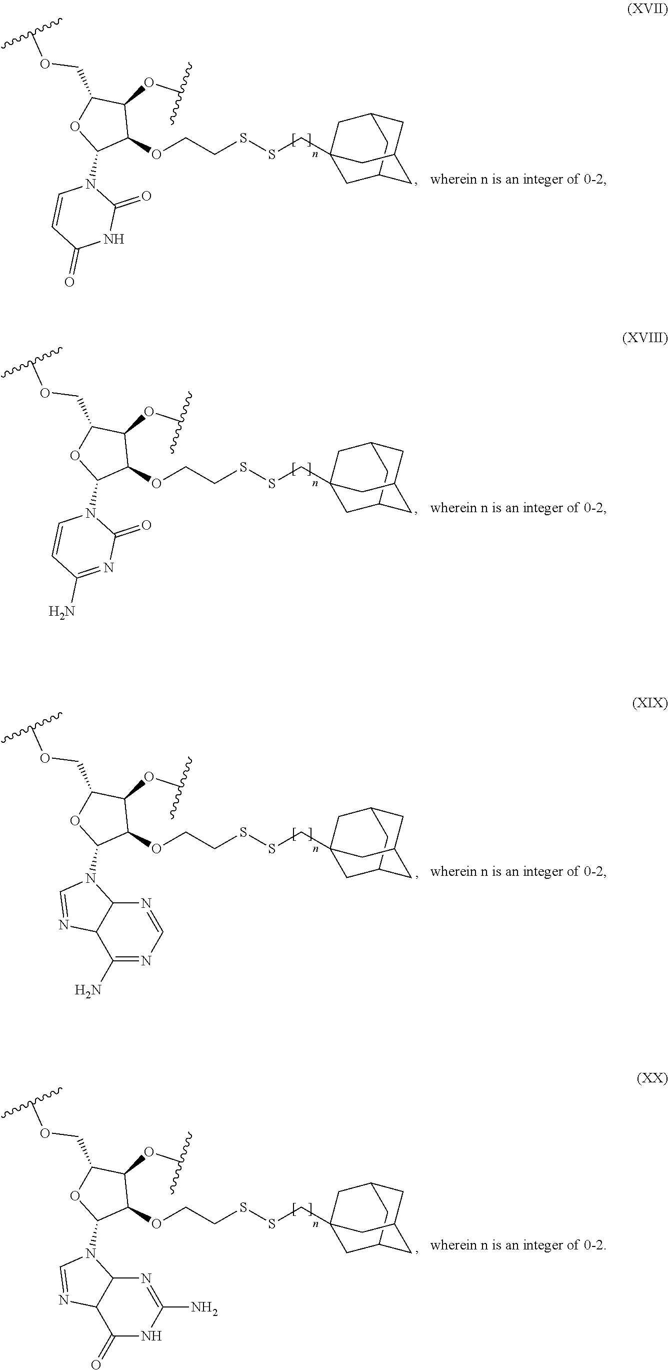

In some embodiments, n is 0 in Formula XVII-XX. In some embodiments, n is 1 in Formula XVII-XX. In some embodiments, n is 2 in Formula XVII-XX. In some embodiments, the compound comprising any one of Formula XVII-XX is a nucleoside, nucleotide, or analog thereof.

In some embodiments, provided herein are compounds comprising any one of Formula XXII-XXV:

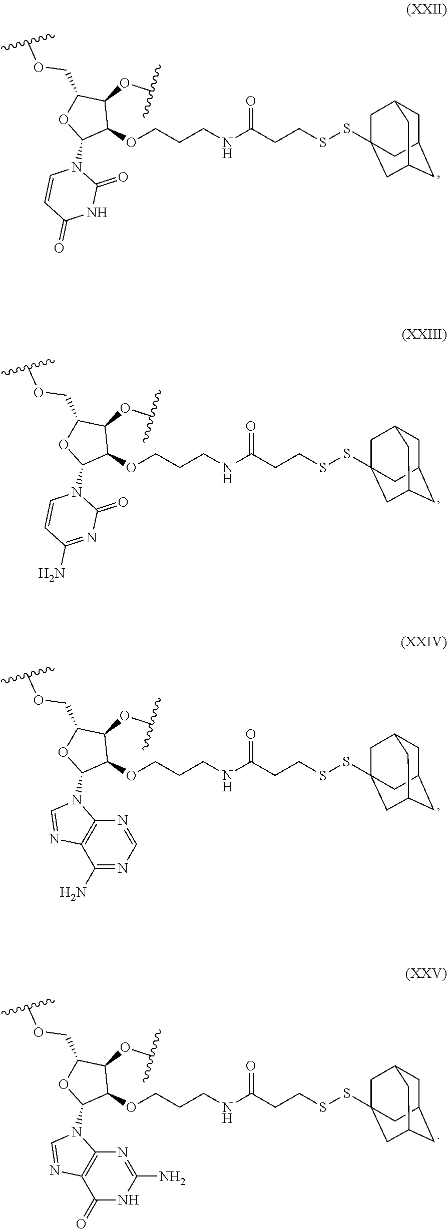 [SMILES: *OC[C@H]1O[C@@H](n2ccc(=O)[nH]c2=O)[C@H](OCCCNC(=O)CCSSC23CC4CC(CC(C4)C2)C3)[C@@H]1O* ; *OC[C@H]1O[C@@H](n2ccc(N)nc2=O)[C@H](OCCCNC(=O)CCSSC23CC4CC(CC(C4)C2)C3)[C@@H]1O* ; *OC[C@H]1O[C@@H](n2cnc3c(N)ncnc32)[C@H](OCCCNC(=O)CCSSC23CC4CC(CC(C4)C2)C3)[C@@H]1O* ; *OC[C@H]1O[C@@H](n2cnc3c(=O)[nH]c(N)nc32)[C@H](OCCCNC(=O)CCSSC23CC4CC(CC(C4)C2)C3)[C@@H]1O*]

In some embodiments, the compound comprising any one of Formula XXII-XXV is a nucleoside, nucleotide, or analog thereof.

In another aspect, provided herein are RNAi agent comprising a sense stand and an antisense strand, wherein the sense strand and the antisense strand form a duplex region, wherein the sense strand or the antisense strand comprises a modified nucleotide of any one of Formula Ia, Ib, Ic, II-IV or XXI:

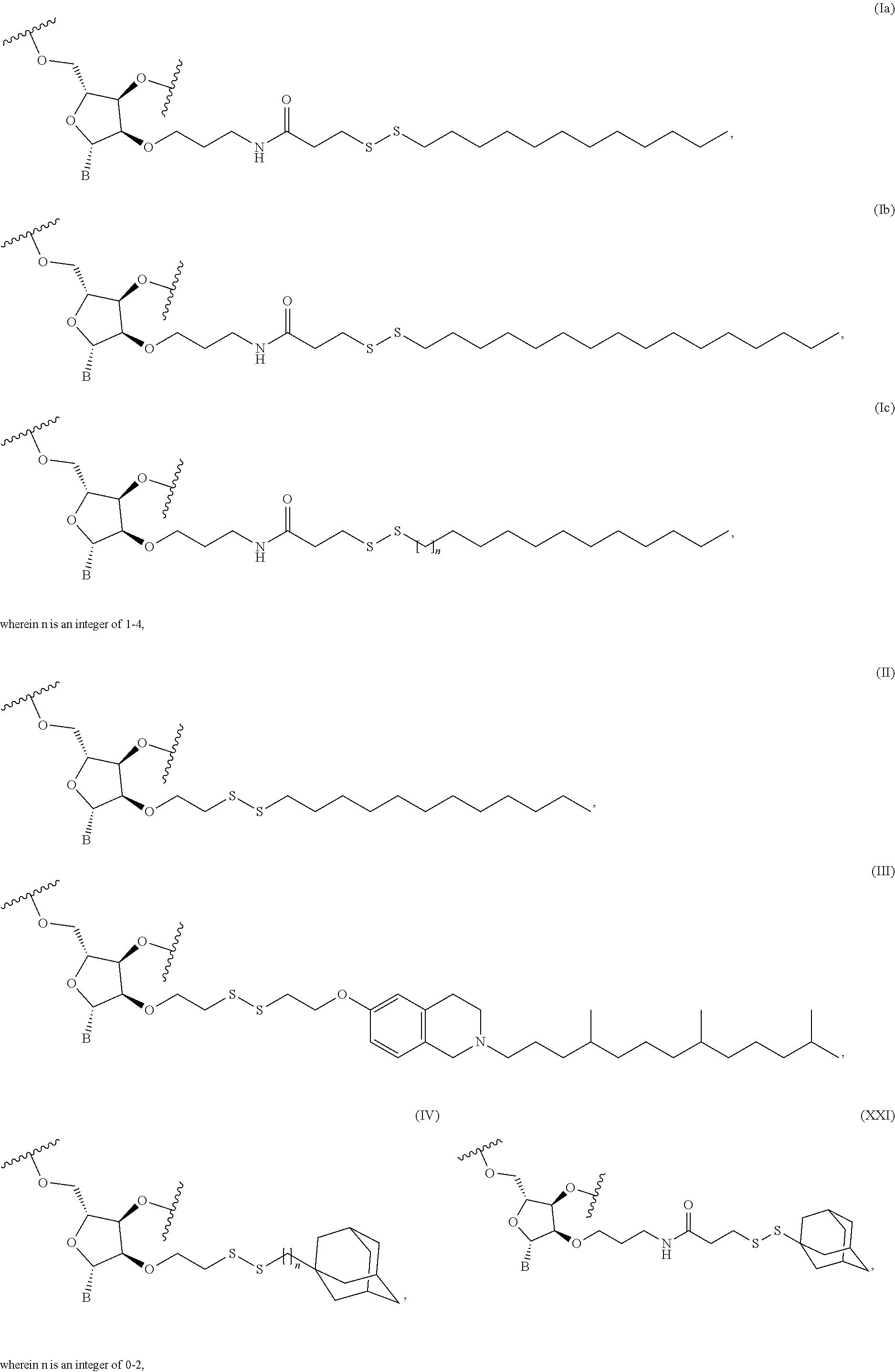

wherein B is a nucleobase selected from adenine, cytosine, guanine, thymine, uracil, or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G. In some embodiments, n is 1 in Formula Ic. In some embodiments, n is 2 in Formula Ic. In some embodiments, n is 3 in Formula Ic. In some embodiments, n is 4 in Formula Ic. In some embodiments, n is 0 in Formula IV. In some embodiments, n is 1 in Formula IV. In some embodiments, n is 2 in Formula IV.

In some embodiments, provided herein are RNAi agent comprising a sense stand and an antisense strand, wherein the sense strand and the antisense strand form a duplex region, wherein the sense strand or the antisense strand comprises a modified nucleotide of Formula Ia

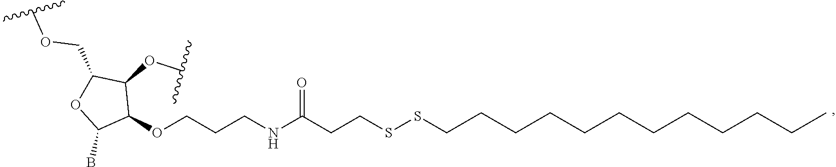

wherein B is a nucleobase selected from adenine, cytosine, guanine, thymine, uracil, or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G.

In some embodiments, provided herein are RNAi agent comprising a sense stand and an antisense strand, wherein the sense strand and the antisense strand form a duplex region, wherein the sense strand or the antisense strand comprises a modified nucleotide of Formula Ib

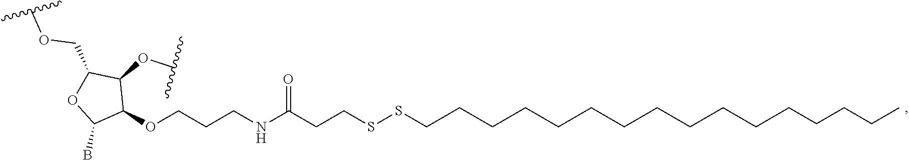

wherein B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G.

In some embodiments, provided herein are RNAi agent comprising a sense stand and an antisense strand, wherein the sense strand and the antisense strand form a duplex region, wherein the sense strand or the antisense strand comprises a modified nucleotide of Formula Ic

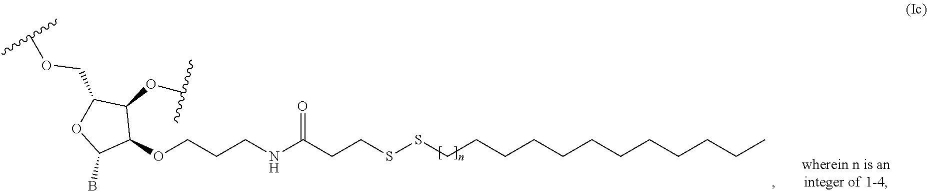

wherein B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G. In some embodiments, n is 1 in Formula I(c). In some embodiments, n is 2 in Formula I(c). In some embodiments, n is 3 in Formula I(c). In some embodiments, n is 4 in Formula I(c).

In some embodiments, provided herein are RNAi agent comprising a sense stand and an antisense strand, wherein the sense strand and the antisense strand form a duplex region, wherein the sense strand or the antisense strand comprises a modified nucleotide of Formula II

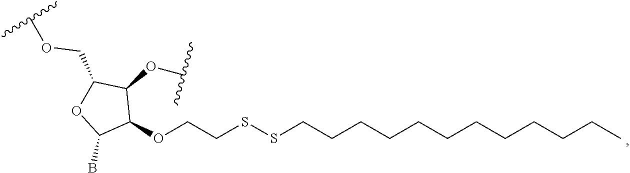

wherein B is a nucleobase selected from adenine, cytosine, guanine, thymine, uracil, or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G.

In some embodiments, provided herein are RNAi agent comprising a sense stand and an antisense strand, wherein the sense strand and the antisense strand form a duplex region, wherein the sense strand or the antisense strand comprises a modified nucleotide of Formula III

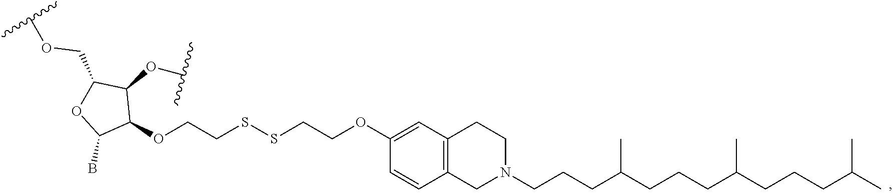

wherein B is a nucleobase selected from adenine, cytosine, guanine, thymine, uracil, or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G.

In some embodiments, provided herein are RNAi agent comprising a sense stand and an antisense strand, wherein the sense strand and the antisense strand form a duplex region, wherein the sense strand or the antisense strand comprises a modified nucleotide of Formula IV

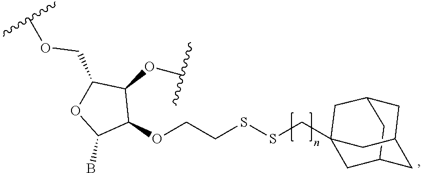

wherein n is an integer of 0-2, and
wherein B is a nucleobase selected from adenine, cytosine, guanine, thymine, uracil, or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G. In some embodiments, n is 0 in Formula IV. In some embodiments, n is 1 in Formula IV. In some embodiments, n is 2 in Formula IV.

In some embodiments, provided herein are RNAi agent comprising a sense stand and an antisense strand, wherein the sense strand and the antisense strand form a duplex region, wherein the sense strand or the antisense strand comprises a modified nucleotide of Formula XXI

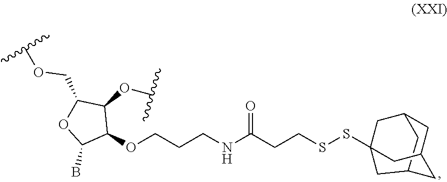

wherein B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G.

In some embodiments, provided herein are RNAi agent comprising a sense stand and an antisense strand, wherein the sense strand and the antisense strand form a duplex region, wherein the sense strand or the antisense strand comprises a modified nucleotide of any one of Formula Va-VIIIa:

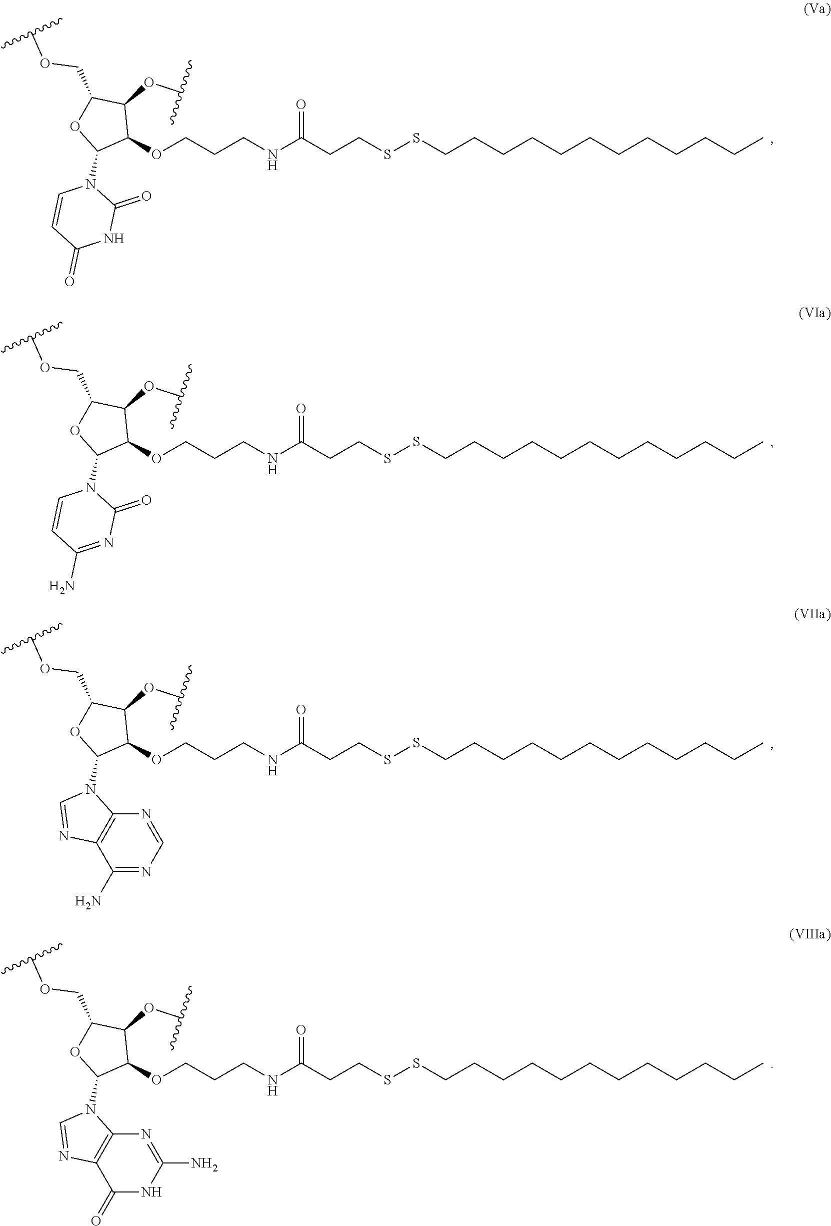 [SMILES: *OC[C@H]1O[C@@H](n2ccc(=O)[nH]c2=O)[C@H](OCCCNC(=O)CCSSCCCCCCCCCCCC)[C@@H]1O* ; *OC[C@H]1O[C@@H](n2ccc(N)nc2=O)[C@H](OCCCNC(=O)CCSSCCCCCCCCCCCC)[C@@H]1O* ; *OC[C@H]1O[C@@H](n2cnc3c(N)ncnc32)[C@H](OCCCNC(=O)CCSSCCCCCCCCCCCC)[C@@H]1O* ; *OC[C@H]1O[C@@H](n2cnc3c(=O)[nH]c(N)nc32)[C@H](OCCCNC(=O)CCSSCCCCCCCCCCCC)[C@@H]1O*]

In some embodiments, provided herein are RNAi agent comprising a sense stand and an antisense strand, wherein the sense strand and the antisense strand form a duplex region, wherein the sense strand or the antisense strand comprises a modified nucleotide of any one of Formula Vb-VIIIb:

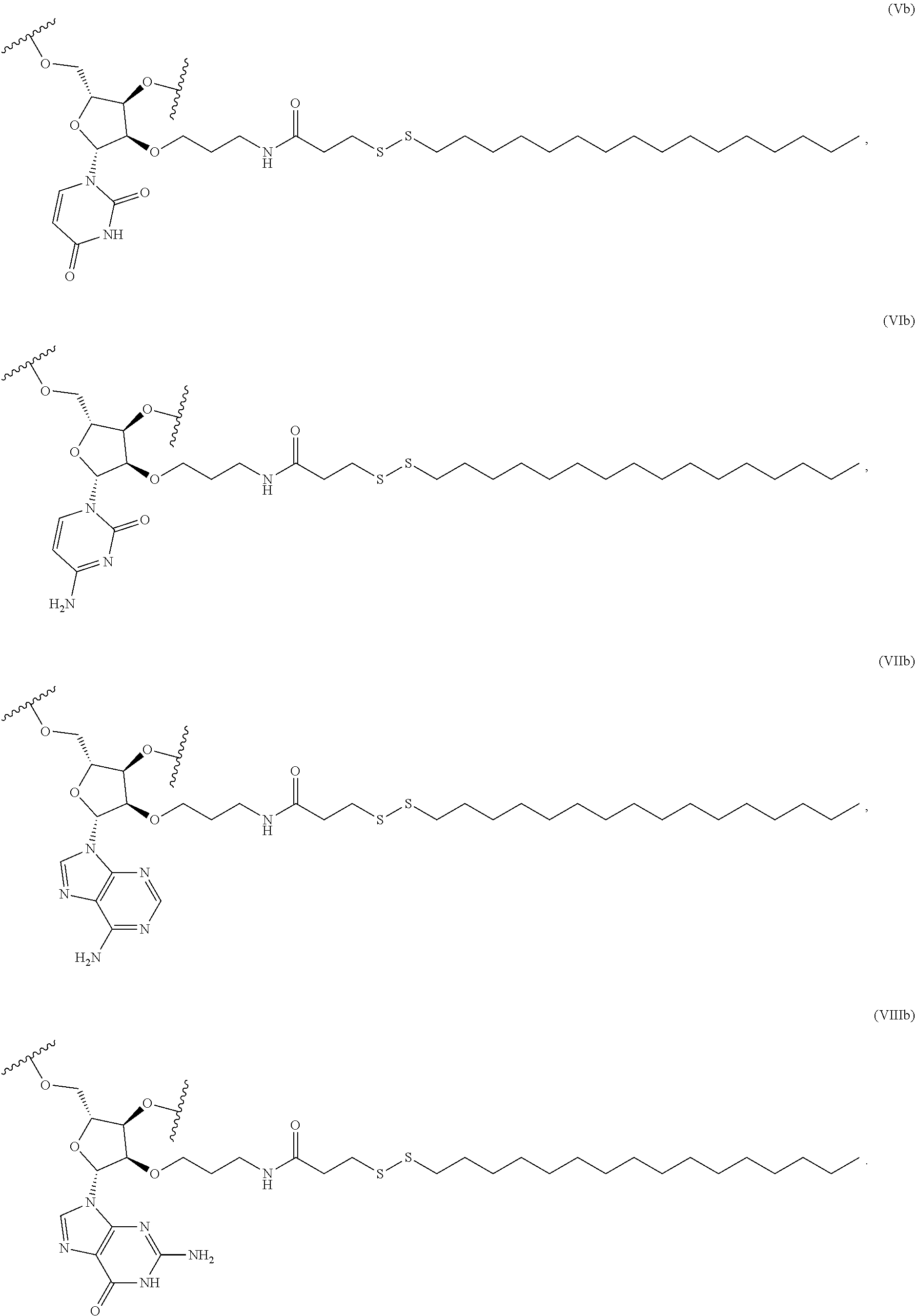 [SMILES: *OC[C@H]1O[C@@H](n2ccc(=O)[nH]c2=O)[C@H](OCCCNC(=O)CCSSCCCCCCCCCCCCCCCC)[C@@H]1O* ; *OC[C@H]1O[C@@H](n2ccc(N)nc2=O)[C@H](OCCCNC(=O)CCSSCCCCCCCCCCCCCCCC)[C@@H]1O* ; *OC[C@H]1O[C@@H](n2cnc3c(N)ncnc32)[C@H](OCCCNC(=O)CCSSCCCCCCCCCCCCCCCC)[C@@H]1O* ; *OC[C@H]1O[C@@H](n2cnc3c(=O)[nH]c(N)nc32)[C@H](OCCCNC(=O)CCSSCCCCCCCCCCCCCCCC)[C@@H]1O*]

In some embodiments, provided herein are RNAi agent comprising a sense stand and an antisense strand, wherein the sense strand and the antisense strand form a duplex region, wherein the sense strand or the antisense strand comprises a modified nucleotide of any one of Formula Vc-VIIIc:

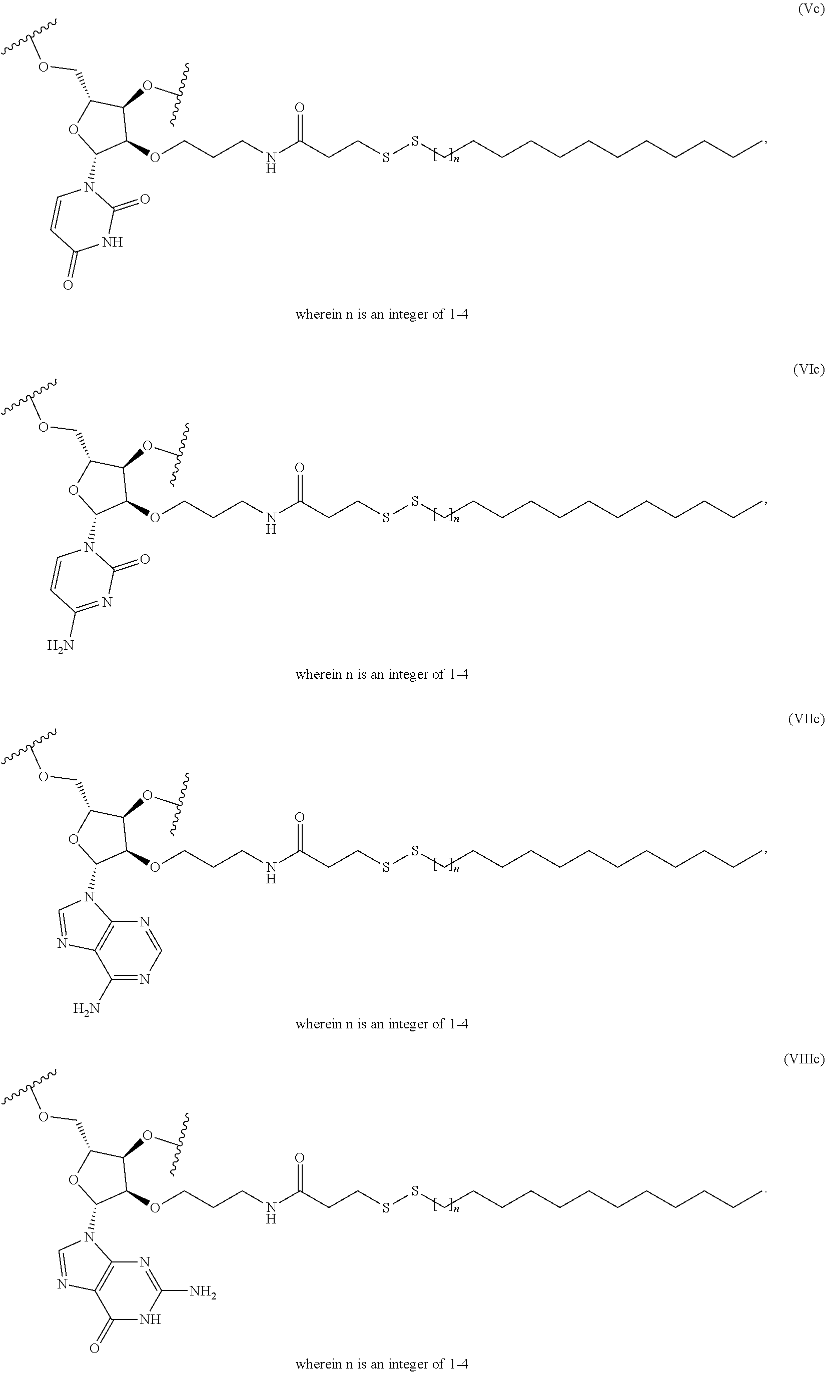

In some embodiments, n is 1 in Formula Vc-VIIIc. In some embodiments, n is 2 in Formula Vc-VIIIc. In some embodiments, n is 3 in Formula Vc-VIIIc. In some embodiments, n is 4 in Formula Vc-VIIIc.

In some embodiments, provided herein are RNAi agent comprising a sense stand and an antisense strand, wherein the sense strand and the antisense strand form a duplex region, wherein the sense strand or the antisense strand comprises a modified nucleotide of any one of Formula IX-XII.

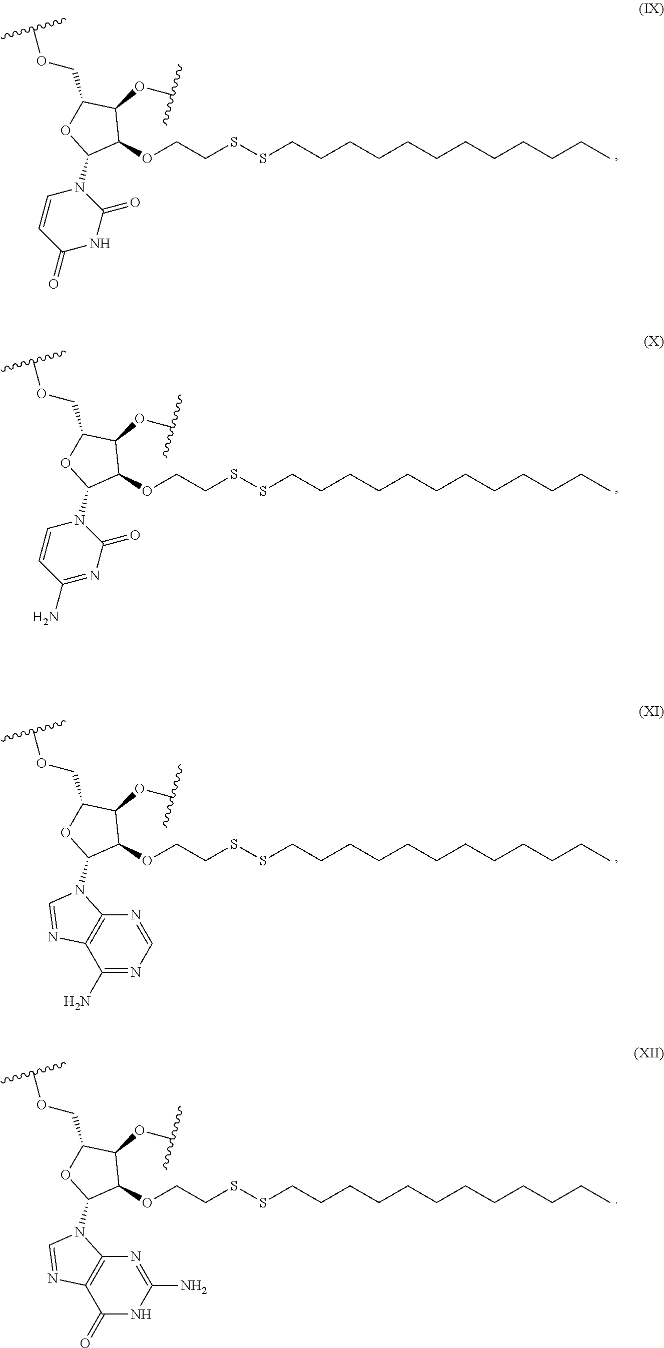 [SMILES: *OC[C@H]1O[C@@H](n2ccc(=O)[nH]c2=O)[C@H](OCCSSCCCCCCCCCCCC)[C@@H]1O* ; *OC[C@H]1O[C@@H](n2ccc(N)nc2=O)[C@H](OCCSSCCCCCCCCCCCC)[C@@H]1O* ; *OC[C@H]1O[C@@H](n2cnc3c(N)ncnc32)[C@H](OCCSSCCCCCCCCCCCC)[C@@H]1O* ; *OC[C@H]1O[C@@H](n2cnc3c(=O)[nH]c(N)nc32)[C@H](OCCSSCCCCCCCCCCCC)[C@@H]1O*]

In some embodiments, provided herein are RNAi agent comprising a sense stand and an antisense strand, wherein the sense strand and the antisense strand form a duplex region, wherein the sense strand or the antisense strand comprises a modified nucleotide of any one of Formula XIII-XVI:

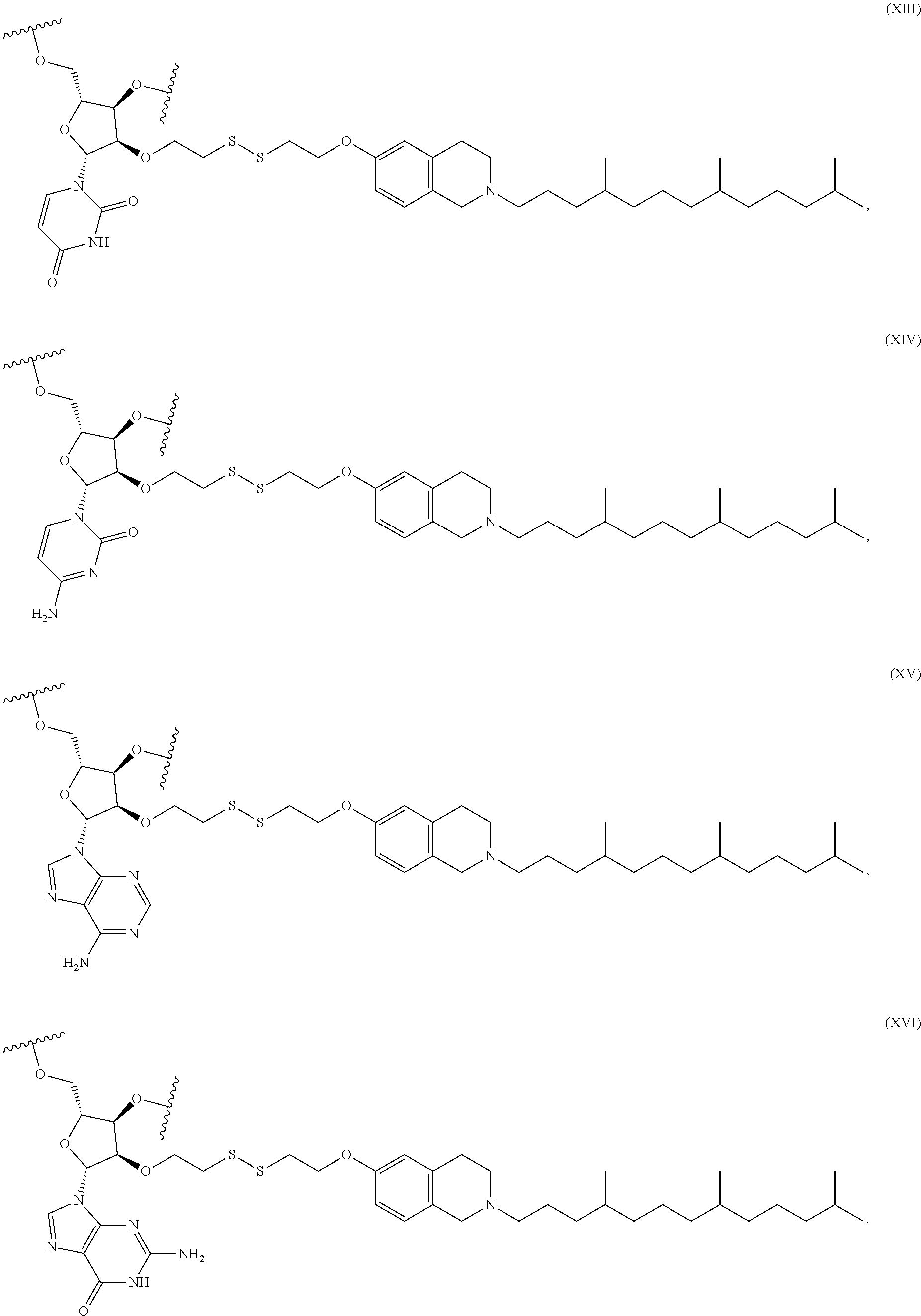 [SMILES: *OC[C@H]1O[C@@H](n2ccc(=O)[nH]c2=O)[C@H](OCCSSCCOc2ccc3c(c2)CCN(CCCC(C)CCCC(C)CCCC(C)C)C3)[C@@H]1O* ; *OC[C@H]1O[C@@H](n2ccc(N)nc2=O)[C@H](OCCSSCCOc2ccc3c(c2)CCN(CCCC(C)CCCC(C)CCCC(C)C)C3)[C@@H]1O* ; *OC[C@H]1O[C@@H](n2cnc3c(N)ncnc32)[C@H](OCCSSCCOc2ccc3c(c2)CCN(CCCC(C)CCCC(C)CCCC(C)C)C3)[C@@H]1O* ; *OC[C@H]1O[C@@H](n2cnc3c(=O)[nH]c(N)nc32)[C@H](OCCSSCCOc2ccc3c(c2)CCN(CCCC(C)CCCC(C)CCCC(C)C)C3)[C@@H]1O*]

In some embodiments, provided herein are RNAi agent comprising a sense stand and an antisense strand, wherein the sense strand and the antisense strand form a duplex region, wherein the sense strand or the antisense strand comprises a modified nucleotide of any one of Formula XVII-XX:

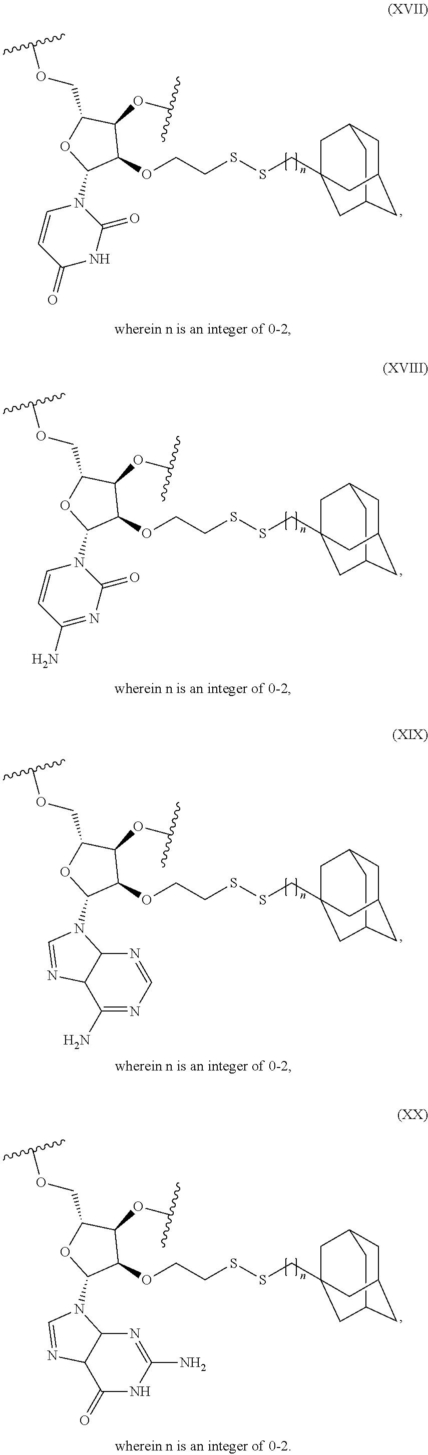

In some embodiments, n is 0 in Formula XVII-XX. In some embodiments, n is 1 in Formula XVII-XX. In some embodiments, n is 2 in Formula XVII-XX.

In some embodiments, provided herein are RNAi agent comprising a sense stand and an antisense strand, wherein the sense strand and the antisense strand form a duplex region, wherein the sense strand or the antisense strand comprises a modified nucleotide of any one of Formula XXII-XXV:

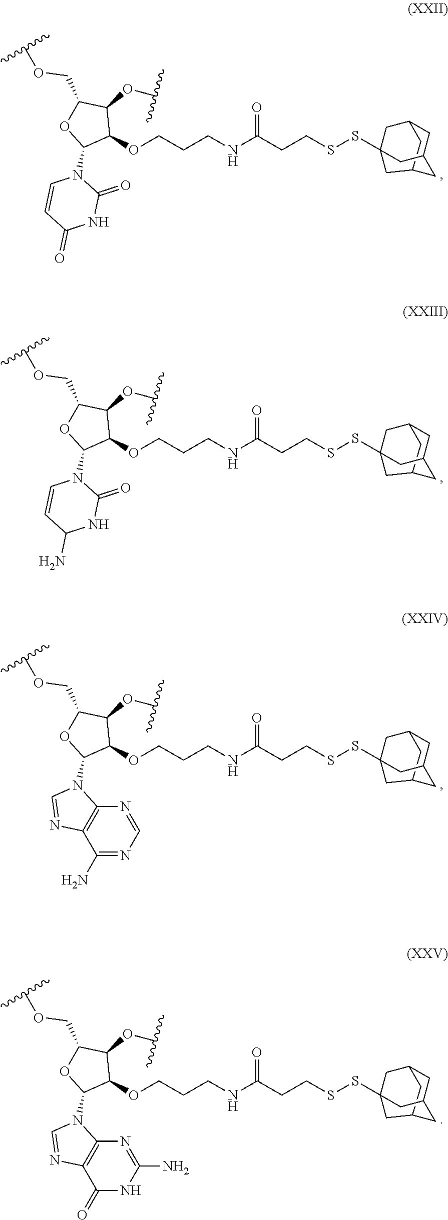 [SMILES: *OC[C@H]1O[C@@H](n2ccc(=O)[nH]c2=O)[C@H](OCCCNC(=O)CCSSC23CC4CC(CC(C4)C2)C3)[C@@H]1O* ; *OC[C@H]1O[C@@H](N2C=CC(N)NC2=O)[C@H](OCCCNC(=O)CCSSC23CC4CC(CC(C4)C2)C3)[C@@H]1O* ; *OC[C@H]1O[C@@H](n2cnc3c(N)ncnc32)[C@H](OCCCNC(=O)CCSSC23CC4CC(CC(C4)C2)C3)[C@@H]1O* ; *OC[C@H]1O[C@@H](n2cnc3c(=O)[nH]c(N)nc32)[C@H](OCCCNC(=O)CCSSC23CC4CC(CC(C4)C2)C3)[C@@H]1O*]

In some embodiments, the sense strand is 15 to 50 nucleotides in length. In some embodiments, the antisense strand is 15 to 30 nucleotides in length. In some embodiments, both the sense strand and the antisense strand are 15 to 30 nucleotides in length, e.g., 20 to 25 nucleotides in length. In some embodiments, the sense strand is 21 nucleotides in length. In some embodiments, the antisense strand is 23 nucleotides in length. In some embodiments, the sense strand is 21 nucleotides in length and the antisense strand is 23 nucleotides in length. In some embodiments, the duplex region is 15 to 21 nucleotides in length. In some embodiments, the duplex region is 21 nucleotides in length. In some embodiments, the sense strand and antisense strand may have overhangs at either the 5′ end or the 3′ end (i.e., 5′ overhang or 3′ overhang). For example, the sense strand and the antisense strand may have 5′ or 3′ overhangs of 1 to 5 nucleotides or 1 to 3 nucleotides. In some embodiments, the antisense strand comprises a 3′ overhang of two nucleotides.

In some embodiments, the sense strand comprises the modified nucleotide of any one of Formula Ia, Ib, Ic, II-IV, Va, Vb, Vc, VIa, VIb, VIc, VIIa, VIIb, VIIc, VIIIa, V1IIb, VIIIc, IX-XXV. In some embodiments, the sense strand comprises the modified nucleotide of any one of Formula Ia, Ib, Ic, II-IV, Va, Vb, Vc, VIa, VIb, VIc, VIIa, VIIb, VIIc, VIIIa, VIIIb, VIIIc, IX-XXV, e.g., at any one of positions 1-6 or 12-21 from the 5′ end. In some embodiments, the sense strand comprises the modified nucleotide of any one of Formula Ia, Ib, Ic, II-IV, Va, Vb, Vc, VIa, VIb, VIc, VIIa, VIIb, VIIc, VIIIa, VIIIb, VIIIc, IX-XXV at position 13 from the 5′ end.

In some embodiments, the antisense strand comprises the modified nucleotide of any one of Formula Ia, Ib, Ic, II-IV, Va, Vb, Vc, VIa, VIb, VIc, VIIa, VIIb, VIIc, VIIIa, VIIIb, VIIIc, IX-XXV. In some embodiments, the antisense strand comprises the modified nucleotide of any one of Formula Ia, Ib, Ic, II-IV, Va, Vb, Vc, VIa, VIb, VIc, VIIa, VIIb, VIIc, VIIIa, VIIIb, VIIIc, IX-XXV, e.g., at any one of positions 6-10 or 15-18 from the 5′ end.

In some embodiments, the sense strand and antisense strand further comprise one or more 2′-fluoro modified nucleotides and 2′-O-methyl modified nucleotides. In some embodiments, the sense strand comprises four 2′-fluoro modified nucleotides at positions 7, 9, 10, and 11 from the 5′ end of the sense strand. In some embodiments, the sense strand comprises four and only four 2′-fluoro modified nucleotides at positions 7, 9, 10, and 11 from the 5′ end of the sense strand. In some embodiments, the nucleotides at the other positions of the sense strand are 2′-O-methyl modified nucleotides.

In some embodiments, the antisense strand comprises four 2′-fluoro modified nucleotides at positions 2, 6, 14, and 16 from the 5′ end of the antisense strand. In some embodiments, the antisense strand comprises four and only four 2′-fluoro modified nucleotides at positions 2, 6, 14, and 16 from the 5′ end of the antisense strand. In some embodiments, the nucleotides at the other positions of the antisense strand are 2′-O-methyl modified nucleotides.

In some embodiments, the sense strand comprises three 2′-fluoro modified nucleotides at positions 9, 10, and 11 from the 5′ end of the sense strand. In some embodiments, the sense strand comprises three and only three 2′-fluoro modified nucleotides at positions 9, 10, and 11 from the 5′ end of the sense strand. In some embodiments, the nucleotides at the other positions of the sense strand are 2′-O-methyl modified nucleotides.

In some embodiments, the antisense strand comprises five 2′-fluoro modified nucleotides at positions 2, 5, 7, 14, and 16 from the 5′ end of the antisense strand. In some embodiments, the antisense strand comprises five and only five 2′-fluoro modified nucleotides at positions 2, 5, 7, 14, and 16 from the 5′ end of the antisense strand. In some embodiments, the nucleotides at the other positions of the antisense strand are 2′-O-methyl modified nucleotides.

In some embodiments, the antisense strand comprises five 2′-fluoro modified nucleotides at positions 2, 5, 8, 14, and 16 from the 5′ end of the antisense strand. In some embodiments, the antisense strand comprises five and only five 2′-fluoro modified nucleotides at positions 2, 5, 8, 14, and 16 from the 5′ end of the antisense strand. In some embodiments, the nucleotides at the other positions of the antisense strand are 2′-O-methyl modified nucleotides.

In some embodiments, the antisense strand comprises five 2′-fluoro modified nucleotides at positions 2, 3, 7, 14, and 16 from the 5′ end of the antisense strand. In some embodiments, the antisense strand comprises five and only five 2′-fluoro modified nucleotides at positions 2, 3, 7, 14, and 16 from the 5′ end of the antisense strand. In some embodiments, the nucleotides at the other positions of the antisense strand are 2′-O-methyl modified nucleotides.

In some embodiments, the sense strand and the antisense strand comprise one or more modified internucleotide linkages, e.g., phosphorothioate linkages. In some embodiments, the sense strand comprises four or five phosphorothioate linkages. In some embodiments, the antisense strand comprises four or five phosphorothioate linkages.

In some embodiments, the antisense strand comprises a phosphate analog at 5′ end. In some embodiments, the antisense strand comprises a 5′-vinylphosphonate at 5′ end.

In some embodiments, the sense strand comprises an abasic moiety or inverted abasic moiety, e.g., an abasic or inverted abasic moiety from Table 3.

In some embodiments, the antisense strand is complementary to a target mRNA selected from SNCA, MAPT, APP, ATXN2, ATXN3, SARM1, APOE, BACE1, FMR1, LRRK2, HIT, SOD1, SCN10A, SCN9A or CACNA1B mRNA. In some embodiments, the antisense strand is complementary to SNCA mRNA. In some embodiments, the antisense strand is complementary to MAPT mRNA.

Exemplary sense strand and antisense strand sequences of RNAi agents targeting human SNCA mRNA (SNCA RNAi agents) are provided in Table 1.

TABLE 1
Nucleic Acid Sequences of Exemplary SNCA RNAi Agents

| | | | | | Start position of |
|---|---|---|---|---|---|
| | | | | | antisense strand |
| SNCA | | | | | target region of |
| RNAi | | | | | human SNCA |
| Agent | Sense Strand | SEQ ID | Antisense Strand | SEQ ID | transcript |
| No. | (5′ to 3′) | NO | (5′ to 3′) | NO | NM_000345.4 |
| 1 | CUGUACAAGUGCUCAG | 1 | UGGAACUGAGCACUUGUA | 2 | 701 |
| | UUCCA | | CAGGA | | |
| 2 | mC*mU*mGmUmAmCfA | 3 | VPmU*fG*mGmAmAfCmUm | 4 | 701 |
| | mAfGfUfGmC(Uads)mCm | | GmAmGmCmAmCfUmUfGm | | |
| | AmGmUmUmC*mC*mA | | UmAmCmAmG*mG*mA | | |
| 3 | (Cads)*mU*mGmUmAmCf | 5 | VPmU*fG*mGmAmAfCmUm | 4 | 701 |
| | AmAfGfUfGmCmUmCmA | | GmAmGmCmAmCfUmUfGm | | |
| | mGmUmUmC*mC*mA | | UmAmCmAmG*mG*mA | | |
| 4 | (Css)*mU*mGmUmAmCm | 6 | VPmU*fG*mGmAfAmCmUf | 7 | 701 |
| | AmAfGfUfGmCmUmCmA | | GmAmGmCmAmCfUmUfGm | | |
| | mGmUmUmC*mC*mA | | UmAmCmAmG*mG*mA | | |
| 5 | mC*mU*mGmUmA(Css)m | 8 | VPmU*fG*mGmAfAmCmUf | 7 | 701 |
| | AmAfGfUfGmCmUmCmA | | GmAmGmCmAmCfUmUfGm | | |
| | mGmUmUmC*mC*mA | | UmAmCmAmG*mG*mA | | |
| 6 | mC*mU*mGmUmAmCmA | 9 | VPmU*fG*mGmAfAmCmUf | 7 | 701 |
| | mAfGfUfG(Css)mUmCmA | | GmAmGmCmAmCfUmUfGm | | |
| | mGmUmUmC*mC*mA | | UmAmCmAmG*mG*mA | | |
| 7 | mC*mU*mGmUmAmCmA | 10 | VPmU*fG*mGmAfAmCmUf | 7 | 701 |
| | mAfGfUfGmCmU(Css)mA | | GmAmGmCmAmCfUmUfGm | | |
| | mGmUmUmC*mC*mA | | UmAmCmAmG*mG*mA | | |
| 8 | mC*mU*mGmUmAmCmA | 11 | VPmU*fG*mGmAfAmCmUf | 7 | 701 |
| | mAfGfUfGmCmUmCmAm | | GmAmGmCmAmCfUmUfGm | | |
| | GmUmU(Css)*mC*mA | | UmAmCmAmG*mG*mA | | |
| 9 | mC*mU*mGmUmAmCmA | 12 | VPmU*fG*mGmAfAmCmUf | 7 | 701 |
| | mAfGfUfGmCmUmCmAm | | GmAmGmCmAmCfUmUfGm | | |
| | GmUmUmC*(Css)*mA | | UmAmCmAmG*mG*mA | | |
| 10 | mC*(Uss)*mGmUmAmCm | 13 | VPmU*fG*mGmAfAmCmUf | 7 | 701 |
| | AmAfGfUfGmCmUmCmA | | GmAmGmCmAmCfUmUfGm | | |
| | mGmUmUmC*mC*mA | | UmAmCmAmG*mG*mA | | |
| 11 | mC*mU*mG(Uss)mAmCm | 14 | VPmU*fG*mGmAfAmCmUf | 7 | 701 |
| | AmAfGfUfGmCmUmCmA | | GmAmGmCmAmCfUmUfGm | | |
| | mGmUmUmC*mC*mA | | UmAmCmAmG*mG*mA | | |
| 12 | mC*mU*mGmUmAmCmA | 15 | VPmU*fG*mGmAfAmCmUf | 7 | 701 |
| | mAfG(Uss)fGmCmUmCm | | GmAmGmCmAmCfUmUfGm | | |
| | AmGmUmUmC*mC*mA | | UmAmCmAmG*mG*mA | | |
| 13 | mC*mU*mGmUmAmCmA | 16 | VPmU*fG*mGmAfAmCmUf | 7 | 701 |
| | mAfGfUfGmC(Uss)mCmA | | GmAmGmCmAmCfUmUfGm | | |
| | mGmUmUmC*mC*mA | | UmAmCmAmG*mG*mA | | |
| 14 | mC*mU*mGmUmAmCmA | 17 | VPmU*fG*mGmAfAmCmUf | 7 | 701 |
| | mAfGfUfGmCmUmCmAm | | GmAmGmCmAmCfUmUfGm | | |
| | G(Uss)mUmC*mC*mA | | UmAmCmAmG*mG*mA | | |
| 15 | mC*mU*mGmUmAmCmA | 18 | VPmU*fG*mGmAfAmCmUf | 7 | 701 |
| | mAfGfUfGmCmUmCmAm | | GmAmGmCmAmCfUmUfGm | | |
| | GmU(Uss)mC*mC*mA | | UmAmCmAmG*mG*mA | | |
| 16 | mC*mU*mGmUmAmCmA | 19 | VPmU*fG*mGmAfAmCmUf | 7 | 701 |
| | mAfGfUfGmC(Uads)mCm | | GmAmGmCmAmCfUmUfGm | | |
| | AmGmUmUmC*mC*mA | | UmAmCmAmG*mG*mA | | |
| 17 | mC*mU*mGmUmAmCfA | 20 | VPmU*fG*mGmAmAfCmUm | 4 | 701 |
| | mAfGfUfGmC(Uss)mCmA | | GmAmGmCmAmCfUmUfGm | | |
| | mGmUmUmC*mC*mA | | UmAmCmAmG*mG*mA | | |
| 18 | mC*mU*mGmUmAmCfA | 37 | VPmU*fG*mGmAfAmCmUf | 7 | 701 |
| | mAfGfUfGmC(UL1)mCm | | GmAmGmCmAmCfUmUfGm | | |
| | AmGmUmUmC*mC*mA | | UmAmCmAmG*mG*mA | | |
| 19 | mC*mU*mGmUmAmCmA | 38 | VPmU*fG*mGmAfAmCmUf | 7 | 701 |
| | mAfGfUfGmC(UL2)mCm | | GmAmGmCmAmCfUmUfGm | | |
| | AmGmUmUmC*mC*mA | | UmAmCmAmG*mG*mA | | |
| 20 | mC*mU*mGmUmAmCmA | 19 | VPmU*fG*mGmAmAmCmU | 66 | 701 |
| | mAfGfUfGmC(Uads)mCm | | mGmAmGmCmAmCfUmUfG | | |
| | AmGmUmUmC*mC*mA | | mUmAmCmAmG*mG*mA | | |
| 21 | mC*mU*mGmUmAmCmA | 67 | VPmU*fG*mGmAfAmCmUf | 7 | 701 |
| | mAfGfUfGmC(UL3)mCm | | GmAmGmCmAmCfUmUfGm | | |
| | AmGmUmUmC*mC*mA | | UmAmCmAmG*mG*mA | | |
| 22 | mG*mU*mAmCAmAfGUf | 68 | VPmU*fG*mGmAfAmCmUf | 7 | 701 |
| | GmC(Uads)mCmAmGmU | | GmAmGmCmAmCfUmUfGm | | |
| | mUmC*mC*mA | | UmAmCmAmG*mG*mA | | |
| 23 | mC*(Uads)*mGmUmAmC | 69 | VPmU*fG*mGmAfAmCmUf | 7 | 701 |
| | mAmAfGfUfGmCmUmCm | | GmAmGmCmAmCfUmUfGm | | |
| | AmGmUmUmC*mC*mA | | UmAmCmAmG*mG*mA | | |
| 24 | mC*mU*(Uads)mUmAmC | 70 | VPmU*fG*mGmAfAmCmUf | 7 | 701 |
| | mAmAfGfUfGmCmUmCm | | GmAmGmCmAmCfUmUfGm | | |
| | AmGmUmUmC*mC*mA | | UmAmCmAmG*mG*mA | | |
| 25 | mC*mU*mG(Uads)mAmC | 71 | VPmU*fG*mGmAfAmCmUf | 7 | 701 |
| | mAmAfGfUfGmCmUmCm | | GmAmGmCmAmCfUmUfGm | | |
| | AmGmUmUmC*mC*mA | | UmAmCmAmG*mG*mA | | |
| 26 | mC*mU*mGmUmA(Cads) | 72 | VPmU*fG*mGmAfAmCmUf | 7 | 701 |
| | mAmAfGfUfGmCmUmCm | | GmAmGmCmAmCfUmUfGm | | |
| | AmGmUmUmC*mC*mA | | UmAmCmAmG*mG*mA | | |
| 27 | mC*mU*mGmUmAmCmA | 73 | VPmU*fG*mGmAfAmCmUf | 7 | 701 |
| | mAfG(Uads)fGmCmUmCm | | GmAmGmCmAmCfUmUfGm | | |
| | AmGmUmUmC*mC*mA | | UmAmCmAmG*mG*mA | | |
| 28 | mC*mU*mGmUmAmCmA | 74 | VPmU*fG*mGmAfAmCmUf | 7 | 701 |
| | mAfGfUfG(Cads)mUmCm | | GmAmGmCmAmCfUmUfGm | | |
| | AmGmUmUmC*mC*mA | | UmAmCmAmG*mG*mA | | |
| 29 | mC*mU*mGmUmAmCmA | 75 | VPmU*fG*mGmAfAmCmUf | 7 | 701 |
| | mAfGfUfGmCmU(Cads)m | | GmAmGmCmAmCfUmUfGm | | |
| | AmGmUmUmC*mC*mA | | UmAmCmAmG*mG*mA | | |
| 30 | mC*mU*mGmUmAmCmA | 76 | VPmU*fG*mGmAfAmCmUf | 7 | 701 |
| | mAfGfUfGmCmUmCmA | | GmAmGmCmAmCfUmUfGm | | |
| | (Uads)mUmUmC*mC*mA | | UmAmCmAmG*mG*mA | | |
| 31 | mC*mU*mGmUmAmCmA | 77 | VPmU*fG*mGmAfAmCmUf | 7 | 701 |
| | mAfGfUfGmCmUmCmAm | | GmAmGmCmAmCfUmUfGm | | |
| | G(Uads)mUmC*mC*mA | | UmAmCmAmG*mG*mA | | |
| 32 | mC*mU*mGmUmAmCmA | 78 | VPmU*fG*mGmAfAmCmUf | 7 | 701 |
| | mAfGfUfGmCmUmCmAm | | GmAmGmCmAmCfUmUfGm | | |
| | GmU(Uads)mC*mC*mA | | UmAmCmAmG*mG*mA | | |
| 33 | mC*mU*mGmUmAmCmA | 79 | VPmU*fG*mGmAfAmCmUf | 7 | 701 |
| | mAfGfUfGmCmUmCmAm | | GmAmGmCmAmCfUmUfGm | | |
| | GmUmU(Uads)*mC*mA | | UmAmCmAmG*mG*mA | | |
| 34 | mC*mU*mGmUmAmCmA | 80 | VPmU*fG*mGmAfAmCmUf | 7 | 701 |
| | mAfGfUfGmCmUmCmAm | | GmAmGmCmAmCfUmUfGm | | |
| | GmUmUmC*(Uads)*mA | | UmAmCmAmG*mG*mA | | |
| 35 | mC*mU*mGmUmAmCmA | 81 | VPmU*fG*mGmAfAmCmUf | 7 | 701 |
| | mAfGfUfGmC(UadsII)mC | | GmAmGmCmAmCfUmUfGm | | |
| | mAmGmUmUmC*mC*mA | | UmAmCmAmG*mG*mA | | |
| 36 | mC*mU*mGmUmAmCmA | 16 | VPmU*fG*mGmAfAmCfUm | 82 | 701 |
| | mAfGfUfGmC(Uss)mCmA | | GmAmGmCmAmCfUmUfGm | | |
| | mGmUmUmC*mC*mA | | UmAmCmAmG*mG*mA | | |
| 37 | mC*mU*mGmUmAmCmA | 9 | VPmU*fG*mGmAfAmCfUm | 82 | 701 |
| | mAfGfUfG(Css)mUmCmA | | GmAmGmCmAmCfUmUfGm | | |
| | mGmUmUmC*mC*mA | | UmAmCmAmG*mG*mA | | |
| 38 | UGUACAAGUGCUCAGU | 83 | UUGGAACUGAGCACUUGU | 84 | 702 |
| | UCCAA | | ACAGG | | |
| 39 | GUACAAGUGCUCAGUU | 85 | UUGGAACUGAGCACUUGU | 86 | 702 |
| | CCAA | | ACAG | | |
| 40 | mU*mG*mUmAmCmAfA | 87 | VPmU*fU*mGmGmAfAmCm | 88 | 702 |
| | mGfUfGfCmU(Css)mAmG | | UmGmAmGmCmAfCmUfUm | | |
| | mUmUmCmC*mA*mA | | GmUmAmCmA*mG*mG | | |
| 41 | mU*mG*mUmAmCmAmA | 89 | VPmU*fU*mGmGfAmAmCf | 90 | 702 |
| | mGfUfGfCmU(Css)mAmG | | UmGmAmGmCmAfCmUfUm | | |
| | mUmUmCmC*mA*mA | | GmUmAmCmA*mG*mG | | |
| 42 | iAbmG*mU*mAmCmAmA | 91 | VPmU*fU*mGmGfAmAmCf | 92 | 702 |
| | mGfUfGfCmU(Css)mAmG | | UmGmAmGmCmAfCmUfUm | | |
| | mUmUmCmC*mA*mA | | GmUmAmC*mA*mG | | |
| 43 | iAbmG*mU*mAmCmAmA | 91 | VPmU*fU*mGmGmAmAmC | 93 | 702 |
| | mGfUfGfCmU(Css)mAmG | | mUmGmAmGmCmAfCmUfU | | |
| | mUmUmCmC*mA*mA | | mGmUmAmC*mA*mG | | |
| 44 | AGUGACUACCACUUAU | 94 | UAGAAAUAAGUGGUAGU | 95 | 926 |
| | UUCUA | | CACUUA | | |
| 45 | GUGACUACCACUUAUU | 96 | UUAGAAAUAAGUGGUAG | 97 | 927 |
| | UCUAA | | UCACUU | | |
| 46 | GAGCAAGUGACAAAUG | 98 | UCCAACAUUUGUCACUUG | 99 | 408 |
| | UUGGA | | CUCUU | | |
| 47 | UUCCAAUGUGCCCAGU | 100 | UCAUGACUGGGCACAUUG | 101 | 717 |
| | CAUGA | | GAACU | | |
| 48 | AAGUGACUACCACUUA | 102 | UGAAAUAAGUGGUAGUC | 103 | 397 |
| | UUUCA | | ACUUAG | | |
| 49 | GACCAAAGAGCAAGUG | 104 | UUUGUCACUUGCUCUUUG | 105 | 921 |
| | ACAAA | | GUCUU | | |
| 50 | mA*mG*mUmGmAmCmU | 106 | VPmU*fA*mGmAfAmAmUf | 107 | 926 |
| | mAfCfCfAmC(Uads)mUm | | AmAmGmUmGmGfUmAfGm | | |
| | AmUmUmUmC*mU*mA | | UmCmAmCmU*mU*mA | | |
| 51 | mG*mU*mGmAmCmUmA | 108 | VPmU*fU*mAmGfAmAmAf | 109 | 927 |
| | mCfCfAfCmU(Uads)mAm | | UmAmAmGmUmGfGmUfAm | | |
| | UmUmUmCmU*mA*mA | | GmUmCmAmC*mU*mU | | |
| 52 | mG*mA*mGmCmAmAmG | 110 | VPmU*fC*mCmAfAmCmAfU | 111 | 408 |
| | mUfGfAfCmA(Aads)mAm | | mUmUmGmUmCfAmCfUmU | | |
| | UmGmUmUmG*mG*mA | | mGmCmUmC*mU*mU | | |
| 53 | mU*mU*mCmCmAmAmU | 112 | VPmU*fC*mAmUfGmAmCfU | 113 | 717 |
| | mGfUfGfCmC(Cads)mAm | | mGmGmGmCmAfCmAfUmU | | |
| | GmUmCmAmU*mG*mA | | mGmGmAmA*mC*mU | | |
| 54 | mA*mA*mGmUmGmAmC | 114 | VPmU*fG*mAmAfAmUmAf | 115 | 397 |
| | mUfAfCfCmA(Cads)mUm | | AmGmUmGmGmUfAmGfUm | | |
| | UmAmUmUmU*mC*mA | | CmAmCmUmU*mA*mG | | |
| 55 | mG*mA*mCmCmAmAmA | 116 | VPmU*fU*mUmGfUmCmAfC | 117 | 921 |
| | mGfAfGfCmA(Aads)mGm | | mUmUmGmCmUfCmUfUmU | | |
| | UmGmAmCmA*mA*mA | | mGmGmUmC*mU*mU | | |
| 56 | mG*mU*mGmAmCmUfA | 118 | VPmU*fU*mAmGmAfAmAm | 119 | 927 |
| | mCfCfAfCmU(Uads)mAm | | UmAmAmGmUmGfGmUfAm | | |
| | UmUmUmCmU*mA*mA | | GmUmCmAmC*mU*mU | | |
| 57 | iAbmA*mG*mUmGmAmC | 120 | VPmU*fA*mGmAmAfAmUm | 121 | 926 |
| | fUmAfCfCfAmC(Uads)mU | | AmAmGmUmGmGfUmAfGm | | |
| | mAmUmUmUmC*mU*mA | | UmCmAmCmU*mU*mA | | |
| 58 | mG*mU*mGmAmCmUmA | 108 | VPmU*fU*mAmGmAmAmA | 122 | 927 |
| | mCfCfAfCmU(Uads)mAm | | mUmAmAmGmUmGfGmUfA | | |
| | UmUmUmCmU*mA*mA | | mGmUmCmAmC*mU*mU | | |
| 59 | iAbmA*mG*mUmGmAmC | 123 | VPmU*fA*mGmAmAmAmU | 124 | 926 |
| | mUmAfCfCfAmC(Uads)m | | mAmAmGmUmGmGfUmAfG | | |
| | UmAmUmUmUmC*mU*m | | mUmCmAmCmU*mU*mA | | |
| | A | | | | |

Abbreviations-“m” indicates 2′-OMe; “f” indicated 2′-fluoro; “*” indicates phosphorothioate linkage; “VP” indicates 5′-vinylphosphonate; “ads” indicates Formula I(a); “ss” indicates Formula II; “L1” indicates Formula III; “L2” indicates Formula IV where n is 0; “L3” indicates Formula XXI; “adsII” indicates Formula I(b); “iAb” indicates inverted abasic in Table 3.

In some embodiments, provided herein are SNCA RNAi agents comprising a sense strand and an antisense strand that comprise a pair of nucleic acid sequences selected from the group consisting of:
(a) the sense strand comprises a first nucleic acid sequence having at least 9000 (e.g., about 90%, about 91%, about 92%, about 93%, about 94%, about 95%, about 96%, about 97%, about 98%, about 99%, or about 1000%) sequence identity to SEQ ID NO: 1, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 2;
(b) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 83, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 84;
(c) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 85, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 86;
(d) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 94, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 95;
(e) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 96, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 97;
(f) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 98, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 99;
(g) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 100, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 101;
(h) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 102, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 103; and
(i) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 104, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 105.

In some embodiments, provided herein are SNCA RNAi agents comprising a sense strand and an antisense strand that comprise a pair of nucleic acid sequences selected from the group consisting of:
(a) the sense strand comprises SEQ ID NO: 1, and the antisense strand comprises SEQ ID NO: 2;
(b) the sense strand comprises any one of SEQ ID NOs: 3, 5, or 20, and the antisense strand comprises SEQ ID NO: 4;
(c) the sense strand comprises any one of SEQ ID NOs: 6, 8-19, 37, 38, or 67-81, and the antisense strand comprises SEQ ID NO: 7;
(d) the sense strand comprises SEQ ID NO: 19, and the antisense strand comprises SEQ ID NO: 66;
(e) the sense strand comprises SEQ ID NO: 9 or 16, and the antisense strand comprises SEQ ID NO: 82;
(f) the sense strand comprises SEQ ID NO: 83, and the antisense strand comprises SEQ ID NO: 84;
(g) the sense strand comprises SEQ ID NO: 85, and the antisense strand comprises SEQ ID NO: 86;
(h) the sense strand comprises SEQ ID NO: 87, and the antisense strand comprises SEQ ID NO: 88;
(i) the sense strand comprises SEQ ID NO: 89, and the antisense strand comprises SEQ ID NO: 90;
(j) the sense strand comprises SEQ ID NO: 91, and the antisense strand comprises SEQ ID NO: 92 or 93;
(k) the sense strand comprises SEQ ID NO: 94, and the antisense strand comprises SEQ ID NO: 95;
(l) the sense strand comprises SEQ ID NO: 96, and the antisense strand comprises SEQ ID NO: 97;
(m) the sense strand comprises SEQ ID NO: 98, and the antisense strand comprises SEQ ID NO: 99;
(n) the sense strand comprises SEQ ID NO: 100, and the antisense strand comprises SEQ ID NO: 101;
(o) the sense strand comprises SEQ ID NO: 102, and the antisense strand comprises SEQ ID NO: 103;
(p) the sense strand comprises SEQ ID NO: 104, and the antisense strand comprises SEQ ID NO: 105;
(q) the sense strand comprises SEQ ID NO: 106, and the antisense strand comprises SEQ ID NO: 107;
(r) the sense strand comprises SEQ ID NO: 108, and the antisense strand comprises SEQ ID NO: 109 or 122;
(s) the sense strand comprises SEQ ID NO: 110, and the antisense strand comprises SEQ ID NO: 111;
(t) the sense strand comprises SEQ ID NO: 112, and the antisense strand comprises SEQ ID NO: 113;
(u) the sense strand comprises SEQ ID NO: 114, and the antisense strand comprises SEQ ID NO: 115;
(v) the sense strand comprises SEQ ID NO: 116, and the antisense strand comprises SEQ ID NO: 117;
(w) the sense strand comprises SEQ ID NO: 118, and the antisense strand comprises SEQ ID NO: 119;
(x) the sense strand comprises SEQ ID NO: 120, and the antisense strand comprises SEQ ID NO: 121; and
(y) the sense strand comprises SEQ ID NO: 123, and the antisense strand comprises SEQ ID NO: 124.

In some embodiments, provided herein are SNCA RNAi agent comprising a sense strand comprising SEQ ID NO: 3, and an antisense strand comprising SEQ ID NO: 4. In some embodiments, provided herein are SNCA RNAi agent comprising a sense strand comprising SEQ ID NO: 19, and an antisense strand comprising SEQ ID NO: 7. In some embodiments, provided herein are SNCA RNAi agent comprising a sense strand comprising SEQ ID NO: 87, and an antisense strand comprising SEQ ID NO: 88. In some embodiments, provided herein are SNCA RNAi agent comprising a sense strand comprising SEQ ID NO: 89, and an antisense strand comprising SEQ ID NO: 90.

In some embodiments, provided herein are SNCA RNAi agents comprising a sense strand and an antisense strand that consist of a pair of nucleic acid sequences selected from the group consisting of:
(a) the sense strand consists of any one of SEQ ID NOs: 3, 5, or 20, and the antisense strand consists of SEQ ID NO: 4;
(b) the sense strand consists of any one of SEQ ID NOs: 6, 8-19, 37, 38, or 67-81, and the antisense strand consists of SEQ ID NO: 7;
(c) the sense strand consists of SEQ ID NO: 19, and the antisense strand consists of SEQ ID NO: 66;
(d) the sense strand consists of SEQ ID NO: 9 or 16, and the antisense strand consists of SEQ ID NO: 82;
(e) the sense strand consists of SEQ ID NO: 87, and the antisense strand consists of SEQ ID NO: 88;
(f) the sense strand consists of SEQ ID NO: 89, and the antisense strand consists of SEQ ID NO: 90;
(g) the sense strand consists of SEQ ID NO: 91, and the antisense strand consists of SEQ ID NO: 92 or 93;
(h) the sense strand consists of SEQ ID NO: 106, and the antisense strand consists of SEQ ID NO: 107;
(i) the sense strand consists of SEQ ID NO: 108, and the antisense strand consists of SEQ ID NO: 109 or 122;
(j) the sense strand consists of SEQ ID NO: 110, and the antisense strand consists of SEQ ID NO: 111;
(k) the sense strand consists of SEQ ID NO: 112, and the antisense strand consists of SEQ ID NO: 113;
(l) the sense strand consists of SEQ ID NO: 114, and the antisense strand consists of SEQ ID NO: 115;
(m) the sense strand consists of SEQ ID NO: 116, and the antisense strand consists of SEQ ID NO: 117;
(n) the sense strand consists of SEQ ID NO: 118, and the antisense strand consists of SEQ ID NO: 119;
(o) the sense strand consists of SEQ ID NO: 120, and the antisense strand consists of SEQ ID NO: 121; and
(p) the sense strand consists of SEQ ID NO: 123, and the antisense strand consists of SEQ ID NO: 124.

In some embodiments, provided herein are SNCA RNAi agent comprising a sense strand consisting of SEQ ID NO: 3, and an antisense strand consisting of SEQ ID NO: 4. In some embodiments, provided herein are SNCA RNAi agent comprising a sense strand consisting of SEQ ID NO: 19, and an antisense strand consisting of SEQ ID NO: 7. In some embodiments, provided herein are SNCA RNAi agent comprising a sense strand consisting of SEQ ID NO: 87, and an antisense strand consisting of SEQ ID NO: 88. In some embodiments, provided herein are SNCA RNAi agent comprising a sense strand consisting of SEQ ID NO: 89, and an antisense strand consisting of SEQ ID NO: 90.

Exemplary sense strand and antisense strand sequences of RNAi agents targeting human MAPT mRNA (MAPT RNAi agent) are provided in Table 2.

TABLE 2
Nucleic Acid Sequences of Exemplary MAPT RNAi Agents

| | | | | | Start |
|---|---|---|---|---|---|
| | | | | | position |
| | | | | | of antisense |
| | | | | | strand |
| | | | | | target |
| | | | | | region of |
| MAPT | | | | | human MAPT |
| RNAi | | SEQ | | SEQ | transcript |
| Agent | Sense Strand | ID | Antisense Strand | ID | NM_ |
| No. | (5′ to 3′) | NO | (5′ to 3′) | NO | 001123067.4 |
| 1 | GUGGAAGUAAAAUCUG | 21 | UUUCUCAGAUUUUACUU | 22 | 1070 |
| | AGAAA | | CCACCU | | |
| 2 | CCAAGUGUGGCUCAUU | 23 | UGCCUAAUGAGCCACACU | 24 | 1020 |
| | AGGCA | | UGGAG | | |
| 3 | UGCAAAUAGUCUACAA | 25 | UUGGUUUGUAGACUAUU | 26 | 978* |
| | ACCAA | | UGCACC | | |
| 4 | mG*mU*mGmGmAmAmG | 27 | VPmU*fU*mUmCfUmCmAf | 28 | 1070 |
| | mUfAfAfAmA(Uads)mCmU | | GmAmUmUmUmUfAmCfUm | | |
| | mGmAmGmA*mA*mA | | UmCmCmAmC*mC*mU | | |
| 5 | mC*mC*mAmAmGmUmG | 29 | VPmU*fG*mCmCfUmAmAf | 30 | 1020 |
| | mUfGfGfC(Uads)mCmAmU | | UmGmAmGmCmCfAmCfAm | | |
| | mUmAmGmG*mC*mA | | CmUmUmGmG*mA*mG | | |
| 6 | mU*mG*mCmAmAmAmU | 31 | VPmU*fU*mGmGfUmUmUf | 32 | 978* |
| | mAfGfUfC(Uads)mAmCmA | | GmUmAmGmAmCfUmAfUm | | |
| | mAmAmCmC*mA*mA | | UmUmGmCmA*mC*mC | | |
| 7 | mG*(Uads)*mGmGmAmA | 33 | VPmU*fU*mUmCfUmCmAf | 28 | 1070 |
| | mGmUfAfAfAmAmUmCm | | GmAmUmUmUmUfAmCfUm | | |
| | UmGmAmGmA*mA*mA | | UmCmCmAmC*mC*mU | | |
| 8 | mC*mC*mAmAmGmUmG | 34 | VPmU*fG*mCmCfUmAmAf | 30 | 1020 |
| | mUfGfGfCmU(Cads)mAmU | | UmGmAmGmCmCfAmCfAm | | |
| | mUmAmGmG*mC*mA | | CmUmUmGmG*mA*mG | | |
| 9 | (Cads)*mC*mAmAmGmU | 35 | VPmU*fG*mCmCfUmAmAf | 30 | 1020 |
| | mGmUfGfGfCmUmCmAm | | UmGmAmGmCmCfAmCfAm | | |
| | UmUmAmGmG*mC*mA | | CmUmUmGmG*mA*mG | | |
| 10 | (Uads)*mG*mCmAmAmA | 36 | VPmU*fU*mGmGfUmUmUf | 32 | 978* |
| | mUmAfGfUfCmUmAmCm | | GmUmAmGmAmCfUmAfUm | | |
| | AmAmAmCmC*mA*mA | | UmUmGmCmA*mC*mC | | |
| 11 | mG*mU*mGmGmAmAmG | 39 | VPmU*fU*mUmCfUmCmAf | 28 | 1070 |
| | mUfAfAfAmA(Uss)mCmU | | GmAmUmUmUmUfAmCfUm | | |
| | mGmAmGmA*mA*mA | | UmCmCmAmC*mC*mU | | |
| 12 | mG*mU*mGmGmAmAmG | 40 | VPmU*fU*mUmCfUmCmAf | 28 | 1070 |
| | mUnfAfAmA(Uss)mCmUm | | GmAmUmUmUmUfAmCfUm | | |
| | GmAmGmA*mA*mA | | UmCmCmAmC*mC*mU | | |
| 13 | mG*mU*mGmGmAmAmG | 39 | VPmU*fU*mUmCmUfCmAm | 41 | 1070 |
| | mUfAfAfAmA(Uss)mCmU | | GmAfUmUmUmUfAmCfUm | | |
| | mGmAmGmA*mA*mA | | UfCmCfAmC*mC*mU | | |
| 14 | mG*mU*mGmGmAmAmG | 40 | VPmU*fU*mUmCmUfCmAm | 41 | 1070 |
| | mUnfAfAmA(Uss)mCmUm | | GmAfUmUmUmUfAmCfUm | | |
| | GmAmGmA*mA*mA | | UfCmCfAmC*mC*mU | | |
| 15 | mC*mC*mAmAmGmUmG | 42 | VPmU*fG*mCmCfUmAmAf | 30 | 1020 |
| | mUfGfGfC(Uss)mCmAmU | | UmGmAmGmCmCfAmCfAm | | |
| | mUmAmGmG*mC*mA | | CmUmUmGmG*mA*mG | | |
| 16 | mU*mG*mCmAmAmAmU | 43 | VPmU*fU*mGmGfUmUmUf | 32 | 978* |
| | mAfGfUfC(Uss)mAmCmA | | GmUmAmGmAmCfUmAfUm | | |
| | mAmAmCmC*mA*mA | | UmUmGmCmA*mC*mC | | |
| 17 | mC*mC*mAmGmGmUmG | 44 | VPmU*fC*mAmGfAmUmUf | 45 | 1066 |
| | mGfAfAfG(Uss)mAmAmA | | UmUmAmCmUmUfCmCfAm | | |
| | mAmUmCmU*mG*mA | | CmCmUmGmG*mC*mC | | |
| 18 | mC*mC*mAmGmGmUmG | 46 | VPmU*fC*mAmGfAmUmUf | 45 | 1066 |
| | mGfAfAfGmUmAmAmAm | | UmUmAmCmUmUfCmCfAm | | |
| | AmUmC(Uss)*mG*mA | | CmCmUmGmG*mC*mC | | |
| 19 | mG*mU*mGmGmAmAmG( | 47 | VPmU*fU*mUmCfUmCmAf | 28 | 1070 |
| | Uss)fAfAfAmAmUmCmUm | | GmAmUmUmUmUfAmCfUm | | |
| | GmAmGmA*mA*mA | | UmCmCmAmC*mC*mU | | |
| 20 | mG*(Uss)*mGmGmAmAm | 48 | VPmU*fU*mUmCfUmCmAf | 28 | 1070 |
| | GmUfAfAfAmAmUmCmU | | GmAmUmUmUmUfAmCfUm | | |
| | mGmAmGmA*mA*mA | | UmCmCmAmC*mC*mU | | |
| 21 | mG*mU*mGmGmAmAmG | 49 | VPmU*fU*mUmCfUmCmAf | 28 | 1070 |
| | mUfAfAfAmAmUmC(Uss) | | GmAmUmUmUmUfAmCfUm | | |
| | mGmAmGmA*mA*mA | | UmCmCmAmC*mC*mU | | |
| 22 | mC*mC*mAmAmG(Uss)m | 50 | VPmU*fG*mCmCfUmAmAf | 30 | 1020 |
| | GmUfGfGfCmUmCmAmU | | UmGmAmGmCmCfAmCfAm | | |
| | mUmAmGmG*mC*mA | | CmUmUmGmG*mA*mG | | |
| 23 | mC*mC*mAmAmGmUmG | 51 | VPmU*fG*mCmCfUmAmAf | 30 | 1020 |
| | mUfGfGfCmUmCmA(Uss) | | UmGmAmGmCmCfAmCfAm | | |
| | mUmAmGmG*mC*mA | | CmUmUmGmG*mA*mG | | |
| 24 | (Uss)*mG*mCmAmAmAm | 52 | VPmU*fU*mGmGfUmUmUf | 32 | 978* |
| | UmAfGfUfCmUmAmCmA | | GmUmAmGmAmCfUmAfUm | | |
| | mAmAmCmC*mA*mA | | UmUmGmCmA*mC*mC | | |
| 25 | mC*mC*mAmAmGmUmG | 53 | VPmU*fG*mCmCfUmAmAf | 30 | 1020 |
| | mUfGfGfCmU(Css)mAmU | | UmGmAmGmCmCfAmCfAm | | |
| | mUmAmGmG*mC*mA | | CmUmUmGmG*mA*mG | | |
| 26 | mC*mC*mAmAmGmUmG | 53 | VPmU*fG*mCmCmUfAmAm | 54 | 1020 |
| | mUfGfGfCmU(Css)mAmU | | UmGfAmGmCmCfAmCfAmC | | |
| | mUmAmGmG*mC*mA | | fUmUfGmG*mA*mG | | |
| 27 | mC*mC*mAmAmGmUmG | 53 | VPmU*fG*mCmCfUmAfAm | 55 | 1020 |
| | mUfGfGfCmU(Css)mAmU | | UmGmAmGmCmCfAmCfAm | | |
| | mUmAmGmG*mC*mA | | CmUmUmGmG*mA*mG | | |
| 28 | CCAGGUGGAAGUAAAA | 56 | UCAGAUUUUACUUCCACC | 57 | 1066 |
| | UCUGA | | UGGCC | | |
| 29 | AUUAGGCAACAUCCAU | 125 | UUAUGAUGGAUGUUGCC | 126 | 1034 |
| | CAUAA | | UAAUGA | | |
| 30 | GGCUUUGGCUCGGGAC | 127 | UUGAAGUCCCGAGCCAAA | 128 | 1539 |
| | UUCAA | | GCCGA | | |
| 31 | GCAAAUAGUCUACAAA | 129 | UCUGGUUUGUAGACUAU | 130 | 979 |
| | CCAGA | | UUGCAC | | |
| 32 | AAAUAAAAAGAUUGAA | 131 | UGGGUUUCAAUCUUUUU | 132 | 1162 |
| | ACCCA | | AUUUCC | | |
| 33 | GCAAGGUGACCUCCAA | 133 | UACACUUGGAGGUCACCU | 134 | 1008 |
| | GUGUA | | UGCUC | | |
| 34 | AGAUUGAAACCCACAA | 135 | UCAGCUUGUGGGUUUCA | 136 | 1170 |
| | GCUGA | | AUCUUU | | |
| 36 | mA*mU*mUmAmGmGmC | 137 | VPmU*fU*mAmUmGmAmU | 138 | 1034 |
| | mAfAfCfAmU(Cads)mCmA | | mGmGmAmUmGmUfUmGfC | | |
| | mUmCmAmU*mA*mA | | mCmUmAmAmU*mG*mA | | |
| 37 | mG*mG*mCmUmUmUmG | 139 | VPmU*fU*mGmAmAmGmU | 140 | 1539 |
| | mGfCfUfCmG(Gads)mGmA | | mCmCmCmGmAmGfCmCfA | | |
| | mCmUmUmC*mA*mA | | mAmAmGmCmC*mG*mA | | |
| 38 | mG*mC*mAmAmAmUmA | 141 | VPmU*fC*mUmGmGmUmU | 142 | 979 |
| | mGfUfCfUmA(Cads)mAmA | | mUmGmUmAmGmAfCmUfA | | |
| | mAmCmCmA*mG*mA | | mUmUmUmGmC*mA*mC | | |
| 39 | mA*mA*mAmUmAmAfAm | 143 | VPmU*fG*mGmGmUmUmU | 144 | 1162 |
| | AfAfGfAmU(Uads)mGmA | | mCmAmAmUmCmUfUmUfU | | |
| | mAmAmCmC*mC*mA | | mUmAmUmUmU*mC*mC | | |
| 40 | mG*mC*mAmAmGmGmU | 145 | VPmU*fA*mCmAmCmUmU | 146 | 1008 |
| | mGfAfCfCmU(Cads)mCmA | | mGmGmAmGmGmUfCmAfC | | |
| | mAmGmUmG*mU*mA | | mCmUmUmGmC*mU*mC | | |
| 41 | mA*mG*mAmUmUmGmA | 147 | VPmU*fC*mAmGmCmUmU | 148 | 1170 |
| | mAfAfCfCmC(Aads)mCmA | | mGmUmGmGmGmUfUmUfC | | |
| | mAmGmCmU*mG*mA | | mAmAmUmCmU*mU*mU | | |
| 42 | mC*mC*mAmAmGmUmG | 34 | VPmU*fG*mCmCfUmAmAf | 149 | 1020 |
| | mUfGfGfCmU(Cads)mAmU | | UmGmAmGmCmCfAmCfAm | | |
| | mUmAmGmG*mC*mA | | CmUmUmGmG*mA | | |
| 43 | mC*mC*mAmAmGmUmG | 34 | VPmU*fG*mCmCfUmAmAf | 150 | 1020 |
| | mUfGfGfCmU(Cads)mAmU | | UmGmAmGmCmCfAmCfAm | | |
| | mUmAmGmG*mC*mA | | CmUmUmGmG | | |
| 44 | mC*mC*mAmAmGmUmG | 34 | VPmU*fG*mCmCmUmAmA | 151 | 1020 |
| | mUfGfGfCmU(Cads)mAmU | | mUmGmAmGmCmCfAmCfA | | |
| | mUmAmGmG*mC*mA | | mCmUmUmGmG*mA*mG | | |
| 45 | mU*mG*mCmAmAmAmU | 31 | VPmU*fU*mGmGmUfUmUm | 152 | 978* |
| | mAfGfUfC(Uads)mAmCmA | | GmUmAmGmAmCfUmAfUm | | |
| | mAmAmCmC*mA*mA | | UmUmGmCmA*mC*mC | | |
| 46 | mU*mG*mCmAmAmAmU | 31 | VPmU*dT*mGmGdTmUmUf | 153 | 978* |
| | mAfGfUfC(Uads)mAmCmA | | GmUmAmGmAmCfUmAfUm | | |
| | mAmAmCmC*mA*mA | | UmUmGmCmA*mC*mC | | |
| 47 | mU*mG*mCmAmAmAmU | 154 | VPmU*fU*mGmGfUmUmUf | 32 | 978* |
| | mAfGfUfC(UadsII)mAmCm | | GmUmAmGmAmCfUmAfUm | | |
| | AmAmAmCmC*mA*mA | | UmUmGmCmA*mC*mC | | |
| 48 | mU*mG*mCmAmAmAmU | 155 | VPmU*fU*mGmGfUmUmUf | 32 | 978* |
| | mAfGfUfCmU(Aads)mCmA | | GmUmAmGmAmCfUmAfUm | | |
| | mAmAmCmC*mA*mA | | UmUmGmCmA*mC*mC | | |
| 49 | mU*mG*mCmAmAmAmU | 31 | VPmU*fU*mGmGmUmUmU | 156 | 978* |
| | mAfGfUfC(Uads)mAmCmA | | mGmUmAmGmAmCfUmAfU | | |
| | mAmAmCmC*mA*mA | | mUmUmGmCmA*mC*mC | | |
| 50 | mU*mG*mCmAmAmAmU | 31 | VPmU*dT*mGmGdTmUmUd | 157 | 978* |
| | mAfGfUfC(Uads)mAmCmA | | GmUmAmGmAmCfUmAfUm | | |
| | mAmAmCmC*mA*mA | | UmUmGmCmA*mC*mC | | |
| 51 | mU*mG*mCmAmAmAmU | 31 | VPmU*fU*mGmGnmUmUfG | 158 | 978* |
| | mAfGfUfC(Uads)mAmCmA | | mUmAmGmAmCfUmAfUmU | | |
| | mAmAmCmC*mA*mA | | mUmGmCmA*mC*mC | | |
| 52 | mU*mG*mCmAmAmAmU | 31 | VPmU*fU*mGmGfUmUnfGm | 159 | 978* |
| | mAfGfUfC(Uads)mAmCmA | | UmAmGmAmCfUmAfUmUm | | |
| | mAmAmCmC*mA*mA | | UmGmCmA*mC*mC | | |
| 53 | mU*mG*mCmAmAmAfUm | 160 | VPmU*fU*mGmGmUfUmUm | 152 | 978* |
| | AfGfUfC(Uads)mAmCmAm | | GmUmAmGmAmCfUmAfUm | | |
| | AmAmCmC*mA*mA | | UmUmGmCmA*mC*mC | | |
| 54 | mU*mG*mCmAmAmAmU | 161 | VPmU*fU*mGmGfUmUmUf | 32 | 978* |
| | mAfGfUfCmUmA(Uads)mA | | GmUmAmGmAmCfUmAfUm | | |
| | mAmAmCmC*mA*mA | | UmUmGmCmA*mC*mC | | |
| 55 | mU*mG*mCmAmAmAmU | 162 | VPmU*fU*mGmGfUmUmUf | 32 | 978* |
| | mAfGfUfCmUmAmCmAm | | GmUmAmGmAmCfUmAfUm | | |
| | AmAmC(Cads)mA*mA | | UmUmGmCmA*mC*mC | | |
| 56 | mU*mG*(Cads)mAmAmA | 163 | VPmU*fU*mGmGfUmUmUf | 32 | 978* |
| | mUmAfGfUfCmUmAmCm | | GmUmAmGmAmCfUmAfUm | | |
| | AmAmAmCmC*mA*mA | | UmUmGmCmA*mC*mC | | |
| 57 | mU*mG*mCmAmAmAmU | 31 | VPmU*fU*mGmGfUmUfUm | 164 | 978* |
| | mAfGfUfC(Uads)mAmCmA | | GmUmAmGmAmCfUmAfUm | | |
| | mAmAmCmC*mA*mA | | UmUmGmCmA*mC*mC | | |
| 58 | mU*mG*mCmAmAmAmU | 31 | VPmU*fU*mGmGfUmUmUf | 165 | 978* |
| | mAfGfUfC(Uads)mAmCmA | | GmUmAmGmAmCfUmAfUm | | |
| | mAmAmCmC*mA*mA | | UmUmGmC*mA*mC | | |
| 59 | mU*mG*mCmAmAmAmU | 166 | VPmU*fU*mGmGmUmUmU | 156 | 978* |
| | mAfGfUfC(UL3)mAmCmA | | mGmUmAmGmAmCfUmAfU | | |
| | mAmAmCmC*mA*mA | | mUmUmGmCmA*mC*mC | | |
| 60 | mU*mG*mCmAmAmAmU | 43 | VPmU*fU*mGmGmUmUmU | 156 | 978* |
| | mAfGfUfC(Uss)mAmCmA | | mGmUmAmGmAmCfUmAfU | | |
| | mAmAmCmC*mA*mA | | mUmUmGmCmA*mC*mC | | |

* The last nucleotide does not match the transcript.
Abbreviations - “m” indicates 2′-OMe; “f” indicated 2′-fluoro; “*” indicates phosphorothioate linkage; “VP” indicates 5′-vinylphosphonate; “n” indicates abasic nucleotide; “ads” indicates Formula I(a); “ss” indicates Formula II; “L3” indicates Formula XXI; “adsII” indicates Formula I(b).

TABLE 3
Abasic or inverted abasic (iAb) moieties

| | Structure |
|---|---|
| 1 (abasic) | 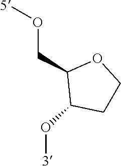 |
| 2 (iAb) | 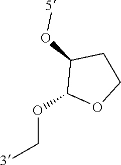 |

“5′” and “3′” indicate the 5′ to 3′ direction of the sequences.

In some embodiments, provided herein are MAPT RNAi agents comprising a sense strand and an antisense strand that comprise a pair of nucleic acid sequences selected from the group consisting of:
(a) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 21, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 22;
(b) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 23, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 24;
(c) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 25, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 26;
(d) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 56, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO:57;
(e) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 125, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 126;
(f) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO:127, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 128;
(g) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO:129, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 130;
(h) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 131, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 132;
(i) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 133, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 134; and
(j) the sense strand comprises a first nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 135, and the antisense strand comprises a second nucleic acid sequence having at least 90% (e.g., about 90%, about 91%, about 92%, about 93%, about 94%, about 95%, about 96%, about 97%, about 98%, about 99%, or about 100%) sequence identity to SEQ ID NO: 136.

In some embodiments, provided herein are MAPT RNAi agents comprising a sense strand and an antisense strand that comprise a pair of nucleic acid sequences selected from the group consisting of:
(a) the sense strand comprises SEQ ID NO: 21, and the antisense strand comprises SEQ ID NO: 22;
(b) the sense strand comprises SEQ ID NO: 23, and the antisense strand comprises SEQ ID NO: 24;
(c) the sense strand comprises SEQ ID NO: 25, and the antisense strand comprises SEQ ID NO: 26;
(d) the sense strand comprises any one of SEQ ID NOs: 27, 33, 39, 40, 47-49, and the antisense strand comprises SEQ ID NO: 28;
(e) the sense strand comprises any one of SEQ ID NOs: 29, 34, 35, 42, 50-51, 53, and the antisense strand comprises SEQ ID NO: 30;
(f) the sense strand comprises SEQ ID NO: 31, 36, 43, 52, 154, 155, 161-163, and the antisense strand comprises SEQ ID NO: 32;
(g) the sense strand comprises SEQ ID NO: 39 or 40, and the antisense strand comprises SEQ ID NO: 41;
(h) the sense strand comprises SEQ ID NO: 44 or 46, and the antisense strand comprises SEQ ID NO: 45;
(i) the sense strand comprises SEQ ID NO: 53, and the antisense strand comprises SEQ ID NO: 54 or 55;
(j) the sense strand comprises SEQ ID NO: 56, and the antisense strand comprises SEQ ID NO: 57;
(k) the sense strand comprises SEQ ID NO: 125, and the antisense strand comprises SEQ ID NO: 126;
(l) the sense strand comprises SEQ ID NO: 127, and the antisense strand comprises SEQ ID NO: 128;
(m) the sense strand comprises SEQ ID NO: 129, and the antisense strand comprises SEQ ID NO: 130;
(n) the sense strand comprises SEQ ID NO: 131, and the antisense strand comprises SEQ ID NO: 132;
(o) the sense strand comprises SEQ ID NO: 133, and the antisense strand comprises SEQ ID NO: 134;
(p) the sense strand comprises SEQ ID NO: 135, and the antisense strand comprises SEQ ID NO: 136;
(q) the sense strand comprises SEQ ID NO: 137, and the antisense strand comprises SEQ ID NO: 138;
(r) the sense strand comprises SEQ ID NO: 139, and the antisense strand comprises SEQ ID NO: 140;
(s) the sense strand comprises SEQ ID NO: 141, and the antisense strand comprises SEQ ID NO: 142;
(t) the sense strand comprises SEQ ID NO: 143, and the antisense strand comprises SEQ ID NO: 144;
(u) the sense strand comprises SEQ ID NO: 145, and the antisense strand comprises SEQ ID NO: 146;
(v) the sense strand comprises SEQ ID NO: 147, and the antisense strand comprises SEQ ID NO: 148;
(w) the sense strand comprises SEQ ID NO: 34, and the antisense strand comprises any one of SEQ ID NO: 149, 150, 151;
(x) the sense strand comprises SEQ ID NO: 31, and the antisense strand comprises any one of SEQ ID NO: 152, 153, 156-159, 164, 165;
(y) the sense strand comprises SEQ ID NO: 160, and the antisense strand comprises SEQ ID NO: 152; and
(z) the sense strand comprises SEQ ID NO: 43 or 166, and the antisense strand comprises SEQ ID NO: 156.

In some embodiments, provided herein are MAPT RNAi agents comprising a sense strand and an antisense strand that consist of a pair of nucleic acid sequences selected from the group consisting of:
(a) the sense strand consists of any one of SEQ ID NOs: 27, 33, 39, 40, 47-49, and the antisense strand consists of SEQ ID NO: 28;
(b) the sense strand consists of any one of SEQ ID NOs: 29, 34, 35, 42, 50-51, 53, and the antisense strand consists of SEQ ID NO: 30;
(c) the sense strand consists of SEQ ID NO: 31, 36, 43, 52, 154, 155, 161-163, and the antisense strand consists of SEQ ID NO: 32;
(d) the sense strand consists of SEQ ID NO: 39 or 40, and the antisense strand consists of SEQ ID NO: 41;
(e) the sense strand consists of SEQ ID NO: 44 or 46, and the antisense strand consists of SEQ ID NO: 45;
(f) the sense strand consists of SEQ ID NO: 53, and the antisense strand consists of SEQ ID NO: 54 or 55;
(g) the sense strand consists of SEQ ID NO: 137, and the antisense strand consists of SEQ ID NO: 138;
(h) the sense strand consists of SEQ ID NO: 139, and the antisense strand consists of SEQ ID NO: 140;
(i) the sense strand consists of SEQ ID NO: 141, and the antisense strand consists of SEQ ID NO: 142;
(j) the sense strand consists of SEQ ID NO: 143, and the antisense strand consists of SEQ ID NO: 144;
(k) the sense strand consists of SEQ ID NO: 145, and the antisense strand consists of SEQ ID NO: 146;
(l) the sense strand consists of SEQ ID NO: 147, and the antisense strand consists of SEQ ID NO: 148;
(m) the sense strand consists of SEQ ID NO: 34, and the antisense strand consists of any one of SEQ ID NO: 149, 150, 151;
(n) the sense strand consists of SEQ ID NO: 31, and the antisense strand consists of any one of SEQ ID NO: 152, 153, 156-159, 164, 165;
(o) the sense strand consists of SEQ ID NO: 160, and the antisense strand consists of SEQ ID NO: 152; and
(p) the sense strand consists of SEQ ID NO: 43 or 166, and the antisense strand consists of SEQ ID NO: 156.

The sense strand and antisense strand of RNAi agent can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene) amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP—IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a duplex.

In another aspect, provided herein are pharmaceutical compositions comprising a compound or RNAi agent described herein and a pharmaceutically acceptable carrier. Such pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 23rd edition (2020), A. Loyd et al., Academic Press).

In a further aspect, provided herein are methods of treating a neurodegenerative disease in a patient in need thereof; such methods comprise administering to the patient an effective amount of a compound, RNAi agent or pharmaceutical composition described herein.

In some embodiments, the neurodegenerative disease is a synucleinopathy selected from Parkinson's disease, Alzheimer's disease, multiple system atrophy, or Lewy body dementia.

In some embodiments, the neurodegenerative disease is a tauopathy selected from Alzheimer's disease, frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's discase, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), or globular glial tauopathies (GGT).

In some embodiments, the compound, RNAi agent or pharmaceutical composition is administered to the patient intrathecally, intracerebroventricularly, or via intracisternal magna injection.

Also provided herein are methods of inhibiting or reducing a target mRNA in a cell, the method comprising contacting the cell comprising the target mRNA with the compound, RNAi agent, or pharmaceutical composition described herein. In some embodiments, the cell is a mammalian cell. In some embodiments, the cell is a human cell. In some embodiments, the cell is in a subject. In some embodiments a subject is a human subject.

The dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are compounds, RNAi agents or pharmaceutical compositions for use in a therapy. Also provided herein are compounds, RNAi agents, or pharmaceutical compositions for use in the treatment of a neurodegenerative disease, e.g., synucleinopathy or tauopathy. Also provided herein are uses of compounds or RNAi agents in the manufacture of a medicament for the treatment of a neurodegenerative disease, e.g., synucleinopathy or tauopathy.

As used herein, the terms “a,” “an,” “the,” and similar terms used in the context of the present disclosure (especially in the context of the claims) are to be construed to cover both the singular and plural unless otherwise indicated herein or clearly contradicted by the context.

As used herein, the term “alkyl” means saturated linear or branched-chain monovalent hydrocarbon radical, containing the indicated number of carbon atoms. For example, “C₁-C₂₀ alkyl” means a radical having 1-20 carbon atoms in a linear or branched arrangement.

As used herein, “antisense strand” means an oligonucleotide that is complementary to a region of a target sequence. Likewise, and as used herein, “sense strand” means an oligonucleotide that is complementary to a region of an antisense strand.

As used herein, “complementary” means a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another.

Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, a “delivery moiety” refers to a chemical moiety that facilitates the entry of an oligonucleotide or RNAi agent into a cell. The delivery moiety can be lipid, cholesterol, vitamin E, carbohydrate, amino sugar, polypeptide or protein.

As used herein, “duplex,” in reference to nucleic acids or oligonucleotides, means a structure formed through complementary base pairing of two antiparallel sequences of nucleotides (i.e., in opposite directions), whether formed by two separate nucleic acid strands or by a single, folded strand (e.g., via a hairpin).

An “effective amount” refers to an amount necessary (for periods of time and for the means of administration) to achieve the desired therapeutic result. An effective amount of a RNAi agent may vary according to factors such as the disease state, age, sex, and weight of the individual, and the ability of the RNAi agent to elicit a desired response in the individual. An effective amount is also one in which any toxic or detrimental effects of the RNAi agent are outweighed by the therapeutically beneficial effects.

The term “knockdown” or “expression knockdown” refers to reduced mRNA or protein expression of a gene after treatment of a reagent, e.g., a RNAi agent.

As used herein, “modified internucleotide linkage” means an internucleotide linkage having one or more chemical modifications when compared with a reference internucleotide linkage having a phosphodiester bond. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc. In some embodiments, the modified internucleotide linkage is phosphorothioate linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide. In some embodiments, the modified nucleotide is a 2′-fluoro modified nucleotide, 2′-O-methyl modified nucleotide, or 2′-O-alkyl modified nucleotide, e.g., 2′-O—C16 alkyl modified nucleotide. In some embodiments, the modified nucleotide has a phosphate analog, e.g., 5′-vinylphosphonate. In some embodiments, the modified nucleotide is an abasic moiety or inverted abasic moiety.

As used herein, the term “synucleinopathy” refers to a disease characterized by fibrillary aggregates of alpha-synuclein protein in the cytoplasm of selective populations of neurons and glia in the central and/or peripheral nervous systems.

As used herein, the term “tauopathy” refers to a disease associated with abnormal tau protein expression, secretion, phosphorylation, cleavage, and/or aggregation.

As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group, which can serve as a monomeric unit of nucleic acid polymers such as deoxyribonucleic acid (DNA) and ribonucleic acid (RNA).

As used herein, “oligonucleotide” means a polymer of linked nucleotides, each of which can be modified or unmodified. An oligonucleotide is typically less than about 100 nucleotides in length.

As used herein, “overhang” means the unpaired nucleotide or nucleotides that protrude from the duplex structure of a double stranded oligonucleotide. An overhang may include one or more unpaired nucleotides extending from a duplex region at the 5′ terminus or 3′ terminus of a double stranded oligonucleotide. The overhang can be a 3′ or 5′ overhang on the antisense strand or sense strand of a double stranded oligonucleotide.

The term “patient”, as used herein, refers to a human patient.

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. A 5′ phosphate analog can include a phosphatase-resistant linkage. Examples of phosphate analogs include 5′ methylene phosphonate (5′-MP) and 5′-(E)-vinylphosphonate (5′-VP). In some embodiments, the phosphate analog is 5′-VP.

The term “% sequence identity” or “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Clustal W2.0, Clustal X2.0, or Megalign (DNASTAR) software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

As used herein, “RNAi,” “RNAi agent,” “iRNA,” “iRNA agent,” and “RNA interference agent” means an agent that mediates sequence-specific degradation of a target mRNA by RNA interference, e.g., via RNA-induced silencing complex (RISC) pathway. In some embodiments, the RNAi agent has a sense strand and an antisense strand, and the sense strand and the antisense strand form a duplex. In some embodiments, the sense strand has a delivery moiety, e.g., a delivery moiety conjugated to the 5′ or 3′ end of the sense strand or a nucleotide of the sense strand.

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “SNCA” refers to an alpha-synuclein (SNCA) mRNA transcript. The nucleic acid sequence of a human SNCA mRNA transcript can be found at NM_000345.4:

```
(SEQ ID NO: 58)
      1 GGCGACGACC AGAAGGGGCC CAAGAGAGGG GGCGAGCGAC CGAGCGCCGC GACGCGGAAG
    61 TGAGGTGCGT GCGGGCTGCA GCGCAGACCC CGGCCCGGCC CCTCCGAGAG CGTCCTGGGC
  121 GCTCCCTCAC GCCTTGCCTT CAAGCCTTCT GCCTTTCCAC CCTCGTGAGC GGAGAACTGG
  181 GAGTGGCCAT TCGACGACAG TGTGGTGTAA AGGAATTCAT TAGCCATGGA TGTATTCATG
  241 AAAGGACTTT CAAAGGCCAA GGAGGGAGTT GTGGCTGCTG CTGAGAAAAC CAAACAGGGT
  301 GTGGCAGAAG CAGCAGGAAA GACAAAAGAG GGTGTTCTCT ATGTAGGCTC CAAAACCAAG
  361 GAGGGAGTGG TGCATGGTGT GGCAACAGTG GCTGAGAAGA CCAAAGAGCA AGTGACAAAT
  421 GTTGGAGGAG CAGTGGTGAC GGGTGTGACA GCAGTAGCCC AGAAGACAGT GGAGGGAGCA
  481 GGGAGCATTG CAGCAGCCAC TGGCTTTGTC AAAAAGGACC AGTTGGGCAA GAATGAAGAA
  541 GGAGCCCCAC AGGAAGGAAT TCTGGAAGAT ATGCCTGTGG ATCCTGACAA TGAGGCTTAT
  601 GAAATGCCTT CTGAGGAAGG GTATCAAGAC TACGAACCTG AAGCCTAAGA AATATCTTTG
  661 CTCCCAGTTT CTTGAGATCT GCTGACAGAT GTTCCATCCT GTACAAGTGC TCAGTTCCAA
  721 TGTGCCCAGT CATGACATTT CTCAAAGTTT TTACAGTGTA TCTCGAAGTC TTCCATCAGC
  781 AGTGATTGAA GTATCTGTAC CTGCCCCCAC TCAGCATTTC GGTGCTTCCC TTTCACTGAA
  841 GTGAATACAT GGTAGCAGGG TCTTTGTGTG CTGTGGATTT TGTGGCTTCA ATCTACGATG
  901 TTAAAACAAA TTAAAAACAC CTAAGTGACT ACCACTTATT TCTAAATCCT CACTATTTTT
  961 TTGTTGCTGT TGTTCAGAAG TTGTTAGTGA TTTGCTATCA TATATTATAA GATTTTTAGG
1021 TGTCTTTTAA TGATACTGTC TAAGAATAAT GACGTATTGT GAAATTTGTT AATATATATA
1081 ATACTTAAAA ATATGTGAGC ATGAAACTAT GCACCTATAA ATACTAAATA TGAAATTTTA
1141 CCATTTTGCG ATGTGTTTTA TTCACTTGTG TTTGTATATA AATGGTGAGA ATTAAAATAA
1201 AACGTTATCT CATTGCAAAA ATATTTTATT TTTATCCCAT CTCACTTTAA TAATAAAAAT
1261 CATGCTTATA AGCAACATGA ATTAAGAACT GACACAAAGG ACAAAAATAT AAAGTTATTA
1321 ATAGCCATTT GAAGAAGGAG GAATTTTAGA AGAGGTAGAG AAAATGGAAC ATTAACCCTA
1381 CACTCGGAAT TCCCTGAAGC AACACTGCCA GAAGTGTGTT TTGGTATGCA CTGGTTCCTT
1441 AAGTGGCTGT GATTAATTAT TGAAAGTGGG GTGTTGAAGA CCCCAACTAC TATTGTAGAG
1501 TGGTCTATTT CTCCCTTCAA TCCTGTCAAT GTTTGCTTTA CGTATTTTGG GGAACTGTTG
1561 TTTGATGTGT ATGTGTTTAT AATTGTTATA CATTTTTAAT TGAGCCTTTT ATTAACATAT
1621 ATTGTTATTT TTGTCTCGAA ATAATTTTTT AGTTAAAATC TATTTTGTCT GATATTGGTG
1681 TGAATGCTGT ACCTTTCTGA CAATAAATAA TATTCGACCA TGAATAAAAA AAAAAAAAAA
1741 GTGGGTTCCC GGGAACTAAG CAGTGTAGAA GATGATTTTG ACTACACCCT CCTTAGAGAG
1801 CCATAAGACA CATTAGCACA TATTAGCACA TTCAAGGCTC TGAGAGAATG TGGTTAACTT
1861 TGTTTAACTC AGCATTCCTC ACTTTTTTTT TTTAATCATC AGAAATTCTC TCTCTCTCTC
1921 TCTCTTTTTC TCTCGCTCTC TTTTTTTTTT TTTTTTTACA GGAAATGCCT TTAAACATCG
1981 TTGGAACTAC CAGAGTCACC TTAAAGGAGA TCAATTCTCT AGACTGATAA AAATTTCATG
2041 GCCTCCTTTA AATGTTGCCA AATATATGAA TTCTAGGATT TTTCCTTAGG AAAGGTTTTT
2101 CTCTTTCAGG GAAGATCTAT TAACTCCCCA TGGGTGCTGA AAATAAACTT GATGGTGAAA
2161 AACTCTGTAT AAATTAATTT AAAAATTATT TGGTTTCTCT TTTTAATTAT TCTGGGGCAT
2221 AGTCATTTCT AAAAGTCACT AGTAGAAAGT ATAATTTCAA GACAGAATAT TCTAGACATG
2281 CTAGCAGTTT ATATGTATTC ATGAGTAATG TGATATATAT TGGGCGCTGG TGAGGAAGGA
2341 AGGAGGAATG AGTGACTATA AGGATGGTTA CCATAGAAAC TTCCTTTTTT ACCTAATTGA
2401 AGAGAGACTA CTACAGAGTG CTAAGCTGCA TGTGTCATCT TACACTAGAG AGAAATGGTA
2461 AGTTTCTTGT TTTATTTAAG TTATGTTTAA GCAAGGAAAG GATTTGTTAT TGAACAGTAT
2521 ATTTCAGGAA GGTTAGAAAG TGGCGGTTAG GATATATTTT AAATCTACCT AAAGCAGCAT
2581 ATTTTAAAAA TTTAAAAGTA TTGGTATTAA ATTAAGAAAT AGAGGACAGA ACTAGACTGA
2641 TAGCAGTGAC CTAGAACAAT TTGAGATTAG GAAAGTTGTG ACCATGAATT TAAGGATTTA
2701 TGTGGATACA AATTCTCCTT TAAAGTGTTT CTTCCCTTAA TATTTATCTG ACGGTAATTT
2761 TTGAGCAGTG AATTACTTTA TATATCTTAA TAGTTTATTT GGGACCAAAC ACTTAAACAA
2821 AAAGTTCTTT AAGTCATATA AGCCTTTTCA GGAAGCTTGT CTCATATTCA CTCCCGAGAC
2881 ATTCACCTGC CAAGTGGCCT GAGGATCAAT CCAGTCCTAG GTTTATTTTG CAGACTTACA
2941 TTCTCCCAAG TTATTCAGCC TCATATGACT CCACGGTCGG CTTTACCAAA ACAGTTCAGA
3001 GTGCACTTTG GCACACAATT GGGAACAGAA CAATCTAATG TGTGGTTTGG TATTCCAAGT
3061 GGGGTCTTTT TCAGAATCTC TGCACTAGTG TGAGATGCAA ACATGTTTCC TCATCTTTCT
3121 GGCTTATCCA GTATGTAGCT ATTTGTGACA TAATAAATAT ATACATATAT GAAAATA.
```

The amino acid sequence of a human SNCA protein can be found at NP_000336.1:

```
(SEQ ID NO: 59)
    1 MDVFMKGLSK AKEGVVAAAE KTKQGVAEAA
        GKTKEGVLYV GSKTKEGVVH GVATVAEKTK
  61 EQVTNVGGAV VTGVTAVAQK TVEGAGSIAA
        ATGFVKKDQL GKNEEGAPQE GILEDMPVDP
121 DNEA YEMPSE EGYQDYEPEA
```

The nucleic acid sequence of a mouse SNCA mRNA transcript can be found at NM_001042451.2; and the amino acid sequence of a mouse SNCA protein can be found at NP_001035916.1. The nucleic acid sequence of a rat SNCA mRNA transcript can be found at NM 019169.3; and the amino acid sequence of a rat SNCA protein can be found at NP_062042.1. The nucleic acid sequence of a monkey SNCA mRNA transcript can be found at XM_005555422.2; and the amino acid sequence of a monkey SNCA protein can be found at XP_005555479.1.

As used herein, “MAPT” refers to a human MAPT mRNA transcript, encoding a microtubule associated protein Tau. The nucleotide sequences of human MAPT transcript variants and amino acid sequences of human Tau protein isoforms can be found at:
i. MAPT transcript variant 1→Tau protein isoform 1: NM_016835.5 (nucleotide sequence)→NP_058519.3 (amino acid sequence);
ii. MAPT transcript variant 2→Tau protein isoform 2: NM_005910.6 (nucleotide sequence)→NP_005901.2 (amino acid sequence);
iii. MAPT transcript variant 3→Tau protein isoform 3: NM_016834.5 (nucleotide sequence)→NP_058518.1 (amino acid sequence);
iv. MAPT transcript variant 4→Tau protein isoform 4: NM_016841.5 (nucleotide sequence)→NP_058525.1 (amino acid sequence);
v. MAPT transcript variant 5→Tau protein isoform 5: NM_001123067.4 (nucleotide sequence)→NP_001116539.1 (amino acid sequence);
vi. MAPT transcript variant 6→Tau protein isoform 6: NM_001123066.4 (nucleotide sequence)→NP_001116538.2 (amino acid sequence);
vii. MAPT transcript variant 7→Tau protein isoform 7: NM_001203251.2 (nucleotide sequence)→NP_001190180.1 (amino acid sequence);
viii. MAPT transcript variant 8→Tau protein isoform 8: NM_001203252.2 (nucleotide sequence)→NP_001190181.1 (amino acid sequence);
ix. MAPT transcript variant 9→Tau protein isoform 9: NM_001377265.1 (nucleotide sequence)→NP_001364194.1 (amino acid sequence);
x. MAPT transcript variant 10→Tau protein isoform 10: NM_001377266.1 (nucleotide sequence)→NP_001364195.1 (amino acid sequence);
xi. MAPT transcript variant 11→Tau protein isoform 11: NM_001377267.1 (nucleotide sequence)→NP_001364196.1 (amino acid sequence);
xii. MAPT transcript variant 12→Tau protein isoform 4: NM_001377268.1 (nucleotide sequence)→NP_001364197.1 (amino acid sequence).

The nucleotide sequence of the human MAPT transcript variant 6 (encoding 2N4R Tau) can be found at NM_001123066.4:

```
(SEQ ID NO: 60)
      1 GCAGTCACCG CCACCCACCA GCTCCGGCAC CAACAGCAGC GCCGCTGCCA CCGCCCACCT
    61 TCTGCCGCCG CCACCACAGC CACCTTCTCC TCCTCCGCTG TCCTCTCCCG TCCTCGCCTC
  121 TGTCGACTAT CAGGTGAACT TTGAACCAGG ATGGCTGAGC CCCGCCAGGA GTTCGAAGTG
  181 ATGGAAGATC ACGCTGGGAC GTACGGGTTG GGGGACAGGA AAGATCAGGG GGGCTACACC
  241 ATGCACCAAG ACCAAGAGGG TGACACGGAC GCTGGCCTGA AAGAATCTCC CCTGCAGACC
  301 CCCACTGAGG ACGGATCTGA GGAACCGGGC TCTGAAACCT CTGATGCTAA GAGCACTCCA
  361 ACAGCGGAAG ATGTGACAGC ACCCTTAGTG GATGAGGGAG CTCCCGGCAA GCAGGCTGCC
  421 GCGCAGCCCC ACACGGAGAT CCCAGAAGGA ACCACAGCTG AAGAAGCAGG CATTGGAGAC
  481 ACCCCCAGCC TGGAAGACGA AGCTGCTGGT CACGTGACCC AAGAGCCTGA AAGTGGTAAG
  541 GTGGTCCAGG AAGGCTTCCT CCGAGAGCCA GGCCCCCCAG GTCTGAGCCA CCAGCTCATG
  601 TCCGGCATGC CTGGGGCTCC CCTCCTGCCT GAGGGCCCCA GAGAGGCCAC ACGCCAACCT
  661 TCGGGGACAG GACCTGAGGA CACAGAGGGC GGCCGCCACG CCCCTGAGCT GCTCAAGCAC
  721 CAGCTTCTAG GAGACCTGCA CCAGGAGGGG CCGCCGCTGA AGGGGGCAGG GGGCAAAGAG
  781 AGGCCGGGGA GCAAGGAGGA GGTGGATGAA GACCGCGACG TCGATGAGTC CTCCCCCCAA
  841 GACTCCCCTC CCTCCAAGGC CTCCCCAGCC CAAGATGGGC GGCCTCCCCA GACAGCCGCC
  901 AGAGAAGCCA CCAGCATCCC AGGCTTCCCA GCGGAGGGTG CCATCCCCCT CCCTGTGGAT
  961 TTCCTCTCCA AAGTTTCCAC AGAGATCCCA GCCTCAGAGC CCGACGGGCC CAGTGTAGGG
1021 CGGGCCAAAG GGCAGGATGC CCCCCTGGAG TTCACGTTTC ACGTGGAAAT CACACCCAAC
1081 GTGCAGAAGG AGCAGGCGCA CTCGGAGGAG CATTTGGGAA GGGCTGCATT TCCAGGGGCC
1141 CCTGGAGAGG GGCCAGAGGC CCGGGGCCCC TCTTTGGGAG AGGACACAAA AGAGGCTGAC
1201 CTTCCAGAGC CCTCTGAAAA GCAGCCTGCT GCTGCTCCGC GGGGGAAGCC CGTCAGCCGG
1261 GTCCCTCAAC TCAAAGCTCG CATGGTCAGT AAAAGCAAAG ACGGGACTGG AAGCGATGAC
1321 AAAAAAGCCA AGACATCCAC ACGTTCCTCT GCTAAAACCT TGAAAAATAG GCCTTGCCTT
1381 AGCCCCAAAC ACCCCACTCC TGGTAGCTCA GACCCTCTGA TCCAACCCTC CAGCCCTGCT
1441 GTGTGCCCAG AGCCACCTTC CTCTCCTAAA TACGTCTCTT CTGTCACTTC CCGAACTGGC
1501 AGTTCTGGAG CAAAGGAGAT GAAACTCAAG GGGGCTGATG GTAAAACGAA GATCGCCACA
1561 CCGCGGGGAG CAGCCCCTCC AGGCCAGAAG GGCCAGGCCA ACGCCACCAG GATTCCAGCA
1621 AAAACCCCGC CCGCTCCAAA GACACCACCC AGCTCTGCGA CTAAGCAAGT CCAGAGAAGA
1681 CCACCCCCTG CAGGGCCCAG ATCTGAGAGA GGTGAACCTC CAAAATCAGG GGATCGCAGC
1741 GGCTACAGCA GCCCCGGCTC CCCAGGCACT CCCGGCAGCC GCTCCCGCAC CCCGTCCCTT
1801 CCAACCCCAC CCACCCGGGA GCCCAAGAAG GTGGCAGTGG TCCGTACTCC ACCCAAGTCG
1861 CCGTCTTCCG CCAAGAGCCG CCTGCAGACA GCCCCCGTGC CCATGCCAGA CCTGAAGAAT
1921 GTCAAGTCCA AGATCGGCTC CACTGAGAAC CTGAAGCACC AGCCGGGAGG CGGGAAGGTG
1981 CAGATAATTA ATAAGAAGCT GGATCTTAGC AACGTCCAGT CCAAGTGTGG CTCAAAGGAT
2041 AATATCAAAC ACGTCCCGGG AGGCGGCAGT GTGCAAATAG TCTACAAACC AGTTGACCTG
2101 AGCAAGGTGA CCTCCAAGTG TGGCTCATTA GGCAACATCC ATCATAAACC AGGAGGTGGC
2161 CAGGTGGAAG TAAAATCTGA GAAGCTTGAC TTCAAGGACA GAGTCCAGTC GAAGATTGGG
2221 TCCCTGGACA ATATCACCCA CGTCCCTGGC GGAGGAAATA AAAAGATTGA AACCCACAAG
2281 CTGACCTTCC GCGAGAACGC CAAAGCCAAG ACAGACCACG GGGCGGAGAT CGTGTACAAG
2341 TCGCCAGTGG TGTCTGGGGA CACGTCTCCA CGGCATCTCA GCAATGTCTC CTCCACCGGC
2401 AGCATCGACA TGGTAGACTC GCCCCAGCTC GCCACGCTAG CTGACGAGGT GTCTGCCTCC
2461 CTGGCCAAGC AGGGTTTGTG ATCAGGCCCC TGGGGCGGTC AATAATTGTG GAGAGGAGAG
2521 AATGAGAGAG TGTGGAAAAA AAAAGAATAA TGACCCGGCC CCCGCCCTCT GCCCCCAGCT
2581 GCTCCTCGCA GTTCGGTTAA TTGGTTAATC ACTTAACCTG CTTTTGTCAC TCGGCTTTGG
2641 CTCGGGACTT CAAAATCAGT GATGGGAGTA AGAGCAAATT TCATCTTTCC AAATTGATGG
2701 GTGGGCTAGT AATAAAATAT TTAAAAAAAA ACATTCAAAA ACATGGCCAC ATCCAACATT
2761 TCCTCAGGCA ATTCCTTTTG ATTCTTTTTT CTTCCCCCTC CATGTAGAAG AGGGAGAAGG
2821 AGAGGCTCTG AAAGCTGCTT CTGGGGGATT TCAAGGGACT GGGGGTGCCA ACCACCTCTG
2881 GCCCTGTTGT GGGGGTGTCA CAGAGGCAGT GGCAGCAACA AAGGATTTGA AACTTGGTGT
2941 GTTCGTGGAG CCACAGGCAG ACGATGTCAA CCTTGTGTGA GTGTGACGGG GGTTGGGGTG
3001 GGGCGGGAGG CCACGGGGGA GGCCGAGGCA GGGGCTGGGC AGAGGGGAGA GGAAGCACAA
3061 GAAGTGGGAG TGGGAGAGGA AGCCACGTGC TGGAGAGTAG ACATCCCCCT CCTTGCCGCT
3121 GGGAGAGCCA AGGCCTATGC CACCTGCAGC GTCTGAGCGG CCGCCTGTCC TTGGTGGCCG
3181 GGGGTGGGGG CCTGCTGTGG GTCAGTGTGC CACCCTCTGC AGGGCAGCCT GTGGGAGAAG
3241 GGACAGCGGG TAAAAAGAGA AGGCAAGCTG GCAGGAGGGT GGCACTTCGT GGATGACCTC
3301 CTTAGAAAAG ACTGACCTTG ATGTCTTGAG AGCGCTGGCC TCTTCCTCCC TCCCTGCAGG
3361 GTAGGGGGCC TGAGTTGAGG GGCTTCCCTC TGCTCCACAG AAACCCTGTT TTATTGAGTT
3421 CTGAAGGTTG GAACTGCTGC CATGATTTTG GCCACTTTGC AGACCTGGGA CTTTAGGGCT
3481 AACCAGTTCT CTTTGTAAGG ACTTGTGCCT CTTGGGAGAC GTCCACCCGT TTCCAAGCCT
3541 GGGCCACTGG CATCTCTGGA GTGTGTGGGG GTCTGGGAGG CAGGTCCCGA GCCCCCTGTC
3601 CTTCCCACGG CCACTGCAGT CACCCCGTCT GCGCCGCTGT GCTGTTGTCT GCCGTGAGAG
3661 CCCAATCACT GCCTATACCC CTCATCACAC GTCACAATGT CCCGAATTCC CAGCCTCACC
3721 ACCCCTTCTC AGTAATGACC CTGGTTGGTT GCAGGAGGTA CCTACTCCAT ACTGAGGGTG
3781 AAATTAAGGG AAGGCAAAGT CCAGGCACAA GAGTGGGACC CCAGCCTCTC ACTCTCAGTT
3841 CCACTCATCC AACTGGGACC CTCACCACGA ATCTCATGAT CTGATTCGGT TCCCTGTCTC
3901 CTCCTCCCGT CACAGATGTG AGCCAGGGCA CTGCTCAGCT GTGACCCTAG GTGTTTCTGC
3961 CTTGTTGACA TGGAGAGAGC CCTTTCCCCT GAGAAGGCCT GGCCCCTTCC TGTGCTGAGC
4021 CCACAGCAGC AGGCTGGGTG TCTTGGTTGT CAGTGGTGGC ACCAGGATGG AAGGGCAAGG
4081 CACCCAGGGC AGGCCCACAG TCCCGCTGTC CCCCACTTGC ACCCTAGCTT GTAGCTGCCA
4141 ACCTCCCAGA CAGCCCAGCC CGCTGCTCAG CTCCACATGC ATAGTATCAG CCCTCCACAC
4201 CCGACAAAGG GGAACACACC CCCTTGGAAA TGGTTCTTTT CCCCCAGTCC CAGCTGGAAG
4261 CCATGCTGTC TGTTCTGCTG GAGCAGCTGA ACATATACAT AGATGTTGCC CTGCCCTCCC
4321 CATCTGCACC CTGTTGAGTT GTAGTTGGAT TTGTCTGTTT ATGCTTGGAT TCACCAGAGT
4381 GACTATGATA GTGAAAAGAA AAAAAAAAAA AAAAAAGGAC GCATGTATCT TGAAATGCTT
4441 GTAAAGAGGT TTCTAACCCA CCCTCACGAG GTGTCTCTCA CCCCCACACT GGGACTCGTG
4501 TGGCCTGTGT GGTGCCACCC TGCTGGGGCC TCCCAAGTTT TGAAAGGCTT TCCTCAGCAC
4561 CTGGGACCCA ACAGAGACCA GCTTCTAGCA GCTAAGGAGG CCGTTCAGCT GTGACGAAGG
4621 CCTGAAGCAC AGGATTAGGA CTGAAGCGAT GATGTCCCCT TCCCTACTTC CCCTTGGGGC
4681 TCCCTGTGTC AGGGCACAGA CTAGGTCTTG TGGCTGGTCT GGCTTGCGGC GCGAGGATGG
4741 TTCTCTCTGG TCATAGCCCG AAGTCTCATG GCAGTCCCAA AGGAGGCTTA CAACTCCTGC
4801 ATCACAAGAA AAAGGAAGCC ACTGCCAGCT GGGGGGATCT GCAGCTCCCA GAAGCTCCGT
4861 GAGCCTCAGC CACCCCTCAG ACTGGGTTCC TCTCCAAGCT CGCCCTCTGG AGGGGCAGCG
4921 CAGCCTCCCA CCAAGGGCCC TGCGACCACA GCAGGGATTG GGATGAATTG CCTGTCCTGG
4981 ATCTGCTCTA GAGGCCCAAG CTGCCTGCCT GAGGAAGGAT GACTTGACAA GTCAGGAGAC
5041 ACTGTTCCCA AAGCCTTGAC CAGAGCACCT CAGCCCGCTG ACCTTGCACA AACTCCATCT
5101 GCTGCCATGA GAAAAGGGAA GCCGCCTTTG CAAAACATTG CTGCCTAAAG AAACTCAGCA
5161 GCCTCAGGCC CAATTCTGCC ACTTCTGGTT TGGGTACAGT TAAAGGCAAC CCTGAGGGAC
5221 TTGGCAGTAG AAATCCAGGG CCTCCCCTGG GGCTGGCAGC TTCGTGTGCA GCTAGAGCTT
5281 TACCTGAAAG GAAGTCTCTG GGCCCAGAAC TCTCCACCAA GAGCCTCCCT GCCGTTCGCT
5341 GAGTCCCAGC AATTCTCCTA AGTTGAAGGG ATCTGAGAAG GAGAAGGAAA TGTGGGGTAG
5401 ATTTGGTGGT GGTTAGAGAT ATGCCCCCCT CATTACTGCC AACAGTTTCG GCTGCATTTC
5461 TTCACGCACC TCGGTTCCTC TTCCTGAAGT TCTTGTGCCC TGCTCTTCAG CACCATGGGC
5521 CTTCTTATAC GGAAGGCTCT GGGATCTCCC CCTTGTGGGG CAGGCTCTTG GGGCCAGCCT
5581 AAGATCATGG TTTAGGGTGA TCAGTGCTGG CAGATAAATT GAAAAGGCAC GCTGGCTTGT
5641 GATCTTAAAT GAGGACAATC CCCCCAGGGC TGGGCACTCC TCCCCTCCCC TCACTTCTCC
5701 CACCTGCAGA GCCAGTGTCC TTGGGTGGGC TAGATAGGAT ATACTGTATG CCGGCTCCTT
5761 CAAGCTGCTG ACTCACTTTA TCAATAGTTC CATTTAAATT GACTTCAGTG GTGAGACTGT
5821 ATCCTGTTTG CTATTGCTTG TTGTGCTATG GGGGGAGGGG GGAGGAATGT GTAAGATAGT
5881 TAACATGGGC AAAGGGAGAT CTTGGGGTGC AGCACTTAAA CTGCCTCGTA ACCCTTTTCA
5941 TGATTTCAAC CACATTTGCT AGAGGGAGGG AGCAGCCACG GAGTTAGAGG CCCTTGGGGT
6001 TTCTCTTTTC CACTGACAGG CTTTCCCAGG CAGCTGGCTA GTTCATTCCC TCCCCAGCCA
6061 GGTGCAGGCG TAGGAATATG GACATCTGGT TGCTTTGGCC TGCTGCCCTC TTTCAGGGGT
6121 CCTAAGCCCA CAATCATGCC TCCCTAAGAC CTTGGCATCC TTCCCTCTAA GCCGTTGGCA
6181 CCTCTGTGCC ACCTCTCACA CTGGCTCCAG ACACACAGCC TGTGCTTTTG GAGCTGAGAT
6241 CACTCGCTTC ACCCTCCTCA TCTTTGTTCT CCAAGTAAAG CCACGAGGTC GGGGCGAGGG
6301 CAGAGGTGAT CACCTGCGTG TCCCATCTAC AGACCTGCAG CTTCATAAAA CTTCTGATTT
6361 CTCTTCAGCT TTGAAAAGGG TTACCCTGGG CACTGGCCTA GAGCCTCACC TCCTAATAGA
6421 CTTAGCCCCA TGAGTTTGCC ATGTTGAGCA GGACTATTTC TGGCACTTGC AAGTCCCATG
6481 ATTTCTTCGG TAATTCTGAG GGTGGGGGGA GGGACATGAA ATCATCTTAG CTTAGCTTTC
6541 TGTCTGTGAA TGTCTATATA GTGTATTGTG TGTTTTAACA AATGATTTAC ACTGACTGTT
6601 GCTGTAAAAG TGAATTTGGA AATAAAGTTA TTACTCTGAT TAAA.
```

The corresponding amino acid sequence of human Tau protein isoform 6 can be found at NP_001116538.2:

```
(SEQ ID NO: 61)
    1 MAEPRQEFEV MEDHAGTYGL GDRKDQGGYT MHQDQEGDTD AGLKESPLQT PTEDGSEEPG
  61 SETSDAKSTP TAEDVTAPLV DEGAPGKQAA AQPHTEIPEG TTAEEAGIGD TPSLEDEAAG
121 HVTQEPESGK VVQEGFLREP GPPGLSHQLM SGMPGAPLLP EGPREATRQP SGTGPEDTEG
181 GRHAPELLKH QLLGDLHQEG PPLKGAGGKE RPGSKEEVDE DRDVDESSPQ DSPPSKASPA
241 QDGRPPQTAA REATSIPGFP AEGAIPLPVD FLSKVSTEIP ASEPDGPSVG RAKGQDAPLE
301 FTFHVEITPN VQKEQAHSEE HLGRAAFPGA PGEGPEARGP SLGEDTKEAD LPEPSEKQPA
361 AAPRGKPVSR VPQLKARMVS KSKDGTGSDD KKAKTSTRSS AKTLKNRPCL SPKHPTPGSS
421 DPLIQPSSPA VCPEPPSSPK YVSSVTSRTG SSGAKEMKLK GADGKTKIAT PRGAAPPGQK
481 GQANATRIPA KTPPAPKTPP SSATKQVQRR PPPAGPRSER GEPPKSGDRS GYSSPGSPGT
541 PGSRSRTPSL PTPPTREPKK VAVVRTPPKS PSSAKSRLQT APVPMPDLKN VKSKIGSTEN
601 LKHQPGGGKV QIINKKLDLS NVQSKCGSKD NIKHVPGGGS VQIVYKPVDL SKVTSKCGSL
661 GNIHHKPGGG QVEVKSEKLD FKDRVQSKIG SLDNITHVPG GGNKKIETHK LTFRENAKAK
721 TDHGAEIVYK SPVVSGDTSP RHLSNVSSTG SIDMVDSPQL ATLADEVSAS LAKQGL
```

The nucleotide sequence of a human MAPT transcript variant 5 (encoding 1N4R Tau) can be found at NM_001123067.4:

```
(SEQ ID NO: 62)
      1 GCAGTCACCG CCACCCACCA GCTCCGGCAC CAACAGCAGC GCCGCTGCCA CCGCCCACCT
    61 TCTGCCGCCG CCACCACAGC CACCTTCTCC TCCTCCGCTG TCCTCTCCCG TCCTCGCCTC
  121 TGTCGACTAT CAGGTGAACT TTGAACCAGG ATGGCTGAGC CCCGCCAGGA GTTCGAAGTG
  181 ATGGAAGATC ACGCTGGGAC GTACGGGTTG GGGGACAGGA AAGATCAGGG GGGCTACACC
  241 ATGCACCAAG ACCAAGAGGG TGACACGGAC GCTGGCCTGA AAGAATCTCC CCTGCAGACC
  301 CCCACTGAGG ACGGATCTGA GGAACCGGGC TCTGAAACCT CTGATGCTAA GAGCACTCCA
  361 ACAGCGGAAG CTGAAGAAGC AGGCATTGGA GACACCCCCA GCCTGGAAGA CGAAGCTGCT
  421 GGTCACGTGA CCCAAGCTCG CATGGTCAGT AAAAGCAAAG ACGGGACTGG AAGCGATGAC
  481 AAAAAAGCCA AGGGGGCTGA TGGTAAAACG AAGATCGCCA CACCGCGGGG AGCAGCCCCT
  541 CCAGGCCAGA AGGGCCAGGC CAACGCCACC AGGATTCCAG CAAAAACCCC GCCCGCTCCA
  601 AAGACACCAC CCAGCTCTGG TGAACCTCCA AAATCAGGGG ATCGCAGCGG CTACAGCAGC
  661 CCCGGCTCCC CAGGCACTCC CGGCAGCCGC TCCCGCACCC CGTCCCTTCC AACCCCACCC
  721 ACCCGGGAGC CCAAGAAGGT GGCAGTGGTC CGTACTCCAC CCAAGTCGCC GTCTTCCGCC
  781 AAGAGCCGCC TGCAGACAGC CCCCGTGCCC ATGCCAGACC TGAAGAATGT CAAGTCCAAG
  841 ATCGGCTCCA CTGAGAACCT GAAGCACCAG CCGGGAGGCG GGAAGGTGCA GATAATTAAT
  901 AAGAAGCTGG ATCTTAGCAA CGTCCAGTCC AAGTGTGGCT CAAAGGATAA TATCAAACAC
  961 GTCCCGGGAG GCGGCAGTGT GCAAATAGTC TACAAACCAG TTGACCTGAG CAAGGTGACC
1021 TCCAAGTGTG GCTCATTAGG CAACATCCAT CATAAACCAG GAGGTGGCCA GGTGGAAGTA
1081 AAATCTGAGA AGCTTGACTT CAAGGACAGA GTCCAGTCGA AGATTGGGTC CCTGGACAAT
1141 ATCACCCACG TCCCTGGCGG AGGAAATAAA AAGATTGAAA CCCACAAGCT GACCTTCCGC
1201 GAGAACGCCA AAGCCAAGAC AGACCACGGG GCGGAGATCG TGTACAAGTC GCCAGTGGTG
1261 TCTGGGGACA CGTCTCCACG GCATCTCAGC AATGTCTCCT CCACCGGCAG CATCGACATG
1321 GTAGACTCGC CCCAGCTCGC CACGCTAGCT GACGAGGTGT CTGCCTCCCT GGCCAAGCAG
1381 GGTTTGTGAT CAGGCCCCTG GGGCGGTCAA TAATTGTGGA GAGGAGAGAA TGAGAGAGTG
1441 TGGAAAAAAA AAGAATAATG ACCCGGCCCC CGCCCTCTGC CCCCAGCTGC TCCTCGCAGT
1501 TCGGTTAATT GGTTAATCAC TTAACCTGCT TTTGTCACTC GGCTTTGGCT CGGGACTTCA
1561 AAATCAGTGA TGGGAGTAAG AGCAAATTTC ATCTTTCCAA ATTGATGGGT GGGCTAGTAA
1621 TAAAATATTT AAAAAAAAAC ATTCAAAAAC ATGGCCACAT CCAACATTTC CTCAGGCAAT
1681 TCCTTTTGAT TCTTTTTTCT TCCCCCTCCA TGTAGAAGAG GGAGAAGGAG AGGCTCTGAA
1741 AGCTGCTTCT GGGGGATTTC AAGGGACTGG GGGTGCCAAC CACCTCTGGC CCTGTTGTGG
1801 GGGTGTCACA GAGGCAGTGG CAGCAACAAA GGATTTGAAA CTTGGTGTGT TCGTGGAGCC
1861 ACAGGCAGAC GATGTCAACC TTGTGTGAGT GTGACGGGGG TTGGGGTGGG GCGGGAGGCC
1921 ACGGGGGAGG CCGAGGCAGG GGCTGGGCAG AGGGGAGAGG AAGCACAAGA AGTGGGAGTG
1981 GGAGAGGAAG CCACGTGCTG GAGAGTAGAC ATCCCCCTCC TTGCCGCTGG GAGAGCCAAG
2041 GCCTATGCCA CCTGCAGCGT CTGAGCGGCC GCCTGTCCTT GGTGGCCGGG GGTGGGGGCC
2101 TGCTGTGGGT CAGTGTGCCA CCCTCTGCAG GGCAGCCTGT GGGAGAAGGG ACAGCGGGTA
2161 AAAAGAGAAG GCAAGCTGGC AGGAGGGTGG CACTTCGTGG ATGACCTCCT TAGAAAAGAC
2221 TGACCTTGAT GTCTTGAGAG CGCTGGCCTC TTCCTCCCTC CCTGCAGGGT AGGGGGCCTG
2281 AGTTGAGGGG CTTCCCTCTG CTCCACAGAA ACCCTGTTTT ATTGAGTTCT GAAGGTTGGA
2341 ACTGCTGCCA TGATTTTGGC CACTTTGCAG ACCTGGGACT TTAGGGCTAA CCAGTTCTCT
2401 TTGTAAGGAC TTGTGCCTCT TGGGAGACGT CCACCCGTTT CCAAGCCTGG GCCACTGGCA
2461 TCTCTGGAGT GTGTGGGGGT CTGGGAGGCA GGTCCCGAGC CCCCTGTCCT TCCCACGGCC
2521 ACTGCAGTCA CCCCGTCTGC GCCGCTGTGC TGTTGTCTGC CGTGAGAGCC CAATCACTGC
2581 CTATACCCCT CATCACACGT CACAATGTCC CGAATTCCCA GCCTCACCAC CCCTTCTCAG
2641 TAATGACCCT GGTTGGTTGC AGGAGGTACC TACTCCATAC TGAGGGTGAA ATTAAGGGAA
2701 GGCAAAGTCC AGGCACAAGA GTGGGACCCC AGCCTCTCAC TCTCAGTTCC ACTCATCCAA
2761 CTGGGACCCT CACCACGAAT CTCATGATCT GATTCGGTTC CCTGTCTCCT CCTCCCGTCA
2821 CAGATGTGAG CCAGGGCACT GCTCAGCTGT GACCCTAGGT GTTTCTGCCT TGTTGACATG
2881 GAGAGAGCCC TTTCCCCTGA GAAGGCCTGG CCCCTTCCTG TGCTGAGCCC ACAGCAGCAG
2941 GCTGGGTGTC TTGGTTGTCA GTGGTGGCAC CAGGATGGAA GGGCAAGGCA CCCAGGGCAG
3001 GCCCACAGTC CCGCTGTCCC CCACTTGCAC CCTAGCTTGT AGCTGCCAAC CTCCCAGACA
3061 GCCCAGCCCG CTGCTCAGCT CCACATGCAT AGTATCAGCC CTCCACACCC GACAAAGGGG
3121 AACACACCCC CTTGGAAATG GTTCTTTTCC CCCAGTCCCA GCTGGAAGCC ATGCTGTCTG
3181 TTCTGCTGGA GCAGCTGAAC ATATACATAG ATGTTGCCCT GCCCTCCCCA TCTGCACCCT
3241 GTTGAGTTGT AGTTGGATTT GTCTGTTTAT GCTTGGATTC ACCAGAGTGA CTATGATAGT
3301 GAAAAGAAAA AAAAAAAAAA AAAAGGACGC ATGTATCTTG AAATGCTTGT AAAGAGGTTT
3361 CTAACCCACC CTCACGAGGT GTCTCTCACC CCCACACTGG GACTCGTGTG GCCTGTGTGG
3421 TGCCACCCTG CTGGGGCCTC CCAAGTTTTG AAAGGCTTTC CTCAGCACCT GGGACCCAAC
3481 AGAGACCAGC TTCTAGCAGC TAAGGAGGCC GTTCAGCTGT GACGAAGGCC TGAAGCACAG
3541 GATTAGGACT GAAGCGATGA TGTCCCCTTC CCTACTTCCC CTTGGGGCTC CCTGTGTCAG
3601 GGCACAGACT AGGTCTTGTG GCTGGTCTGG CTTGCGGCGC GAGGATGGTT CTCTCTGGTC
3661 ATAGCCCGAA GTCTCATGGC AGTCCCAAAG GAGGCTTACA ACTCCTGCAT CACAAGAAAA
3721 AGGAAGCCAC TGCCAGCTGG GGGGATCTGC AGCTCCCAGA AGCTCCGTGA GCCTCAGCCA
3781 CCCCTCAGAC TGGGTTCCTC TCCAAGCTCG CCCTCTGGAG GGGCAGCGCA GCCTCCCACC
3841 AAGGGCCCTG CGACCACAGC AGGGATTGGG ATGAATTGCC TGTCCTGGAT CTGCTCTAGA
3901 GGCCCAAGCT GCCTGCCTGA GGAAGGATGA CTTGACAAGT CAGGAGACAC TGTTCCCAAA
3961 GCCTTGACCA GAGCACCTCA GCCCGCTGAC CTTGCACAAA CTCCATCTGC TGCCATGAGA
4021 AAAGGGAAGC CGCCTTTGCA AAACATTGCT GCCTAAAGAA ACTCAGCAGC CTCAGGCCCA
4081 ATTCTGCCAC TTCTGGTTTG GGTACAGTTA AAGGCAACCC TGAGGGACTT GGCAGTAGAA
4141 ATCCAGGGCC TCCCCTGGGG CTGGCAGCTT CGTGTGCAGC TAGAGCTTTA CCTGAAAGGA
4201 AGTCTCTGGG CCCAGAACTC TCCACCAAGA GCCTCCCTGC CGTTCGCTGA GTCCCAGCAA
4261 TTCTCCTAAG TTGAAGGGAT CTGAGAAGGA GAAGGAAATG TGGGGTAGAT TTGGTGGTGG
4321 TTAGAGATAT GCCCCCCTCA TTACTGCCAA CAGTTTCGGC TGCATTTCTT CACGCACCTC
4381 GGTTCCTCTT CCTGAAGTTC TTGTGCCCTG CTCTTCAGCA CCATGGGCCT TCTTATACGG
4441 AAGGCTCTGG GATCTCCCCC TTGTGGGGCA GGCTCTTGGG GCCAGCCTAA GATCATGGTT
4501 TAGGGTGATC AGTGCTGGCA GATAAATTGA AAAGGCACGC TGGCTTGTGA TCTTAAATGA
4561 GGACAATCCC CCCAGGGCTG GGCACTCCTC CCCTCCCCTC ACTTCTCCCA CCTGCAGAGC
4621 CAGTGTCCTT GGGTGGGCTA GATAGGATAT ACTGTATGCC GGCTCCTTCA AGCTGCTGAC
4681 TCACTTTATC AATAGTTCCA TTTAAATTGA CTTCAGTGGT GAGACTGTAT CCTGTTTGCT
4741 ATTGCTTGTT GTGCTATGGG GGGAGGGGGG AGGAATGTGT AAGATAGTTA ACATGGGCAA
4801 AGGGAGATCT TGGGGTGCAG CACTTAAACT GCCTCGTAAC CCTTTTCATG ATTTCAACCA
4861 CATTTGCTAG AGGGAGGGAG CAGCCACGGA GTTAGAGGCC CTTGGGGTTT CTCTTTTCCA
4921 CTGACAGGCT TTCCCAGGCA GCTGGCTAGT TCATTCCCTC CCCAGCCAGG TGCAGGCGTA
4981 GGAATATGGA CATCTGGTTG CTTTGGCCTG CTGCCCTCTT TCAGGGGTCC TAAGCCCACA
5041 ATCATGCCTC CCTAAGACCT TGGCATCCTT CCCTCTAAGC CGTTGGCACC TCTGTGCCAC
5101 CTCTCACACT GGCTCCAGAC ACACAGCCTG TGCTTTTGGA GCTGAGATCA CTCGCTTCAC
5161 CCTCCTCATC TTTGTTCTCC AAGTAAAGCC ACGAGGTCGG GGCGAGGGCA GAGGTGATCA
5221 CCTGCGTGTC CCATCTACAG ACCTGCAGCT TCATAAAACT TCTGATTTCT CTTCAGCTTT
5281 GAAAAGGGTT ACCCTGGGCA CTGGCCTAGA GCCTCACCTC CTAATAGACT TAGCCCCATG
5341 AGTTTGCCAT GTTGAGCAGG ACTATTTCTG GCACTTGCAA GTCCCATGAT TTCTTCGGTA
5401 ATTCTGAGGG TGGGGGGAGG GACATGAAAT CATCTTAGCT TAGCTTTCTG TCTGTGAATG
5461 TCTATATAGT GTATTGTGTG TTTTAACAAA TGATTTACAC TGACTGTTGC TGTAAAAGTG
5521 AATTTGGAAA TAAAGTTATT ACTCTGATTA AA.
```

The corresponding amino acid sequence of human Tau protein isoform 5 can be found at NP_001116539.1:

```
(SEQ ID NO: 63)
    1 MAEPRQEFEV MEDHAGTYGL GDRKDQGGYT
        MHQDQEGDTD AGLKESPLQT PTEDGSEEPG
  61 SETSDAKSTP TAEAEEAGIG DTPSLEDEAA
        GHVTQARMVS KSKDGTGSDD KKAKGADGKT
121 KIATPRGAAP PGQKGQANAT RIPAKTPPAP
        KTPPSSGEPP KSGDRSGYSS PGSPGTPGSR
181 SRTPSLPTPP TREPKKVAVV RTPPKSPSSA
        KSRLQTAPVP MPDLKNVKSK IGSTENLKHQ
241 PGGGKVQIIN KKLDLSNVQS KCGSKDNIKH
        VPGGGSVQIV YKPVDLSKVT SKCGSLGNIH
301 HKPGGGQVEV KSEKLDFKDR VQSKIGSLDN
        ITHVPGGGNK KIETHKLTFR ENAKAKTDHG
361 AEIVYKSPVV SGDTSPRHLS NVSSTGSIDM
        VDSPQLATLA DEVSASLAKQ GL
```

The nucleotide sequence of the human MAPT transcript variant 4 (encoding 0N3R Tau) can be found at NM_016841.5:

```
      1 GCAGTCACCG CCACCCACCA GCTCCGGCAC CAACAGCAGC GCCGCTGCCA CCGCCCACCT
    61 TCTGCCGCCG CCACCACAGC CACCTTCTCC TCCTCCGCTG TCCTCTCCCG TCCTCGCCTC
  121 TGTCGACTAT CAGGTGAACT TTGAACCAGG ATGGCTGAGC CCCGCCAGGA GTTCGAAGTG
  181 ATGGAAGATC ACGCTGGGAC GTACGGGTTG GGGGACAGGA AAGATCAGGG GGGCTACACC
  241 ATGCACCAAG ACCAAGAGGG TGACACGGAC GCTGGCCTGA AAGCTGAAGA AGCAGGCATT
  301 GGAGACACCC CCAGCCTGGA AGACGAAGCT GCTGGTCACG TGACCCAAGC TCGCATGGTC
  361 AGTAAAAGCA AAGACGGGAC TGGAAGCGAT GACAAAAAAG CCAAGGGGGC TGATGGTAAA
  421 ACGAAGATCG CCACACCGCG GGGAGCAGCC CCTCCAGGCC AGAAGGGCCA GGCCAACGCC
  481 ACCAGGATTC CAGCAAAAAC CCCGCCCGCT CCAAAGACAC CACCCAGCTC TGGTGAACCT
  541 CCAAAATCAG GGGATCGCAG CGGCTACAGC AGCCCCGGCT CCCCAGGCAC TCCCGGCAGC
  601 CGCTCCCGCA CCCCGTCCCT TCCAACCCCA CCCACCCGGG AGCCCAAGAA GGTGGCAGTG
  661 GTCCGTACTC CACCCAAGTC GCCGTCTTCC GCCAAGAGCC GCCTGCAGAC AGCCCCCGTG
  721 CCCATGCCAG ACCTGAAGAA TGTCAAGTCC AAGATCGGCT CCACTGAGAA CCTGAAGCAC
  781 CAGCCGGGAG GCGGGAAGGT GCAAATAGTC TACAAACCAG TTGACCTGAG CAAGGTGACC
  841 TCCAAGTGTG GCTCATTAGG CAACATCCAT CATAAACCAG GAGGTGGCCA GGTGGAAGTA
  901 AAATCTGAGA AGCTTGACTT CAAGGACAGA GTCCAGTCGA AGATTGGGTC CCTGGACAAT
  961 ATCACCCACG TCCCTGGCGG AGGAAATAAA AAGATTGAAA CCCACAAGCT GACCTTCCGC
1021 GAGAACGCCA AAGCCAAGAC AGACCACGGG GCGGAGATCG TGTACAAGTC GCCAGTGGTG
1081 TCTGGGGACA CGTCTCCACG GCATCTCAGC AATGTCTCCT CCACCGGCAG CATCGACATG
1141 GTAGACTCGC CCCAGCTCGC CACGCTAGCT GACGAGGTGT CTGCCTCCCT GGCCAAGCAG
1201 GGTTTGTGAT CAGGCCCCTG GGGCGGTCAA TAATTGTGGA GAGGAGAGAA TGAGAGAGTG
1261 TGGAAAAAAA AAGAATAATG ACCCGGCCCC CGCCCTCTGC CCCCAGCTGC TCCTCGCAGT
1321 TCGGTTAATT GGTTAATCAC TTAACCTGCT TTTGTCACTC GGCTTTGGCT CGGGACTTCA
1381 AAATCAGTGA TGGGAGTAAG AGCAAATTTC ATCTTTCCAA ATTGATGGGT GGGCTAGTAA
1441 TAAAATATTT AAAAAAAAAC ATTCAAAAAC ATGGCCACAT CCAACATTTC CTCAGGCAAT
1501 TCCTTTTGAT TCTTTTTTCT TCCCCCTCCA TGTAGAAGAG GGAGAAGGAG AGGCTCTGAA
1561 AGCTGCTTCT GGGGGATTTC AAGGGACTGG GGGTGCCAAC CACCTCTGGC CCTGTTGTGG
1621 GGGTGTCACA GAGGCAGTGG CAGCAACAAA GGATTTGAAA CTTGGTGTGT TCGTGGAGCC
1681 ACAGGCAGAC GATGTCAACC TTGTGTGAGT GTGACGGGGG TTGGGGTGGG GCGGGAGGCC
1741 ACGGGGGAGG CCGAGGCAGG GGCTGGGCAG AGGGGAGAGG AAGCACAAGA AGTGGGAGTG
1801 GGAGAGGAAG CCACGTGCTG GAGAGTAGAC ATCCCCCTCC TTGCCGCTGG GAGAGCCAAG
1861 GCCTATGCCA CCTGCAGCGT CTGAGCGGCC GCCTGTCCTT GGTGGCCGGG GGTGGGGGCC
1921 TGCTGTGGGT CAGTGTGCCA CCCTCTGCAG GGCAGCCTGT GGGAGAAGGG ACAGCGGGTA
1981 AAAAGAGAAG GCAAGCTGGC AGGAGGGTGG CACTTCGTGG ATGACCTCCT TAGAAAAGAC
2041 TGACCTTGAT GTCTTGAGAG CGCTGGCCTC TTCCTCCCTC CCTGCAGGGT AGGGGGCCTG
2101 AGTTGAGGGG CTTCCCTCTG CTCCACAGAA ACCCTGTTTT ATTGAGTTCT GAAGGTTGGA
2161 ACTGCTGCCA TGATTTTGGC CACTTTGCAG ACCTGGGACT TTAGGGCTAA CCAGTTCTCT
2221 TTGTAAGGAC TTGTGCCTCT TGGGAGACGT CCACCCGTTT CCAAGCCTGG GCCACTGGCA
2281 TCTCTGGAGT GTGTGGGGGT CTGGGAGGCA GGTCCCGAGC CCCCTGTCCT TCCCACGGCC
2341 ACTGCAGTCA CCCCGTCTGC GCCGCTGTGC TGTTGTCTGC CGTGAGAGCC CAATCACTGC
2401 CTATACCCCT CATCACACGT CACAATGTCC CGAATTCCCA GCCTCACCAC CCCTTCTCAG
2461 TAATGACCCT GGTTGGTTGC AGGAGGTACC TACTCCATAC TGAGGGTGAA ATTAAGGGAA
2521 GGCAAAGTCC AGGCACAAGA GTGGGACCCC AGCCTCTCAC TCTCAGTTCC ACTCATCCAA
2581 CTGGGACCCT CACCACGAAT CTCATGATCT GATTCGGTTC CCTGTCTCCT CCTCCCGTCA
2641 CAGATGTGAG CCAGGGCACT GCTCAGCTGT GACCCTAGGT GTTTCTGCCT TGTTGACATG
2701 GAGAGAGCCC TTTCCCCTGA GAAGGCCTGG CCCCTTCCTG TGCTGAGCCC ACAGCAGCAG
2761 GCTGGGTGTC TTGGTTGTCA GTGGTGGCAC CAGGATGGAA GGGCAAGGCA CCCAGGGCAG
2821 GCCCACAGTC CCGCTGTCCC CCACTTGCAC CCTAGCTTGT AGCTGCCAAC CTCCCAGACA
2881 GCCCAGCCCG CTGCTCAGCT CCACATGCAT AGTATCAGCC CTCCACACCC GACAAAGGGG
2941 AACACACCCC CTTGGAAATG GTTCTTTTCC CCCAGTCCCA GCTGGAAGCC ATGCTGTCTG
3001 TTCTGCTGGA GCAGCTGAAC ATATACATAG ATGTTGCCCT GCCCTCCCCA TCTGCACCCT
3061 GTTGAGTTGT AGTTGGATTT GTCTGTTTAT GCTTGGATTC ACCAGAGTGA CTATGATAGT
3121 GAAAAGAAAA AAAAAAAAAA AAAAGGACGC ATGTATCTTG AAATGCTTGT AAAGAGGTTT
3181 CTAACCCACC CTCACGAGGT GTCTCTCACC CCCACACTGG GACTCGTGTG GCCTGTGTGG
3241 TGCCACCCTG CTGGGGCCTC CCAAGTTTTG AAAGGCTTTC CTCAGCACCT GGGACCCAAC
3301 AGAGACCAGC TTCTAGCAGC TAAGGAGGCC GTTCAGCTGT GACGAAGGCC TGAAGCACAG
3361 GATTAGGACT GAAGCGATGA TGTCCCCTTC CCTACTTCCC CTTGGGGCTC CCTGTGTCAG
3421 GGCACAGACT AGGTCTTGTG GCTGGTCTGG CTTGCGGCGC GAGGATGGTT CTCTCTGGTC
3481 ATAGCCCGAA GTCTCATGGC AGTCCCAAAG GAGGCTTACA ACTCCTGCAT CACAAGAAAA
3541 AGGAAGCCAC TGCCAGCTGG GGGGATCTGC AGCTCCCAGA AGCTCCGTGA GCCTCAGCCA
3601 CCCCTCAGAC TGGGTTCCTC TCCAAGCTCG CCCTCTGGAG GGGCAGCGCA GCCTCCCACC
3661 AAGGGCCCTG CGACCACAGC AGGGATTGGG ATGAATTGCC TGTCCTGGAT CTGCTCTAGA
3721 GGCCCAAGCT GCCTGCCTGA GGAAGGATGA CTTGACAAGT CAGGAGACAC TGTTCCCAAA
3781 GCCTTGACCA GAGCACCTCA GCCCGCTGAC CTTGCACAAA CTCCATCTGC TGCCATGAGA
3841 AAAGGGAAGC CGCCTTTGCA AAACATTGCT GCCTAAAGAA ACTCAGCAGC CTCAGGCCCA
3901 ATTCTGCCAC TTCTGGTTTG GGTACAGTTA AAGGCAACCC TGAGGGACTT GGCAGTAGAA
3961 ATCCAGGGCC TCCCCTGGGG CTGGCAGCTT CGTGTGCAGC TAGAGCTTTA CCTGAAAGGA
4021 AGTCTCTGGG CCCAGAACTC TCCACCAAGA GCCTCCCTGC CGTTCGCTGA GTCCCAGCAA
4081 TTCTCCTAAG TTGAAGGGAT CTGAGAAGGA GAAGGAAATG TGGGGTAGAT TTGGTGGTGG
4141 TTAGAGATAT GCCCCCCTCA TTACTGCCAA CAGTTTCGGC TGCATTTCTT CACGCACCTC
4201 GGTTCCTCTT CCTGAAGTTC TTGTGCCCTG CTCTTCAGCA CCATGGGCCT TCTTATACGG
4261 AAGGCTCTGG GATCTCCCCC TTGTGGGGCA GGCTCTTGGG GCCAGCCTAA GATCATGGTT
4321 TAGGGTGATC AGTGCTGGCA GATAAATTGA AAAGGCACGC TGGCTTGTGA TCTTAAATGA
4381 GGACAATCCC CCCAGGGCTG GGCACTCCTC CCCTCCCCTC ACTTCTCCCA CCTGCAGAGC
4441 CAGTGTCCTT GGGTGGGCTA GATAGGATAT ACTGTATGCC GGCTCCTTCA AGCTGCTGAC
4501 TCACTTTATC AATAGTTCCA TTTAAATTGA CTTCAGTGGT GAGACTGTAT CCTGTTTGCT
4561 ATTGCTTGTT GTGCTATGGG GGGAGGGGGG AGGAATGTGT AAGATAGTTA ACATGGGCAA
4621 AGGGAGATCT TGGGGTGCAG CACTTAAACT GCCTCGTAAC CCTTTTCATG ATTTCAACCA
4681 CATTTGCTAG AGGGAGGGAG CAGCCACGGA GTTAGAGGCC CTTGGGGTTT CTCTTTTCCA
4741 CTGACAGGCT TTCCCAGGCA GCTGGCTAGT TCATTCCCTC CCCAGCCAGG TGCAGGCGTA
4801 GGAATATGGA CATCTGGTTG CTTTGGCCTG CTGCCCTCTT TCAGGGGTCC TAAGCCCACA
4861 ATCATGCCTC CCTAAGACCT TGGCATCCTT CCCTCTAAGC CGTTGGCACC TCTGTGCCAC
4921 CTCTCACACT GGCTCCAGAC ACACAGCCTG TGCTTTTGGA GCTGAGATCA CTCGCTTCAC
4981 CCTCCTCATC TTTGTTCTCC AAGTAAAGCC ACGAGGTCGG GGCGAGGGCA GAGGTGATCA
5041 CCTGCGTGTC CCATCTACAG ACCTGCAGCT TCATAAAACT TCTGATTTCT CTTCAGCTTT
5101 GAAAAGGGTT ACCCTGGGCA CTGGCCTAGA GCCTCACCTC CTAATAGACT TAGCCCCATG
5161 AGTTTGCCAT GTTGAGCAGG ACTATTTCTG GCACTTGCAA GTCCCATGAT TTCTTCGGTA
5221 ATTCTGAGGG TGGGGGGAGG GACATGAAAT CATCTTAGCT TAGCTTTCTG TCTGTGAATG
5281 TCTATATAGT GTATTGTGTG TTTTAACAAA TGATTTACAC TGACTGTTGC TGTAAAAGTG
5341 AATTTGGAAA TAAAGTTATT ACTCTGATTA AA
(SEQ ID NO: 64).
```

The corresponding amino acid sequence of human Tau protein isoform 4 can be found at NP_058525.1:

```
(SEQ ID NO: 65)
    1 MAEPRQEFEV MEDHAGTYGL GDRKDQGGYT
        MHQDQEGDTD AGLKAEEAGI GDTPSLEDEA
  61 AGHVTQARMV SKSKDGTGSD DKKAKGADGK
        TKIATPRGAA PPGQKGQANA TRIPAKTPPA
121 PKTPPSSGEP PKSGDRSGYS SPGSPGTPGS
        RSRTPSLPTP PTREPKKVAV VRTPPKSPSS
181 AKSRLQTAPV PMPDLKNVKS KIGSTENLKH
        QPGGGKVQIV YKPVDLSKVT SKCGSLGNIH
241 HKPGGGQVEV KSEKLDFKDR VQSKIGSLDN
        ITHVPGGGNK KIETHKLTFR ENAKAKTDHG
301 AEIVYKSPVV SGDTSPRHLS NVSSTGSIDM
        VDSPQLATLA DEVSASLAKQ GL
```

As used herein, “subject” means a mammal, including cat, dog, mouse, rat, chimpanzee, ape, monkey, and human. Preferably the subject is a human.

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

### EXAMPLES

### Example 1. Synthesis of the Compounds and RNAi Agents

Certain abbreviations are defined as follows: “ACN” refers to acetonitrile, “AEX” refers to anion exchange; “C/D” refers to cleavage and deprotection; “CPG” refers to controlled pore glass; “aCSF” refers to artificial cerebral spinal fluid; “DCM” refers to dichloromethane; “DEA” refers to diethylamine; “DIPEA” refers to N,N-diisopropylethylamine; “DMA” refers to dimethylacetamide; “DMAP” refers to 4-dimethylaminopyridine; “DMF” refers to dimethylformamide; “DMSO” refers to dimethyl sulfoxide; “DMT” refers to 4,4′-dimethoxytrityl; “EDCI” refers to 1-ethyl-3-(3-dimethylaminopropyl)carbodiimide; “ES/MS” refers to electrospray mass spectrometry; “EtOAc” refers to ethyl acetate; “EtOH” refers to ethanol and ethyl alcohol; “IP-RP” refers to ion-pair reverse phase; “LC/MS” refers to liquid chromatography-mass spectrometry; “MeOH” refers to methanol and methyl alcohol; “MPA” refers to mobile phase A; “MPB” refers to mobile phase B; “MWCO” refers to molecular weight cut-off, “NaOAc” refers to sodium acetate; “NHS” refers to N-hydroxysuccinimide; “NMR” refers to nuclear magnetic resonance; “PBS” phosphate-buffered saline; “PVDF” refers to polyvinylidene fluoride; “RP” refers to reverse phase; “siRNA” refers to small interfering ribonucleic acid; “TCEP” refers to tris(2-carboxyethyl)phosphine; “TEA” refers to triethylamine; “TFA” refers to trifluoracetic acid; “THE” refers to tetrahydrofuran; “UPLC” refers to ultra-performance liquid chromatography; and “UV” refers to ultraviolet.

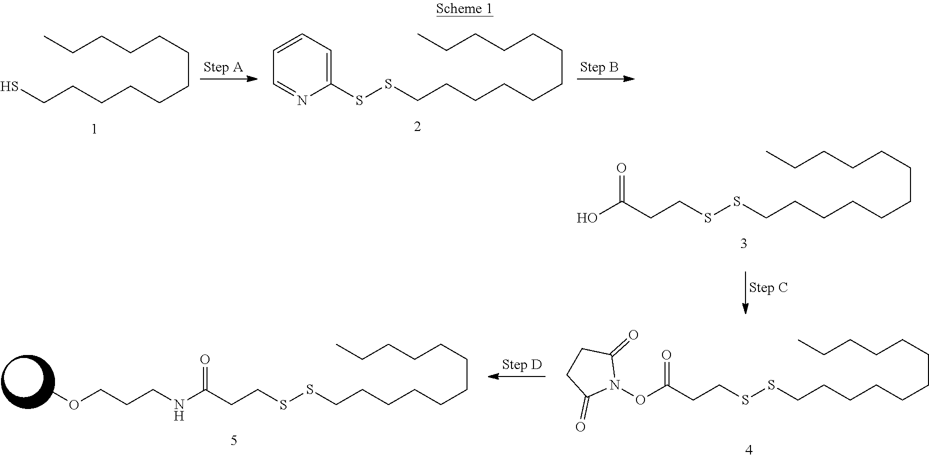 [SMILES: CCCCCCCCCCCCS ; CCCCCCCCCCCCSSc1ccccn1 ; CCCCCCCCCCCCSSCCC(=O)O ; CCCCCCCCCCCCSSCCC(=O)NCCCOC ; CCCCCCCCCCCCSSCCC(=O)ON1C(=O)CCC1=O]

Scheme 1, step A depicts the reaction of compound (1) with 2,2′-dipyridyl disulfide in a solvent system such as MeOH and THE to give compound (2). Step B shows the reaction of compound (2) with 3-sulfanylpropionic acid in a solvent such as MeOH to give compound (3). Step C shows the addition of NHS to compound (3) using a coupling reagent such as EDCI and a catalyst such as DMAP in a solvent such as DCM to give compound (4). Step D shows the addition of compound (4) to an appropriate modified sense strand in the presence of a borate buffer to give compound (5).

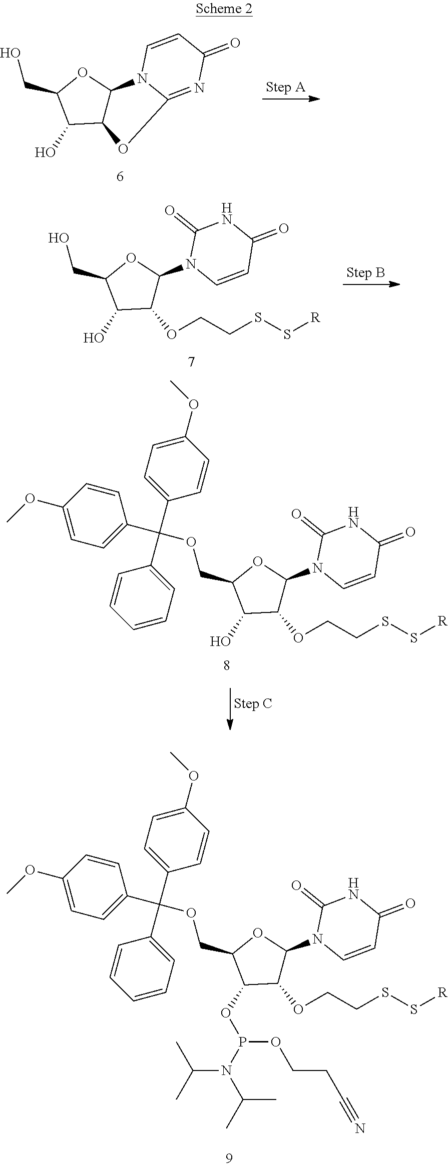

Scheme 2, step A depicts the ring opening addition of an appropriate substituted (disulfanyl)ethanol reagent to compound (6) using boron trifluoride diethyl etherate in a solvent such as DMA to give compound (7). Step B shows the protection of compound (7) with dimethoxytrityl chloride using a base such as TEA and a catalyst such as DMAP in a solvent such as pyridine to give compound (8). Step C depicts the addition of 2-cyanoethyl-N,N-diisopropylchlorophosphoramidite to compound (8) using a base such as DIPEA in a solvent such as DCM to give compound (9).

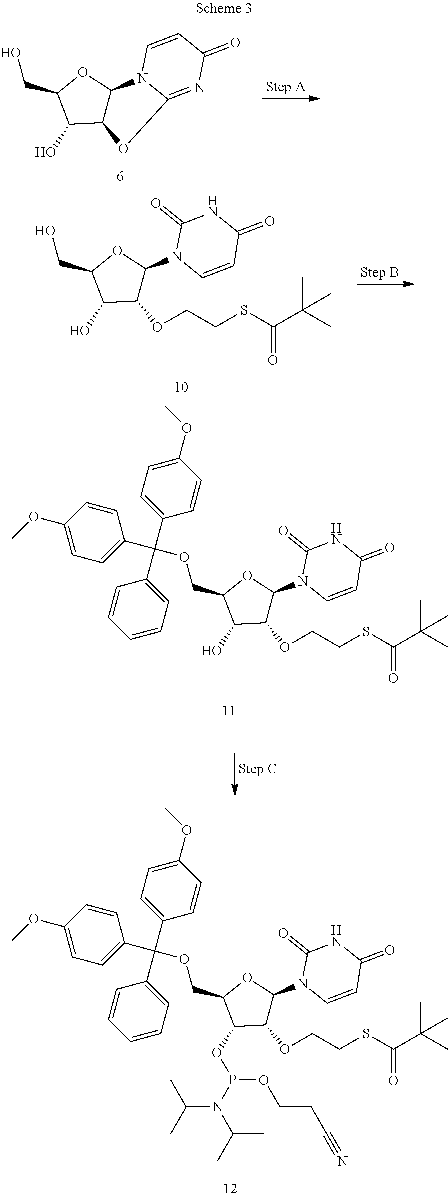

Scheme 3, steps A-C show the transformation of compound (6) to compound (12) and are essentially analogous to the processes found in Scheme 2, steps A-C.

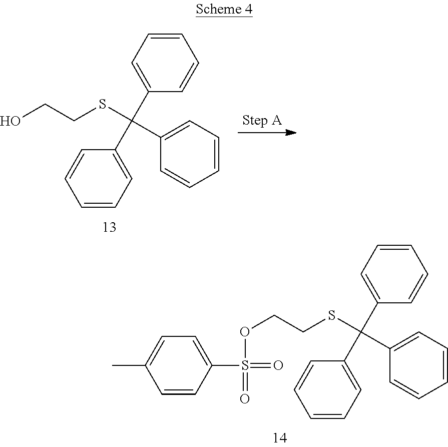 [SMILES: OCCSC(c1ccccc1)(c1ccccc1)c1ccccc1 ; Cc1ccc(S(=O)(=O)OCCSC(c2ccccc2)(c2ccccc2)c2ccccc2)cc1]

Scheme 4, step A depicts the tosylation of compound (13) using p-toluenesulfonyl chloride and a base such as pyridine in a solvent such as DCM to give compound (14).

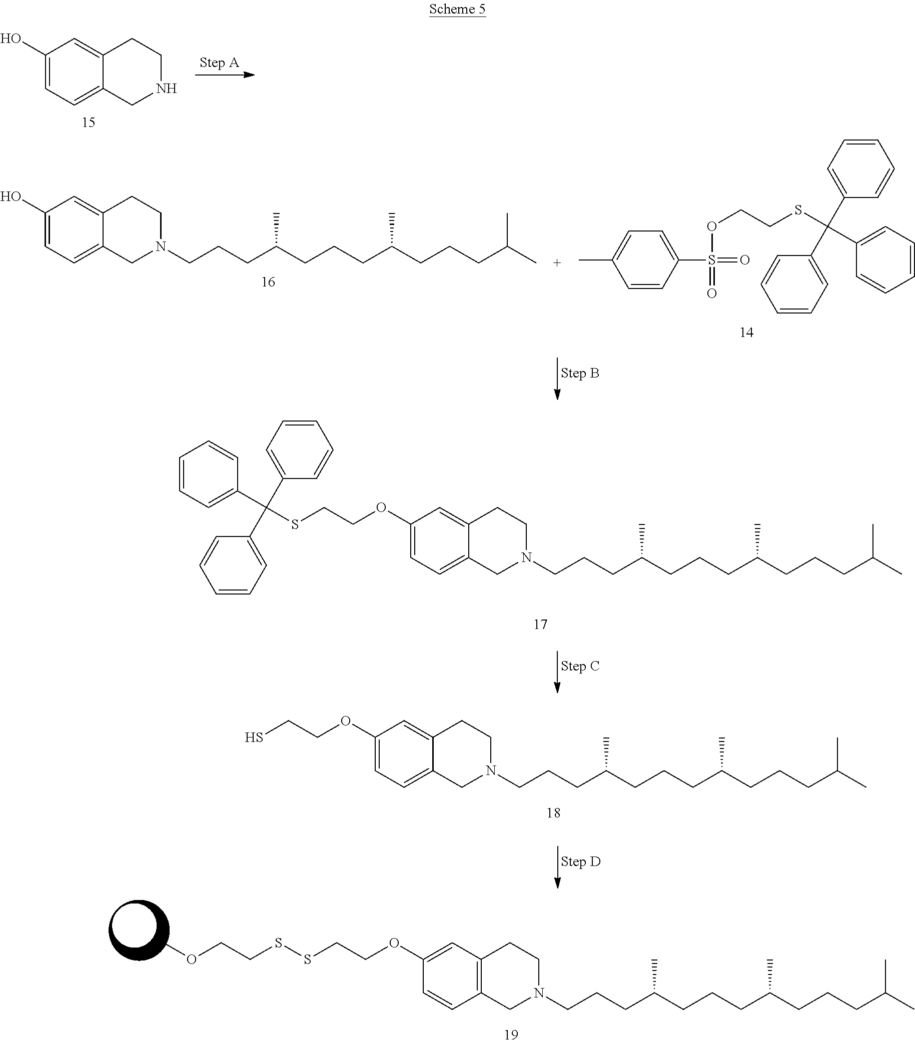

Scheme 5, step A shows the alkylation of compound (15) with (4R,8R)-1-iodo-4,8,12-trimethyltridecane using a base such as potassium carbonate in a solvent such as DMF to give compound (16). Step B shows the coupling of compounds (14) and (16) using a base such as cesium carbonate in a solvent such as DMF to give compound (17). Step C depicts the deprotection of compound (17) through use of TFA and triethylsilane in a solvent such as DCM to give compound (18). Step D shows the coupling of compound (18) to an appropriate modified sense strand partner in the presence of TCEP to give compound (19).

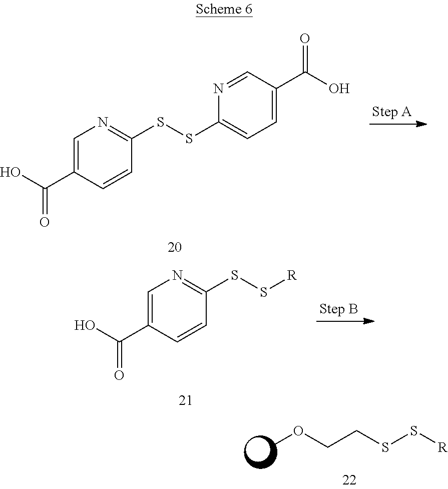

Scheme 6, step A depicts the reaction of compound (20) with an appropriate thiol such as 2-((3r,5r,7r)-adamantan-1-yl)ethane-1-thiol or dodecane-1-thiol in the presence of borate buffer to give compound (21). Step B shows the addition of compound (21) to an appropriate modified sense strand partner in the presence of AMA solution to give compound (22).

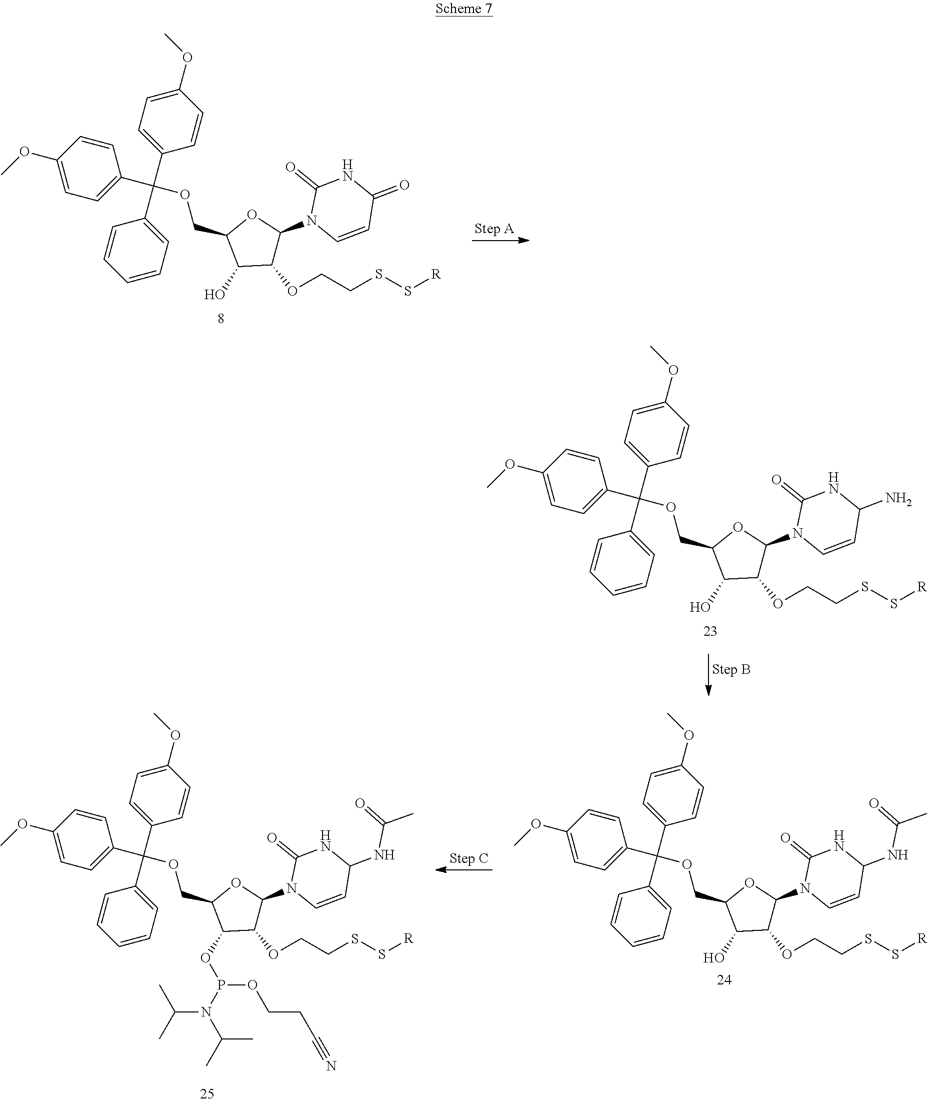

Scheme 7, step A depicts the conversion of compound (8) to compound (23) by first adding chlorotrimethylsilane in a solvent such as pyridine followed by treatment with 1,2,4-triazole, TEA, and phosphoryl chloride before finally adding ammonia to give compound (23). Step B shows the acylation of compound (23) using acetic anhydride in a solvent such as DMF to give compound (24). Step C shows the conversion of compound (24) to compound (25) and is essentially analogous to the processes found in Scheme 2, step C.

### Preparation 1

### 2-(Dodecyldisulfaneyl)pyridine

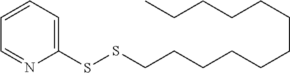 [SMILES: CCCCCCCCCCCCSSc1ccccn1]

1-Dodecanethiol (12.7 g, 61.4 mmol) was added to a solution of 2,2′-dipyridyl disulfide (20.5 g, 92.1 mmol) in MeOH (90 mL) and THF (5 mL). The mixture was stirred at ambient temperature for 16 hours then concentrated in vacuo. The resulting residue was purified via silica gel flash chromatography eluting with 0-15% EtOAc in hexanes to give the title compound as a colorless oil (14.35 g, 75%). ES/MS (m/z): 312 (M+H).

The compound in Table 15 were prepared in a manner essentially analogous to that found in Preparation 1.

TABLE 15

| Prep | Chemical Name | Structure | ES/MS (m/z) |
|---|---|---|---|
| 1a | 2-(Hexadecyldisulfaneyl)pyridine | 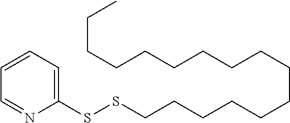 [SMILES: CCCCCCCCCCCCCCCCSSc1ccccn1] | 368.4 (M + H) |

### Preparation 2

### 3-(Dodecyldisulfaneyl)propanoic acid

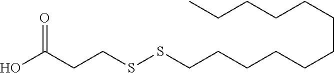 [SMILES: CCCCCCCCCCCCSSCCC(=O)O]

3-Sulfanylpropionic acid (7.58 g, 71.44 mmol) was added to a solution of 2-(dodecyldisulfaneyl)pyridine (18.55 g, 59.5 mmol) in MeOH (60 mL). The reaction was stirred at ambient temperature for 1 hour, then concentrated in vacuo. The resulting residue was purified via silica gel flash chromatography eluting with 5-30% EtOAc in hexanes to give the title compound as a colorless oil (14 g, 76%). ¹H NMR (DMSO-d₆) δ 2.86 (t, 2H, J=7.0 Hz), 2.71 (t, 2H, J=7.0 Hz), 2.62 (t, 2H, J=7.0 Hz), 1.61 (quint, 2H), 1.33 (q, 2H), 1.28 (s, 16H), 0.90 (t, 3H, J=6.8 Hz).

The compound in Table 16 were prepared in a manner essentially analogous to that found in Preparation 2.

TABLE 16

| Prep | Name | Structure | ¹H NMR (DMSO-d₆ |
|---|---|---|---|
| 2a | 3- (Hexadecyldisulfaneyl)propanoic acid | 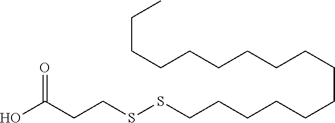 [SMILES: CCCCCCCCCCCCCCCCSSCCC(=O)O] | δ 12.35 (s, 1H), 2.86 (t, 2H, J = 7.0 Hz), 2.71 (t, 2H, J = 7.0 Hz), 2.62 (t, 2H, J = 7.0 Hz), 1.61 (quint, 2H), 1.33 (q, 2H), 1.28 (s, 24H), 0.90 (t, 3H, J = 6.8 Hz). |

### Preparation 3

### 2,5-Dioxopyrrolidin-1-yl 3-(dodecyldisulfaneyl)propanoate

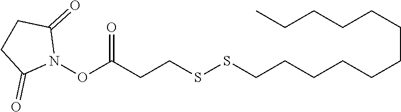 [SMILES: CCCCCCCCCCCCSSCCC(=O)ON1C(=O)CCC1=O]

NHS (1.35 g, 11.7 mmol) was added to a solution of 3-(dodecyldisulfaneyl)propanoic acid (3.0 g, 9.8 mmol), EDCI (2.25 g, 11.7 mmol), and DMAP (0.24 g, 2 mmol) in DCM (39 mL). The mixture was stirred at ambient temperature for 3 hours, then concentrated in vacuo. The resulting residue was purified via silica gel flash chromatography eluting with 0-40% EtOAc in hexanes to give the title compound as a white solid (3.2 g, 81%). ¹H NMR (DMSO-d₆) δ 3.10 (t, 2H, J=6.3 Hz), 2.99 (t, 2H, J=6.3 Hz), 2.80 (s, 4H), 2.75 (t, 2H, J=7.0 Hz), 1.61 (quint, 2H), 1.33 (q, 2H), 1.28 (s, 16H), 0.90 (t, 3H, J=6.8 Hz).

The compound in Table 17 were prepared in a manner essentially analogous to that found in Preparation 3.

TABLE 17

| Prep | Name | Structure | ¹H NMR (DMSO-d₆) |
|---|---|---|---|
| 3a | 2,5-Dioxopyrrolidin-1-yl 3- (hexadecyldisulfaneyl)propanoate | 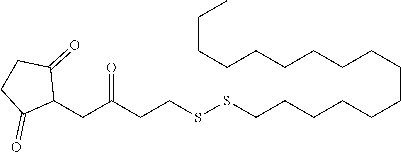 [SMILES: CCCCCCCCCCCCCCCCSSCCC(=O)CC1C(=O)CCC1=O] | δ 3.62 (s, 4H), 2.90 (t, 2H, J = 6.3 Hz), 2.71 (m, 4H), 1.61 (quint, 2H), 1.33 (q, 2H), 1.28 (s, 24H), 0.90 (t, 3H, J = 6.8 Hz). |

### Preparation 4

### 1-((2R,3R,4R,5R)-3-(2-(tert-Butyldisulfaneyl)ethoxy)-4-hydroxy-5-(hydroxymethyl)tetrahydrofuran-2-yl)pyrimidine-2,4(1H,3H)-dione

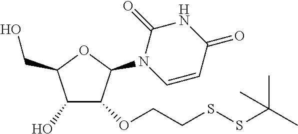 [SMILES: CC(C)(C)SSCCO[C@@H]1[C@H](O)[C@@H](CO)O[C@H]1n1ccc(=O)[nH]c1=O]

To a suspension of 2,2′-anhydro-1-(beta-D-arabinofuranosyl)uracil (4.80 g, 20.8 mmol), 2-(tert-butyldisulfanyl)ethanol (3.80 g, 22.9 mmol), and DMA (21 mL) was added boron trifluoride diethyl etherate (4.0 mL, 31.2 mmol). The mixture was heated to 130° C. for 24 hours, then cooled to ambient temperature and diluted with EtOAc (150 mL). The solution was washed with saturated aqueous sodium chloride (4×50 mL). Silica gel (10 g) was added to the organics, then concentrated in vacuo to a dry powder, and purified via silica gel flash chromatography eluting with 50-100% (5% MeOH/EtOAc) in hexanes to give the title compound as a thick, colorless oil (2.10 g, 25%). ¹H NMR (CD₃CN) δ 7.89 (d, 1H), 5.86 (d, 1H), 5.63 (d, 1H), 4.19 (q, 1H), 4.03-3.67 (m, 6H), 3.31 (t, 1H), 3.22 (d, 1H), 2.95 (t, 2H), 1.35 (s, 9H).

### Preparation 5

### 1-((2R,3R,4R,5R)-5-((Bis(4-methoxyphenyl)(phenyl)methoxy)methyl)-3-(2-(tert-butyldisulfaneyl)ethoxy)-4-hydroxytetrahydrofuran-2-yl)pyrimidine-2,4(1H,3H)-dione

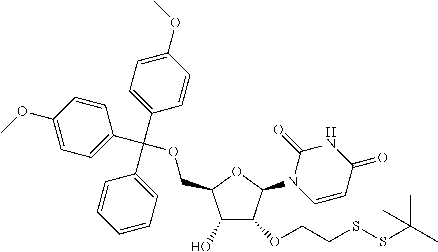 [SMILES: COc1ccc(C(OC[C@H]2O[C@@H](n3ccc(=O)[nH]c3=O)[C@H](OCCSSC(C)(C)C)[C@@H]2O)(c2ccccc2)c2ccc(OC)cc2)cc1]

A solution of 1-((2R,3R,4R,5R)-3-(2-(tert-butyldisulfaneyl)ethoxy)-4-hydroxy-5-(hydroxymethyl)tetrahydrofuran-2-yl)pyrimidine-2,4(1H,3H)-dione (1.95 g, 5.0 mmol), 4,4′-dimethoxytrityl chloride (2.23 g, 6.5 mmol), TEA (0.91 mL, 6.5 mmol), DMAP (123 mg, 1.0 mmol), and pyridine (14 mL) was stirred at ambient temperature for 5 hours. The reaction was then quenched with MeOH (10 mL) and concentrated in vacuo. The residue was suspended in DCM (25 mL), added to silica gel (10 g), concentrated in vacuo to a dry powder, and purified via silica gel flash chromatography eluting with 20-70% EtOAc in hexanes to give the title compound as a white foam (2.70 g, 78%). ¹H NMR (CD₃CN) δ 7.76 (d, 1H), 7.46 (d, 2H), 7.40-7.25 (m, 7H), 6.92 (d, 4H), 5.86 (d, 1H), 5.28 (d, 1H), 4.36 (q, 1H), 4.05-3.87 (m, 4H), 3.80 (s, 6H), 3.45-3.35 (m, 2H), 3.23 (d, 1H), 2.98 (t, 2H), 1.35 (s, 9H).

### Preparation 6

### (2R,3R,4R,5R)-5-(4-Acetamido-2-oxopyrimidin-1(2H)-yl)-2-((bis(4-methoxyphenyl)(phenyl)methoxy)methyl)-4-(2-(tert-butyldisulfaneyl)ethoxy)tetrahydrofuran-3-yl (2-cyanoethyl) diisopropylphosphoramidite

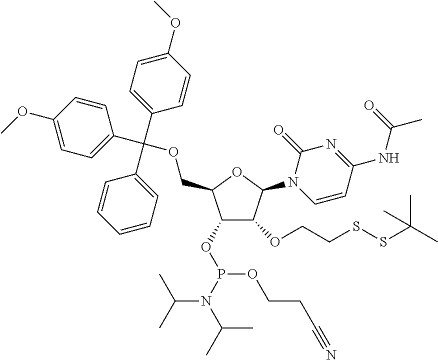 [SMILES: COc1ccc(C(OC[C@H]2O[C@@H](n3ccc(NC(C)=O)nc3=O)[C@H](OCCSSC(C)(C)C)[C@@H]2OP(OCCC#N)N(C(C)C)C(C)C)(c2ccccc2)c2ccc(OC)cc2)cc1]

Starting from 1-((2R,3R,4R,5R)-5-((bis(4-methoxyphenyl)(phenyl)methoxy)methyl)-3-(2-(tert-butyldisulfaneyl)ethoxy)-4-hydroxytetrahydrofuran-2-yl)pyrimidine-2,4(1H,3H)-dione, the title compound was synthesized using methods similar to those described in WO2019/217459.

Step 1: A mixture of 1-((2R,3R,4R,5R)-5-((bis(4-methoxyphenyl)(phenyl)methoxy)methyl)-3-(2-(tert-butyldisulfaneyl)ethoxy)-4-hydroxytetrahydrofuran-2-yl)pyrimidine-2,4(1H,3H)-dione (2.928 g, 4.21 mmol), pyridine (32.5 mL, 401.6 mmol), and chlorotrimethylsilane (2.14 mL, 16.85 mmol) was stirred at ambient temperature for 30 minutes. After this time, 1,2,4-triazole (3.26 g, 47.19 mmol) and triethylamine (8.7 mL, 62.36 mmol) were added and the mixture was stirred for 10 minutes before cooling to 0° C. Phosphoryl chloride (0.98 mL, 10.53 mmol) was added and the reaction mixture was left to stir at 0° C. for 2 hrs. Ammonia (10.53 mL, 465 mmol) was then added, and the mixture was allowed to stir at ambient temperature for 4.5 hours. The reaction mixture was quenched with 50/50 water/saturated aqueous sodium chloride, extracted with EtOAC (3×), dried over sodium sulfate, and concentrated in vacuo. The resulting residue was purified via silica gel flash chromatography eluting with 0-100% MeOH in EtOAc to give 4-amino-1-[(2R,3R,4R,5R)-5-[[bis(4-methoxyphenyl)-phenyl-methoxy]methyl]-3-[2-(tert-butyldisulfanyl)ethoxy]-4-hydroxy-tetrahydrofuran-2-yl]pyrimidin-2-one as a tan foam (2.26 g, 77%). ES/MS (m/z): 692 (M−H).

Step 2: Acetic anhydride (0.62 mL, 6.51 mmol) was added to a solution of 4-amino-1-[(2R,3R,4R,5R)-5-[[bis(4-methoxyphenyl)-phenyl-methoxy]methyl]-3-[2-(tert-butyldisulfanyl)ethoxy]-4-hydroxy-tetrahydrofuran-2-yl]pyrimidin-2-one (2.26 g, 3.26 mmol) in DMF (20 mL) and stirred at ambient temperature for 22 hours. The reaction was then quenched with water and extracted with DCM (3×). The combined organics were washed with water and saturated aqueous sodium chloride, dried over sodium sulfate, filtered, and concentrated in vacuo. The resulting residue was purified via silica gel flash chromatography eluting with 0-100% MeOH in EtOAc to give N-[1-[(2R,3R,4R,5R)-5-[[bis(4-methoxyphenyl)-phenyl-methoxy]methyl]-3-[2-(tert-butyldisulfanyl)ethoxy]-4-hydroxy-tetrahydrofuran-2-yl]-2-oxo-pyrimidin-4-yl]acetamide (837 mg, 35%). ES/MS (m/z): 734 (M−H).

Step 3: N-[1-[(2R,3R,4R,5R)-5-[[bis(4-methoxyphenyl)-phenyl-methoxy]methyl]-3-[2-(tert-butyldisulfanyl)ethoxy]-4-hydroxy-tetrahydrofuran-2-yl]-2-oxo-pyrimidin-4-yl]acetamide (0.8374 g, 1.138 mmol), DCM (7.539 g, 0.2 M), DIPEA (0.450 g, 3.414 mmol), and 2-cyanoethyl N,N-diisopropylchlorophosphoramidite (0.472 g, 1.934 mmol) were added together and stirred at ambient temperature. After one hour, additional 2-cyanoethyl N,N-diisopropylchlorophosphoramidite (0.11 mL, 0.455 mmol) was added to the mixture After one hour at ambient temperature, DCM (25 mL) was added. The mixture was washed with saturated aqueous sodium bicarbonate (3×), dried over sodium sulfate, filtered, and concentrated in vacuo. The resulting residue was purified via silica gel flash chromatography eluting with 50-100% EtOAc in hexane to give the title compound (717 mg, 67%). ¹H NMR (CD₃CN) 8.45 (d, 0.5H), 8.36 (d, 0.5H), 7.52-7.44 (m, 3H), 7.41-7.26 (m, 6H), 6.97-6.87 (m, 5H), 5.91-5.86 (m, 1H), 4.61-4.53 (m, 0.5H), 4.48-4.41 (m, 0.5H), 4.23-3.40 (m, 19H), 3.05-2.95 (m, 2H), 2.66 (t, 1H), 2.53 (t, 1H), 1.37-1.03 (m, 21H). ³¹P NMR (CD₃CN) 149.7, 148.7.

### Preparation 7

### (2R,3R,4R,5R)-2-((Bis(4-methoxyphenyl)(phenyl)methoxy)methyl)-4-(2-(tert-butyldisulfaneyl)ethoxy)-5-(2,4-dioxo-3,4-dihydropyrimidin-1(2H)-yl)tetrahydrofuran-3-yl (2-cyanoethyl) diisopropylphosphoramidite

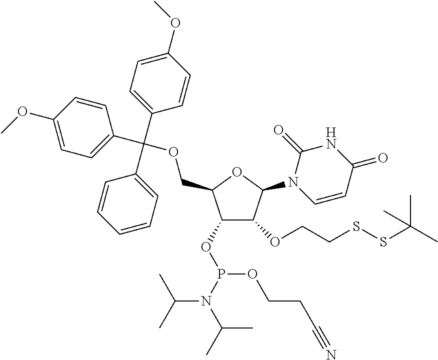 [SMILES: COc1ccc(C(OC[C@H]2O[C@@H](n3ccc(=O)[nH]c3=O)[C@H](OCCSSC(C)(C)C)[C@@H]2OP(OCCC#N)N(C(C)C)C(C)C)(c2ccccc2)c2ccc(OC)cc2)cc1]

A solution of 1-((2R,3R,4R,5R)-5-((bis(4-methoxyphenyl)(phenyl)methoxy)methyl)-3-(2-(tert-butyldisulfaneyl)ethoxy)-4-hydroxytetrahydrofuran-2-yl)pyrimidine-2,4(1H,3H)-dione (2.70 g, 3.90 mmol), 2-cyanoethyl-N,N-diisopropylchlorophosphoramidite (1.52 mL, 6.6 mmol), DIPEA (2.05 mL, 11.7 mmol), and DCM (20 mL) was stirred at ambient temperature. After 1 hour, additional 2-cyanoethyl-N,N-diisopropylchlorophosphoramidite (0.36 mL, 1.6 mmol) was added. After 1 hour, the crude reaction was poured into a slurry of silica gel (10 g) in 20 mL of 1% TEA/DCM, concentrated in vacuo to a dry powder, and purified via silica gel flash chromatography eluting with 20-70% EtOAc in hexanes containing 1% TEA to give the title compound as a white foam (2.60 g, 75%). ¹H NMR (CD₃CN) δ 7.84 (d, 0.5H), 7.76 (d, 0.5H), 7.52-7.25 (m, 9H), 6.96-6.86 (m, 4H), 5.91-5.85 (m, 1H), 5.27-5.21 (m, 1H), 4.56-4.41 (m, 1H), 4.21-3.35 (m, 17H), 2.98-2.91 (m, 2H), 2.73-2.67 (m, 1H), 2.58-2.52 (m, 1H), 1.34 (d, 9H), 1.26-0.97 (m, 12H). ³¹P NMR (CD₄CN) δ 149.7, 149.1.

### Preparation 8

### 1-((2R,3R,4R,5R)-3-(2-(((3S,5S,7S)-adamantan-1-yl)disulfaneyl)ethoxy)-4-hydroxy-5-(hydroxymethyl)tetrahydrofuran-2-yl)pyrimidine-2,4(1H,3H)-dione

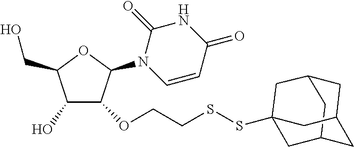 [SMILES: O=c1ccn([C@@H]2O[C@H](CO)[C@@H](O)[C@H]2OCCSSC23CC4CC(CC(C4)C2)C3)c(=O)[nH]1]

To a suspension of 2,2′-anhydro-1-(beta-D-arabinofuranosyl)uracil (1.70 g, 7.37 mmol), 2-(1-adamantyldisulfanyl)ethanol (2.70 g, 11.0 mmol), and DMA (8 mL) was added boron trifluoride diethyl etherate (1.4 mL, 11.0 mmol). The mixture was heated to 130° C. for 12 hours, then cooled to ambient temperature. The mixture was diluted with EtOAc (50 mL) and washed with saturated aqueous sodium chloride (4×20 mL). Silica gel (10 g) was added to the organics, concentrated in vacuo to a dry powder, and purified via silica gel flash chromatography eluting with 50-100% (5% MeOH/EtOAc) in hexanes to give the title compound as a thick, light brown oil (0.76 g, 22%). ¹H NMR (CD₃CN) δ 7.89 (d, 1H), 5.86 (d, 1H), 5.64 (d, 1H), 4.23-4.15 (m, 1H), 4.03-3.67 (m, 6H), 3.29 (br s, 1H), 3.21 (br s, 1H), 2.91 (t, 2H), 2.11-2.05 (m, 3H), 1.90-1.85 (m, 6H), 1.78-1.67 (m, 6H).

### Preparation 9

### 1-((2R,3R,4R,5R)-3-(2-(((3S,5S,7S)-Adamantan-1-yl)disulfaneyl)ethoxy)-5-((bis(4-methoxyphenyl)(phenyl)methoxy)methyl)-4-hydroxytetrahydrofuran-2-yl)pyrimidine-2,4(1H,3H)-dione

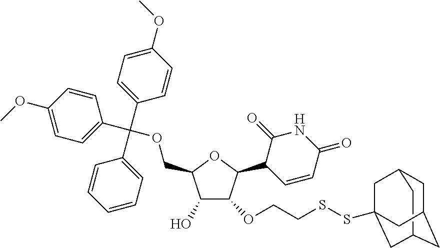 [SMILES: COc1ccc(C(OC[C@H]2O[C@@H](C3C=CC(=O)NC3=O)[C@H](OCCSSC34CC5CC(CC(C5)C3)C4)[C@@H]2O)(c2ccccc2)c2ccc(OC)cc2)cc1]

A solution of 1-((2R,3R,4R,5R)-3-(2-(((3S,5S,7S)-adamantan-1-yl)disulfaneyl)ethoxy)-4-hydroxy-5-(hydroxymethyl)tetrahydrofuran-2-yl)pyrimidine-2,4(1H,3H)-dione (0.76 g, 1.6 mmol), 4,4′-dimethoxytrityl chloride (0.73 g, 2.1 mmol), TEA (0.30 mL, 2.1 mmol), DMAP (40 mg, 0.32 mmol), and pyridine (5 mL) was stirred at ambient temperature for 16 hours. The reaction was then quenched with MeOH (1 mL) and concentrated in vacuo. The residue was suspended in DCM (5 mL), added to silica gel (5 g), concentrated in vacuo to a dry powder, and purified via silica gel flash chromatography eluting with 20-70% EtOAc in hexanes to give the title compound as a white foam (0.80 g, 64%). ¹H NMR (CD₃CN) δ 7.75 (d, 1H), 7.46 (d, 2H), 7.40-7.25 (m, 7H), 6.92 (d, 4H), 5.86 (d, 1H), 5.28 (d, 1H), 4.36 (q, 1H), 4.05-3.87 (m, 4H), 3.80 (s, 6H), 3.46-3.34 (m, 2H), 3.24 (d, 1H), 2.93 (t, 2H), 2.11-2.05 (m, 3H), 1.90-1.85 (m, 6H), 1.78-1.67 (m, 6H).

### Preparation 10

### (2R,3R,4R,5R)-4-(2-(((3 S,5S,7S)-Adamantan-1-yl)disulfaneyl)ethoxy)-2-((bis(4-methoxyphenyl)(phenyl)methoxy)methyl)-5-(2,4-dioxo-3,4-dihydropyrimidin-1(2H)-yl)tetrahydrofuran-3-yl (2-cyanoethyl) diisopropylphosphoramidite

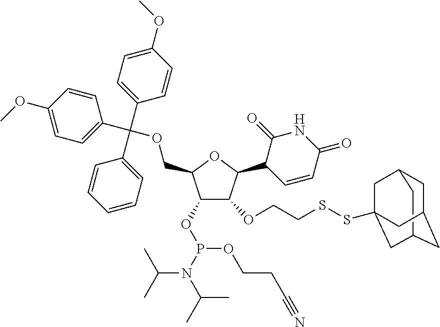 [SMILES: COc1ccc(C(OC[C@H]2O[C@@H](C3C=CC(=O)NC3=O)[C@H](OCCSSC34CC5CC(CC(C5)C3)C4)[C@@H]2OP(OCCC#N)N(C(C)C)C(C)C)(c2ccccc2)c2ccc(OC)cc2)cc1]

A solution of 1-((2R,3R,4R,5R)-3-(2-(((3S,5S,7S)-adamantan-1-yl)disulfaneyl)ethoxy)-5-((bis(4-methoxyphenyl)(phenyl)methoxy)methyl)-4-hydroxytetrahydrofuran-2-yl)pyrimidine-2,4(1H,3H)-dione (0.69 g, 0.89 mmol), 2-cyanoethyl-N,N-diisopropylchlorophosphoramidite (0.35 mL, 1.5 mmol), DIPEA (0.47 mL, 2.7 mmol), and DCM (5 mL) was stirred at ambient temperature. After 1 hour, additional 2-cyanoethyl-N,N-diisopropylchlorophosphoramidite (0.12 mL, 0.53 mmol) was added. After 1 hour, the crude reaction was poured into a slurry of silica gel (3 g) in 10 mL of 1% TEA/DCM, concentrated in vacuo to a dry powder, and purified via silica gel flash chromatography eluting with 20-70% EtOAc in hexanes containing 1% TEA to give the title compound as a white foam (0.63 g, 73%). ¹H NMR (CD₃CN) δ 7.84 (d, 0.5H), 7.75 (d, 0.5H), 7.52-7.25 (m, 9H), 6.96-6.86 (m, 4H), 5.91-5.85 (m, 1H), 5.29-5.21 (m, 1H), 4.56-4.41 (m, 1H), 4.21-3.35 (m, 17H), 2.96-2.85 (m, 2H), 2.73-2.67 (m, 1H), 2.58-2.52 (m, 1H), 2.11-2.05 (m, 3H), 1.90-1.85 (m, 6H), 1.78-1.67 (m, 6H), 1.26-0.97 (m, 12H). ³¹P NMR (CD₃CN) δ 149.7, 149.1.

### Preparation 11

### S-(2-(((2R,3R,4R,5R)-2-(2,4-Dioxo-3,4-dihydropyrimidin-1(2H)-yl)-4-hydroxy-5-(hydroxymethyl)tetrahydrofuran-3-yl)oxy)ethyl) 2,2-dimethylpropanethioate

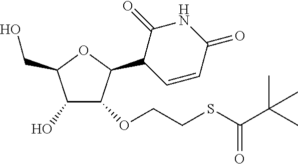 [SMILES: CC(C)(C)C(=O)SCCO[C@@H]1[C@H](O)[C@@H](CO)O[C@H]1C1C=CC(=O)NC1=O]

To a suspension of 2,2′-anhydro-1-(beta-D-arabinofuranosyl)uracil (1.5 g, 6.6 mmol), S-(2-hydroxyethyl) 2,2-dimethylpropanethioate (4.3 g, 26.53 mmol), and DMA (7.37 mL) was added boron trifluoride diethyl etherate (4.38 mL, 16.6 mmol). The mixture was heated to 100° C. for 6 hours, then cooled to ambient temperature and concentrated in vacuo to remove excess ether. The resulting residue was purified via silica gel flash chromatography eluting with 0-100% (0.1% formic acid/water) in ACN to give the title compound as a white foam (0.5 g, 19.4%). ¹H NMR (CDCl₃) δ 7.72 (d, 1H), 5.73 (m, 2H), 4.31 (t, 1H), 4.17 (dd, 1H), 4.07-3.93 (m, 4H), 3.70 (dt, 1H), 3.10 (m, 2H), 1.24 (s, 9H).

### Preparation 12

### S-(2-(((2R,3R,4R,5R)-5-((Bis(4-methoxyphenyl)(phenyl)methoxy)methyl)-2-(2,4-dioxo-3,4-dihydropyrimidin-1(2H)-yl)-4-hydroxytetrahydrofuran-3-yl)oxy)ethyl) 2,2-dimethylpropanethioate

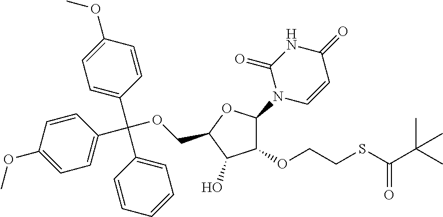 [SMILES: COc1ccc(C(OC[C@H]2O[C@@H](n3ccc(=O)[nH]c3=O)[C@H](OCCSC(=O)C(C)(C)C)[C@@H]2O)(c2ccccc2)c2ccc(OC)cc2)cc1]

A solution of S-(2-(((2R,3R,4R,5R)-2-(2,4-dioxo-3,4-dihydropyrimidin-1(2H)-yl)-4-hydroxy-5-(hydroxymethyl)tetrahydrofuran-3-yl)oxy)ethyl) 2,2-dimethylpropanethioate (2.0 g, 5.1 mmol), 4,4′-dimethoxytrityl chloride (1.92 g, 5.7 mmol), DMAP (6.3 mg, 51.5 μmol), and pyridine (14.3 mL) was stirred at ambient temperature for 14.5 hours. The reaction was then concentrated in vacuo. The residue was loaded onto silica gel and purified via silica gel flash chromatography eluting with 0-100% EtOAc containing 1% TEA in hexanes containing 1% TEA to give the title compound as a white foam (2.92 g, 82.1%). ¹H NMR (DMSO-d₆) δ 11.38 (s, 1H), 8.57 (m, 1H), 7.78 (tt, 1H), 7.70 (d, 1H), 7.40-7.23 (m, 10H), 6.90 (d, 4H), 5.79 (d, 1H), 5.29 (d, 1H), 5.19 (d, 1H), 4.18 (q, 1H), 3.97 m, 2H), 3.74 (s, 6H), 3.61 (m, 1H), 3.26 (m, 2H), 3.02 (m, 2H), 1.16 (s, 9H).

### Preparation 13

### S-(2-(((2R,3R,4R,5R)-5-((Bis(4-methoxyphenyl)(phenyl)methoxy)methyl)-4-(((2-cyanoethoxy)(diisopropylamino)phosphaneyl)oxy)-2-(2,4-dioxo-3,4-dihydropyriimidin-1(2H)-yl)tetrahydrofuran-3-yl)oxy)ethyl) 2,2-dimethylpropanethioate

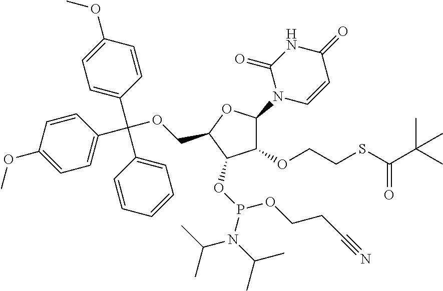 [SMILES: COc1ccc(C(OC[C@H]2O[C@@H](n3ccc(=O)[nH]c3=O)[C@H](OCCSC(=O)C(C)(C)C)[C@@H]2OP(OCCC#N)N(C(C)C)C(C)C)(c2ccccc2)c2ccc(OC)cc2)cc1]

A solution of S-(2-(((2R,3R,4R,5R)-5-((bis(4-methoxyphenyl)(phenyl)methoxy)methyl)-2-(2,4-dioxo-3,4-dihydropyrimidin-1(2H)-yl)-4-hydroxytetrahydrofuran-3-yl)oxy)ethyl) 2,2-dimethylpropanethioate (2.9 g, 4.2 mmol), 2-cyanoethyl-N,N-diisopropylchlorophosphoramidite (1.13 mL, 5.07 mmol), DIPEA (1.84 mL, 10.57 mmol), and DCM (42.3 mL) was stirred at ambient temperature. After 1 hour, additional 2-cyanoethyl-N,N-diisopropylchlorophosphoramidite (0.94 mL, 4.23 mmol) was added. After 1 hour, additional 2-cyanoethyl-N,N-diisopropylchlorophosphoramidite (0.19 mL, 0.85 mmol) was added. After 10 minutes, the crude reaction was loaded onto silica gel and purified via silica gel flash chromatography eluting with 0-100% EtOAc in hexanes containing 1% TEA to give the title compound as a white foam (2.32 g, 61.5%). ¹H NMR (DMSO-d₆) δ 11.38 (s, 1H), 7.77 (q, 1H), 7.41-7.22 (m, 9H), 6.9 (m, 4H), 5.8 (t, 1H), 5.27 (dd, 1H), 4.39 (m, 1H), 4.18-4.07 (m, 1H), 3.84-3.50 (m, 12H), 3.01 (m, 2H), 2.79 (t, 1H), 1.25-1.10 (m, 21H). ³¹P NMR (DMSO-d₆) δ 149.3, 148.5.

### Preparation 14

### 2-(Tritylthio)ethyl 4-methylbenzenesulfonate

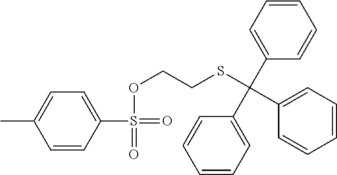 [SMILES: Cc1ccc(S(=O)(=O)OCCSC(c2ccccc2)(c2ccccc2)c2ccccc2)cc1]

A solution of 2-(tritylthio)ethanol (1.00 g, 3.03 mmol), DCM (9 mL), p-toluenesulfonyl chloride (0.8665 g, 4.545 mmol), and pyridine (0.50 mL, 6.06 mmol) was stirred at ambient temperature for 16 hours. The mixture was diluted with water (50 mL) then extracted with EtOAc (3×75 mL). The combined organic layer was washed with saturated aqueous sodium chloride (2×150 mL), dried with NaSO₄, and concentrated in vacuo. The crude reaction was diluted with DCM, loaded onto silica gel, and purified via silica gel flash chromatography eluting with 5-40% EtOAc in hexanes to give the title compound as a brown oil (330 mg, 23%). H NMR (CDCl₃) 7.75-7.67 (m, 2H), 7.38-7.17 (m, 17H), 3.62 (t, 2H), 2.52 (t, 2H), 2.47 (s, 3H).

### Preparation 15

### 2-((4R,8R)-4,8,12-Trimethyltridecyl)-1,2,3,4-tetrahydroisoquinolin-6-ol

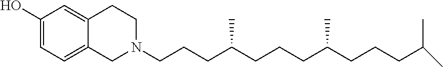 [SMILES: CC(C)CCC[C@@H](C)CCC[C@@H](C)CCCN1CCc2cc(O)ccc2C1]

Potassium carbonate (0.51 g, 3.7 mmol) was added to a solution of 1,2,3,4-tetrahydroisoquinolin-6-ol (0.50 g, 3.4 mmol) in DMF (25 mL). Then (4R,8R)-1-iodo-4,8,12-trimethyltridecane (1.3 g, 3.7 mmol) was added to the reaction. The mixture was stirred at 65° C. for 4 hours then cooled to ambient temperature and concentrated in vacuo. The resulting crude material was purified via silica gel flash chromatography eluting with a gradient of 0-100% EtOAc in hexanes to give the title compound as a white solid (0.81 g, 65%). ¹H NMR (CDCl₃) δ 6.89 (d, 1H), 6.61 (dd, 1H), 6.53 (d, 1H), 3.61 (s, 2H), 2.91-2.68 (m, 4H), 2.53 (t, 2H), 1.77-1.00 (m, 19H), 0.94-0.81 (m, 12H).

### Preparation 16

### 2-((4R, 8R)-4,8,12-Trimethyltridecyl)-6-(2-(tritylthio)ethoxy)-1,2,3,4-tetrahydroisoquinoline

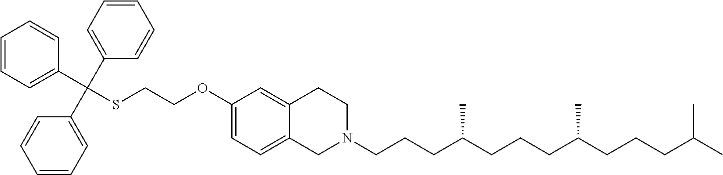 [SMILES: CC(C)CCC[C@@H](C)CCC[C@@H](C)CCCN1CCc2cc(OCCSC(c3ccccc3)(c3ccccc3)c3ccccc3)ccc2C1]

A solution of 2-((4R,8R)-4,8,12-trimethyltridecyl)-1,2,3,4-tetrahydroisoquinolin-6-ol (0.20 g, 0.54 mmol), DMF (2.1 mL), cesium carbonate (0.35 g, 1.10 mmol), and 2-(tritylthio)ethyl 4-methylbenzenesulfonate (0.33 g, 0.70 mmol) was stirred at 45° C. for 3 hours. The reaction was concentrated in vacuo then diluted with DCM, loaded onto silica gel, and purified via silica gel flash chromatography eluting with 0-40% EtOAc in hexanes to give the title compound as a viscous, light yellow oil (0.143 g, 39%). ¹H NMR (CDCl₃) 7.48-7.43 (m, 8H), 7.32-7.21 (m, 7H), 6.89 (d, 1H), 6.53-6.49 (m, 2H), 3.71 (t, 2H), 3.55 (s, 2H), 2.88-2.82 (m, 2H), 2.72-2.66 (m, 2H), 2.63 (t, 2H), 2.50-2.44 (m, 2H), 1.69-0.78 (m, 31H).

### Preparation 17

### 2-((2-((4R,8R)-4,8,12-Trimethyltridecyl)-1,2,3,4-tetrahydroisoquinolin-6-yl)oxy)ethane-1-thiol

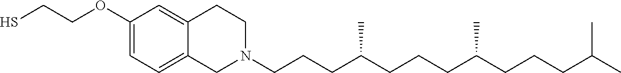 [SMILES: CC(C)CCC[C@@H](C)CCC[C@@H](C)CCCN1CCc2cc(OCCS)ccc2C1]

A solution of 2-((4R,8R)-4,8,12-trimethyltridecyl)-6-(2-(tritylthio)ethoxy)-1,2,3,4-tetrahydroisoquinoline (0.1426 g, 0.21 mmol), DCM (0.7 mL), TFA (0.41 mL, 5.3 mmol), and triethylsilane (0.07 mL, 0.4 mmol) was stirred at ambient temperature for 1 hour. The reaction was concentrated in vacuo then diluted with EtOAc (75 mL). The organic layer was washed with saturated aqueous NaHCO₃ (1×50 mL) and the aqueous was back-extracted with EtOAc (1×75 mL). The organic layers were combined, dried with NaSO₄, and concentrated in vacuo. The resulting material was diluted with DCM, then loaded onto silica gel and purified via silica gel flash chromatography eluting with 0-100% EtOAc in hexanes containing 0.5% TEA to give the title compound as a clear oil (0.065 g, 71%). ¹H NMR (CDCl₃) 7.04 (d, 1H), 6.83 (dd, 1H), 6.73 (d, 1H), 4.62 (d, 1H), 4.11 (t, 2H), 3.99 (d, 1H), 3.80-3.72 (m, 1H), 3.38-2.87 (m, 7H), 2.26-0.70 (m, 31H).

### Preparation 18

### 6-((2-((3r,5r,7r)-Adamantan-1-yl)ethyl)disulfaneyl)nicotinic acid

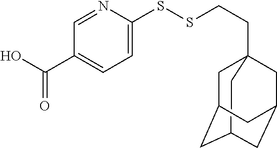 [SMILES: O=C(O)c1ccc(SSCCC23CC4CC(CC(C4)C2)C3)nc1]

6-[(5-Carboxy-2-pyridyl)disulfanyl]pyridine-3-carboxylic acid (617 mg, 2 mmol) was stirred in THE (10 mL) and 20× borate buffer (10 mL) until all solid was dissolved. 2-((3r,5r,7r)-adamantan-1-yl)ethane-1-thiol (196 mg, 1 mmol) was added in one portion and the reaction was stirred at ambient temperature for 3 hours. The reaction was then concentrated to −5 mL of total volume and the residue was purified with reverse phase flash chromatography (C18 column) eluting with a gradient of 0-70% acetonitrile/10 mM ammonium bicarbonate to give the title compound as a white solid (180 mg, 52%). ¹H NMR (DMSO-d₆) 8.82 (d, 1H), 8.18 (dd, 1H), 7.74 (d, 1H), 2.86-2.77 (m, 2H), 1.94-1.84 (m, 3H), 1.68-1.53 (m, 6H), 1.48-1.37 (m, 8H).

### Preparation 19

### 6-(Dodecyldisulfaneyl)nicotinic acid

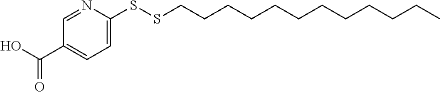 [SMILES: CCCCCCCCCCCCSSc1ccc(C(=O)O)cn1]

The title compound is prepared from dodecane-1-thiol in a manner essentially analogous to the procedure found in Preparation 18. ¹H NMR (DMSO-d₆) 8.91 (d, 1H), 8.27 (dd, 1H), 7.91 (d, 1H), 2.87 (t, 2H), 1.67-1.55 (m, 2H), 1.40-1.14 (m, 18H), 0.86 (t, 3H).

### C12 ADS Linked siRNA

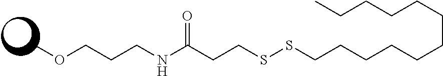 [SMILES: CCCCCCCCCCCCSSCCC(=O)NCCCOC]

A sense strand synthesized using conditions found in the protocols below (3.1 g, 0.44 mmol) in 4× borate buffer water (113 mL) was treated with a solution of 2,5-dioxopyrrolidin-1-yl 3-(dodecyldisulfaneyl)propanoate (5.3 g, 4.4 mmol) in ACN (113 mL). The solution was shaken for 1.5 hours at 30° C. The reaction was quenched by diluting with water and adjusting the pH=7 with 1.2M aqueous HCl. The solution was then concentrated via Genevac to remove the organic solvent and afford the crude oligonuleotides.

The crude oligonucleotides were purified via AKTA™ Pure purification system using reverse phase on a source 15RPC column (MPA: 50 mM NaOAc with 10% ACN and MPB: 80% ACN/water). In all cases, fractions which contained a mass purity greater than 85% without impurities >5% were combined.

The purified oligonucleotides were desalted using 15 mL 3K MWCO centrifugal spin tubes at 3500×g for ~30 minutes. The oligonucleotides were rinsed with RNAse free water until the eluent conductivity reached <100 usemi/cm. After desalting was complete, 2-3 mL of RNAse free water was added then aspirated 10× and the retainment was transferred to a 50 mL falcon tube. This was repeated until complete transfer of oligo by measuring concentration of compound on filter via nanodrop. The final oligonucleotide was then nano filtered 2× via 15 mL 100K MWCO centrifugal spin tubes at 3500×g for 2 min. The final desalted oligonucleotides were analyzed for concentration (nano drop at A260), characterized by IP-RP, LCMS for mass purity, and UPLC for UV-purity. ES/MS (m/z): 7324.6(M+H).

The compound below was prepared in a manner essentially analogous to the preparation found in C12 ADS linked siRNA.

TABLE 18

| Name | Structure | ES/MS (m/z) |
|---|---|---|
| C16 ADS linked siRNA | 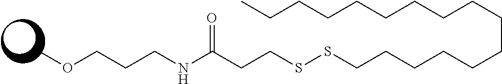 [SMILES: CCCCCCCCCCCCCCCCSSCCC(=O)NCCCOC] | 7380.9 |

### SS-C12 Linked siRNA

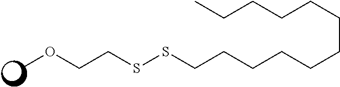 [SMILES: CCCCCCCCCCCCSSCCOC]

Post-oligosynthesis (sense strand synthesized using conditions found in the protocols below), CPG with loaded oligo on it was washed with diethylamine and then dried under vacuum. 50 μmol of loaded CPG was added to a 50 mL falcon tube and 50 mgs of 6-(dodecyldisulfaneyl)nicotinic acid was added to the same tube followed by 15 mL of AMA solution (29% ammonium hydroxide in water:40% methylamine in water, 1:1) and shook at ambient temperature. After 1 hour >80% of the desired product mass was observed. The solution was then concentrated on Genevac to remove the organics and afford the crude oligonucleotides. The crude oligonucleotides were filtered using 0.2 micron syringe filters and then purified via AKTA™ Pure purification system using anion exchange (AEX) a source 15Q column. For AEX, a Source™ 15Q column with MPA: 20 mM NaH₂PO₄ with 15% ACN, pH 7.4 and MPB: 20 mM NaH₂PO₄ with 1M NaBr, 15% ACN, pH 7.4 was used. In all cases, fractions which contained a mass purity greater than 85% without impurities >5% where combined.

The purified oligonucleotides were desalted using 15 mL 3K MWCO centrifugal spin tubes at 3500×g for ~30 min. The oligonucleotides were rinsed with RNAse free water until the eluent conductivity reached <100 μS/cm. After desalting was complete, 2-3 mL of RNAse free water was added then aspirated 10×, the retainment was transferred to a 50 mL falcon tube, this was repeated until complete transfer of oligo by measuring concentration of compound on filter via nanodrop. The final desalted oligonucleotides were analyzed for concentration (nano drop at A260), characterized by IP-RP LCMS for mass purity and UPLC for UV-purity. ES/MS (m/e): 7239.6.

The compound in Table 4 was prepared in a manner essentially analogous to that found in USS-C12 linked siRNA preparation.

TABLE 4
Exemplary modified nucleotide

| Name | Structure | ES/MS (m/z) |
|---|---|---|
| SS-C2-adamantyl linked siRNA | 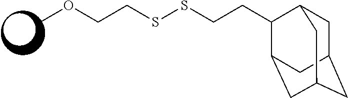 | 7206.1 (M + H) |

### SS-Adamantyl Linked siRNA

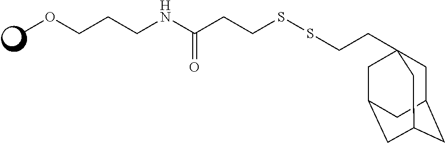 [SMILES: COCCCNC(=O)CCSSCCC12CC3CC(CC(C3)C1)C2]

A sense strand (0.0077 mmol in 15 mL water) synthesized using conditions found in the protocols below was added to 20× borate buffer (2.25 mL), then was treated with a solution of 2,5-dioxopyrrolidin-1-yl 3-(pyridine-2-yldisulfaneyl)propanoate (0.0241 g, 0.0772 mmol) (CAS No. 68181-17-9) in MeCN (3.75 mL). The solution was shaken for 30 mins at ambient temperature. The solution was then diluted to 40 mL using RNAse free water to bring concentration of organic solvent to ≤10%. Excess 2,5-dioxopyrrolidin-1-yl 3-(pyridine-2-yldisulfaneyl)propanoate was removed using 15 mL 3K MWCO centrifugal spin tubes at 3500×g for ~30 minutes. The oligonucleotides were rinsed with RNAse free water three times. After removing 2,5-dioxopyrrolidin-1-yl 3-(pyridine-2-yldisulfaneyl)propanoate, 1 mL of RNAse free water was added then aspirated 10× and the retentate was transferred to a 5 mL falcon tube. This was repeated until complete transfer of oligo by measuring concentration of compound on filter via nanodrop. The final oligonucleotide was analyzed for concentration (nano drop at A260), characterized by IP-RP, LCMS for mass purity, and UPLC for UV-purity. ES/MS (m/z): 7196.02 (M+H).

The sense strand synthesized above (0.0035 mmol in 1.4 mL water) was treated with a solution of 1-adamantanethiol (0.0119 g, 0.0705 mmol) (CAS No. 34301-54-7) in THF (1.40 mL). The solution was shaken for 16 hours at 50° C. The solution was then concentrated via Genevac to remove the organic solvent and afford the crude oligonucleotide. The crude oligonucleotide was purified via AKTA™ Pure purification system using reverse phase on a source 15RPC 10×200 mm column (MPA: 10 mM NaOAc with 2% ACN and MPB: 80% ACN in water). Under a 2-50% gradient over 8 column volumes, desired product eluted at 10%. In all cases, fractions which contained a mass purity greater than 85% without impurities >5% were combined. The solution was then concentrated via Genevac to remove the organic solvent and afford the purified oligonucleotide. The purified oligonucleotide was desalted using 15 mL 3K MWCO centrifugal spin tubes at 3500×g for ~30 minutes. The oligonucleotides were rinsed with RNAse free water until the eluent conductivity reached <100 usemi/cm. After desalting was complete, 1 mL of RNAse free water was added then aspirated 10× and the retentate was transferred to a 5 mL falcon tube. This was repeated until complete transfer of oligo by measuring concentration of compound on filter via nanodrop. The final desalted oligonucleotide was analyzed for concentration (nano drop at A260), characterized by IP-RP, LCMS for mass purity, and UPLC for UV-purity. ES/MS (m/z): 7253.15 (M+H).

### SS-C2-Tetraisoquinoline Linked siRNA

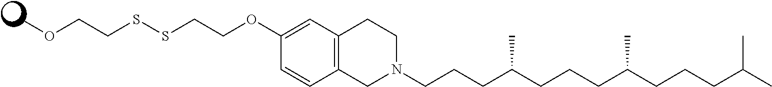 [SMILES: COCCSSCCOc1ccc2c(c1)CCN(CCC[C@H](C)CCC[C@H](C)CCCC(C)C)C2]

A sense strand synthesized using conditions found in the protocols below (1 mM solution in water) was treated with 10 equivalents of TCEP. The reaction was allowed to shake at 45° C. for 18 hours. The solution was then transferred to a 15 mL 3K MWCO centrifugal spin filter and spun at 3500×g for ~30 minutes. After addition of 15 mL of water, this process was repeated. The aqueous solution of siRNA (0.5 mM) was treated with an ACN solution of dipyridyl disulfide (20 equivalents). The final ACN content was 20%. After 1 hour, the reaction was diluted with water to bring the ACN content to 10%. The solution was then transferred to a 15 mL 3K MWCO centrifugal spin filter and spun at 3500×g for ~30 minutes. After addition of 15 mL of water this process was repeated. The aqueous solution of siRNA (1 mM) was treated with 2-((2-((4R,8R)-4,8,12-trimethyltridecyl)-1,2,3,4-tetrahydroisoquinolin-6-yl)oxy)ethane-1-thiol (10 equivalents) dissolved in THF. The concentration of the thiol solution in THF was calculated such that the final THE content was 60%. The solution was shaken at 45° C. for 48 hours. THF was then removed via vacuum centrifugation and the conjugated siRNA was purified via reverse phase chromatography (Source15 RPC column; MPA: 50 mM NaOAc with 10% ACN and MPB: 50 mM NaOAc with 80% ACN). The purified oligonucleotide was desalted using 15 mL 3K MWCO centrifugal spin tubes at 3500×g for ~30 minutes. The oligonucleotides were rinsed with RNAse free water until the eluent conductivity reached <100 usemi/cm. The final oligonucleotide was then nano filtered 2× via 15 mL 100K MWCO centrifugal spin tubes at 3500×g for 2 min. The final desalted oligonucleotides were analyzed for concentration (nano drop at A260), characterized by IP-RP LCMS for mass purity, and UPLC for UV-purity. ES/MS (m/z): 7423.6 (M+H).

### Synthesis of dsRNA

Single strands (sense and antisense) of the RNA duplexes were synthesized on solid support via a MerMade™ 12. The sequences of the sense and antisense strands were shown in Tables 1 and 2. The oligonucleotides were synthesized via phosphoramidite chemistry at either 5, 10, 25 or 50 μmol scales.

All single strands were synthesized from commercially available standard support mA. Standard reagents were used in the oligo synthesis (Table 5), where 0.1M xanthane hydride in pyridine was used as the sulfurization reagent and 20% DEA in ACN was used as an auxiliary wash post synthesis. All monomers (Table 6) were made at 0.1M in ACN and contained a molecular sieves trap bag.

The oligonucleotides were cleaved and deprotected (C/D) at 45° C. for 20 hours. The sense strands were C/D from the CPG using ammonia hydroxide (28-30%, cold), whereas 3% DEA in ammonia hydroxide (28-30%, cold) was used for the antisense strands. C/D was determined complete by IP-RP LCMS when the resulting mass data confirmed the identity of sequence. Dependent on scale, the CPG was filtered via 0.45 um PVDF syringeless filter, 0.22 um PVDF Steriflip® vacuum filtration or 0.22 um PVDF Stericup® Quick release. The CPG was back washed/rinsed with either 30% ACN/RNAse free water or 30% EtOH/RNAse free water then filtered through the same filtering device and combined with the first filtrate. This was repeated twice. The material was then divided evenly into 50 mL falcon tubes to remove organics via Genevac™. After concentration, the crude oligonucleotides were diluted back to synthesized scale with RNAse free water and filtered either by 0.45 μm PVDF syringeless filter, 0.22 μm PVDF Steriflip® vacuum filtration or 0.22 μm PVDF Stericup® Quick release.

The crude oligonucleotides were purified via AKTA™ Pure purification system using either ion-exchange (AEX) or reverse phase (RP) a source 15Q-RP column. For AEX, an ES Industry Source™ 15Q column maintaining column temperature at 65° C. with MPA: 20 mM NaH₂PO₄, 15% ACN, pH 7.4 and MPB: 20 mM NaH₂PO₄, 1M NaBr, 15% ACN, pH 7.4. For RP, a Source™ 15Q-RP column with MPA: 50 mM NaOAc with 10% ACN and MPB: 50 mM NaOAc with 80% ACN. In all cases, fractions which contained a mass purity greater than 85% without impurities >5% where combined.

The purified oligonucleotides were desalted using 15 mL 3K MWCO centrifugal spin tubes at 3500×g for ~30 min. The oligonucleotides were rinsed with RNAse free water until the eluent conductivity reached <100 msemi/cm. After desalting was complete, 2-3 mL of RNAse free water was added then aspirated 10×, the retainment was transferred to a 50 mL falcon tube, this was repeated until complete transfer of oligo by measuring concentration of compound on filter via nanodrop. The final oligonucleotide was then nano filtered 2× via 15 mL 100K MWCO centrifugal spin tubes at 3500×g for 2 min. The final desalted oligonucleotides were analyzed for concentration (nano drop at A260), characterized by IP-RP LCMS for mass purity and UPLC for UV-purity.

For the preparation of duplexes, equimolar amounts of sense and antisense strand were combined and heated at 65° C. for 10 minutes then slowly cooled to ambient temperature over 40 minutes. Integrity of the duplex was confirmed by UPLC analysis and characterized by LCMS using IP-RP. All duplexes were nano filtered then endotoxin levels measured via Charles River Endosafe® Cartridge Device to give the final compounds of RNAi conjugates. For in vivo analysis, the appropriate amount of duplex was lyophilized then reconstituted in 1×PBS for rodent studies and aCSF for non-human primate studies.

The molecular weight of exemplary SNCA and MAPT RNAi agents are shown in Tables 7 and 8.

TABLE 5
Oligonucleotide Synthesis Reagents
Reagents
Activator Solution (0.5M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water,
70:20:10)
Deblock Solution, 3% TCA in DCM (w/v)
Acetonitrile (Anhydrosolv, Water max. 10 ppm)
Xanthane Hydride (0.1M in Pyridine)
Diethylamine (20% in Acetonitrile)

TABLE 6
Phosphoramidites

| Phosphoramidite | Abbreviation | Supplier | Catalog # | CAS |
|---|---|---|---|---|
| DMT-2′-F-A(Bz)-CE Phosphoamidite | fA | Hongene | PD1-001 | 136834-22-5 |
| DMT-2′-F-C(Ac)-CE Phosphoamidite | fC | Hongene | PD3-001 | 159414-99-0 |
| DMT-2′-F-G(iBu)-CE Phosphoamidite | fG | Hongene | PD2-002 | 144089-97-4 |
| DMT-2′-F-U-CE Phosphoamidite | fU | Hongene | PD5-001 | 146954-75-8 |
| DMT-2′-O-Me-A(Bz)-CE Phosphoamidite | mA | Hongene | PR1-001 | 110782-31-5 |
| DMT-2′-O-Me-C(Ac)-CE Phosphoamidite | mC | Hongene | PR3-001 | 199593-09-4 |
| DMT-2′-O-Me-G(iBu)-CE Phosphoamidite | mG | Hongene | PR2-002 | 150780-67-9 |
| DMT-2′-O-Me-U-CE Phosphoamidite | mU | Hongene | PR5-001 | 110764-79-9 |
| 5′bis(POM) vinyl phosphate-2′-Ome-U3′CE | POM-VPmU | Hongene | PR5-032 | BVPMUP- |
| phosphoroamidite | | | | 23B2A1 |
| DMT-dT-CE Phosphoamidite | dT | Hongene | PD4-002 | 98796-51-1 |
| DMT-dC(Bz)-CE Phosphoamidite | dC | Hongene | PD3-003 | 102212-98-6 |
| DMT-dG(iBu)-CE Phosphoamiditc | dG | Hongene | PD2-004 | 93183-15-4 |
| DMT-dA(Bz)-CE Phosphoamidite | dA | Hongene | PD1-004 | 98796-53-3 |
| 2′-O-Trifluoroacetamido propyl Uridine CED | Upa | Chemgenes | ANP-7115 | 165381-49-7 |
| phosphoramidite | | | | |
| 2′-O-Trifluoroacetamido propyl Cytidine CED | Cpa | Chemgenes | ANP-7116 | 165381-54-4 |
| phosphoramidite | | | | |
| 2′-O-Trifluoroacetamido propyl Adenosine(Bz) | Apa | Hongene | PR1-108 | NA |
| CED phosphoramidite | | | | |
| 2′-O-Trifluoroacetamido propyl Guanosine(iBu) | Gpa | Hongene | PR2-105 | NA |
| CED phosphoramidite | | | | |
| Reverse Abasic phosphoroamidite | iAb | Chemgenes | ANP-1422 | 401813-16-9 |
| Abasic phosphoroamidite | n | Chemgenes | ANP-7058 | 129821-76-7 |
| (2R,3R,4R,5R)-5-(4-Acetamido-2-oxopyrimidin- | NA | Lilly | NA | NA |
| 1(2H)-yl)-2-((bis(4- | | | | |
| methoxyphenyl)(phenyl)methoxy)methyl)-4-(2- | | | | |
| (tert-butyldisulfaneyl)ethoxy)tetrahydrofuran-3-yl | | | | |
| (2-cyanoethyl) diisopropylphosphoramidite | | | | |
| (2R,3R,4R,5R)-2-((Bis(4- | NA | Lilly | NA | NA |
| methoxyphenyl)(phenyl)methoxy)methyl)-4-(2- | | | | |
| (tert-butyldisulfaneyl)ethoxy)-5-(2,4-dioxo-3,4- | | | | |
| dihydropyrimidin-1(2H)-yl)tetrahydrofuran-3-yl | | | | |
| (2-cyanoethyl) diisopropylphosphoramidite | | | | |
| (2R,3R,4R,5R)-4-(2-(((3S,5S,7S)-Adamantan-1- | NA | Lilly | NA | NA |
| yl)disulfaneyl)ethoxy)-2-((bis(4- | | | | |
| methoxyphenyl)(phenyl)methoxy)methyl)-5-(2,4- | | | | |
| dioxo-3,4-dihydropyrimidin-1(2H)- | | | | |
| yl)tetrahydrofuran-3-yl (2-cyanoethyl) | | | | |
| diisopropylphosphoramidite | | | | |
| S-(2-(((2R,3R,4R,5R)-5-((Bis(4- | NA | Lilly | NA | NA |
| methoxyphenyl)(phenyl)methoxy)methyl)-4-(((2- | | | | |
| cyanoethoxy)(diisopropylamino)phosphaneyl)oxy)- | | | | |
| 2-(2,4-dioxo-3,4-dihydropyrimidin-1(2H)- | | | | |
| yl)tetrahydrofuran-3-yl)oxy)ethyl) 2,2- | | | | |
| dimethylpropanethioate | | | | |

TABLE 7
Molecular Weight of Exemplary SNCA RNAi Agents

| SNCA | | | |
|---|---|---|---|
| RNAi | | | |
| Agent | | MW Cal. | MW Obs. |
| No. | Strand | (g/mol) | (g/mol) |
| 2 | S: SEQ ID NO: 3 | 7275.22 | 7277.6 |
| | AS: SEQ ID NO: 4 | 7825.18 | 7826.4 |
| 3 | S: SEQ ID NO: 5 | 7275.22 | 7277.3 |
| | AS: SEQ ID NO: 4 | 7825.18 | 7826.6 |
| 4 | S: SEQ ID NO: 6 | 7202.15 | 7203.6 |
| | AS: SEQ ID NO: 7 | 7813.15 | 7813.14 |
| 5 | S: SEQ ID NO: 8 | 7202.15 | 7203.6 |
| | AS: SEQ ID NO: 7 | 7813.15 | 7813.14 |
| 6 | S: SEQ ID NO: 9 | 7202.15 | 7203.5 |
| | AS: SEQ ID NO: 7 | 7813.15 | 7813.14 |
| 7 | S: SEQ ID NO: 10 | 7202.15 | 7203.6 |
| | AS: SEQ ID NO: 7 | 7813.15 | 7813.14 |
| 8 | S: SEQ ID NO: 11 | 7202.15 | 7203.6 |
| | AS: SEQ ID NO: 7 | 7813.15 | 7813.14 |
| 9 | S: SEQ ID NO: 12 | 7202.15 | 7203.5 |
| | AS: SEQ ID NO: 7 | 7813.15 | 7813.14 |
| 10 | S: SEQ ID NO: 13 | 7202.15 | 7203.6 |
| | AS: SEQ ID NO: 7 | 7813.15 | 7813.14 |
| 11 | S: SEQ ID NO: 14 | 7202.15 | 7203.6 |
| | AS: SEQ ID NO: 7 | 7813.15 | 7813.14 |
| 12 | S: SEQ ID NO: 15 | 7214.18 | 7215.8 |
| | AS: SEQ ID NO: 7 | 7813.15 | 7813.14 |
| 13 | S: SEQ ID NO: 16 | 7198.22 | 7203.6 |
| | AS: SEQ ID NO: 7 | 7813.15 | 7813.14 |
| 14 | S: SEQ ID NO: 17 | 7198.22 | 7203.5 |
| | AS: SEQ ID NO: 7 | 7813.15 | 7813.14 |
| 15 | S: SEQ ID NO: 18 | 7198.22 | 7203.6 |
| | AS: SEQ ID NO: 7 | 7813.15 | 7813.14 |
| 16 | S: SEQ ID NO: 19 | 7287.25 | 7287.26 |
| | AS: SEQ ID NO: 7 | 7813.15 | 7813.14 |
| 17 | S: SEQ ID NO: 20 | 7190.11 | 7192.0 |
| | AS: SEQ ID NO: 4 | 7825.18 | 7826.6 |
| 18 | S: SEQ ID NO: 37 | 7421.45 | 7423.6 |
| | AS: SEQ ID NO: 7 | 7813.15 | 7813.14 |
| 19 | S: SEQ ID NO: 38 | 7168.05 | 7169.7 |
| | AS: SEQ ID NO: 7 | 7813.15 | 7813.14 |
| 20 | S: SEQ ID NO: 19 | 7287.25 | 7287.5 |
| | AS: SEQ ID NO: 66 | 7837.21 | 7837.10 |
| 21 | S: SEQ ID NO: 67 | 7253.15 | 7252.4 |
| | AS: SEQ ID NO: 7 | 7813.15 | 7813.14 |
| 22 | S: SEQ ID NO: 68 | 6631.84 | 6631.1 |
| | AS: SEQ ID NO: 7 | 7813.15 | 7813.14 |
| 23 | S: SEQ ID NO: 69 | 7287.25 | 7286.6 |
| | AS: SEQ ID NO: 7 | 7813.15 | 7813.14 |
| 24 | S: SEQ ID NO: 70 | 7248.21 | 7247.5 |
| | AS: SEQ ID NO: 7 | 7813.15 | 7813.14 |
| 25 | S: SEQ ID NO: 71 | 7287.25 | 7286.7 |
| | AS: SEQ ID NO: 7 | 7813.15 | 7813.14 |
| 26 | S: SEQ ID NO: 72 | 7287.25 | 7286.8 |
| | AS: SEQ ID NO: 7 | 7813.15 | 7813.14 |
| 27 | S: SEQ ID NO: 73 | 7299.29 | 7298.6 |
| | AS: SEQ ID NO: 7 | 7813.15 | 7813.14 |
| 28 | S: SEQ ID NO: 74 | 7287.25 | 7286.6 |
| | AS: SEQ ID NO: 7 | 7813.15 | 7813.14 |
| 29 | S: SEQ ID NO: 75 | 7287.25 | 7286.6 |
| | AS: SEQ ID NO: 7 | 7813.15 | 7813.14 |
| 30 | S: SEQ ID NO: 76 | 7248.21 | 7247.6 |
| | AS: SEQ ID NO: 7 | 7813.15 | 7813.14 |
| 31 | S: SEQ ID NO: 77 | 7287.25 | 7286.7 |
| | AS: SEQ ID NO: 7 | 7813.15 | 7813.14 |
| 32 | S: SEQ ID NO: 78 | 7287.25 | 7286.6 |
| | AS: SEQ ID NO: 7 | 7813.15 | 7813.14 |
| 33 | S: SEQ ID NO: 79 | 7287.25 | 7286.6 |
| | AS: SEQ ID NO: 7 | 7813.15 | 7813.14 |
| 34 | S: SEQ ID NO: 80 | 7287.25 | 7286.6 |
| | AS: SEQ ID NO: 7 | 7813.15 | 7813.14 |
| 35 | S: SEQ ID NO: 81 | 7343.36 | 7342.6 |
| | AS: SEQ ID NO: 7 | 7813.15 | 7813.14 |
| 36 | S: SEQ ID NO: 16 | 7202.15 | 7202.2 |
| | AS: SEQ ID NO: 82 | 7813.15 | 7812.2 |
| 37 | S: SEQ ID NO: 9 | 7202.15 | 7202.2 |
| | AS: SEQ ID NO: 82 | 7813.15 | 7812.2 |
| 40 | S: SEQ ID NO: 87 | 7299.24 | 7298.9 |
| | AS: SEQ ID NO: 88 | 7802.14 | 7802.10 |
| 41 | S: SEQ ID NO: 89 | 7311.28 | 7310.6 |
| | AS: SEQ ID NO: 90 | 7790.11 | 7789.7 |
| 42 | S: SEQ ID NO: 91 | 7171.18 | 7170.9 |
| | AS: SEQ ID NO: 92 | 7430.87 | 7430.4 |
| 43 | S: SEQ ID NO: 91 | 7171.18 | 7170.9 |
| | AS: SEQ ID NO: 93 | 7454.95 | 7454.3 |
| 50 | S: SEQ ID NO: 106 | 7233.20 | 7233.40 |
| | AS: SEQ ID NO: 107 | 7783.12 | 7782.50 |
| 51 | S: SEQ ID NO: 108 | 7233.20 | 7233.40 |
| | AS: SEQ ID NO: 109 | 7760.08 | 7759.60 |
| 52 | S: SEQ ID NO: 110 | 7477.43 | 7477.30 |
| | AS: SEQ ID NO: 111 | 7545.87 | 7545.90 |
| 53 | S: SEQ ID NO: 112 | 7287.25 | 7286.80 |
| | AS: SEQ ID NO: 113 | 7750.08 | 7749.50 |
| 54 | S: SEQ ID NO: 114 | 7256.24 | 7256.20 |
| | AS: SEQ ID NO: 115 | 7799.12 | 7798.50 |
| 55 | S: SEQ ID NO: 116 | 7466.50 | 7466.40 |
| | AS: SEQ ID NO: 117 | 7556.80 | 7556.50 |
| 56 | S: SEQ ID NO: 118 | 7221.16 | 7221.10 |
| | AS: SEQ ID NO: 119 | 7772.11 | 7772.50 |
| 57 | S: SEQ ID NO: 120 | 7401.26 | 7401.20 |
| | AS: SEQ ID NO: 121 | 7795.15 | 7795.10 |
| 58 | S: SEQ ID NO: 108 | 7233.20 | 7233.20 |
| | AS: SEQ ID NO: 122 | 7784.15 | 7784.10 |
| 59 | S: SEQ ID NO: 123 | 7413.29 | 7413.30 |
| | AS: SEQ ID NO: 124 | 7807.19 | 7807.30 |

“S” means the sense strand; “AS” means the antisense strand.

TABLE 8
Molecular Weight of Exemplary MAPT RNAi Agents

| MAPT | | | |
|---|---|---|---|
| RNAi | | | |
| Agent | | MW Cal. | MW Obs. |
| No. | Strand | (g/mol) | (g/mol) |
| 4 | S: SEQ ID NO: 27 | 7485.45 | 7484.3 |
| | AS: SEQ ID NO: 28 | 7506.84 | 7505.9 |
| 5 | S: SEQ ID NO: 29 | 7366.32 | 7365.8 |
| | AS: SEQ ID NO: 30 | 7749.1 | 7748.5 |
| 6 | S: SEQ ID NO: 31 | 7325.36 | 7324.6 |
| | AS: SEQ ID NO: 32 | 7665.95 | 7665.6 |
| 7 | S: SEQ ID NO: 33 | 7485.45 | 7484.8 |
| | AS: SEQ ID NO: 28 | 7506.84 | 7506.2 |
| 8 | S: SEQ ID NO: 34 | 7366.32 | 7365.7 |
| | AS: SEQ ID NO: 30 | 7749.1 | 7748.3 |
| 9 | S: SEQ ID NO: 35 | 7366.32 | 7365.8 |
| | AS: SEQ ID NO: 30 | 7749.1 | 7748.5 |
| 10 | S: SEQ ID NO: 36 | 7325.36 | 7324.5 |
| | AS: SEQ ID NO: 32 | 7665.95 | 7665.1 |
| 11 | S: SEQ ID NO: 39 | 7400.35 | 7401.4 |
| | AS: SEQ ID NO: 28 | 7506.84 | 7507.7 |
| 12 | S: SEQ ID NO: 40 | 7249.25 | 7250.5 |
| | AS: SEQ ID NO: 28 | 7506.84 | 7508 |
| 13 | S: SEQ ID NO: 39 | 7400.35 | 7401.6 |
| | AS: SEQ ID NO: 41 | 7482.76 | 7484 |
| 14 | S: SEQ ID NO: 40 | 7249.25 | 7250.7 |
| | AS: SEQ ID NO: 41 | 7482.76 | 7484.1 |
| 15 | S: SEQ ID NO: 42 | 7281.21 | 7280.2 |
| | AS: SEQ ID NO: 30 | 7749.1 | 7748.1 |
| 16 | S: SEQ ID NO: 43 | 7240.25 | 7239.6 |
| | AS: SEQ ID NO: 32 | 7665.95 | 7665.2 |
| 17 | S: SEQ ID NO: 44 | 7352.3 | 7351.3 |
| | AS: SEQ ID NO: 45 | 7583.93 | 7582.9 |
| 18 | S: SEQ ID NO: 46 | 7352.3 | 7351.2 |
| | AS: SEQ ID NO: 45 | 7583.93 | 7582.9 |
| 19 | S: SEQ ID NO: 47 | 7400.35 | 7399.7 |
| | AS: SEQ ID NO: 28 | 7506.84 | 7506.1 |
| 20 | S: SEQ ID NO: 48 | 7400.35 | 7399.2 |
| | AS: SEQ ID NO: 28 | 7506.84 | 7506.2 |
| 21 | S: SEQ ID NO: 49 | 7400.35 | 7399.6 |
| | AS: SEQ ID NO: 28 | 7506.84 | 7506.1 |
| 22 | S: SEQ ID NO: 50 | 7281.21 | 7280.4 |
| | AS: SEQ ID NO: 30 | 7749.1 | 7748.2 |
| 23 | S: SEQ ID NO: 51 | 7281.21 | 7280.2 |
| | AS: SEQ ID NO: 30 | 7749.1 | 7748.1 |
| 24 | S: SEQ ID NO: 52 | 7240.25 | 7239.4 |
| | AS: SEQ ID NO: 32 | 7665.95 | 7665.1 |
| 25 | S: SEQ ID NO: 53 | 7281.21 | 7282.7 |
| | AS: SEQ ID NO: 30 | 7749.1 | 7750.6 |
| 26 | S: SEQ ID NO: 53 | 7281.21 | 7282.8 |
| | AS: SEQ ID NO: 54 | 7725.03 | 7726.5 |
| 27 | S: SEQ ID NO: 53 | 7281.21 | 7282.6 |
| | AS: SEQ ID NO: 55 | 7749.1 | 7750.6 |
| 36 | S: SEQ ID NO: 137 | 7302.3 | 7302.5 |
| | AS: SEQ ID NO: 138 | 7777.1 | 7777.2 |
| 37 | S: SEQ ID NO: 139 | 7359.3 | 7359.2 |
| | AS: SEQ ID NO: 140 | 7795.2 | 7795.4 |
| 38 | S: SEQ ID NO: 141 | 7364.4 | 7364.4 |
| | AS: SEQ ID NO: 142 | 7690 | 7690.3 |
| 39 | S: SEQ ID NO: 143 | 7396.4 | 7396.6 |
| | AS: SEQ ID NO: 144 | 7588.9 | 7588.9 |
| 40 | S: SEQ ID NO: 145 | 7389.4 | 7389.3 |
| | AS: SEQ ID NO: 146 | 7687.1 | 7687.2 |
| 41 | S: SEQ ID NO: 147 | 7380.4 | 7380.2 |
| | AS: SEQ ID NO: 148 | 7667 | 7667.2 |
| 42 | S: SEQ ID NO: 34 | 7366.32 | 7365.7 |
| | AS: SEQ ID NO: 149 | 7373.80 | 7373.3 |
| 43 | S: SEQ ID NO: 34 | 7366.32 | 7365.7 |
| | AS: SEQ ID NO: 150 | 7014.50 | 7014 |
| 44 | S: SEQ ID NO: 34 | 7366.32 | 7365.7 |
| | AS: SEQ ID NO: 151 | 7773.17 | 7773.4 |
| 45 | S: SEQ ID NO: 31 | 7325.36 | 7324.6 |
| | AS: SEQ ID NO: 152 | 7677.98 | 7677.8 |
| 46 | S: SEQ ID NO: 31 | 7325.36 | 7324.6 |
| | AS: SEQ ID NO: 153 | 7658.02 | 7657.9 |
| 47 | S: SEQ ID NO: 154 | 7381.46 | 7380.9 |
| | AS: SEQ ID NO: 32 | 7665.95 | 7665.6 |
| 48 | S: SEQ ID NO: 155 | 7325.36 | 7325.8 |
| | AS: SEQ ID NO: 32 | 7665.95 | 7665.6 |
| 49 | S: SEQ ID NO: 31 | 7325.36 | 7324.6 |
| | AS: SEQ ID NO: 156 | 7690.02 | 7689.4 |
| 50 | S: SEQ ID NO: 31 | 7325.36 | 7324.6 |
| | AS: SEQ ID NO: 157 | 7640.03 | 7639.7 |
| 51 | S: SEQ ID NO: 31 | 7325.36 | 7324.6 |
| | AS: SEQ ID NO: 158 | 7537.89 | 7538.3 |
| 52 | S: SEQ ID NO: 31 | 7325.36 | 7324.6 |
| | AS: SEQ ID NO: 159 | 7525.85 | 7526.3 |
| 53 | S: SEQ ID NO: 160 | 7313.32 | 7314.5 |
| | AS: SEQ ID NO: 152 | 7677.98 | 7679.1 |
| 54 | S: SEQ ID NO: 161 | 7326.34 | 7325.9 |
| | AS: SEQ ID NO: 32 | 7665.95 | 7665.6 |
| 55 | S: SEQ ID NO: 162 | 7309.29 | 7308.8 |
| | AS: SEQ ID NO: 32 | 7665.95 | 7665.6 |
| 56 | S: SEQ ID NO: 163 | 7325.36 | 7324.5 |
| | AS: SEQ ID NO: 32 | 7665.95 | 7665.6 |
| 57 | S: SEQ ID NO: 31 | 7325.36 | 7324.6 |
| | AS: SEQ ID NO: 164 | 7665.95 | 7666.2 |
| 58 | S: SEQ ID NO: 31 | 7325.36 | 7324.6 |
| | AS: SEQ ID NO: 165 | 7346.74 | 7347.1 |
| 59 | S: SEQ ID NO: 166 | 7206.15 | 7206.1 |
| | AS: SEQ ID NO: 156 | 7690.02 | 7690 |
| 60 | S: SEQ ID NO: 43 | 7240.25 | 7239 |
| | AS: SEQ ID NO: 156 | 7690.02 | 7689.8 |

“S” means the sense strand; “AS” means the antisense strand.

### Example 2. In Vitro Characterization of the RNAi Agents

Selected RNAi agents were tested in vitro for target mRNA inhibition in cultured cells, including 293T cells, mouse cortical neurons (MCN) and/or human induced pluripotent stem cells (hiPSC).

### Materials and Methods

293T Luciferase Transfection, RNAi Treatment and Analysis: 293T cells transfected with the pMIR-luciferase construct (Invitrogen, Waltham, MA) containing the target sequence were plated overnight at 37° C.; 5% CO₂. Cells were transfected on day two with siRNAs using RNAiMAX (Invitrogen, Waltham, MA) using the protocol provided by the manufacturer. Cells were incubated at 37° C.; 5% C02 for 48 hrs. Plates were cooled to room temperature followed by the addition of an equal volume of Bio-Glo (Promega, Madison, WI) to each well. Plates were incubated in the dark at room temperature and read on a BioTek Neos2 plate reader (Agilent, Santa Clara, CA).

Mouse Primary Cortical Neuron (MCN) Culture and RNAi Treatment and Analysis: Mouse primary cortical neurons were isolated from wild type C57BL6 mouse embryos at E18, or from hTau C57BL6 transgenic mouse embryos expressing human tau transgene at E18. Cells were plated in poly-D-lysine coated 96-well plates at a density of 40k cells/well and cultured in NbActivl (BrainBits, LLC) containing 1% Antibiotic/Antimycotic (Corning) for 7 days at 37° C. in a tissue culture incubator in a humidified chamber with 5% CO₂. On Day 7, half of the medium was removed from each well and 2× concentration of RNAi in culture media with 2% FBS was added for treatment as CRC and incubated with cells for additional 7, 14 or 21 days. Half media change was done every 7 days with fresh culture media. At the end of RNAi treatment, RT-qPCR was performed to quantify SNCA or MAPT mRNA levels using TaqMan Fast Advanced Cell-to-CT kit. Specifically, cells were lysed, cDNA was generated on Mastercycler X50a (Eppendorf), and qPCR was carried out on QuantStudio 7 Flex Real-Time PCR System (Applied Biosystems). Alpha-synuclein (ThermoFisher, Mm00447333_ml), human MAPT (ThermoFisher, Hs00902194_ml) gene expression levels were normalized by f-actin (ThermoFisher, Mm02619580_g1) using respective probes.

Human Induced Pluripotent Stem Cell-derived Neuron (hiPSC Neuron) Culture and RNAi Treatment and Analysis: Doxycycline-inducible Neurogenin2 (NGN2) human induced Pluripotent Stem Cells (hiPSC) were developed by Bioneer for Eli Lilly. The hiPSC were doxycycline-induced for three days (DIV3) to initiate neuronal differentiation and plated on 96-well PDL and laminin coated plates at 30k/well and grown in Neuronal Differentiation Media (NDM) consisting of DMEM/F12 (Life Technologies 11330-057), Neurobasal media (Gibco 15240062), antibiotics, supplements, growth factors and doxycycline in an incubator (37° C./5% CO2). Cells were half-fed every seven days, and on DIV21, RNAi agent was serially diluted in NDM, and cells were treated with RNAi by aspirating 75 mL and adding 75 mL of 2× RNAi concentration for a final of 1× RNAi according to dilutions. Cells were half-fed every seven days after treatment by removing half of media and adding back fresh NDM. Cell lysates were harvested at DIV35 (14 days later) or DIV42 (21 days later) and RT-qPCR was performed using TaqMan Fast Advanced Cells-to-C_{T} Kit (ThermoFisher, A35377) and to determine mRNA knock down using SNCA probe (ThermoFisher, Hs00240907_ml), or MAPT probe (ThermoFisher, Hs00902194_ml), as gene of interest, and ACTb probe as the housekeeping gene (ThermoFisher, Hs99999903_ml).

### Results

Tables 9A-9C summarize the in vitro activities of selected SNCA RNAi agents. As shown in Tables 9A-9C, the tested RNAi agents knock down SNCA expression in several different cell lines.

TABLE 9A
In vitro activities of selected SNCA RNAi agents
in Mouse Primary Cortical Neurons

| | | MCN, 7 d |
|---|---|---|
| SNCA | | % KD |
| RNAi | MCN, 7 d | (knockdown) |
| Agent | IC50 | of SNCA |
| No. | (nM) | at 4 nM |
| 2 | 3.3 | 58.8 |
| 3 | 4.43 | 54.3 |
| 4 | 5.3 | 25.1 |
| 5 | 5.5 | 49.6 |
| 6 | 2.6 | 57.6 |
| 7 | 2.1 | 59.5 |
| 8 | 2.8 | 54.7 |
| 9 | 2.4 | 63 |
| 10 | 3.2 | 57.8 |
| 11 | 3.8 | 50.3 |
| 12 | 1.7 | 60.1 |
| 13 | 2.8 | 65 |
| 14 | 2.3 | 62.5 |
| 15 | 2 | 66.6 |
| 16 | 2.9 | 49.1 |
| 17 | 1.46 | 73.7 |
| 18 | 1.25 | 81.7 |
| 19 | 2.44 | 71.6 |
| 20 | 1.82 | 72.5 |
| 21 | 2.23 | 70.87 |
| 22 | 1.67 | 65.4 |
| 23 | 1.47 | 69.8 |
| 24 | 3.76 | 54.5 |
| 25 | 2.32 | 62.9 |
| 26 | 2.6 | 58.9 |
| 27 | 34.1 | 32.1 |
| 28 | 0.99 | 75.7 |
| 29 | 3.76 | 58.1 |
| 30 | 1.51 | 73.5 |
| 31 | 2.24 | 61.9 |
| 32 | 3.23 | 59.6 |
| 33 | 2.25 | 60.4 |
| 34 | 5.01 | 49.7 |
| 35 | 5.95 | 38.4 |
| 36 | 2.502 | 64.8 |
| 37 | 1.78 | 63 |
| 41 | 2.24 | 59.25 |
| 50 | 2.62 | 61.99 |
| 51 | 1.25 | 76.12 |
| 52 | 33.5 | 20.71 |
| 54 | 5.5 | 38.17 |
| 40 | 3.45 | 54.13 |
| 42 | 2.82 | 57.86 |
| 43 | 3.82 | 56.11 |
| 56 | 2.24 | 64.56 |
| 57 | 3.49 | 49.16 |
| 58 | 2.79 | 55.69 |
| 59 | 3.73 | 52.29 |

TABLE 9B
In vitro activities of selected SNCA RNAi
agent in T293 Luciferase Assay

| | | T293 |
|---|---|---|
| | T293 | Luciferase |
| SNCA | Luciferase | Assay, |
| RNAi | Assay, | % KD |
| Agent | IC 50 | (knockdown) |
| No. | (nM) | of SNCA |
| 2 | 0.092 | 85.92 |
| 3 | 0.0706 | 69.69 |
| 16 | 0.044 | 85.07 |
| 17 | 0.083 | 73.5 |
| 18 | 0.012 | 88.69 |
| 19 | 0.025 | 87.38 |
| 20 | 0.19 | 86.72 |
| 21 | 0.136 | 74.62 |
| 22 | 0.133 | 67.62 |
| 23 | 0.028 | 75.92 |
| 24 | 0.064 | 75.97 |
| 25 | 0.061 | 78.72 |
| 26 | 0.057 | 45.75 |
| 27 | 0.32 | 56.79 |
| 28 | 0.045 | 83.14 |
| 29 | 0.061 | 87.5 |
| 30 | 0.042 | 71.94 |
| 31 | 0.025 | 79.84 |
| 32 | 0.021 | 80.97 |
| 33 | 0.078 | 75.8 |
| 34 | 0.116 | 76.49 |
| 35 | 0.032 | 78.18 |
| 40 | 0.0356 | 87.75 |
| 41 | 0.0912 | 84.94 |
| 42 | 0.0991 | 85.43 |
| 43 | 0.0889 | 86.4 |

TABLE 9C
In vitro activities of selected SNCA RNAi
agent in hiPSC Neurons

| | | hiPSC |
|---|---|---|
| | | Neurons, |
| | hiPSC | 21 d |
| SNCA | Neurons, | % KD |
| RNAi | 21 d | (knockdown) |
| Agent | IC50 | of SNCA |
| No. | (nM) | at 1 uM |
| 2 | 70.48 | 92.62 |

Table 10 summarizes the in vitro activities of selected MAPT RNAi agents. As shown in Table 10, the tested RNAi agents knock down MAPT expression in mouse cortical neurons.

TABLE 10
In vitro activities of MAPT RNAi agent
in Mouse Primary Cortical Neurons

| | | MCN, |
|---|---|---|
| | | 7 d |
| MAPT | MCN, | % KD |
| RNAi | 7 d | (knockdown) |
| Agent | IC50 | of MAPT |
| No. | (nM) | at 1 mM |
| 4 | 8.8 | 90 |
| 5 | 26 | 91 |
| 6 | 36.2 | 90 |
| 7 | 8.8 | 89 |
| 8 | 8.1 | 93 |
| 9 | 23 | 91 |
| 10 | 72 | 87 |
| 11 | 1.2 | 90 |
| 12 | 8.5 | 85 |
| 13 | 1.2 | 87 |
| 14 | 2.2 | 86 |
| 15 | 4.7 | 91 |
| 16 | 7.7 | 89 |
| 17 | 22.3 | 91 |
| 18 | 36.8 | 91 |
| 19 | 8.4 | 89 |
| 20 | 12 | 90 |
| 21 | 8.5 | 89 |
| 22 | 7.6 | 92 |
| 23 | 6.3 | 92 |
| 24 | 66.9 | 91 |
| 25 | 4.4 | 83 |
| 26 | 9.2 | 82 |
| 27 | 4 | 82 |
| 36 | 7.6 | 87 |
| 37 | 35.9 | 75 |
| 38 | 47.9 | 81 |
| 39 | 19.4 | 79 |
| 40 | 98.4 | 70 |
| 41 | 23.9 | 80 |
| 42 | 82.3 | 82 |
| 43 | 28.2 | 84 |
| 44 | 2.8 | 87 |
| 45 | 23.3 | 87 |
| 46 | 56 | 80 |
| 47 | 41.8 | 83 |
| 48 | 11.8 | 84 |
| 49 | 6 | 86 |
| 50 | 36.4 | 87 |
| 51 | 11.9 | 88.2 |
| 52 | 8.3 | 68 |
| 53 | 6 | 83 |
| 54 | 8.6 | 81 |
| 55 | 11.2 | 87 |
| 56 | 12.6 | 84 |
| 57 | 18.4 | 83 |
| 58 | 19.5 | 83 |
| 59 | 10 | 92 |
| 60 | 5 | 94 |

### Example 3. In Vivo Characterization of Selected RNAi Agents

The efficacy of selected RNAi agents was also studied in Sprague Dawley rats. Six rats received intrathecal delivery of 300 μg or 100 g of the SNCA RNAi agent or PBS (phosphate buffered saline) and were sacrificed 7 days after the infusion. Rat SNCA mRNA expression in spinal cord and brain were measured and analyzed by qPCR. The results are shown in Table 11A.

Similar studies were done using 0.4 mg, 1.2 mg, or 2.4 mg of the SNCA RNAi agent and the rat were sacrificed 2 months after the administration of SNCA RNAi agent. Rat SNCA mRNA expression in spinal cord and brain were measured and analyzed by qPCR. The results are shown in Table 11B.

TABLE 11A
The Percentage Knockdown (KD) of
SNCA mRNA in Rats

| | | | Rat IT | |
|---|---|---|---|---|
| | | | 7 d | |
| | Rat IT, | Rat IT, | % KD | Rat IT |
| SNCA | 7 d | 7 d | Lumbar | 7 d |
| RNAi | % KD | % KD | Dorsal | % KD |
| Agent | Brain | Frontal | Root | Spinal |
| No. | stem | Cortex | Ganglia | Cord |
| 2 | 63.50 | 63.76 | 73.38 | 91.67 |
| (300 μg) | | | | |
| 17 | 77.44 | 76.94 | 75.52 | 84.75 |
| (300 μg) | | | | |
| 18 | 85.08 | 92.64 | 81.57 | 53.98 |
| (100 μg) | | | | |
| 19 | 61.41 | 84.96 | 67.43 | 54.56 |
| (100 μg) | | | | |
| 21 | 0.33 | 84.9 | 0.78 | 73.8 |
| (100 μg) | | | | |
| 20 | 53.6 | 34.2 | N.D. | 79.1 |
| (100 μg) | | | | |
| 40 | 57.7 | 31.43 | N.D. | 78.51 |
| (100 μg) | | | | |
| 43 | 49.53 | 23.04 | N.D. | 75.98 |
| (100 μg) | | | | |
| 56 | 46.94 | 50.84 | N.D. | 71.8 |
| (100 μg) | | | | |
| 57 | 39.86 | 52.21 | N.D. | 68.32 |
| (100 μg) | | | | |
| 58 | 46.93 | 35.1 | N.D. | 73.26 |
| (100 μg) | | | | |
| 59 | 52.4 | 38.66 | N.D. | 70.22 |
| (100 μg) | | | | |

N.D. means not determined.

TABLE 11B
The Percentage Knockdown (KD) of SNCA mRNA in Rats

| | | Rat IT, | Rat IT, | Rat IT, | Rat IT, | |
|---|---|---|---|---|---|---|
| | | 2 months | 2 months | 2 months | 2 months | |
| | | % KD | % KD | % KD | % KD | |
| SNCA | | Cervical | Thoracic | Thoracic | Lumber | Rat IT, |
| RNAi | | Spinal | Spinal | Spinal | Spinal | 2 months |
| Agent | | Cord | Cord | Cord | Cord | % KD |
| No. | Dose | (SC1) | (SC4) | (SC7) | (SC10) | Cerebellum |
| 40 | 0.4 mg | 45.68 | 69.19 | 79.14 | 87.73 | 39.34 |
| 40 | 1.2 mg | 57.15 | 73.98 | 73.45 | 80.18 | 5.94 |
| 40 | 2.4 mg | 73.57 | 79.11 | 83.90 | 92.17 | 30.90 |
| 41 | 0.4 mg | 57.46 | 72.50 | 77.30 | 90.89 | 6.75 |
| 41 | 1.2 mg | 77.30 | 78.64 | 80.81 | 91.10 | 30.56 |
| 41 | 2.4 mg | 84.68 | 81.08 | 85.87 | 91.71 | 50.08 |

The efficacy of selected SNCA RNAi agents was studied in wildtype C56BL/6N mice. 59 mice received intracerebroventricular (ICV) injection of 30 pig of the RNAi agent or PBS (phosphate buffered saline), and were sacrificed 21 days after the injection. Mouse SNCA mRNA expression in spinal cord and brain were measured and analyzed by quantitative PCR (qPCR). The results are shown in Table 1 IC.

TABLE 11C
The Percentage Knockdown (KD) of SNCA mRNA in Mice

| | Mouse ICV, | Mouse ICV, | | Mouse ICV, |
|---|---|---|---|---|
| SNCA | 21 d | 21 d | Mouse ICV, | 21 d |
| RNAi | % KD | % KD | 21 d | % KD |
| Agent | Brain | Frontal | % KD | Spinal |
| No. | stem | Cortex | Hippocampus | Cord |
| 41 | 72 | 23 | 25 | 49 |
| 42 | 80 | 46 | 37 | 70 |
| 50 | 64.30 | 7.81 | N.D. | 27.12 |
| 51 | 30.07 | 14.25 | N.D. | 27.35 |
| 52 | 52.37 | 43.05 | 29.25 | 20.35 |
| 53 | 65.22 | 26.79 | 18.85 | 56.49 |
| 54 | 41.98 | 7.83 | 2.85 | 35.27 |
| 55 | 39.82 | 17.30 | 11.27 | 32.48 |

N.D. means not detected.

The efficacy of selected MAPT RNAi agents was also studied in hTau transgenic mice expressing human MAPT RNA and lacking murine MAPT RNA (Andorfer et al., J Neurochem 2003, 86, 582-590). Six mice received intracerebroventricular (ICV) injection of 100 μg or 250 μg of the MAPT RNAi agent or PBS (phosphate buffered saline) and were sacrificed on Day 14, 35 or 59 after the injection. MAPT mRNA expression in the brain was measured and analyzed by quantitative PCR (qPCR). The results are shown in Tables 1 ID-11F.

TABLE 11D
The Percentage Knockdown (KD) of MAPT mRNA in hTau
mice 14 days after 100 μg of MAPT RNAi agent treatment

| | Mouse ICV, | Mouse ICV, | | Mouse ICV, |
|---|---|---|---|---|
| MAPT | 14 d | 14 d | Mouse ICV, | 14 d |
| RNAi | % KD | % KD | 14 d | % KD |
| Agent | Brain | Frontal | % KD | Spinal |
| No. | stem | Cortex | Hippocampus | Cord |
| 4 | 43 | 72 | 72 | 55 |
| 6 | 36 | 70 | 59 | 61 |
| 8 | 60 | 83 | 71 | 77 |
| 11 | 39 | 29 | 42 | 22 |
| 12 | 74 | 49 | 79 | 67 |
| 47 | 59 | 79 | 73 | 76 |
| 48 | 36 | 75 | 54 | 66 |
| 53 | 42 | 23 | 40 | 35 |
| 59 | 60 | 58 | 60 | 57 |
| 60 | 60 | 42 | 60 | 61 |

TABLE 11E
The Percentage Knockdown (KD) of MAPT mRNA in hTau
mice 35 days after 100 μg of MAPT RNAi agent treatment

| | Mouse ICV, | Mouse ICV, | | Mouse ICV, |
|---|---|---|---|---|
| MAPT | 35 d | 35 d | Mouse ICV, | 35 d |
| RNAi | % KD | % KD | 35 d | % KD |
| Agent | Brain | Frontal | % KD | Spinal |
| No. | stem | Cortex | Hippocampus | Cord |
| 6 | 69 | 80 | 80 | 86 |
| 49 | 59 | 79 | 66 | 79 |
| 50 | 36 | 71 | 58 | 71 |
| 51 | 29 | 53 | 43 | 55 |
| 52 | 18 | 42 | 32 | 50 |

TABLE 11F
The Percentage Knockdown (KD) of MAPT mRNA in hTau
mice 59 days after 250 μg of MAPT RNAi agent treatment

| | Mouse ICV, | Mouse ICV, | | Mouse ICV, |
|---|---|---|---|---|
| MAPT | 59 d | 59 d | Mouse ICV, | 59 d |
| RNAi | % KD | % KD | 59 d | % KD |
| Agent | Brain | Frontal | % KD | Spinal |
| No. | stem | Cortex | Hippocampus | Cord |
| 4 | 41 | 52 | 60 | 45 |
| 5 | 58 | 67 | 66 | 61 |
| 6 | 68 | 86 | 82 | 84 |
| 8 | 64 | 77 | 71 | 70 |
| 45 | 84 | 84 | 86 | 91 |
| 46 | 61 | 72 | 58 | 68 |
| 47 | 65 | 83 | 77 | 78 |

### RNAi Agent Tissue Distribution and Microgliosis Analysis

Fixed rat right hemisphere brains and spinal cords (the fourth cervical segment [C4 or SC2], the fourth thoracic segment [T4 or SC5], the eleventh thoracic segment [Ti 1 or SC8] and the first lumbar segment [L1 or SC10]) were stored in cold (4° C.) ix PBS (Phosphate Buffered Saline, CAS Number: 7732-18-5) until tissue processing. Samples were processed on a Leica ASP6025S Tissue Processor and embedded using Leica HistoCore Arcadia H— Heated Paraffin Embedding Station and HistoCore Arcadia C—Cold Plate. The brains were embedded sagittally and spinal cords transversely. Blocks were stored at room temperature until sectioning.

Blocks were sectioned using HistoCore AUTOCUT—Automated Rotary Microtome (Leica Biosystems, 149AUTO00C1). Briefly, blocks were trimmed to fully expose the tissue and 5 μm thick sections were taken and placed on Fisherbrand™ SuperfrostT^{M} Plus Microscope Slides (Fisherbrand, 12-550-15). Brains were sectioned in steps from midline (0 um, 500 μm and 1000 μm from midline) and spinal cords were sectioned serially. Slides were dried overnight at room temperature before staining.

Slides were stained on Leica BOND RX (Leica Biosystems, 21.2821). For each brain, one slide was stained from each step level and for spinal cords one serial section was stained. All slides were stained using Advanced Cell Diagnostics (ACD) miRNAscope™ LS Reagent Kit—RED (Advanced Cell Diagnostics, 324600). A probe was used (Advanced Cell Diagnostics, 1063228-S1, for Eli Lilly & Co.) for detection of the anti-sense siRNA strand. Other reagents used included miRNAscope™ LS Negative Control Probe—SR-Scramble-S1 (Advanced Cell Diagnostics, 727888-S1) and BOND Polymer Refine Red Detection (Leica Biosystems, DS9390). All slides were stained according to the manufacturer's protocol for miRNAscope™ with slight modification. Washes in steps 75, 85 and 92 were modified to open washes. Once stained, slides were washed in DI water for 2 minutes, dried at 60° C. for 30 minutes, and coverslipped.

Slides were scanned on Leica Aperio GT450 Slide Scanner and uploaded to Aperio eSlide Manager for analysis. Using Aperio ImageScope, the frontal cortex, brain stem, C4, T4, T11, and L1 were delineating manually, and an image analysis algorithm was run on each delineated region to calculate “percent pixel positivity”. Briefly, an algorithm was adapted from the Aperio ImageScope “Positive Pixel Count 2002-08-11” algorithm. Outputs of the algorithm included pixel positivity, where a positive pixel equates to the anti-sense strand of the siRNA molecule and all other pixels were negative pixels. The “percent pixel positivity” is the number ofpositive pixels in the image, divided by the number of total pixels in the image, including negative pixels, multiplied by 100. Results are shown in Table 12, which shows the tested RNAi agent has good distribution profile across brain and spinal cord.

TABLE 12
RNAi agent tissue distribution measured by miRNAscope ™ percent pixel positivity.

| | Rat IT, 7 d | Rat IT, 7 d | Rat IT, 7 d | Rat IT, 7 d | Rat IT, 7 d | Rat IT, 7 d |
|---|---|---|---|---|---|---|
| | % Pixel | % Pixel | % Pixel | % Pixel | % Pixel | % Pixel |
| | Positivity | Positivity | Positivity | Positivity | Positivity | Positivity |
| | L1 Spinal | T11 Spinal | T4 Spinal | C4 Spinal | Brain | Frontal |
| | Cord | Cord | Cord | Cord | Stem | Cortex |
| SNCA | 5.55 | 7.50 | 8.19 | 4.75 | 1.38 | 0.37 |
| RNAi | | | | | | |
| agent | | | | | | |
| No. 18 | | | | | | |
| SNCA | 8.78 | 7.11 | 7.76 | 3.46 | 0.86 | 0.46 |
| RNAi | | | | | | |
| agent | | | | | | |
| No. 17 | | | | | | |

Additional slides were stained with anti-Ibal antibody (FUJIFILM Wako, 013-27691, 1:2000) diluted in BOND Primary Antibody Diluent (Leica, AR9352) using IHC Protocol F (Leica) and BOND Polymer Refine Detection Kit (Leica, DS9800). Briefly, after blocking with H₂O₂ (34 (v/v)), the primary antibody was applied. The polymer (Anti-rabbit Poly-HRP-IgG (<25 μg/mL) containing 100 (v/v) animal serum in tris-buffered saline/0.1% ProClin™ 950) was applied followed by the DAB Part 1 (66 mM 3,3′-Diaminobenzidine tetrahydrochloride hydrate, in a stabilizer solution) Part B (pr.imr (v/v) Hydrogen Peroxide in a stabilizer solution) and Hematoxylin (<0.1% Hematoxylin) counterstain. After staining, slides were dehydrated using a Leica mT5010 Autostainer XL and coverslipped with Surgipath Micromount mounting medium (Leica, 3801731). Slides were scanned on Leica Aperio GT450 Slide Scanner and uploaded to Aperio eSlide Manager for analysis. Using Aperio ImageScope, images were opened and assessed for microgliosis using the scoring parameters shown in Table 13.

TABLE 13
Microgliosis Scoring

| Microgliosis | |
|---|---|
| Scoring | Description |
| No | No activated microglia. |
| Inflammation | |
| Minimal | 3-5 focal lesions of activated microglia across the tissue. |
| Inflammation | Microglia primary processes thicken, and cell bodies |
| | begins to round. Microglia upregulate the marker IBA1. |
| Mild | 5-10 focal lesions of activated microglia across the tissue. |
| Inflammation | Microglia primary process have thickened and begin to |
| | retract into the cell body. Microglia have lost most |
| | secondary and tertiary processes. The number of microglia |
| | increases, and they continue to upregulate the marker IBA1. |
| Moderate | Diffuse activated microglia across the tissue. Microglia |
| Inflammation | have become ameboid in shape. There may be small |
| | primary process, but no secondary, or tertiary processes. |
| | Microglia numbers continue to increase, and they continue |
| | to upregulate IBA1 marker expression. |
| Severe | Widespread, diffuse activated microglia across the tissue. |
| Inflammation | Microglia are ameboid with no processes. Microglia |
| | numbers have increased and there is high expression of the |
| | marker IBA1. |

Results of microgliosis assessments are shown in Table 14.

TABLE 14
Microgliosis Assessments

| | | SNCA RNAi | SNCA RNAi | SNCA RNAi | SNCA RNAi |
|---|---|---|---|---|---|
| Tissue Region | Dose | Agent No. 2 | Agent No. 16 | Agent No. 40 | Agent No. 41 |
| Lumbar Spinal | 0.4 mg | No Inflammation | No Inflammation | No Inflammation | No Inflammation |
| Cord | | (4/5 no | (4/5 no | (5/5 no | (5/5 no |
| | | inflammation; | inflammation; | inflammation) | inflammation) |
| | | 1/5 minimal) | 1/5 minimal) | | |
| Thoracic Spinal | 0.4 mg | No Inflammation | No Inflammation | No Inflammation | No Inflammation |
| Cord | | (4/5 no | (4/5 no | (5/5 no | (5/5 no |
| | | inflammation; | inflammation; | inflammation) | inflammation) |
| | | 1/5 minimal) | 1/5 minimal) | | |
| Cervical Spinal | 0.4 mg | No Inflammation | No Inflammation | No Inflammation | No Inflammation |
| Cord | | (5/5 no | (5/5 no | (5/5 no | (5/5 no |
| | | inflammation) | inflammation) | inflammation) | inflammation) |
| Cerebellum | 0.4 mg | No Inflammation | No Inflammation | No Inflammation | No Inflammation |
| (Deep White | | (5/5 no | (5/5 no | (5/5 no | (5/5 no |
| Matter) | | inflammation) | inflammation) | inflammation) | inflammation) |
| Cerebellum | 0.4 mg | No Inflammation | No Inflammation | No Inflammation | No Inflammation |
| (Molecular | | (5/5 no | (5/5 no | (5/5 no | (5/5 no |
| Layer) | | inflammation) | inflammation) | inflammation) | inflammation) |
| Cerebellum | 0.4 mg | No Inflammation | No Inflammation | No Inflammation | No Inflammation |
| (Granular Layer) | | (5/5 no | (5/5 no | (5/5 no | (5/5 no |
| | | inflammation) | inflammation) | inflammation) | inflammation) |
| Brainstem | 0.4 mg | No Inflammation | No Inflammation | No Inflammation | No Inflammation |
| (White Matter) | | (4/5 no | (4/5 no | (4/5 no | (5/5 no |
| | | inflammation; | inflammation; | inflammation; | inflammation) |
| | | 1/5 minimal | 1/5 minimal | 1/5 minimal | |
| | | inflammation) | inflammation) | inflammation) | |
| Brainstem | 0.4 mg | No Inflammation | No Inflammation | No Inflammation | No Inflammation |
| (Grey Matter) | | (5/5 no | (5/5 no | (5/5 no | (5/5 no |
| | | inflammation) | inflammation) | inflammation) | inflammation) |
| Mid brain | 0.4 mg | No Inflammation | (5/5 no inflammation) | (5/5 no inflammation) | (5/5 no inflammation) |
| (White Matter) | | (5/5 no | No Inflammation | No Inflammation | No Inflammation |
| | | inflammation) | | | |
| Mid brain | 0.4 mg | No Inflammation | No Inflammation | No Inflammation | No Inflammation |
| (Grey Matter) | | (5/5 no | (5/5 no | (5/5 no | (5/5 no |
| | | inflammation) | inflammation) | inflammation) | inflammation) |
| Hind Cortex | 0.4 mg | No Inflammation | No Inflammation | No Inflammation | No Inflammation |
| | | (5/5 no | (5/5 no | (5/5 no | (5/5 no |
| | | inflammation) | inflammation) | inflammation) | inflammation) |
| Frontal Cortex | 0.4 mg | No Inflammation | No Inflammation | No Inflammation | No Inflammation |
| | | (5/5 no | (5/5 no | (5/5 no | (5/5 no |
| | | inflammation) | inflammation) | inflammation) | inflammation) |
| Hippocampus | 0.4 mg | No Inflammation | No Inflammation | No Inflammation | No Inflammation |
| | | (5/5 no | (5/5 no | (5/5 no | (5/5 no |
| | | inflammation) | inflammation) | inflammation) | inflammation) |
| Striatum | 0.4 mg | No Inflammation | No Inflammation | No Inflammation | No Inflammation |
| | | (5/5 no | (5/5 no | (5/5 no | (5/5 no |
| | | inflammation) | inflammation) | inflammation) | inflammation) |
| Thalamus | 0.4 mg | No Inflammation | No Inflammation | No Inflammation | No Inflammation |
| | | (5/5 no | (5/5 no | (5/5 no | (5/5 no |
| | | inflammation) | inflammation) | inflammation) | inflammation) |
| Corpus Callosum | 0.4 mg | No Inflammation | No Inflammation | No Inflammation | No Inflammation |
| | | (5/5 no | (5/5 no | (5/5 no | (5/5 no |
| | | inflammation) | inflammation) | inflammation) | inflammation) |
| Olfactory bulb | 0.4 mg | No Inflammation | No Inflammation | No Inflammation | No Inflammation |
| (White Matter | | (5/5 no | (5/5 no | (5/5 no | (5/5 no |
| Tract) | | inflammation) | inflammation) | inflammation) | inflammation) |
| Lumbar Spinal | 1.2 mg | Minimal | Mild | No Inflammation | No Inflammation |
| Cord | | (4/5 minimal; | (2/5 minimal; | (5/5 no | (5/5 no |
| | | 1/5 no | 2/5 mild; | inflammation) | inflammation) |
| | | inflammation) | 1/5 moderate) | | |
| Thoracic Spinal | 1.2 mg | Minimal | Mild | No Inflammation | No Inflammation |
| Cord | | (4/5 minimal; | (2/5 minimal; | (5/5 no | (5/5 no |
| | | 1/5 no | 2/5 mild; | inflammation) | inflammation) |
| | | inflammation) | 1/5 moderate) | | |
| Cervical Spinal | 1.2 mg | Minimal | Minimal | No Inflammation | No Inflammation |
| Cord | | (3/5 minimal; | (4/5 minimal; | (5/5 no | (5/5 no |
| | | 2/5 no | 1/5 mild) | inflammation) | inflammation) |
| | | inflammation) | | | |
| Cerebellum | 1.2 mg | No Inflammation | Minimal | No Inflammation | No Inflammation |
| (Deep White | | (4/5 no | (2/5 minimal; | (5/5 no | (4/4 no |
| Matter) | | inflammation; | 1/5 mild; | inflammation) | inflammation) |
| | | 1/5 minimal | 2/5 moderate) | | |
| | | inflammation) | | | |
| Cerebellum | 1.2 mg | No Inflammation | No Inflammation | No Inflammation | No Inflammation |
| (Molecular | | (5/5 no | (4/5 no | (5/5 no | (4/4 no |
| Layer) | | inflammation) | inflammation; | inflammation) | inflammation) |
| | | | 1/5 minimal | | |
| | | | inflammation) | | |
| Cerebellum | 1.2 mg | No Inflammation | No Inflammation | No Inflammation | No Inflammation |
| (Granular Layer) | | (5/5 no | (4/5 no | (5/5 no | (4/4 no |
| | | inflammation) | inflammation; | inflammation) | inflammation) |
| | | | 1/5 minimal | | |
| | | | inflammation) | | |
| Brainstem | 1.2 mg | No Inflammation | No Inflammation | No Inflammation | No Inflammation |
| (White Matter) | | (5/5 no | (5/5 no | (5/5 no | (4/4 no |
| | | inflammation) | inflammation) | inflammation) | inflammation) |
| Brainstem | 1.2 mg | No Inflammation | No Inflammation | No Inflammation | No Inflammation |
| (Grey Matter) | | (5/5 no | (5/5 no | (5/5 no | (4/4 no |
| | | inflammation) | inflammation) | inflammation) | inflammation) |
| Mid brain | 1.2 mg | No Inflammation | No Inflammation | No Inflammation | No Inflammation |
| (White Matter) | | (5/5 no | (5/5 no | (5/5 no | (4/4 no |
| | | inflammation) | inflammation) | inflammation) | inflammation) |
| Mid brain | 1.2 mg | No Inflammation | No Inflammation | No Inflammation | No Inflammation |
| (Grey Matter) | | (5/5 no | (5/5 no | (5/5 no | (4/4 no |
| | | inflammation) | inflammation) | inflammation) | inflammation) |
| Hind Cortex | 1.2 mg | No Inflammation | No Inflammation | No Inflammation | No Inflammation |
| | | (5/5 no | (5/5 no | (5/5 no | (4/4 no |
| | | inflammation) | inflammation) | inflammation) | inflammation) |
| Frontal Cortex | 1.2 mg | No Inflammation | No Inflammation | No Inflammation | No Inflammation |
| | | (5/5 no | (5/5 no | (5/5 no | (4/4 no |
| | | inflammation) | inflammation) | inflammation) | inflammation) |
| Hippocampus | 1.2 mg | No Inflammation | No Inflammation | No Inflammation | No Inflammation |
| | | (5/5 no | (5/5 no | (5/5 no | (4/4 no |
| | | inflammation) | inflammation) | inflammation) | inflammation) |
| Striatum | 1.2 mg | No Inflammation | No Inflammation | No Inflammation | No Inflammation |
| | | (5/5 no | (5/5 no | (5/5 no | (4/4 no |
| | | inflammation) | inflammation) | inflammation) | inflammation) |
| Thalamus | 1.2 mg | No Inflammation | No Inflammation | No Inflammation | No Inflammation |
| | | (5/5 no | (5/5 no | (5/5 no | (4/4 no |
| | | inflammation) | inflammation) | inflammation) | inflammation) |
| Corpus Callosum | 1.2 mg | No Inflammation | No Inflammation | No Inflammation | No Inflammation |
| | | (4/5 no | (5/5 no | (5/5 no | (4/4 no |
| | | inflammation: | inflammation) | inflammation) | inflammation) |
| | | 1/5 minimal | | | |
| | | inflammation) | | | |
| Olfactory bulb | 1.2 mg | No Inflammation | No Inflammation | No Inflammation | No Inflammation |
| (White Matter | | (5/5 no | (5/5 no | (5/5 no | (4/4 no |
| Tract) | | inflammation) | inflammation) | inflammation) | inflammation) |
| Lumbar Spinal | 2.4 mg | Mild | Moderate | No Inflammation | No Inflammation |
| Cord | | (3/5 mild; | (3/5 moderate; | (5/5 no | (5/5 no |
| | | 2/5 minimal) | 1/5 mild; | inflammation) | inflammation) |
| | | | 1/5 minimal) | | |
| Thoracic Spinal | 2.4 mg | Mild | Moderate | No Inflammation | No Inflammation |
| Cord | | (3/5 mild; | (3/5 moderate; | (5/5 no | (5/5 no |
| | | 2/5 minimal) | 1/5 mild; | inflammation) | inflammation) |
| | | | 1/5 minimal) | | |
| Cervical Spinal | 2.4 mg | Minimal | Mild | No Inflammation | No Inflammation |
| Cord | | (5/5 minimal) | (3/5 mild; | (5/5 no | (5/5 no |
| | | | 2/5 minimal) | inflammation) | inflammation) |
| Cerebellum | 2.4 mg | Minimal | Mild | No Inflammation | No Inflammation |
| (Deep White | | (1/5 mild; 2/5 | (4/5 animals mild; | (5/5 no | (4/5 no |
| Matter) | | animals minimal; | 1/5 no | inflammation) | inflammation; |
| | | 2/5 no | inflammation) | | 1/5 minimal) |
| | | inflammation) | | | |
| Cerebellum | 2.4 mg | No Inflammation | Minimal | No Inflammation | No Inflammation |
| (Molecular | | (5/5 no | (4/5 animals | (5/5 no | (5/5 no |
| Layer) | | inflammation) | minimal; 1/5 no | inflammation) | inflammation) |
| | | | inflammation) | | |
| Cerebellum | 2.4 mg | No Inflammation | Minimal | No Inflammation | No Inflammation |
| (Granular Layer) | | (5/5 no | (4/5 animals | (5/5 no | (5/5 no |
| | | inflammation) | minimal; 1/5 no | inflammation) | inflammation) |
| | | | inflammation) | | |
| Brainstem | 2.4 mg | No/Minimal | Minimal | No Inflammation | No Inflammation |
| (White Matter) | | inflammation | (5/5 animals | (5/5 no | (4/5 no |
| | | (3/5 no | minimal) | inflammation) | inflammation; |
| | | inflammation; | | | 1/5 minimal) |
| | | 2/5 minimal) | | | |
| Brainstem | 2.4 mg | No Inflammation | No Inflammation | No Inflammation | No Inflammation |
| (Grey Matter) | | (5/5 no | (5/5 no | (5/5 no | (5/5 no |
| | | inflammation) | inflammation) | inflammation) | inflammation) |
| Mid brain | 2.4 mg | Minimal | No/Minimal | No Inflammation | No Inflammation |
| (White Matter) | | (3/5 animals | inflammation | (5/5 no | (5/5 no |
| | | minimal; 2/5 no | (3/5 no | inflammation) | inflammation) |
| | | inflammation) | inflammation; | | |
| | | | 2/5 minimal) | | |
| Mid brain | 2.4 mg | No Inflammation | No Inflammation | No Inflammation | No Inflammation |
| (Grey Matter) | | (5/5 no | (5/5 no | (4/5 no | (5/5 no |
| | | inflammation) | inflammation) | inflammation; | inflammation) |
| | | | | 1/5 minimal) | |
| Hind Cortex | 2.4 mg | No Inflammation | Minimal | No Inflammation | No Inflammation |
| | | (5/5 no | (3/5 animals | (5/5 no | (5/5 no |
| | | inflammation) | minimal; 3/5 no | inflammation) | inflammation) |
| | | | inflammation) | | |
| Frontal Cortex | 2.4 mg | No Inflammation | No inflammation | No Inflammation | No Inflammation |
| | | (5/5 no | (4/5 no | (5/5 no | (5/5 no |
| | | inflammation) | inflammation; | inflammation) | inflammation) |
| | | | 1/5 minimal) | | |
| Hippocampus | 2.4 mg | No Inflammation | No Inflammation | No Inflammation | No Inflammation |
| | | (4/5 no | (5/5 no | (5/5 no | (5/5 no |
| | | inflammation; | inflammation) | inflammation) | inflammation) |
| | | 1/5 minimal | | | |
| | | inflammation) | | | |
| Striatum | 2.4 mg | No Inflammation | No Inflammation | No Inflammation | No Inflammation |
| | | (5/5 no | (5/5 no | (5/5 no | (5/5 no |
| | | inflammation) | inflammation) | inflammation) | inflammation) |
| Thalamus | 2.4 mg | No Inflammation | No Inflammation | No Inflammation | No Inflammation |
| | | (5/5 no | (5/5 no | (5/5 no | (5/5 no |
| | | inflammation) | inflammation) | inflammation) | inflammation) |
| Corpus Callosum | 2.4 mg | Minimal | Minimal | No Inflammation | No Inflammation |
| | | (4/5 animals | (4/5 animals | (5/5 no | (5/5 no |
| | | minimal; 1/5 no | minimal; 1/5 no | inflammation) | inflammation) |
| | | inflammation) | inflammation) | | |
| Olfactory bulb | 2.4 mg | No Inflammation | No Inflammation | No Inflammation | No Inflammation |
| (White Matter | | (4/5 no | (5/5 no | (5/5 no | (5/5 no |
| Tract) | | inflammation; | inflammation) | inflammation) | inflammation) |
| | | 1/5 minimal | | | |
| | | inflammation) | | | |

SEQUENCE LISTING

| SEQ ID NO | Sequence |
|---|---|
| 1 | CUGUACAAGUGCUCAGUUCCA |
| 2 | UGGAACUGAGCACUUGUACAGGA |
| 3 | mC*mU*mGmUmAmCfAmAfGfUfGmC(Uads)mCmAmGmUmUmC*mC*mA |
| 4 | VPmU*fG*mGmAmAfCmUmGmAmGmCmAmCfUmUfGmUmAmCmAmG*mG*mA |
| 5 | (Cads)*mU*mGmUmAmCfAmAfGfUfGmCmUmCmAmGmUmUmC*mC*mA |
| 6 | (Css)*mU*mGmUmAmCmAmAfGfUfGmCmUmCmAmGmUmUmC*mC*mA |
| 7 | VPmU*fG*mGmAfAmCmUfGmAmGmCmAmCfUmUfGmUmAmCmAmG*mG*mA |
| 8 | mC*mU*mGmUmA(Css)mAmAfGfUfGmCmUmCmAmGmUmUmC*mC*mA |
| 9 | mC*mU*mGmUmAmCmAmAfGfUfG(Css)mUmCmAmGmUmUmC*mC*mA |
| 10 | mC*mU*mGmUmAmCmAmAfGfUfGmCmU(Css)mAmGmUmUmC*mC*mA |
| 11 | mC*mU*mGmUmAmCmAmAfGfUfGmCmUmCmAmGmUmU(Css)*mC*mA |
| 12 | mC*mU*mGmUmAmCmAmAfGfUfGmCmUmCmAmGmUmUmC*(Css)*mA |
| 13 | mC*(Uss)*mGmUmAmCmAmAfGfUfGmCmUmCmAmGmUmUmC*mC*mA |
| 14 | mC*mU*mG(Uss)mAmCmAmAfGfUfGmCmUmCmAmGmUmUmC*mC*mA |
| 15 | mC*mU*mGmUmAmCmAmAfG(Uss)fGmCmUmCmAmGmUmUmC*mC*mA |
| 16 | mC*mU*mGmUmAmCmAmAfGfUfGmC(Uss)mCmAmGmUmUmC*mC*mA |
| 17 | mC*mU*mGmUmAmCmAmAfGfUfGmCmUmCmAmG(Uss)mUmC*mC*mA |
| 18 | mC*mU*mGmUmAmCmAmAfGfUfGmCmUmCmAmGmU(Uss)mC*mC*mA |
| 19 | mC*mU*mGmUmAmCmAmAfGfUfGmC(Uads)mCmAmGmUmUmC*mC*mA |
| 20 | mC*mU*mGmUmAmCfAmAfGfUfGmC(Uss)mCmAmGmUmUmC*mC*mA |
| 21 | GUGGAAGUAAAAUCUGAGAAA |
| 22 | UUUCUCAGAUUUUACUUCCACCU |
| 23 | CCAAGUGUGGCUCAUUAGGCA |
| 24 | UGCCUAAUGAGCCACACUUGGAG |
| 25 | UGCAAAUAGUCUACAAACCAA |
| 26 | UUGGUUUGUAGACUAUUUGCACC |
| 27 | mG*mUmGmGmAmAmGmUfAfAfAmA(Uads)mCmUmGmAmGmA*mA*mA |
| 28 | VPmU*fU*mUmCfUmCmAfGmAmUmUmUmUfAmCfUmUmCmCmAmC*mC*mU |
| 29 | mC*mC*mAmAmGmUmGmUfGfGfC(Uads)mCmAmUmUmAmGmG*mC*mA |
| 30 | VPmU*fG*mCmCfUmAmAfUmGmAmGmCmCfAmCfAmCmUmUmGmG*mA*mG |
| 31 | mU*mG*mCmAmAmAmUmAfGfUfC(Uads)mAmCmAmAmAmCmC*mA*mA |
| 32 | VPmU*fU*mGmGfUmUmUfGmUmAmGmAmCfUmAfUmUmUmGmCmA*mC*mC |
| 33 | mG*(Uads)*mGmGmAmAmGmUfAfAfAmAmUmCmUmGmAmGmA*mA*mA |
| 34 | mC*mC*mAmAmGmUmGmUfGfGfCmU(Cads)mAmUmUmAmGmG*mC*mA |
| 35 | (Cads)*mC*mAmAmGmUmGmUfGfGfCmUmCmAmUmUmAmGmG*mC*mA |
| 36 | (Uads)*mG*mCmAmAmAmUmAfGfUfCmUmAmCmAmAmAmCmC*mA*mA |
| 37 | mC*mU*mGmUmAmCfAmAfGfUfGmC(UL1)mCmAmGmUmUmC*mC*mA |
| 38 | mC*mU*mGmUmAmCmAmAfGfUfGmC(UL2)mCmAmGmUmUmC*mC*mA |
| 39 | mG*mU*mGmGmAmAmGmUfAfAfAmA(Uss)mCmUmGmAmGmA*mA*mA |
| 40 | mG*mU*mGmGmAmAmGmUnfAfAmA(Uss)mCmUmGmAmGmA*mA*mA |
| 41 | VPmU*fU*mUmCmUfCmAmGmAfUmUmUmUfAmCfUmUfCmCfAmC*mC*mU |
| 42 | mC*mC*mAmAmGmUmGmUfGfGfC(Uss)mCmAmUmUmAmGmG*mC*mA |
| 43 | mU*mG*mCmAmAmAmUmAfGfUfC(Uss)mAmCmAmAmAmCmC*mA*mA |
| 44 | mC*mC*mAmGmGmUmGmGfAfAfG(Uss)mAmAmAmAmUmCmU*mG*mA |
| 45 | VPmU*fC*mAmGfAmUmUfUmUmAmCmUmUfCmCfAmCmCmUmGmG*mC*mC |
| 46 | mC*mC*mAmGmGmUmGmGfAfAfGmUmAmAmAmAmUmC(Uss)*mG*mA |
| 47 | mG*mU*mGmGmAmAmG(Uss)fAfAfAmAmUmCmUmGmAmGmA*mA*mA |
| 48 | mG*(Uss)*mGmGmAmAmGmUfAfAfAmAmUmCmUmGmAmGmA*mA*mA |
| 49 | mG*mU*mGmGmAmAmGmUfAfAfAmAmUmC(Uss)mGmAmGmA*mA*mA |
| 50 | mC*mC*mAmAmG(Uss)mGmUfGfGfCmUmCmAmUmUmAmGmG*mC*mA |
| 51 | mC*mC*mAmAmGmUmGmUfGfGfCmUmCmA(Uss)mUmAmGmG*mC*mA |
| 52 | (Uss)*mG*mCmAmAmAmUmAfGfUfCmUmAmCmAmAmAmCmC*mA*mA |
| 53 | mC*mC*mAmAmGmUmGmUfGfGfCmU(Css)mAmUmUmAmGmG*mC*mA |
| 54 | VPmU*fG*mCmCmUfAmAmUmGfAmGmCmCfAmCfAmCfUmUfGmG*mA*mG |
| 55 | VPmU*fG*mCmCfUmAfAmUmGmAmGmCmCfAmCfAmCmUmUmGmG*mA*mG |
| 56 | CCAGGUGGAAGUAAAAUCUGA |
| 57 | UCAGAUUUUACUUCCACCUGGCC |
| 58 | GGCGACGACCAGAAGGGGCCCAAGAGAGGGGGCGAGCGACCGAGCGCCGCGACGC |
| | GGAAGTGAGGTGCGTGCGGGCTGCAGCGCAGACCCCGGCCCGGCCCCTCCGAGAG |
| | CGTCCTGGGCGCTCCCTCACGCCTTGCCTTCAAGCCTTCTGCCTTTCCACCCTCG |
| | TGAGCGGAGAACTGGGAGTGGCCATTCGACGACAGTGTGGTGTAAAGGAATTCAT |
| | TAGCCATGGATGTATTCATGAAAGGACTTTCAAAGGCCAAGGAGGGAGTTGTGGC |
| | TGCTGCTGAGAAAACCAAACAGGGTGTGGCAGAAGCAGCAGGAAAGACAAAAGAG |
| | GGTGTTCTCTATGTAGGCTCCAAAACCAAGGAGGGAGTGGTGCATGGTGTGGCAA |
| | CAGTGGCTGAGAAGACCAAAGAGCAAGTGACAAATGTTGGAGGAGCAGTGGTGAC |
| | GGGTGTGACAGCAGTAGCCCAGAAGACAGTGGAGGGAGCAGGGAGCATTGCAGCA |
| | GCCACTGGCTTTGTCAAAAAGGACCAGTTGGGCAAGAATGAAGAAGGAGCCCCAC |
| | AGGAAGGAATTCTGGAAGATATGCCTGTGGATCCTGACAATGAGGCTTATGAAAT |
| | GCCTTCTGAGGAAGGGTATCAAGACTACGAACCTGAAGCCTAAGAAATATCTTTG |
| | CTCCCAGTTTCTTGAGATCTGCTGACAGATGTTCCATCCTGTACAAGTGCTCAGT |
| | TCCAATGTGCCCAGTCATGACATTTCTCAAAGTTTTTACAGTGTATCTCGAAGTC |
| | TTCCATCAGCAGTGATTGAAGTATCTGTACCTGCCCCCACTCAGCATTTCGGTGC |
| | TTCCCTTTCACTGAAGTGAATACATGGTAGCAGGGTCTTTGTGTGCTGTGGATTT |
| | TGTGGCTTCAATCTACGATGTTAAAACAAATTAAAAACACCTAAGTGACTACCAC |
| | TTATTTCTAAATCCTCACTATTTTTTTGTTGCTGTTGTTCAGAAGTIGTTAGTGA |
| | TTTGCTATCATATATTATAAGATTTTTAGGTGTCTTTTAATGATACTGTCTAAGA |
| | ATAATGACGTATTGTGAAATTTGTTAATATATATAATACTTAAAAATATGTGAGC |
| | ATGAAACTATGCACCTATAAATACTAAATATGAAATTTTACCATTTTGCGATGTG |
| | TTTTATTCACTTGTGTTTGTATATAAATGGTGAGAATTAAAATAAAACGTTATCT |
| | CATTGCAAAAATATTTTATTTTTATCCCATCTCACTTTAATAATAAAAATCATGC |
| | TTATAAGCAACATGAATTAAGAACTGACACAAAGGACAAAAATATAAAGTTATTA |
| | ATAGCCATTTGAAGAAGGAGGAATTTTAGAAGAGGTAGAGAAAATGGAACATTAA |
| | CCCTACACTCGGAATTCCCTGAAGCAACACTGCCAGAAGTGTGTTTTGGTATGCA |
| | CTGGTTCCTTAAGTGGCTGTGATTAATTATTGAAAGTGGGGTGTTGAAGACCCCA |
| | ACTACTATTGTAGAGTGGTCTATTTCTCCCTTCAATCCTGTCAATGTTTGCTTTA |
| | CGTATTTTGGGGAACTGTTGTTTGATGTGTATGTGTTTATAATTGTTATACATTT |
| | TTAATTGAGCCTTTTATTAACATATATTGTTATTTTTGTCTCGAAATAATTTTTT |
| | AGTTAAAATCTATTTTGTCTGATATTGGTGTGAATGCTGTACCTTTCTGACAATA |
| | AATAATATTCGACCATGAATAAAAAAAAAAAAAAAGTGGGTTCCCGGGAACTAAG |
| | CAGTGTAGAAGATGATTTTGACTACACCCTCCTTAGAGAGCCATAAGACACATTA |
| | GCACATATTAGCACATTCAAGGCTCTGAGAGAATGTGGTTAACTTTGTTTAACTC |
| | AGCATTCCTCACTTTTTTTTTTTAATCATCAGAAATTCTCTCTCTCTCTCTCTCT |
| | TTTTCTCTCGCTCTCTTTTTTTTTTTTTTTTTACAGGAAATGCCTTTAAACATCG |
| | TTGGAACTACCAGAGTCACCTTAAAGGAGATCAATTCTCTAGACTGATAAAAATT |
| | TCATGGCCTCCTTTAAATGTTGCCAAATATATGAATTCTAGGATTTTTCCTTAGG |
| | AAAGGTTTTTCTCTTTCAGGGAAGATCTATTAACTCCCCATGGGTGCTGAAAATA |
| | AACTTGATGGTGAAAAACTCTGTATAAATTAATTTAAAAATTATTTGGTTTCTCT |
| | TTTTAATTATTCTGGGGCATAGTCATTTCTAAAAGTCACTAGTAGAAAGTATAAT |
| | TTCAAGACAGAATATTCTAGACATGCTAGCAGTTTATATGTATTCATGAGTAATG |
| | TGATATATATTGGGCGCTGGTGAGGAAGGAAGGAGGAATGAGTGACTATAAGGAT |
| | GGTTACCATAGAAACTTCCTTTTTTACCTAATTGAAGAGAGACTACTACAGAGTG |
| | CTAAGCTGCATGTGTCATCTTACACTAGAGAGAAATGGTAAGTTTCTTGTTTTAT |
| | TTAAGTTATGTTTAAGCAAGGAAAGGATTTGTTATTGAACAGTATATTTCAGGAA |
| | GGTTAGAAAGTGGCGGTTAGGATATATTTTAAATCTACCTAAAGCAGCATATTTT |
| | AAAAATTTAAAAGTATTGGTATTAAATTAAGAAATAGAGGACAGAACTAGACTGA |
| | TAGCAGTGACCTAGAACAATTTGAGATTAGGAAAGTTGTGACCATGAATTTAAGG |
| | ATTTATGTGGATACAAATTCTCCTTTAAAGTGTTTCTTCCCTTAATATTTATCTG |
| | ACGGTAATTTTTGAGCAGTGAATTACTTTATATATCTTAATAGTTTATTTGGGAC |
| | CAAACACTTAAACAAAAAGTTCTTTAAGTCATATAAGCCTTTTCAGGAAGCTTGT |
| | CTCATATTCACTCCCGAGACATTCACCTGCCAAGTGGCCTGAGGATCAATCCAGT |
| | CCTAGGTTTATTTTGCAGACTTACATTCTCCCAAGTTATTCAGCCTCATATGACT |
| | CCACGGTCGGCTTTACCAAAACAGTTCAGAGTGCACTTTGGCACACAATTGGGAA |
| | CAGAACAATCTAATGTGTGGTTTGGTATTCCAAGTGGGGTCTTTTTCAGAATCTC |
| | TGCACTAGTGTGAGATGCAAACATGTTTCCTCATCTTTCTGGCTTATCCAGTATG |
| | TAGCTATTTGTGACATAATAAATATATACATATATGAAAATA |
| 59 | MDVFMKGLSKAKEGVVAAAEKTKQGVAEAAGKTKEGVLYVGSKTKEGVVHGVATV |
| | AEKTKEQVTNVGGAVVTGVTAVAQKTVEGAGSIAAATGFVKKDQLGKNEEGAPQE |
| | GILEDMPVDPDNEAYEMPSEEGYQDYEPEA |
| 60 | GCAGTCACCGCCACCCACCAGCTCCGGCACCAACAGCAGCGCCGCTGCCACCGCC |
| | CACCTTCTGCCGCCGCCACCACAGCCACCTTCTCCTCCTCCGCTGTCCTCTCCCG |
| | TCCTCGCCTCTGTCGACTATCAGGTGAACTTTGAACCAGGATGGCTGAGCCCCGC |
| | CAGGAGTTCGAAGTGATGGAAGATCACGCTGGGACGTACGGGTTGGGGGACAGGA |
| | AAGATCAGGGGGGCTACACCATGCACCAAGACCAAGAGGGTGACACGGACGCTGG |
| | CCTGAAAGAATCTCCCCTGCAGACCCCCACTGAGGACGGATCTGAGGAACCGGGC |
| | TCTGAAACCTCTGATGCTAAGAGCACTCCAACAGCGGAAGATGTGACAGCACCCT |
| | TAGTGGATGAGGGAGCTCCCGGCAAGCAGGCTGCCGCGCAGCCCCACACGGAGAT |
| | CCCAGAAGGAACCACAGCTGAAGAAGCAGGCATTGGAGACACCCCCAGCCTGGAA |
| | GACGAAGCTGCTGGTCACGTGACCCAAGAGCCTGAAAGTGGTAAGGTGGTCCAGG |
| | AAGGCTTCCTCCGAGAGCCAGGCCCCCCAGGTCTGAGCCACCAGCTCATGTCCGG |
| | CATGCCTGGGGCTCCCCTCCTGCCTGAGGGCCCCAGAGAGGCCACACGCCAACCT |
| | TCGGGGACAGGACCTGAGGACACAGAGGGCGGCCGCCACGCCCCTGAGCTGCTCA |
| | AGCACCAGCTTCTAGGAGACCTGCACCAGGAGGGGCCGCCGCTGAAGGGGGCAGG |
| | GGGCAAAGAGAGGCCGGGGAGCAAGGAGGAGGTGGATGAAGACCGCGACGTCGAT |
| | GAGTCCTCCCCCCAAGACTCCCCTCCCTCCAAGGCCTCCCCAGCCCAAGATGGGC |
| | GGCCTCCCCAGACAGCCGCCAGAGAAGCCACCAGCATCCCAGGCTTCCCAGCGGA |
| | GGGTGCCATCCCCCTCCCTGTGGATTTCCTCTCCAAAGTTTCCACAGAGATCCCA |
| | GCCTCAGAGCCCGACGGGCCCAGTGTAGGGCGGGCCAAAGGGCAGGATGCCCCCC |
| | TGGAGTTCACGTTTCACGTGGAAATCACACCCAACGTGCAGAAGGAGCAGGCGCA |
| | CTCGGAGGAGCATTTGGGAAGGGCTGCATTTCCAGGGGCCCCTGGAGAGGGGCCA |
| | GAGGCCCGGGGCCCCTCTTTGGGAGAGGACACAAAAGAGGCTGACCTTCCAGAGC |
| | CCTCTGAAAAGCAGCCTGCTGCTGCTCCGCGGGGGAAGCCCGTCAGCCGGGTCCC |
| | TCAACTCAAAGCTCGCATGGTCAGTAAAAGCAAAGACGGGACTGGAAGCGATGAC |
| | AAAAAAGCCAAGACATCCACACGTTCCTCTGCTAAAACCTTGAAAAATAGGCCTT |
| | GCCTTAGCCCCAAACACCCCACTCCTGGTAGCTCAGACCCTCTGATCCAACCCTC |
| | CAGCCCTGCTGTGTGCCCAGAGCCACCTTCCTCTCCTAAATACGTCTCTTCTGTC |
| | ACTTCCCGAACTGGCAGTTCTGGAGCAAAGGAGATGAAACTCAAGGGGGCTGATG |
| | GTAAAACGAAGATCGCCACACCGCGGGGAGCAGCCCCTCCAGGCCAGAAGGGCCA |
| | GGCCAACGCCACCAGGATTCCAGCAAAAACCCCGCCCGCTCCAAAGACACCACCC |
| | AGCTCTGCGACTAAGCAAGTCCAGAGAAGACCACCCCCTGCAGGGCCCAGATCTG |
| | AGAGAGGTGAACCTCCAAAATCAGGGGATCGCAGCGGCTACAGCAGCCCCGGCTC |
| | CCCAGGCACTCCCGGCAGCCGCTCCCGCACCCCGTCCCTTCCAACCCCACCCACC |
| | CGGGAGCCCAAGAAGGTGGCAGTGGTCCGTACTCCACCCAAGTCGCCGTCTTCCG |
| | CCAAGAGCCGCCTGCAGACAGCCCCCGTGCCCATGCCAGACCTGAAGAATGTCAA |
| | GTCCAAGATCGGCTCCACTGAGAACCTGAAGCACCAGCCGGGAGGCGGGAAGGTG |
| | CAGATAATTAATAAGAAGCTGGATCTTAGCAACGTCCAGTCCAAGTGTGGCTCAA |
| | AGGATAATATCAAACACGTCCCGGGAGGCGGCAGTGTGCAAATAGTCTACAAACC |
| | AGTTGACCTGAGCAAGGTGACCTCCAAGTGTGGCTCATTAGGCAACATCCATCAT |
| | AAACCAGGAGGTGGCCAGGTGGAAGTAAAATCTGAGAAGCTTGACTTCAAGGACA |
| | GAGTCCAGTCGAAGATTGGGTCCCTGGACAATATCACCCACGTCCCTGGCGGAGG |
| | AAATAAAAAGATTGAAACCCACAAGCTGACCTTCCGCGAGAACGCCAAAGCCAAG |
| | ACAGACCACGGGGCGGAGATCGTGTACAAGTCGCCAGTGGTGTCTGGGGACACGT |
| | CTCCACGGCATCTCAGCAATGTCTCCTCCACCGGCAGCATCGACATGGTAGACTC |
| | GCCCCAGCTCGCCACGCTAGCTGACGAGGTGTCTGCCTCCCTGGCCAAGCAGGGT |
| | TTGTGATCAGGCCCCTGGGGCGGTCAATAATTGTGGAGAGGAGAGAATGAGAGAG |
| | TGTGGAAAAAAAAAGAATAATGACCCGGCCCCCGCCCTCTGCCCCCAGCTGCTCC |
| | TCGCAGTTCGGTTAATTGGTTAATCACTTAACCTGCTTTTGTCACTCGGCTTTGG |
| | CTCGGGACTTCAAAATCAGTGATGGGAGTAAGAGCAAATTTCATCTTTCCAAATT |
| | GATGGGTGGGCTAGTAATAAAATATTTAAAAAAAAACATTCAAAAACATGGCCAC |
| | ATCCAACATTTCCTCAGGCAATTCCTTTTGATTCTTTTTTCTTCCCCCTCCATGT |
| | AGAAGAGGGAGAAGGAGAGGCTCTGAAAGCTGCTTCTGGGGGATTTCAAGGGACT |
| | GGGGGTGCCAACCACCTCTGGCCCTGTTGTGGGGGTGTCACAGAGGCAGTGGCAG |
| | CAACAAAGGATTTGAAACTTGGTGTGTTCGTGGAGCCACAGGCAGACGATGTCAA |
| | CCTTGTGTGAGTGTGACGGGGGTTGGGGTGGGGGGGGAGGCCACGGGGGAGGCCG |
| | AGGCAGGGGCTGGGCAGAGGGGAGAGGAAGCACAAGAAGTGGGAGTGGGAGAGGA |
| | AGCCACGTGCTGGAGAGTAGACATCCCCCTCCTTGCCGCTGGGAGAGCCAAGGCC |
| | TATGCCACCTGCAGCGTCTGAGCGGCCGCCTGTCCTTGGTGGCCGGGGGTGGGGG |
| | CCTGCTGTGGGTCAGTGTGCCACCCTCTGCAGGGCAGCCTGTGGGAGAAGGGACA |
| | GCGGGTAAAAAGAGAAGGCAAGCTGGCAGGAGGGTGGCACTTCGTGGATGACCTC |
| | CTTAGAAAAGACTGACCTTGATGTCTTGAGAGCGCTGGCCTCTTCCTCCCTCCCT |
| | GCAGGGTAGGGGGCCTGAGTTGAGGGGCTTCCCTCTGCTCCACAGAAACCCTGTT |
| | TTATTGAGTTCTGAAGGTTGGAACTGCTGCCATGATTTTGGCCACTTTGCAGACC |
| | TGGGACTTTAGGGCTAACCAGTTCTCTTTGTAAGGACTTGTGCCTCTTGGGAGAC |
| | GTCCACCCGTTTCCAAGCCTGGGCCACTGGCATCTCTGGAGTGTGTGGGGGTCTG |
| | GGAGGCAGGTCCCGAGCCCCCTGTCCTTCCCACGGCCACTGCAGTCACCCCGTCT |
| | GCGCCGCTGTGCTGTTGTCTGCCGTGAGAGCCCAATCACTGCCTATACCCCTCAT |
| | CACACGTCACAATGTCCCGAATTCCCAGCCTCACCACCCCTTCTCAGTAATGACC |
| | CTGGTTGGTTGCAGGAGGTACCTACTCCATACTGAGGGTGAAATTAAGGGAAGGC |
| | AAAGTCCAGGCACAAGAGTGGGACCCCAGCCTCTCACTCTCAGTTCCACTCATCC |
| | AACTGGGACCCTCACCACGAATCTCATGATCTGATTCGGTTCCCTGTCTCCTCCT |
| | CCCGTCACAGATGTGAGCCAGGGCACTGCTCAGCTGTGACCCTAGGTGTTTCTGC |
| | CTTGTTGACATGGAGAGAGCCCTTTCCCCTGAGAAGGCCTGGCCCCTTCCTGTGC |
| | TGAGCCCACAGCAGCAGGCTGGGTGTCTTGGTTGTCAGTGGTGGCACCAGGATGG |
| | AAGGGCAAGGCACCCAGGGCAGGCCCACAGTCCCGCTGTCCCCCACTTGCACCCT |
| | AGCTTGTAGCTGCCAACCTCCCAGACAGCCCAGCCCGCTGCTCAGCTCCACATGC |
| | ATAGTATCAGCCCTCCACACCCGACAAAGGGGAACACACCCCCTTGGAAATGGTT |
| | CTTTTCCCCCAGTCCCAGCTGGAAGCCATGCTGTCTGTTCTGCTGGAGCAGCTGA |
| | ACATATACATAGATGTTGCCCTGCCCTCCCCATCTGCACCCTGTTGAGTTGTAGT |
| | TGGATTTGTCTGTTTATGCTTGGATTCACCAGAGTGACTATGATAGTGAAAAGAA |
| | AAAAAAAAAAAAAAAAGGACGCATGTATCTTGAAATGCTTGTAAAGAGGTTTCTA |
| | ACCCACCCTCACGAGGTGTCTCTCACCCCCACACTGGGACTCGTGTGGCCTGTGT |
| | GGTGCCACCCTGCTGGGGCCTCCCAAGTTTTGAAAGGCTTTCCTCAGCACCTGGG |
| | ACCCAACAGAGACCAGCTTCTAGCAGCTAAGGAGGCCGTTCAGCTGTGACGAAGG |
| | CCTGAAGCACAGGATTAGGACTGAAGCGATGATGTCCCCTTCCCTACTTCCCCTT |
| | GGGGCTCCCTGTGTCAGGGCACAGACTAGGTCTTGTGGCTGGTCTGGCTTGCGGC |
| | GCGAGGATGGTTCTCTCTGGTCATAGCCCGAAGTCTCATGGCAGTCCCAAAGGAG |
| | GCTTACAACTCCTGCATCACAAGAAAAAGGAAGCCACTGCCAGCTGGGGGGATCT |
| | GCAGCTCCCAGAAGCTCCGTGAGCCTCAGCCACCCCTCAGACTGGGTTCCTCTCC |
| | AAGCTCGCCCTCTGGAGGGGCAGCGCAGCCTCCCACCAAGGGCCCTGCGACCACA |
| | GCAGGGATTGGGATGAATTGCCTGTCCTGGATCTGCTCTAGAGGCCCAAGCTGCC |
| | TGCCTGAGGAAGGATGACTTGACAAGTCAGGAGACACTGTTCCCAAAGCCTTGAC |
| | CAGAGCACCTCAGCCCGCTGACCTTGCACAAACTCCATCTGCTGCCATGAGAAAA |
| | GGGAAGCCGCCTTTGCAAAACATTGCTGCCTAAAGAAACTCAGCAGCCTCAGGCC |
| | CAATTCTGCCACTTCTGGTTTGGGTACAGTTAAAGGCAACCCTGAGGGACTTGGC |
| | AGTAGAAATCCAGGGCCTCCCCTGGGGCTGGCAGCTTCGTGTGCAGCTAGAGCTT |
| | TACCTGAAAGGAAGTCTCTGGGCCCAGAACTCTCCACCAAGAGCCTCCCTGCCGT |
| | TCGCTGAGTCCCAGCAATTCTCCTAAGTTGAAGGGATCTGAGAAGGAGAAGGAAA |
| | TGTGGGGTAGATTTGGTGGTGGTTAGAGATATGCCCCCCTCATTACTGCCAACAG |
| | TTTCGGCTGCATTTCTTCACGCACCTCGGTTCCTCTTCCTGAAGTTCTTGTGCCC |
| | TGCTCTTCAGCACCATGGGCCTTCTTATACGGAAGGCTCTGGGATCTCCCCCTTG |
| | TGGGGCAGGCTCTTGGGGCCAGCCTAAGATCATGGTTTAGGGTGATCAGTGCTGG |
| | CAGATAAATTGAAAAGGCACGCTGGCTTGTGATCTTAAATGAGGACAATCCCCCC |
| | AGGGCTGGGCACTCCTCCCCTCCCCTCACTTCTCCCACCTGCAGAGCCAGTGTCC |
| | TTGGGTGGGCTAGATAGGATATACTGTATGCCGGCTCCTTCAAGCTGCTGACTCA |
| | CTTTATCAATAGTTCCATTTAAATTGACTTCAGTGGTGAGACTGTATCCTGTTTG |
| | CTATTGCTTGTTGTGCTATGGGGGGAGGGGGGAGGAATGTGTAAGATAGTTAACA |
| | TGGGCAAAGGGAGATCTTGGGGTGCAGCACTTAAACTGCCTCGTAACCCTTTTCA |
| | TGATTTCAACCACATTTGCTAGAGGGAGGGAGCAGCCACGGAGTTAGAGGCCCTT |
| | GGGGTTTCTCTTTTCCACTGACAGGCTTTCCCAGGCAGCTGGCTAGTTCATTCCC |
| | TCCCCAGCCAGGTGCAGGCGTAGGAATATGGACATCTGGTTGCTTTGGCCTGCTG |
| | CCCTCTTTCAGGGGTCCTAAGCCCACAATCATGCCTCCCTAAGACCTTGGCATCC |
| | TTCCCTCTAAGCCGTTGGCACCTCTGTGCCACCTCTCACACTGGCTCCAGACACA |
| | CAGCCTGTGCTTTTGGAGCTGAGATCACTCGCTTCACCCTCCTCATCTTTGTTCT |
| | CCAAGTAAAGCCACGAGGTCGGGGCGAGGGCAGAGGTGATCACCTGCGTGTCCCA |
| | TCTACAGACCTGCAGCTTCATAAAACTTCTGATTTCTCTTCAGCTTTGAAAAGGG |
| | TTACCCTGGGCACTGGCCTAGAGCCTCACCTCCTAATAGACTTAGCCCCATGAGT |
| | TTGCCATGTTGAGCAGGACTATTTCTGGCACTTGCAAGTCCCATGATTTCTTCGG |
| | TAATTCTGAGGGTGGGGGGAGGGACATGAAATCATCTTAGCTTAGCTTTCTGTCT |
| | GTGAATGTCTATATAGTGTATTGTGTGTTTTAACAAATGATTTACACTGACTGTT |
| | GCTGTAAAAGTGAATTTGGAAATAAAGTTATTACTCTGATTAAA |
| 61 | MAEPRQEFEVMEDHAGTYGLGDRKDQGGYTMHQDQEGDTDAGLKESPLQTPTEDG |
| | SEEPGSETSDAKSTPTAEDVTAPLVDEGAPGKQAAAQPHTEIPEGTTAEEAGIGD |
| | TPSLEDEAAGHVTQEPESGKVVQEGFLREPGPPGLSHQLMSGMPGAPLLPEGPRE |
| | ATRQPSGTGPEDTEGGRHAPELLKHQLLGDLHQEGPPLKGAGGKERPGSKEEVDE |
| | DRDVDESSPQDSPPSKASPAQDGRPPQTAAREATSIPGFPAEGAIPLPVDFLSKV |
| | STEIPASEPDGPSVGRAKGQDAPLEFTFHVEITPNVQKEQAHSEEHLGRAAFPGA |
| | PGEGPEARGPSLGEDTKEADLPEPSEKQPAAAPRGKPVSRVPQLKARMVSKSKDG |
| | TGSDDKKAKTSTRSSAKTLKNRPCLSPKHPTPGSSDPLIQPSSPAVCPEPPSSPK |
| | YVSSVTSRTGSSGAKEMKLKGADGKTKIATPRGAAPPGQKGQANATRIPAKTPPA |
| | PKTPPSSATKQVQRRPPPAGPRSERGEPPKSGDRSGYSSPGSPGTPGSRSRTPSL |
| | PTPPTREPKKVAVVRTPPKSPSSAKSRLQTAPVPMPDLKNVKSKIGSTENLKHQP |
| | GGGKVQIINKKLDLSNVQSKCGSKDNIKHVPGGGSVQIVYKPVDLSKVTSKCGSL |
| | GNIHHKPGGGQVEVKSEKLDFKDRVQSKIGSLDNITHVPGGGNKKIETHKLTFRE |
| | NAKAKTDHGAEIVYKSPVVSGDTSPRHLSNVSSTGSIDMVDSPQLATLADEVSAS |
| | LAKQGL |
| 62 | GCAGTCACCGCCACCCACCAGCTCCGGCACCAACAGCAGCGCCGCTGCCACCGCC |
| | CACCTTCTGCCGCCGCCACCACAGCCACCTTCTCCTCCTCCGCTGTCCTCTCCCG |
| | TCCTCGCCTCTGTCGACTATCAGGTGAACTTTGAACCAGGATGGCTGAGCCCCGC |
| | CAGGAGTTCGAAGTGATGGAAGATCACGCTGGGACGTACGGGTTGGGGGACAGGA |
| | AAGATCAGGGGGGCTACACCATGCACCAAGACCAAGAGGGTGACACGGACGCTGG |
| | CCTGAAAGAATCTCCCCTGCAGACCCCCACTGAGGACGGATCTGAGGAACCGGGC |
| | TCTGAAACCTCTGATGCTAAGAGCACTCCAACAGCGGAAGCTGAAGAAGCAGGCA |
| | TTGGAGACACCCCCAGCCTGGAAGACGAAGCTGCTGGTCACGTGACCCAAGCTCG |
| | CATGGTCAGTAAAAGCAAAGACGGGACTGGAAGCGATGACAAAAAAGCCAAGGGG |
| | GCTGATGGTAAAACGAAGATCGCCACACCGCGGGGAGCAGCCCCTCCAGGCCAGA |
| | AGGGCCAGGCCAACGCCACCAGGATTCCAGCAAAAACCCCGCCCGCTCCAAAGAC |
| | ACCACCCAGCTCTGGTGAACCTCCAAAATCAGGGGATCGCAGCGGCTACAGCAGC |
| | CCCGGCTCCCCAGGCACTCCCGGCAGCCGCTCCCGCACCCCGTCCCTTCCAACCC |
| | CACCCACCCGGGAGCCCAAGAAGGTGGCAGTGGTCCGTACTCCACCCAAGTCGCC |
| | GTCTTCCGCCAAGAGCCGCCTGCAGACAGCCCCCGTGCCCATGCCAGACCTGAAG |
| | AATGTCAAGTCCAAGATCGGCTCCACTGAGAACCTGAAGCACCAGCCGGGAGGCG |
| | GGAAGGTGCAGATAATTAATAAGAAGCTGGATCTTAGCAACGTCCAGTCCAAGTG |
| | TGGCTCAAAGGATAATATCAAACACGTCCCGGGAGGCGGCAGTGTGCAAATAGTC |
| | TACAAACCAGTTGACCTGAGCAAGGTGACCTCCAAGTGTGGCTCATTAGGCAACA |
| | TCCATCATAAACCAGGAGGTGGCCAGGTGGAAGTAAAATCTGAGAAGCTTGACTT |
| | CAAGGACAGAGTCCAGTCGAAGATTGGGTCCCTGGACAATATCACCCACGTCCCT |
| | GGCGGAGGAAATAAAAAGATTGAAACCCACAAGCTGACCTTCCGCGAGAACGCCA |
| | AAGCCAAGACAGACCACGGGGCGGAGATCGTGTACAAGTCGCCAGTGGTGTCTGG |
| | GGACACGTCTCCACGGCATCTCAGCAATGTCTCCTCCACCGGCAGCATCGACATG |
| | GTAGACTCGCCCCAGCTCGCCACGCTAGCTGACGAGGTGTCTGCCTCCCTGGCCA |
| | AGCAGGGTTTGTGATCAGGCCCCTGGGGCGGTCAATAATTGTGGAGAGGAGAGAA |
| | TGAGAGAGTGTGGAAAAAAAAAGAATAATGACCCGGCCCCCGCCCTCTGCCCCCA |
| | GCTGCTCCTCGCAGTTCGGTTAATTGGTTAATCACTTAACCTGCTTTTGTCACTC |
| | GGCTTTGGCTCGGGACTTCAAAATCAGTGATGGGAGTAAGAGCAAATTTCATCTT |
| | TCCAAATTGATGGGTGGGCTAGTAATAAAATATTTAAAAAAAAACATTCAAAAAC |
| | ATGGCCACATCCAACATTTCCTCAGGCAATTCCTTTTGATTCTTTTTTCTTCCCC |
| | CTCCATGTAGAAGAGGGAGAAGGAGAGGCTCTGAAAGCTGCTTCTGGGGGATTTC |
| | AAGGGACTGGGGGTGCCAACCACCTCTGGCCCTGTTGTGGGGGTGTCACAGAGGC |
| | AGTGGCAGCAACAAAGGATTTGAAACTTGGTGTGTTCGTGGAGCCACAGGCAGAC |
| | GATGTCAACCTTGTGTGAGTGTGACGGGGGTTGGGGTGGGGGGGGAGGCCACGGG |
| | GGAGGCCGAGGCAGGGGCTGGGCAGAGGGGAGAGGAAGCACAAGAAGTGGGAGTG |
| | GGAGAGGAAGCCACGTGCTGGAGAGTAGACATCCCCCTCCTTGCCGCTGGGAGAG |
| | CCAAGGCCTATGCCACCTGCAGCGTCTGAGCGGCCGCCTGTCCTTGGTGGCCGGG |
| | GGTGGGGGCCTGCTGTGGGTCAGTGTGCCACCCTCTGCAGGGCAGCCTGTGGGAG |
| | AAGGGACAGCGGGTAAAAAGAGAAGGCAAGCTGGCAGGAGGGTGGCACTTCGTGG |
| | ATGACCTCCTTAGAAAAGACTGACCTTGATGTCTTGAGAGCGCTGGCCTCTTCCT |
| | CCCTCCCTGCAGGGTAGGGGGCCTGAGTTGAGGGGCTTCCCTCTGCTCCACAGAA |
| | ACCCTGTTTTATTGAGTTCTGAAGGTTGGAACTGCTGCCATGATTTTGGCCACTT |
| | TGCAGACCTGGGACTTTAGGGCTAACCAGTTCTCTTTGTAAGGACTTGTGCCTCT |
| | TGGGAGACGTCCACCCGTTTCCAAGCCTGGGCCACTGGCATCTCTGGAGTGTGTG |
| | GGGGTCTGGGAGGCAGGTCCCGAGCCCCCTGTCCTTCCCACGGCCACTGCAGTCA |
| | CCCCGTCTGCGCCGCTGTGCTGTTGTCTGCCGTGAGAGCCCAATCACTGCCTATA |
| | CCCCTCATCACACGTCACAATGTCCCGAATTCCCAGCCTCACCACCCCTTCTCAG |
| | TAATGACCCTGGTTGGTTGCAGGAGGTACCTACTCCATACTGAGGGTGAAATTAA |
| | GGGAAGGCAAAGTCCAGGCACAAGAGTGGGACCCCAGCCTCTCACTCTCAGTTCC |
| | ACTCATCCAACTGGGACCCTCACCACGAATCTCATGATCTGATTCGGTTCCCTGT |
| | CTCCTCCTCCCGTCACAGATGTGAGCCAGGGCACTGCTCAGCTGTGACCCTAGGT |
| | GTTTCTGCCTTGTTGACATGGAGAGAGCCCTTTCCCCTGAGAAGGCCTGGCCCCT |
| | TCCTGTGCTGAGCCCACAGCAGCAGGCTGGGTGTCTTGGTTGTCAGTGGTGGCAC |
| | CAGGATGGAAGGGCAAGGCACCCAGGGCAGGCCCACAGTCCCGCTGTCCCCCACT |
| | TGCACCCTAGCTTGTAGCTGCCAACCTCCCAGACAGCCCAGCCCGCTGCTCAGCT |
| | CCACATGCATAGTATCAGCCCTCCACACCCGACAAAGGGGAACACACCCCCTTGG |
| | AAATGGTTCTTTTCCCCCAGTCCCAGCTGGAAGCCATGCTGTCTGTTCTGCTGGA |
| | GCAGCTGAACATATACATAGATGTTGCCCTGCCCTCCCCATCTGCACCCTGTTGA |
| | GTTGTAGTTGGATTTGTCTGTTTATGCTTGGATTCACCAGAGTGACTATGATAGT |
| | GAAAAGAAAAAAAAAAAAAAAAAAGGACGCATGTATCTTGAAATGCTTGTAAAGA |
| | GGTTTCTAACCCACCCTCACGAGGTGTCTCTCACCCCCACACTGGGACTCGTGTG |
| | GCCTGTGTGGTGCCACCCTGCTGGGGCCTCCCAAGTTTTGAAAGGCTTTCCTCAG |
| | CACCTGGGACCCAACAGAGACCAGCTTCTAGCAGCTAAGGAGGCCGTTCAGCTGT |
| | GACGAAGGCCTGAAGCACAGGATTAGGACTGAAGCGATGATGTCCCCTTCCCTAC |
| | TTCCCCTTGGGGCTCCCTGTGTCAGGGCACAGACTAGGTCTTGTGGCTGGTCTGG |
| | CTTGCGGCGCGAGGATGGTTCTCTCTGGTCATAGCCCGAAGTCTCATGGCAGTCC |
| | CAAAGGAGGCTTACAACTCCTGCATCACAAGAAAAAGGAAGCCACTGCCAGCTGG |
| | GGGGATCTGCAGCTCCCAGAAGCTCCGTGAGCCTCAGCCACCCCTCAGACTGGGT |
| | TCCTCTCCAAGCTCGCCCTCTGGAGGGGCAGCGCAGCCTCCCACCAAGGGCCCTG |
| | CGACCACAGCAGGGATTGGGATGAATTGCCTGTCCTGGATCTGCTCTAGAGGCCC |
| | AAGCTGCCTGCCTGAGGAAGGATGACTTGACAAGTCAGGAGACACTGTTCCCAAA |
| | GCCTTGACCAGAGCACCTCAGCCCGCTGACCTTGCACAAACTCCATCTGCTGCCA |
| | TGAGAAAAGGGAAGCCGCCTTTGCAAAACATTGCTGCCTAAAGAAACTCAGCAGC |
| | CTCAGGCCCAATTCTGCCACTTCTGGTTTGGGTACAGTTAAAGGCAACCCTGAGG |
| | GACTTGGCAGTAGAAATCCAGGGCCTCCCCTGGGGCTGGCAGCTTCGTGTGCAGC |
| | TAGAGCTTTACCTGAAAGGAAGTCTCTGGGCCCAGAACTCTCCACCAAGAGCCTC |
| | CCTGCCGTTCGCTGAGTCCCAGCAATTCTCCTAAGTTGAAGGGATCTGAGAAGGA |
| | GAAGGAAATGTGGGGTAGATTTGGTGGTGGTTAGAGATATGCCCCCCTCATTACT |
| | GCCAACAGTTTCGGCTGCATTTCTTCACGCACCTCGGTTCCTCTTCCTGAAGTTC |
| | TTGTGCCCTGCTCTTCAGCACCATGGGCCTTCTTATACGGAAGGCTCTGGGATCT |
| | CCCCCTTGTGGGGCAGGCTCTTGGGGCCAGCCTAAGATCATGGTTTAGGGTGATC |
| | AGTGCTGGCAGATAAATTGAAAAGGCACGCTGGCTTGTGATCTTAAATGAGGACA |
| | ATCCCCCCAGGGCTGGGCACTCCTCCCCTCCCCTCACTTCTCCCACCTGCAGAGC |
| | CAGTGTCCTTGGGTGGGCTAGATAGGATATACTGTATGCCGGCTCCTTCAAGCTG |
| | CTGACTCACTTTATCAATAGTTCCATTTAAATTGACTTCAGTGGTGAGACTGTAT |
| | CCTGTTTGCTATTGCTTGTTGTGCTATGGGGGGAGGGGGGAGGAATGTGTAAGAT |
| | AGTTAACATGGGCAAAGGGAGATCTTGGGGTGCAGCACTTAAACTGCCTCGTAAC |
| | CCTTTTCATGATTTCAACCACATTTGCTAGAGGGAGGGAGCAGCCACGGAGTTAG |
| | AGGCCCTTGGGGTTTCTCTTTTCCACTGACAGGCTTTCCCAGGCAGCTGGCTAGT |
| | TCATTCCCTCCCCAGCCAGGTGCAGGCGTAGGAATATGGACATCTGGTTGCTTTG |
| | GCCTGCTGCCCTCTTTCAGGGGTCCTAAGCCCACAATCATGCCTCCCTAAGACCT |
| | TGGCATCCTTCCCTCTAAGCCGTTGGCACCTCTGTGCCACCTCTCACACTGGCTC |
| | CAGACACACAGCCTGTGCTTTTGGAGCTGAGATCACTCGCTTCACCCTCCTCATC |
| | TTTGTTCTCCAAGTAAAGCCACGAGGTCGGGGCGAGGGCAGAGGTGATCACCTGC |
| | GTGTCCCATCTACAGACCTGCAGCTTCATAAAACTTCTGATTTCTCTTCAGCTTT |
| | GAAAAGGGTTACCCTGGGCACTGGCCTAGAGCCTCACCTCCTAATAGACTTAGCC |
| | CCATGAGTTTGCCATGTTGAGCAGGACTATTTCTGGCACTTGCAAGTCCCATGAT |
| | TTCTTCGGTAATTCTGAGGGTGGGGGGAGGGACATGAAATCATCTTAGCTTAGCT |
| | TTCTGTCTGTGAATGTCTATATAGTGTATTGTGTGTTTTAACAAATGATTTACAC |
| | TGACTGTTGCTGTAAAAGTGAATTTGGAAATAAAGTTATTACTCTGATTAAA |
| 63 | MAEPRQEFEVMEDHAGTYGLGDRKDQGGYTMHQDQEGDTDAGLKESPLQTPTEDG |
| | SEEPGSETSDAKSTPTAEAEEAGIGDTPSLEDEAAGHVTQARMVSKSKDGTGSDD |
| | KKAKGADGKTKIATPRGAAPPGQKGQANATRIPAKTPPAPKTPPSSGEPPKSGDR |
| | SGYSSPGSPGTPGSRSRTPSLPTPPTREPKKVAVVRTPPKSPSSAKSRLQTAPVP |
| | MPDLKNVKSKIGSTENLKHQPGGGKVQIINKKLDLSNVQSKCGSKDNIKHVPGGG |
| | SVQIVYKPVDLSKVTSKCGSLGNIHHKPGGGQVEVKSEKLDFKDRVQSKIGSLDN |
| | ITHVPGGGNKKIETHKLTFRENAKAKTDHGAEIVYKSPVVSGDTSPRHLSNVSST |
| | GSIDMVDSPQLATLADEVSASLAKQGL |
| 64 | GCAGTCACCGCCACCCACCAGCTCCGGCACCAACAGCAGCGCCGCTGCCACCGCC |
| | CACCTTCTGCCGCCGCCACCACAGCCACCTTCTCCTCCTCCGCTGTCCTCTCCCG |
| | TCCTCGCCTCTGTCGACTATCAGGTGAACTTTGAACCAGGATGGCTGAGCCCCGC |
| | CAGGAGTTCGAAGTGATGGAAGATCACGCTGGGACGTACGGGTTGGGGGACAGGA |
| | AAGATCAGGGGGGCTACACCATGCACCAAGACCAAGAGGGTGACACGGACGCTGG |
| | CCTGAAAGCTGAAGAAGCAGGCATTGGAGACACCCCCAGCCTGGAAGACGAAGCT |
| | GCTGGTCACGTGACCCAAGCTCGCATGGTCAGTAAAAGCAAAGACGGGACTGGAA |
| | GCGATGACAAAAAAGCCAAGGGGGCTGATGGTAAAACGAAGATCGCCACACCGCG |
| | GGGAGCAGCCCCTCCAGGCCAGAAGGGCCAGGCCAACGCCACCAGGATTCCAGCA |
| | AAAACCCCGCCCGCTCCAAAGACACCACCCAGCTCTGGTGAACCTCCAAAATCAG |
| | GGGATCGCAGCGGCTACAGCAGCCCCGGCTCCCCAGGCACTCCCGGCAGCCGCTC |
| | CCGCACCCCGTCCCTTCCAACCCCACCCACCCGGGAGCCCAAGAAGGTGGCAGTG |
| | GTCCGTACTCCACCCAAGTCGCCGTCTTCCGCCAAGAGCCGCCTGCAGACAGCCC |
| | CCGTGCCCATGCCAGACCTGAAGAATGTCAAGTCCAAGATCGGCTCCACTGAGAA |
| | CCTGAAGCACCAGCCGGGAGGCGGGAAGGTGCAAATAGTCTACAAACCAGTTGAC |
| | CTGAGCAAGGTGACCTCCAAGTGTGGCTCATTAGGCAACATCCATCATAAACCAG |
| | GAGGTGGCCAGGTGGAAGTAAAATCTGAGAAGCTTGACTTCAAGGACAGAGTCCA |
| | GTCGAAGATTGGGTCCCTGGACAATATCACCCACGTCCCTGGCGGAGGAAATAAA |
| | AAGATTGAAACCCACAAGCTGACCTTCCGCGAGAACGCCAAAGCCAAGACAGACC |
| | ACGGGGCGGAGATCGTGTACAAGTCGCCAGTGGTGTCTGGGGACACGTCTCCACG |
| | GCATCTCAGCAATGTCTCCTCCACCGGCAGCATCGACATGGTAGACTCGCCCCAG |
| | CTCGCCACGCTAGCTGACGAGGTGTCTGCCTCCCTGGCCAAGCAGGGTTTGTGAT |
| | CAGGCCCCTGGGGCGGTCAATAATTGTGGAGAGGAGAGAATGAGAGAGTGTGGAA |
| | AAAAAAAGAATAATGACCCGGCCCCCGCCCTCTGCCCCCAGCTGCTCCTCGCAGT |
| | TCGGTTAATTGGTTAATCACTTAACCTGCTTTTGTCACTCGGCTTTGGCTCGGGA |
| | CTTCAAAATCAGTGATGGGAGTAAGAGCAAATTTCATCTTTCCAAATTGATGGGT |
| | GGGCTAGTAATAAAATATTTAAAAAAAAACATTCAAAAACATGGCCACATCCAAC |
| | ATTTCCTCAGGCAATTCCTTTTGATTCTTTTTTCTTCCCCCTCCATGTAGAAGAG |
| | GGAGAAGGAGAGGCTCTGAAAGCTGCTTCTGGGGGATTTCAAGGGACTGGGGGTG |
| | CCAACCACCTCTGGCCCTGTTGTGGGGGTGTCACAGAGGCAGTGGCAGCAACAAA |
| | GGATTTGAAACTTGGTGTGTTCGTGGAGCCACAGGCAGACGATGTCAACCTTGTG |
| | TGAGTGTGACGGGGGTTGGGGTGGGGGGGGAGGCCACGGGGGAGGCCGAGGCAGG |
| | GGCTGGGCAGAGGGGAGAGGAAGCACAAGAAGTGGGAGTGGGAGAGGAAGCCACG |
| | TGCTGGAGAGTAGACATCCCCCTCCTTGCCGCTGGGAGAGCCAAGGCCTATGCCA |
| | CCTGCAGCGTCTGAGCGGCCGCCTGTCCTTGGTGGCCGGGGGTGGGGGCCTGCTG |
| | TGGGTCAGTGTGCCACCCTCTGCAGGGCAGCCTGTGGGAGAAGGGACAGCGGGTA |
| | AAAAGAGAAGGCAAGCTGGCAGGAGGGTGGCACTTCGTGGATGACCTCCTTAGAA |
| | AAGACTGACCTTGATGTCTTGAGAGCGCTGGCCTCTTCCTCCCTCCCTGCAGGGT |
| | AGGGGGCCTGAGTTGAGGGGCTTCCCTCTGCTCCACAGAAACCCTGTTTTATTGA |
| | GTTCTGAAGGTTGGAACTGCTGCCATGATTTTGGCCACTTTGCAGACCTGGGACT |
| | TTAGGGCTAACCAGTTCTCTTTGTAAGGACTTGTGCCTCTTGGGAGACGTCCACC |
| | CGTTTCCAAGCCTGGGCCACTGGCATCTCTGGAGTGTGTGGGGGTCTGGGAGGCA |
| | GGTCCCGAGCCCCCTGTCCTTCCCACGGCCACTGCAGTCACCCCGTCTGCGCCGC |
| | TGTGCTGTTGTCTGCCGTGAGAGCCCAATCACTGCCTATACCCCTCATCACACGT |
| | CACAATGTCCCGAATTCCCAGCCTCACCACCCCTTCTCAGTAATGACCCTGGTTG |
| | GTTGCAGGAGGTACCTACTCCATACTGAGGGTGAAATTAAGGGAAGGCAAAGTCC |
| | AGGCACAAGAGTGGGACCCCAGCCTCTCACTCTCAGTTCCACTCATCCAACTGGG |
| | ACCCTCACCACGAATCTCATGATCTGATTCGGTTCCCTGTCTCCTCCTCCCGTCA |
| | CAGATGTGAGCCAGGGCACTGCTCAGCTGTGACCCTAGGTGTTTCTGCCTTGTTG |
| | ACATGGAGAGAGCCCTTTCCCCTGAGAAGGCCTGGCCCCTTCCTGTGCTGAGCCC |
| | ACAGCAGCAGGCTGGGTGTCTTGGTTGTCAGTGGTGGCACCAGGATGGAAGGGCA |
| | AGGCACCCAGGGCAGGCCCACAGTCCCGCTGTCCCCCACTTGCACCCTAGCTTGT |
| | AGCTGCCAACCTCCCAGACAGCCCAGCCCGCTGCTCAGCTCCACATGCATAGTAT |
| | CAGCCCTCCACACCCGACAAAGGGGAACACACCCCCTTGGAAATGGTTCTTTTCC |
| | CCCAGTCCCAGCTGGAAGCCATGCTGTCTGTTCTGCTGGAGCAGCTGAACATATA |
| | CATAGATGTTGCCCTGCCCTCCCCATCTGCACCCTGTTGAGTTGTAGTTGGATTT |
| | GTCTGTTTATGCTTGGATTCACCAGAGTGACTATGATAGTGAAAAGAAAAAAAAA |
| | AAAAAAAAAGGACGCATGTATCTTGAAATGCTTGTAAAGAGGTTTCTAACCCACC |
| | CTCACGAGGTGTCTCTCACCCCCACACTGGGACTCGTGTGGCCTGTGTGGTGCCA |
| | CCCTGCTGGGGCCTCCCAAGTTTTGAAAGGCTTTCCTCAGCACCTGGGACCCAAC |
| | AGAGACCAGCTTCTAGCAGCTAAGGAGGCCGTTCAGCTGTGACGAAGGCCTGAAG |
| | CACAGGATTAGGACTGAAGCGATGATGTCCCCTTCCCTACTTCCCCTTGGGGCTC |
| | CCTGTGTCAGGGCACAGACTAGGTCTTGTGGCTGGTCTGGCTTGCGGCGCGAGGA |
| | TGGTTCTCTCTGGTCATAGCCCGAAGTCTCATGGCAGTCCCAAAGGAGGCTTACA |
| | ACTCCTGCATCACAAGAAAAAGGAAGCCACTGCCAGCTGGGGGGATCTGCAGCTC |
| | CCAGAAGCTCCGTGAGCCTCAGCCACCCCTCAGACTGGGTTCCTCTCCAAGCTCG |
| | CCCTCTGGAGGGGCAGCGCAGCCTCCCACCAAGGGCCCTGCGACCACAGCAGGGA |
| | TTGGGATGAATTGCCTGTCCTGGATCTGCTCTAGAGGCCCAAGCTGCCTGCCTGA |
| | GGAAGGATGACTTGACAAGTCAGGAGACACTGTTCCCAAAGCCTTGACCAGAGCA |
| | CCTCAGCCCGCTGACCTTGCACAAACTCCATCTGCTGCCATGAGAAAAGGGAAGC |
| | CGCCTTTGCAAAACATTGCTGCCTAAAGAAACTCAGCAGCCTCAGGCCCAATTCT |
| | GCCACTTCTGGTTTGGGTACAGTTAAAGGCAACCCTGAGGGACTTGGCAGTAGAA |
| | ATCCAGGGCCTCCCCTGGGGCTGGCAGCTTCGTGTGCAGCTAGAGCTTTACCTGA |
| | AAGGAAGTCTCTGGGCCCAGAACTCTCCACCAAGAGCCTCCCTGCCGTTCGCTGA |
| | GTCCCAGCAATTCTCCTAAGTTGAAGGGATCTGAGAAGGAGAAGGAAATGTGGGG |
| | TAGATTTGGTGGTGGTTAGAGATATGCCCCCCTCATTACTGCCAACAGTTTCGGC |
| | TGCATTTCTTCACGCACCTCGGTTCCTCTTCCTGAAGTTCTTGTGCCCTGCTCTT |
| | CAGCACCATGGGCCTTCTTATACGGAAGGCTCTGGGATCTCCCCCTTGTGGGGCA |
| | GGCTCTTGGGGCCAGCCTAAGATCATGGTTTAGGGTGATCAGTGCTGGCAGATAA |
| | ATTGAAAAGGCACGCTGGCTTGTGATCTTAAATGAGGACAATCCCCCCAGGGCTG |
| | GGCACTCCTCCCCTCCCCTCACTTCTCCCACCTGCAGAGCCAGTGTCCTTGGGTG |
| | GGCTAGATAGGATATACTGTATGCCGGCTCCTTCAAGCTGCTGACTCACTTTATC |
| | AATAGTTCCATTTAAATTGACTTCAGTGGTGAGACTGTATCCTGTTTGCTATTGC |
| | TTGTTGTGCTATGGGGGGAGGGGGGAGGAATGTGTAAGATAGTTAACATGGGCAA |
| | AGGGAGATCTTGGGGTGCAGCACTTAAACTGCCTCGTAACCCTTTTCATGATTTC |
| | AACCACATTTGCTAGAGGGAGGGAGCAGCCACGGAGTTAGAGGCCCTTGGGGTTT |
| | CTCTTTTCCACTGACAGGCTTTCCCAGGCAGCTGGCTAGTTCATTCCCTCCCCAG |
| | CCAGGTGCAGGCGTAGGAATATGGACATCTGGTTGCTTTGGCCTGCTGCCCTCTT |
| | TCAGGGGTCCTAAGCCCACAATCATGCCTCCCTAAGACCTTGGCATCCTTCCCTC |
| | TAAGCCGTTGGCACCTCTGTGCCACCTCTCACACTGGCTCCAGACACACAGCCTG |
| | TGCTTTTGGAGCTGAGATCACTCGCTTCACCCTCCTCATCTTTGTTCTCCAAGTA |
| | AAGCCACGAGGTCGGGGCGAGGGCAGAGGTGATCACCTGCGTGTCCCATCTACAG |
| | ACCTGCAGCTTCATAAAACTTCTGATTTCTCTTCAGCTTTGAAAAGGGTTACCCT |
| | GGGCACTGGCCTAGAGCCTCACCTCCTAATAGACTTAGCCCCATGAGTTTGCCAT |
| | GTTGAGCAGGACTATTTCTGGCACTTGCAAGTCCCATGATTTCTTCGGTAATTCT |
| | GAGGGTGGGGGGAGGGACATGAAATCATCTTAGCTTAGCTTTCTGTCTGTGAATG |
| | TCTATATAGTGTATTGTGTGTTTTAACAAATGATTTACACTGACTGTTGCTGTAA |
| | AAGTGAATTTGGAAATAAAGTTATTACTCTGATTAAA |
| 65 | MAEPRQEFEVMEDHAGTYGLGDRKDQGGYTMHQDQEGDTDAGLKAEEAGIGDTPS |
| | LEDEAAGHVTQARMVSKSKDGTGSDDKKAKGADGKTKIATPRGAAPPGQKGQANA |
| | TRIPAKTPPAPKTPPSSGEPPKSGDRSGYSSPGSPGTPGSRSRTPSLPTPPTREP |
| | KKVAVVRTPPKSPSSAKSRLQTAPVPMPDLKNVKSKIGSTENLKHQPGGGKVQIV |
| | YKPVDLSKVTSKCGSLGNIHHKPGGGQVEVKSEKLDFKDRVQSKIGSLDNITHVP |
| | GGGNKKIETHKLTFRENAKAKTDHGAEIVYKSPVVSGDTSPRHLSNVSSTGSIDM |
| | VDSPQLATLADEVSASLAKQGL |
| 66 | VPmU*fG*mGmAmAmCmUmGmAmGmCmAmCfUmUfGmUmAmCmAmG*mG*mA |
| 67 | mC*mU*mGmUmAmCmAmAfGfUfGmC(UL3)mCmAmGmUmUmC*mC*mA |
| 68 | mG*mU*mAmCAmAfGUfGmC(Uads)mCmAmGmUmUmC*mC*mA |
| 69 | mC*(Uads)*mGmUmAmCmAmAfGfUfGmCmUmCmAmGmUmUmC*mC*mA |
| 70 | mC*mU*(Uads)mUmAmCmAmAfGfUfGmCmUmCmAmGmUmUmC*mC*mA |
| 71 | mC*mU*mG(Uads)mAmCmAmAfGfUfGmCmUmCmAmGmUmUmC*mC*mA |
| 72 | mC*mU*mGmUmA(Cads)mAmAfGfUfGmCmUmCmAmGmUmUmC*mC*mA |
| 73 | mC*mU*mGmUmAmCmAmAfG(Uads)fGmCmUmCmAmGmUmUmC*mC*mA |
| 74 | mC*mU*mGmUmAmCmAmAfGfUfG(Cads)mUmCmAmGmUmUmC*mC*mA |
| 75 | mC*mU*mGmUmAmCmAmAfGfUfGmCmU(Cads)mAmGmUmUmC*mC*mA |
| 76 | mC*mU*mGmUmAmCmAmAfGfUfGmCmUmCmA(Uads)mUmUmC*mC*mA |
| 77 | mC*mU*mGmUmAmCmAmAfGfUfGmCmUmCmAmG(Uads)mUmC*mC*mA |
| 78 | mC*mU*mGmUmAmCmAmAfGfUfGmCmUmCmAmGmU(Uads)mC*mC*mA |
| 79 | mC*mU*mGmUmAmCmAmAfGfUfGmCmUmCmAmGmUmU(Uads)*mC*mA |
| 80 | mC*mU*mGmUmAmCmAmAfGfUfGmCmUmCmAmGmUmUmC*(Uads)*mA |
| 81 | mC*mU*mGmUmAmCmAmAfGfUfGmC(UadsII)mCmAmGmUmUmC*mC*mA |
| 82 | VPmU*fG*mGmAfAmCfUmGmAmGmCmAmCfUmUfGmUmAmCmAmG*mG*mA |
| 83 | UGUACAAGUGCUCAGUUCCAA |
| 84 | UUGGAACUGAGCACUUGUACAGG |
| 85 | GUACAAGUGCUCAGUUCCAA |
| 86 | UUGGAACUGAGCACUUGUACAG |
| 87 | mU*mG*mUmAmCmAfAmGfUfGfCmU(Css)mAmGmUmUmCmC*mA*mA |
| 88 | VPmU*fU*mGmGmAfAmCmUmGmAmGmCmAfCmUfUmGmUmAmCmA*mG*mG |
| 89 | mU*mG*mUmAmCmAmAmGfUfGfCmU(Css)mAmGmUmUmCmC*mA*mA |
| 90 | VPmU*fU*mGmGfAmAmCfUmGmAmGmCmAfCmUfUmGmUmAmCmA*mG*mG |
| 91 | iAbmG*mU*mAmCmAmAmGfUfGfCmU(Css)mAmGmUmUmCmC*mA*mA |
| 92 | VPmU*fU*mGmGfAmAmCfUmGmAmGmCmAfCmUfUmGmUmAmC*mA*mG |
| 93 | VPmU*fU*mGmGmAmAmCmUmGmAmGmCmAfCmUfUmGmUmAmC*mA*mG |
| 94 | AGUGACUACCACUUAUUUCUA |
| 95 | UAGAAAUAAGUGGUAGUCACUUA |
| 96 | GUGACUACCACUUAUUUCUAA |
| 97 | UUAGAAAUAAGUGGUAGUCACUU |
| 98 | GAGCAAGUGACAAAUGUUGGA |
| 99 | UCCAACAUUUGUCACUUGCUCUU |
| 100 | UUCCAAUGUGCCCAGUCAUGA |
| 101 | UCAUGACUGGGCACAUUGGAACU |
| 102 | AAGUGACUACCACUUAUUUCA |
| 103 | UGAAAUAAGUGGUAGUCACUUAG |
| 104 | GACCAAAGAGCAAGUGACAAA |
| 105 | UUUGUCACUUGCUCUUUGGUCUU |
| 106 | mA*mG*mUmGmAmCmUmAfCfCfAmC(Uads)mUmAmUmUmUmC*mU*mA |
| 107 | VPmU*fA*mGmAfAmAmUfAmAmGmUmGmGfUmAfGmUmCmAmCmU*mU*mA |
| 108 | mG*mU*mGmAmCmUmAmCfCfAfCmU(Uads)mAmUmUmUmCmU*mA*mA |
| 109 | VPmU*fU*mAmGfAmAmAfUmAmAmGmUmGfGmUfAmGmUmCmAmC*mU*mU |
| 110 | mG*mA*mGmCmAmAmGmUfGfAfCmA(Aads)mAmUmGmUmUmG*mG*mA |
| 111 | VPmU*fC*mCmAfAmCmAfUmUmUmGmUmCfAmCfUmUmGmCmUmC*mU*mU |
| 112 | mU*mU*mCmCmAmAmUmGfUfGfCmC(Cads)mAmGmUmCmAmU*mG*mA |
| 113 | VPmU*fC*mAmUfGmAmCfUmGmGmGmCmAfCmAfUmUmGmGmAmA*mC*mU |
| 114 | mA*mA*mGmUmGmAmCmUfAfCfCmA(Cads)mUmUmAmUmUmU*mC*mA |
| 115 | VPmU*fG*mAmAfAmUmAfAmGmUmGmGmUfAmGfUmCmAmCmUmU*mA*mG |
| 116 | mG*mA*mCmCmAmAmAmGfAfGfCmA(Aads)mGmUmGmAmCmA*mA*mA |
| 117 | VPmU*fU*mUmGfUmCmAfCmUmUmGmCmUfCmUfUmUmGmGmUmC*mU*mU |
| 118 | mG*mU*mGmAmCmUfAmCfCfAfCmU(Uads)mAmUmUmUmCmU*mA*mA |
| 119 | VPmU*fU*mAmGmAfAmAmUmAmAmGmUmGfGmUfAmGmUmCmAmC*mU*mU |
| 120 | iAbmA*mG*mUmGmAmCfUmAfCfCfAmC(Uads)mUmAmUmUmUmC*mU*mA |
| 121 | VPmU*fA*mGmAmAfAmUmAmAmGmUmGmGfUmAfGmUmCmAmCmU*mU*mA |
| 122 | VPmU*fU*mAmGmAmAmAmUmAmAmGmUmGfGmUfAmGmUmCmAmC*mU*mU |
| 123 | iAbmA*mG*mUmGmAmCmUmAfCfCfAmC(Uads)mUmAmUmUmUmC*mU*mA |
| 124 | VPmU*fA*mGmAmAmAmUmAmAmGmUmGmGfUmAfGmUmCmAmCmU*mU*mA |
| 125 | AUUAGGCAACAUCCAUCAUAA |
| 126 | UUAUGAUGGAUGUUGCCUAAUGA |
| 127 | GGCUUUGGCUCGGGACUUCAA |
| 128 | UUGAAGUCCCGAGCCAAAGCCGA |
| 129 | GCAAAUAGUCUACAAACCAGA |
| 130 | UCUGGUUUGUAGACUAUUUGCAC |
| 131 | AAAUAAAAAGAUUGAAACCCA |
| 132 | UGGGUUUCAAUCUUUUUAUUUCC |
| 133 | GCAAGGUGACCUCCAAGUGUA |
| 134 | UACACUUGGAGGUCACCUUGCUC |
| 135 | AGAUUGAAACCCACAAGCUGA |
| 136 | UCAGCUUGUGGGUUUCAAUCUUU |
| 137 | mA*mU*mUmAmGmGmCmAfAfCfAmU(Cads)mCmAmUmCmAmU*mA*mA |
| 138 | VPmU*fU*mAmUmGmAmUmGmGmAmUmGmUfUmGfCmCmUmAmAmU*mG*mA |
| 139 | mG*mG*mCmUmUmUmGmGfCfUfCmG(Gads)mGmAmCmUmUmC*mA*mA |
| 140 | VPmU*fU*mGmAmAmGmUmCmCmCmGmAmGfCmCfAmAmAmGmCmC*mG*mA |
| 141 | mG*mC*mAmAmAmUmAmGfUfCfUmA(Cads)mAmAmAmCmCmA*mG*mA |
| 142 | VPmU*fC*mUmGmGmUmUmUmGmUmAmGmAfCmUfAmUmUmUmGmC*mA*mC |
| 143 | mA*mA*mAmUmAmAfAmAfAfGfAmU(Uads)mGmAmAmAmCmC*mC*mA |
| 144 | VPmU*fG*mGmGmUmUmUmCmAmAmUmCmUfUmUfUmUmAmUmUmU*mC*mC |
| 145 | mG*mC*mAmAmGmGmUmGfAfCfCmU(Cads)mCmAmAmGmUmG*mU*mA |
| 146 | VPmU*fA*mCmAmCmUmUmGmGmAmGmGmUfCmAfCmCmUmUmGmC*mU*mC |
| 147 | mA*mG*mAmUmUmGmAmAfAfCfCmC(Aads)mCmAmAmGmCmU*mG*mA |
| 148 | VPmU*fC*mAmGmCmUmUmGmUmGmGmGmUfUmUfCmAmAmUmCmU*mU*mU |
| 149 | VPmU*fG*mCmCfUmAmAfUmGmAmGmCmCfAmCfAmCmUmUmGmG*mA |
| 150 | VPmU*fG*mCmCfUmAmAfUmGmAmGmCmCfAmCfAmCmUmUmGmG |
| 151 | VPmU*fG*mCmCmUmAmAmUmGmAmGmCmCfAmCfAmCmUmUmGmG*mA*mG |
| 152 | VPmU*fU*mGmGmUfUmUmGmUmAmGmAmCfUmAfUmUmUmGmCmA*mC*mC |
| 153 | VPmU*dT*mGmGdTmUmUfGmUmAmGmAmCfUmAfUmUmUmGmCmA*mC*mC |
| 154 | mU*mG*mCmAmAmAmUmAfGfUfC(UadsII)mAmCmAmAmAmCmC*mA*mA |
| 155 | mUmG*mCmAmAmAmUmAfGfUfCmU(Aads)mCmAmAmAmCmC*mA*mA |
| 156 | VPmU*fU*mGmGmUmUmUmGmUmAmGmAmCfUmAfUmUmUmGmCmA*mC*mC |
| 157 | VPmU*dT*mGmGdTmUmUdGmUmAmGmAmCfUmAfUmUmUmGmCmA*mC*mC |
| 158 | VPmU*fU*mGmGnmUmUfGmUmAmGmAmCfUmAfUmUmUmGmCmA*mC*mC |
| 159 | VPmU*fU*mGmGfUmUnfGmUmAmGmAmCfUmAfUmUmUmGmCmA*mC*mC |
| 160 | mU*mG*mCmAmAmAfUmAfGfUfC(Uads)mAmCmAmAmAmCmC*mA*mA |
| 161 | mU*mG*mCmAmAmAmUmAfGfUfCmUmA(Uads)mAmAmAmCmC*mA*mA |
| 162 | mU*mG*mCmAmAmAmUmAfGfUfCmUmAmCmAmAmAmC(Cads)mA*mA |
| 163 | mU*mG*(Cads)mAmAmAmUmAfGfUfCmUmAmCmAmAmAmCmC*mA*mA |
| 164 | VPmU*fU*mGmGfUmUfUmGmUmAmGmAmCfUmAfUmUmUmGmCmA*mC*mC |
| 165 | VPmU*fU*mGmGfUmUmUfGmUmAmGmAmCfUmAfUmUmUmGmC*mA*mC |
| 166 | mU*mG*mCmAmAmAmUmAfGfUfC(UL3)mAmCmAmAmAmCmC*mA*mA |

1. A compound comprising any one of Formula Ia, Ib, Ic, II-IV, or XXI: 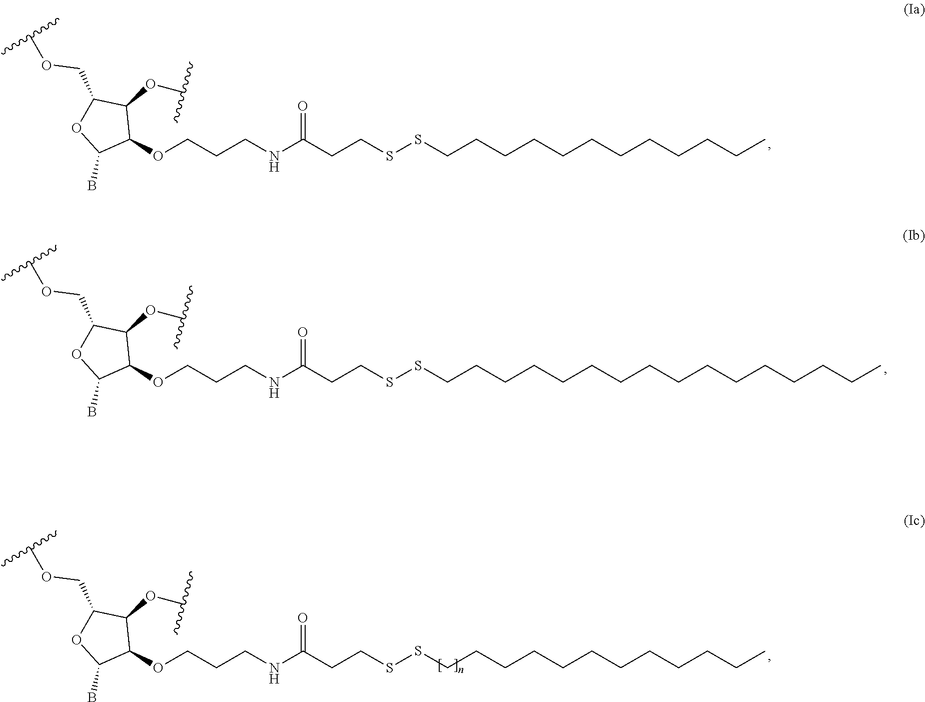
wherein n is an integer of 1-4, 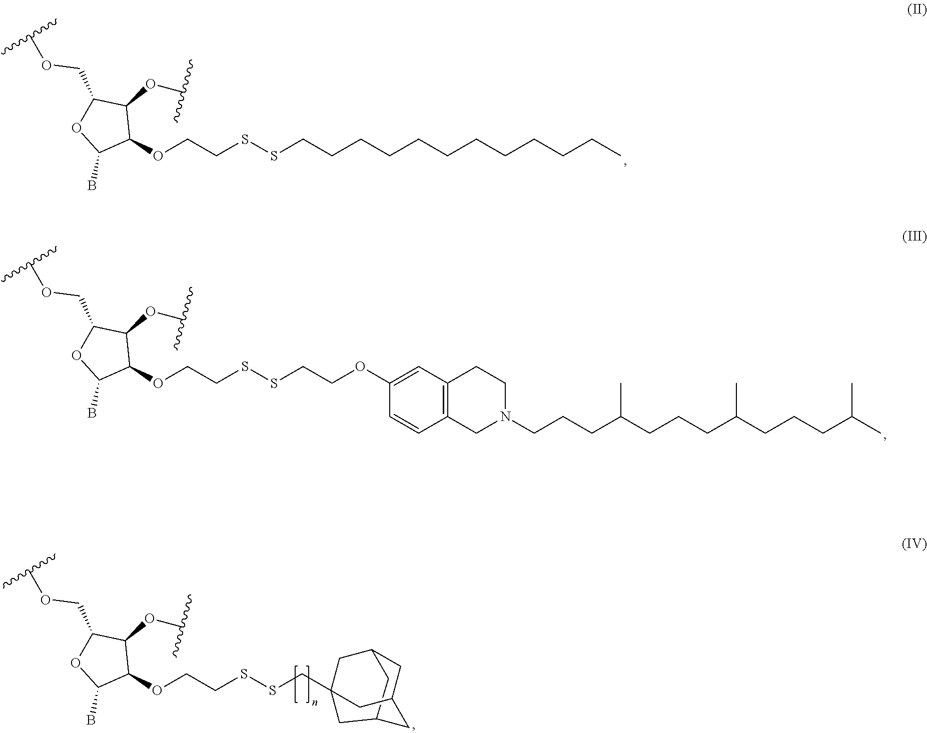
wherein n is an integer of 0-2, 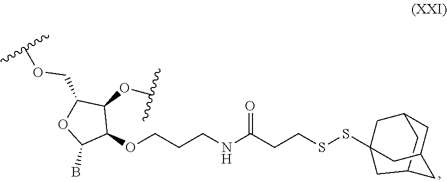
wherein B is a nucleobase selected from adenine, cytosine, guanine, thymine, uracil, or a derivative thereof.

2. The compound of claim 1, wherein the compound comprises Formula Ia, Ib or Ic.

3. The compound of claim 1, wherein the compound comprises Formula II.

4. The compound of claim 1, wherein the compound comprises Formula III.

5. The compound of claim 1, wherein the compound comprises Formula IV.

6. The compound of claim 5, wherein n is 0.

7. The compound of claim 5, wherein n is 2.

8. The compound of claim 1, wherein the compound comprises Formula XXI.

9. The compound of claim 1, wherein the compound is a nucleoside, nucleotide, or analog thereof.

10. An RNAi agent comprising a sense stand and an antisense strand, wherein the sense strand and the antisense strand form a duplex region, wherein the sense strand or the antisense strand comprises a modified nucleotide of any one of Formula Ia, Ib, Ic, II-IV, or XXI: 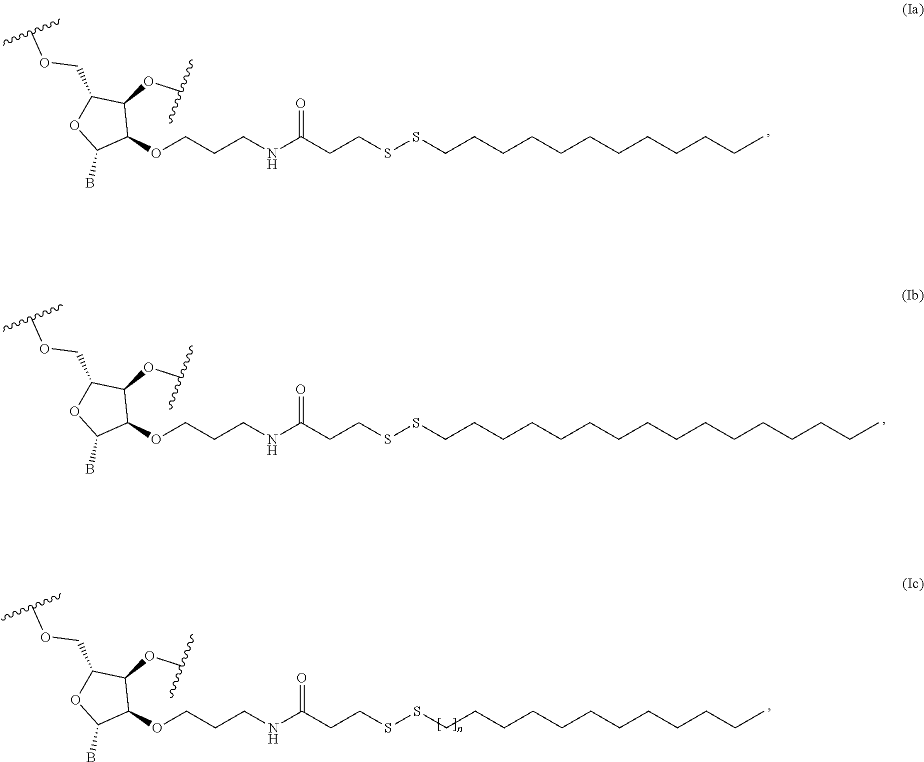
wherein n is an integer of 1-4, 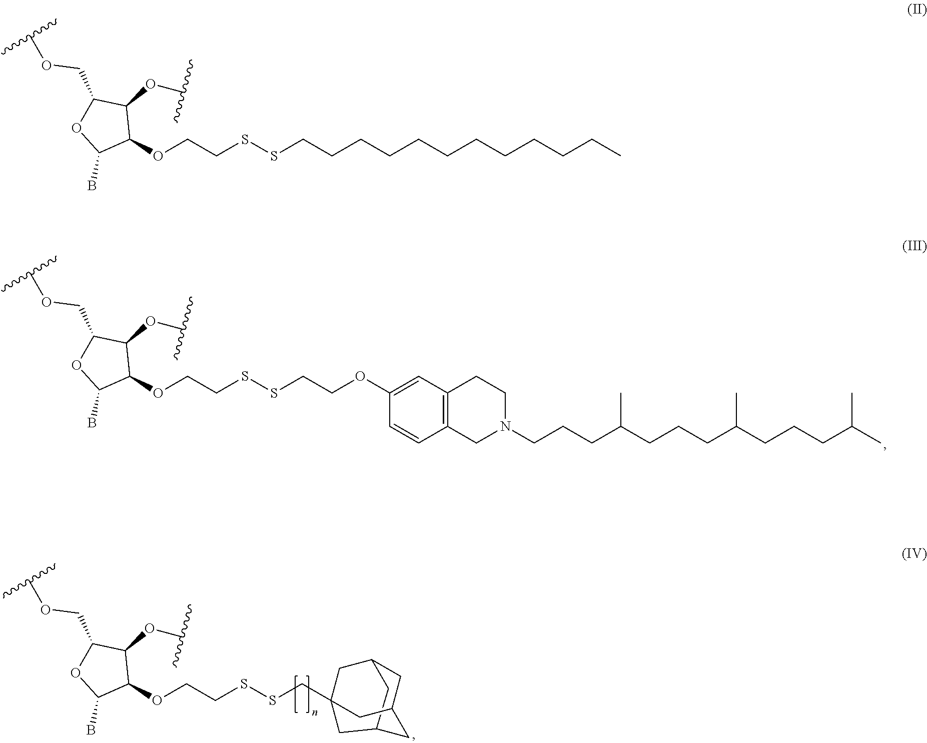
wherein n is an integer of 0-2, 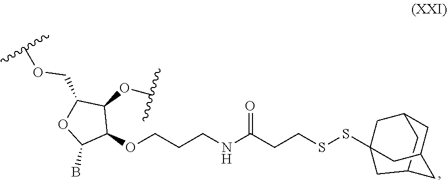
and
wherein B is a nucleobase selected from adenine, cytosine, guanine, thymine, uracil, or a derivative thereof.

11. The RNAi agent of claim 10, wherein the sense strand or the antisense strand comprises a modified nucleotide of Formula Ia, Ib, or Ic.

12. The RNAi agent of claim 10, wherein the sense strand or the antisense strand comprises a modified nucleotide of Formula II.

13. The RNAi agent of claim 10, wherein the sense strand or the antisense strand comprises a modified nucleotide of Formula III.

14. The RNAi agent of claim 10, wherein the sense strand or the antisense strand comprises a modified nucleotide of Formula IV.

15. The RNAi agent of claim 14, wherein n is 0.

16. The RNAi agent of claim 14, wherein n is 2.

17. The RNAi agent of claim 10, wherein the sense strand or the antisense strand comprises a modified nucleotide of Formula XXI.

18. The RNAi agent of claim 10, wherein the sense strand is 15 to 50 nucleotides in length.

19. The RNAi agent of claim 10, wherein the antisense strand is 15 to 30 nucleotides in length.

20. The RNAi agent of claim 10, wherein the sense strand is 21 nucleotides in length.

21. The RNAi agent of claim 10, wherein the antisense strand is 23 nucleotides in length.

22. (canceled)

23. The RNAi agent of claim 10, wherein the sense strand comprises the modified nucleotide of any one of Formula Ia, Ib, Ic, II-IV or XXI at any one of positions 1-6 or 12-21 from the 5′ end.

24. (canceled)

25. The RNAi agent of claim 10, wherein the antisense strand comprises the modified nucleotide of any one of Formula Ia, Ib, Ic, II-IV or XXI at any one of positions 6-10 or 15-18 from the 5′ end.

26. The RNAi agent of claim 10, wherein the sense strand and antisense strand further comprises one or more 2′-fluoro modified nucleotides and 2′-O-methyl modified nucleotides.

27. The RNAi agent of claim 26, wherein the sense strand comprises four 2′-fluoro modified nucleotides at positions 7, 9, 10, and 11 from the 5′ end of the sense strand.

28. The RNAi agent of claim 27, wherein the nucleotides at the other positions of the sense strand are 2′-O-methyl modified nucleotides.

29. The RNAi agent of claim 26, wherein the antisense strand comprises four 2′-fluoro modified nucleotides at positions 2, 6, 14, and 16 from the 5′ end of the antisense strand.

30. The RNAi agent of claim 29, wherein the nucleotides at the other positions of the antisense strand are 2′-O-methyl modified nucleotides.

31. The RNAi agent of claim 26, wherein the sense strand comprises three 2′-fluoro modified nucleotides at positions 9, 10, and 11 from the 5′ end of the sense strand.

32. The RNAi agent of claim 31, wherein the nucleotides at the other positions of the sense strand are 2′-O-methyl modified nucleotides.

33. The RNAi agent of claim 26, wherein the antisense strand comprises five 2′-fluoro modified nucleotides at positions 2, 5, 7, 14, and 16 from the 5′ end of the antisense strand.

34. The RNAi agent of claim 33, wherein the nucleotides at the other positions of the antisense strand are 2′-O-methyl modified nucleotides.

35. The RNAi agent of claim 26, wherein the antisense strand comprises five 2′-fluoro modified nucleotides at positions 2, 5, 8, 14, and 16 from the 5′ end of the antisense strand.

36. The RNAi agent of claim 35, wherein the nucleotides at the other positions of the antisense strand are 2′-O-methyl modified nucleotides.

37. The RNAi agent of claim 26, wherein the antisense strand comprises five 2′-fluoro modified nucleotides at positions 2, 3, 7, 14, and 16 from the 5′ end of the antisense strand.

38. The RNAi agent of claim 37, wherein the nucleotides at the other positions of the antisense strand are 2′-O-methyl modified nucleotides.

39. The RNAi agent of claim 10, wherein the sense strand and the antisense strand comprise one or more modified internucleotide linkages.

40. The RNAi agent of claim 39, wherein the one or more modified internucleotide linkages are phosphorothioate linkages.

41. The RNAi agent of claim 39, wherein the sense strand comprises four or five phosphorothioate linkages.

42. The RNAi agent of claim 39, wherein the antisense strand comprises four or five phosphorothioate linkages.

43. The RNAi agent of claim 10, wherein the antisense strand comprises a phosphate analog at the 5′ end.

44. The RNAi agent of claim 43, wherein the phosphate analog is 5′-vinylphosphonate.

45. The RNAi agent of claim 10, wherein the sense strand comprises an abasic moiety or inverted abasic moiety.

46. The RNAi agent of claim 10, wherein the antisense strand is complementary to a target mRNA selected from SNCA, MAPT, APP, ATXN2, ATXN3, SARM1, APOE, BACE1, FMR1, LRRK2, HTT, SOD1, SCN10A, SCN9A or CACNA1B mRNA.

47. The RNAi agent of claim 46, wherein the antisense strand is complementary to SNCA mRNA.

48. The RNAi agent of claim 47, wherein the sense strand and the antisense strand comprise a pair of nucleic acid sequences selected from the group consisting of:
(a) the sense strand comprises SEQ ID NO: 1, and the antisense strand comprises SEQ ID NO: 2;
(b) the sense strand comprises any one of SEQ ID NOs: 3, 5, or 20, and the antisense strand comprises SEQ ID NO: 4;
(c) the sense strand comprises any one of SEQ ID NOs: 6, 8-19, 37, 38, or 67-81, and the antisense strand comprises SEQ ID NO: 7;
(d) the sense strand comprises SEQ ID NO: 19, and the antisense strand comprises SEQ ID NO: 66;
(e) the sense strand comprises SEQ ID NO: 9 or 16, and the antisense strand comprises SEQ ID NO: 82;
(f) the sense strand comprises SEQ ID NO: 83, and the antisense strand comprises SEQ ID NO: 84;
(g) the sense strand comprises SEQ ID NO: 85, and the antisense strand comprises SEQ ID NO: 86;
(h) the sense strand comprises SEQ ID NO: 87, and the antisense strand comprises SEQ ID NO: 88;
(i) the sense strand comprises SEQ ID NO: 89, and the antisense strand comprises SEQ ID NO: 90;
(j) the sense strand comprises SEQ ID NO: 91, and the antisense strand comprises SEQ ID NO: 92 or 93;
(k) the sense strand comprises SEQ ID NO: 94, and the antisense strand comprises SEQ ID NO: 95;
(l) the sense strand comprises SEQ ID NO: 96, and the antisense strand comprises SEQ ID NO: 97;
(m) the sense strand comprises SEQ ID NO: 98, and the antisense strand comprises SEQ ID NO: 99;
(n) the sense strand comprises SEQ ID NO: 100, and the antisense strand comprises SEQ ID NO: 101;
(o) the sense strand comprises SEQ ID NO: 102, and the antisense strand comprises SEQ ID NO: 103;
(p) the sense strand comprises SEQ ID NO: 104, and the antisense strand comprises SEQ ID NO: 105;
(q) the sense strand comprises SEQ ID NO: 106, and the antisense strand comprises SEQ ID NO: 107;
(r) the sense strand comprises SEQ ID NO: 108, and the antisense strand comprises SEQ ID NO: 109 or 122;
(s) the sense strand comprises SEQ ID NO: 110, and the antisense strand comprises SEQ ID NO: 111;
(t) the sense strand comprises SEQ ID NO: 112, and the antisense strand comprises SEQ ID NO: 113;
(u) the sense strand comprises SEQ ID NO: 114, and the antisense strand comprises SEQ ID NO: 115;
(v) the sense strand comprises SEQ ID NO: 116, and the antisense strand comprises SEQ ID NO: 117;
(w) the sense strand comprises SEQ ID NO: 118, and the antisense strand comprises SEQ ID NO: 119;
(x) the sense strand comprises SEQ ID NO: 120, and the antisense strand comprises SEQ ID NO: 121; and
(y) the sense strand comprises SEQ ID NO: 123, and the antisense strand comprises SEQ ID NO: 124.

49. The RNAi agent of claim 47, wherein the sense strand and the antisense strand consist of a pair of nucleic acid sequences selected from the group consisting of:
(a) the sense strand consists of any one of SEQ ID NOs: 3, 5, or 20, and the antisense strand consists of SEQ ID NO: 4; and
(b) the sense strand consists of any one of SEQ ID NOs: 6, 8-19, 37, 38, or 67-81, and the antisense strand consists of SEQ ID NO: 7;
(c) the sense strand consists of SEQ ID NO: 19, and the antisense strand consists of SEQ ID NO: 66;
(d) the sense strand consists of SEQ ID NO: 9 or 16, and the antisense strand consists of SEQ ID NO: 82;
(e) the sense strand consists of SEQ ID NO: 87, and the antisense strand consists of SEQ ID NO: 88;
(f) the sense strand consists of SEQ ID NO: 89, and the antisense strand consists of SEQ ID NO: 90;
(g) the sense strand consists of SEQ ID NO: 91, and the antisense strand consists of SEQ ID NO: 92 or 93;
(h) the sense strand consists of SEQ ID NO: 106, and the antisense strand consists of SEQ ID NO: 107;
(i) the sense strand consists of SEQ ID NO: 108, and the antisense strand consists of SEQ ID NO: 109 or 122;
(j) the sense strand consists of SEQ ID NO: 110, and the antisense strand consists of SEQ ID NO: 111;
(k) the sense strand consists of SEQ ID NO: 112, and the antisense strand consists of SEQ ID NO: 113;
(l) the sense strand consists of SEQ ID NO: 114, and the antisense strand consists of SEQ ID NO: 115;
(m) the sense strand consists of SEQ ID NO: 116, and the antisense strand consists of SEQ ID NO: 117;
(n) the sense strand consists of SEQ ID NO: 118, and the antisense strand consists of SEQ ID NO: 119;
(o) the sense strand consists of SEQ ID NO: 120, and the antisense strand consists of SEQ ID NO: 121; and
(p) the sense strand consists of SEQ ID NO: 123, and the antisense strand consists of SEQ ID NO: 124.

50. The RNAi agent of claim 46, wherein the antisense strand is complementary to MAPT mRNA.

51. The RNAi agent of claim 50, wherein the sense strand and the antisense strand comprise a pair of nucleic acid sequences selected from the group consisting of:
(a) the sense strand comprises SEQ ID NO: 21, and the antisense strand comprises SEQ ID NO: 22;
(b) the sense strand comprises SEQ ID NO: 23, and the antisense strand comprises SEQ ID NO: 24;
(c) the sense strand comprises SEQ ID NO: 25, and the antisense strand comprises SEQ ID NO: 26;
(d) the sense strand comprises any one of SEQ ID NOs: 27, 33, 39, 40, 47-49, and the antisense strand comprises SEQ ID NO: 28;
(e) the sense strand comprises any one of SEQ ID NOs: 29, 34, 35, 42, 50-51, 53, and the antisense strand comprises SEQ ID NO: 30;
(f) the sense strand comprises any one of SEQ ID NO: 31, 36, 43, 52, 154, 155, 161-163, and the antisense strand comprises SEQ ID NO: 32;
(g) the sense strand comprises SEQ ID NO: 39 or 40, and the antisense strand comprises SEQ ID NO: 41;
(h) the sense strand comprises SEQ ID NO: 44 or 46, and the antisense strand comprises SEQ ID NO: 45;
(i) the sense strand comprises SEQ ID NO: 53, and the antisense strand comprises SEQ ID NO: 54 or 55;
(j) the sense strand comprises SEQ ID NO: 56, and the antisense strand comprises SEQ ID NO: 57;
(k) the sense strand comprises SEQ ID NO: 125, and the antisense strand comprises SEQ ID NO: 126;
(l) the sense strand comprises SEQ ID NO: 127, and the antisense strand comprises SEQ ID NO: 128;
(m) the sense strand comprises SEQ ID NO: 129, and the antisense strand comprises SEQ ID NO: 130;
(n) the sense strand comprises SEQ ID NO: 131, and the antisense strand comprises SEQ ID NO: 132;
(o) the sense strand comprises SEQ ID NO: 133, and the antisense strand comprises SEQ ID NO: 134;
(p) the sense strand comprises SEQ ID NO: 135, and the antisense strand comprises SEQ ID NO: 136;
(q) the sense strand comprises SEQ ID NO: 137, and the antisense strand comprises SEQ ID NO: 138;
(r) the sense strand comprises SEQ ID NO: 139, and the antisense strand comprises SEQ ID NO: 140;
(s) the sense strand comprises SEQ ID NO: 141, and the antisense strand comprises SEQ ID NO: 142;
(t) the sense strand comprises SEQ ID NO: 143, and the antisense strand comprises SEQ ID NO: 144;
(u) the sense strand comprises SEQ ID NO: 145, and the antisense strand comprises SEQ ID NO: 146;
(v) the sense strand comprises SEQ ID NO: 147, and the antisense strand comprises SEQ ID NO: 148;
(w) the sense strand comprises SEQ ID NO: 34, and the antisense strand comprises anyone of SEQ ID NO: 149, 150, 151;
(x) the sense strand comprises SEQ ID NO: 31, and the antisense strand comprises any one of SEQ ID NO: 152, 153, 156-159, 164, 165;
(y) the sense strand comprises SEQ ID NO: 160, and the antisense strand comprises SEQ ID NO: 152; and
(z) the sense strand comprises SEQ ID NO: 43 or 166, and the antisense strand comprises SEQ ID NO: 156.

52. The RNAi agent of claim 50, wherein the sense strand and the antisense strand consist of a pair of nucleic acid sequences selected from the group consisting of:
(a) the sense strand consists of any one of SEQ ID NOs: 27, 33, 39, 40, 47-49, and the antisense strand consists of SEQ ID NO: 28;
(b) the sense strand consists of any one of SEQ ID NOs: 29, 34, 35, 42, 50-51, 53, and the antisense strand consists of SEQ ID NO: 30; and
(c) the sense strand consists of any one of SEQ ID NO: 31, 36, 43, 52, 154, 155, 161-163, and the antisense strand consists of SEQ ID NO: 32;
(d) the sense strand consists of SEQ ID NO: 39 or 40, and the antisense strand consists of SEQ ID NO: 41;
(e) the sense strand consists of SEQ ID NO: 44 or 46, and the antisense strand consists of SEQ ID NO: 45;
(f) the sense strand consists of SEQ ID NO: 53, and the antisense strand consists of SEQ ID NO: 54 or 55;
(g) the sense strand consists of SEQ ID NO: 137, and the antisense strand consists of SEQ ID NO: 138;
(h) the sense strand consists of SEQ ID NO: 139, and the antisense strand consists of SEQ ID NO: 140;
(i) the sense strand consists of SEQ ID NO: 141, and the antisense strand consists of SEQ ID NO: 142;
(j) the sense strand consists of SEQ ID NO: 143, and the antisense strand consists of SEQ ID NO: 144;
(k) the sense strand consists of SEQ ID NO: 145, and the antisense strand consists of SEQ ID NO: 146;
(l) the sense strand consists of SEQ ID NO: 147, and the antisense strand consists of SEQ ID NO: 148;
(m) the sense strand consists of SEQ ID NO: 34, and the antisense strand consists of any one of SEQ ID NO: 149, 150, 151;
(n) the sense strand consists of SEQ ID NO: 31, and the antisense strand consists of any one of SEQ ID NO: 152, 153, 156-159, 164, 165;
(o) the sense strand consists of SEQ ID NO: 160, and the antisense strand consists of SEQ ID NO: 152; and
(p) the sense strand consists of SEQ ID NO: 43 or 166, and the antisense strand consists of SEQ ID NO: 156.

53. A pharmaceutical composition comprising the RNAi agent of claim 10, and a pharmaceutically acceptable carrier.

54. A method of treating a neurodegenerative disease in a patient in need thereof, the method comprising administering to the patient an effective amount of the RNAi agent of claim 10.

55. The method of claim 54, wherein the neurodegenerative disease is a synucleinopathy selected from Parkinson's disease, Alzheimer's disease, multiple system atrophy, or Lewy body dementia.

56. The method of claim 54, wherein the neurodegenerative disease is a tauopathy selected from Alzheimer's disease, frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's discase, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), or globular glial tauopathies (GGT).

57. The method of claim 54, wherein the compound or RNAi agent is administered to the patient intrathecally, intracerebroventricularly, or via intracisternal magna injection.

58.-65. (canceled)
